(19) United States
(12) Patent Application Publication
(10) Pub. No.: US 2022/0348632 A1
(43) Pub. Date: Nov. 3, 2022

(54) NKG2D FUSION PROTEINS AND USES THEREOF

(71) Applicant: Novartis AG, Basel (CH)

(72) Inventors: Ryan Sullivan, Watertown, MA (US); Hilmar Ebersbach, Wallisellen (CH); Emilie Ramondou, Basel (CH); Philip Egger, Dornach (CH)

(21) Appl. No.: 17/761,012

(22) PCT Filed: Sep. 16, 2020
(86) PCT No.: PCT/IB2020/058642

§ 371 Date: Mar. 16, 2022

Related U.S. Application Data
(60) Provisional application No. 62/902,080, filed on Sep. 18, 2019.
(60) Provisional application No. 62/902,071, filed on Sep. 18, 2019.

(51) Int. Cl. C07K 14/705; A61P 35/00

(52) U.S. Cl. CPC C07K 14/7056; A61P 35/00; C07K 2319/00; C07K 2319/30; A61K 38/00

(57) ABSTRACT

Provided are NKG2D fusion proteins and their uses for the treatment of various diseases.

### CROSS REFERENCE TO RELATED APPLICATIONS

This application claims the benefit under 35 U.S.C. § 119(e) of the following United States provisional patent applications: (1) U.S. 62/902,071, filed Sep. 18, 2019 and (2) U.S. 62/902,080, filed Sep. 18, 2019. The disclosures of these provisional applications are incorporated by reference herein in their entirety.

### BACKGROUND

There is a need for novel and effective therapeutics for preventing and treating a broad range of cancer types. Provided herein are solutions to these and other problems in the art.

### BRIEF SUMMARY

Provided herein is a dimeric protein that includes two monomers, where each monomer includes (1) a first NKG2D peptide or variant thereof; (2) a second NKG2D peptide or variant thereof; (3) a first peptide linker connecting said first NKG2D peptide or variant thereof and said second NKG2D peptide or variant thereof; and (4) a fragment crystallizable region (Fc region) of an immunoglobulin (Ig).

In some embodiments, the first NKG2D peptide or variant thereof and the second NKG2D peptide or variant thereof have identical amino acid sequence.

In some embodiments, the first NKG2D peptide or variant thereof and the second NKG2D peptide or variant thereof have different amino acid sequence.

In some embodiments, the first NKG2D peptide or variant thereof comprises, or alternatively, consists essentially of or consists of, an amino acid sequence having at least 85% identity to SEQ ID NO: 1, 2, 3 or 4.

In some embodiments, the first NKG2D peptide or variant thereof comprises, or alternatively, consists essentially of or consists of, an amino acid sequence of SEQ ID NO: 1, 2, 3 or 4.

In some embodiments, the second NKG2D peptide or variant thereof comprises, or alternatively, consists essentially of or consists of, an amino acid sequence having at least 85% identity to SEQ ID NO: 1, 2, 3 or 4.

In some embodiments, the second NKG2D peptide or variant thereof comprises, or alternatively, consists essentially of or consists of, an amino acid sequence of SEQ ID NO: 1, 2, 3 or 4.

In some embodiments, the first peptide linker is a glycine-serine peptide linker.

In some embodiments, the peptide linker is represented by the formula (GGGS)ₙ (SEQ ID NO: 9), wherein n is 1, 2, 3, 4, or 5.

In some embodiments, the peptide linker is GGGSGGGS (SEQ ID NO: 10).

In some embodiments, the Fc region comprises an Fc region of human immunoglobulin G (IgG).

In some embodiments, the Fc region comprises an Fc region of human IgG1.

In some embodiments, the Fc region comprises, or alternatively, consists essentially of or consists of, an amino acid sequence having at least 85% identity to SEQ ID NO: 5, 6, 63, 64, 65, or 68.

In some embodiments, the Fc region comprises, or alternatively, consists essentially of or consists of, an amino acid sequence of SEQ ID NO: 5, 6, 63, 64, 65, or 68.

In some embodiments, the monomer includes, from N-terminus to C-terminus, the first NKG2D peptide or variant thereof, the first peptide linker, the second NKG2D peptide or variant thereof and the Fc region. In some embodiments, the second NKG2D peptide or variant thereof is directly fused with the Fc region without a peptide linker. In some embodiments, the second NKG2D peptide or variant thereof is linked with the Fc region via a second peptide linker.

In some embodiments, the monomer includes, from N-terminus to C-terminus, the Fc region, the first NKG2D peptide or variant thereof, the first peptide linker, and the second NKG2D peptide or variant region. In some embodiments, the Fc region is directly fused with the first NKG2D peptide or variant thereof without a peptide linker. In some embodiments, the Fc region is linked with the first NKG2D peptide or variant thereof via a second peptide linker.

In some embodiments, the monomer further comprising an additional one or two NKG2D peptides or variants thereof.

In some embodiments, the monomer comprises, or alternatively, consists essentially of or consists of, an amino acid sequence having at least 85% identity to any one of SEQ ID NOs: 17, 19, 21, 23, 25, 27, 29, 31, 33, 35, 37, 39, 41, 43, 45, 47, 49, 51, 53, 55, 57, 59, 61, and 69.

In some embodiments, the monomer comprises, or alternatively, consists essentially of or consists of, an amino acid sequence of any one of SEQ ID NOs: 17, 19, 21, 23, 25, 27, 29, 31, 33, 35, 37, 39, 41, 43, 45, 47, 49, 51, 53, 55, 57, 59, 61, and 69.

In some embodiments, the monomer is encoded by a nucleic acid sequence comprising or consisting of a nucleic acid sequence having at least 85% identity to any one of SEQ ID NOs: 18, 20, 22, 24, 26, 28, 30, 32, 34, 36, 38, 40, 42, 44, 46, 48, 50, 52, 54, 56, 58, 60, 62, and 70.

In some embodiments, the monomer is encoded by a nucleic acid sequence comprising or consisting of any one of SEQ ID NOs: 18, 20, 22, 24, 26, 28, 30, 32, 34, 36, 38, 40, 42, 44, 46, 48, 50, 52, 54, 56, 58, 60, 62, and 70.

In some embodiments, the two monomers are covalently linked.

In some embodiments, the monomers are linked through a Cys-Cys bridge.

In some embodiments, the dimeric protein further comprising a drug moiety.

Also provided herein is an isolated polynucleotide comprising a nucleic acid sequence comprising or consisting of any one of SEQ ID NOs: 18, 20, 22, 24, 26, 28, 30, 32, 34, 36, 38, 40, 42, 44, 46, 48, 50, 52, 54, 56, 58, 60, 62, and 70.

Further provided herein is a vector comprising the isolated polynucleotide described herein.

Further provided herein is a composition comprising the dimeric protein described herein, the isolated polynucleotide described herein or the vector described herein.

In some embodiments, the composition further comprising a pharmaceutically acceptable carrier.

Also provided herein is a cell comprising the dimeric protein described herein, the isolated polynucleotide described herein, the vector described herein or the composition described herein.

In some embodiments, the cell is a Chinese hamster ovary (CHO) cell.

In some embodiments, the cell is a modified CHO cell.

In some embodiments, the modified CHO cell comprises an inactivated matriptase gene or a knock-out (deletion) of matriptase gene.

Provided herein is a dimeric protein produced by a cell described herein.

Provided herein is a method for treating a disease, the method comprising administering to a subject in need thereof a dimeric protein described herein or a composition described herein.

In some embodiments, the disease is an NKG2D ligand expressing disease.

In some embodiments, the disease is cancer, autoimmune disease, liver disease, nerve injury, hereditary motor and sensory neuropathy, aging, or viral infection.

In some embodiments, the disease is cancer or autoimmune disease.

In some embodiments, the cancer is an NKG2D ligand expressing cancer.

In some embodiments, the subject has an NKG2D ligand expressing cancer.

In some embodiments, the ligand expressing cancer is lung cancer, ovarian cancer, colon cancer, pancreatic cancer, bladder cancer, breast cancer, hepatocellular carcinoma (HCC), renal cancer, nasopharyngeal carcinoma (NPC), prostate cancer, melanoma, plasma cell cancer, leukemia, lymphoma, glioma, or neuroblastoma.

In some embodiments, the leukemia is acute myeloid leukemia (AML), chronic myeloid leukemia (CML) or chronic lymphocytic leukemia (CLL).

In some embodiments, the method further comprising treating the subject with an additional anti-cancer therapy or an anti-cancer agent.

In some embodiments, the additional anti-cancer therapy is surgery, radiation therapy, chemotherapy, gene therapy, DNA therapy, viral therapy, RNA therapy, adjuvant therapy, or immunotherapy.

In some embodiments, the additional cancer therapy is a chemotherapy that damages DNA.

In some embodiments, the NKG2D ligand is MICA, MICB, ULBP1, ULBP2, ULBP3, ULBP4, ULBP5, or ULBP6.

In some embodiments, the autoimmune disease is rheumatoid arthritis, colitis, celiac disease, multiple sclerosis, alopecia areata, type 1 diabetes, chronic obstructive pulmonary disease, atherosclerosis, or metabolic syndrome associated with type 2 diabetes.

In some embodiments, the liver disease is liver fibrosis, portal vein hypertension, nonalcoholic steatohepatitis (NASH), fatty liver disease, and cirrhosis.

In some embodiments, the nerve injury is traumatic or toxic injuries to peripheral or cranial nerves, spinal cord or to the brain, cranial nerves, traumatic brain injury, stroke, cerebral aneurism, spinal cord injury, or injury related to a underlying genetic disease.

In some embodiments, the hereditary motor and sensory neuropathy is Charcot-Marie-Tooth (e.g. type 1A, type2, type3, with pyramidal features, or type 6) or refsum disease.

In some embodiments, the viral infection is hepatitis, Epstein-Barr, or cytomegalovirus.

Also provided herein is a method for diagnosing an NKG2D ligand expressing disease, the method comprising (1) obtaining a biological sample from a subject having an NKG2D ligand expressing disease; (2) contacting the biological sample with a binding reagent comprising a dimeric protein described herein or a composition described herein; and (3) detecting a binding of the NKG2D ligand to the binding reagent; where a binding of the NKG2D ligand to the binding reagent indicates a diagnosis of the NKG2D ligand expressing disease in the subject.

In some embodiments, the NKG2D ligand is MICA, MICB, ULBP1, ULBP2, ULBP3, ULBP4, ULBP5, or ULBP6.

Provided herein is a dimeric protein described herein, a polynucleotide described herein, a vector described herein, or a composition described herein, for use as a medicament

Provided herein is a dimeric protein described herein, a polynucleotide described herein, a vector described herein, or a composition described herein, for use in the treatment of a disease.

Provided herein is a dimeric protein described herein, a polynucleotide described herein, a vector described herein, or a composition described herein, for use in the treatment of a disease, wherein said disease is an NKG2D ligand expressing disease.

Provided herein is a dimeric protein described herein, a polynucleotide described herein, a vector described herein, or a composition described herein, for use in the treatment of a disease, wherein said disease is cancer or autoimmune disease.

### BRIEF DESCRIPTION OF THE DRAWINGS

FIGS. 1A-1B provide schematics of NKG2D-Fc structure. FIG. 1A provides a diagram of NKG2D (green)-Fc (blue). FIG. 1B provides a model of NKG2D-Fc's interaction with MICA (orange) based on PDB 1HYR.

FIGS. 2A-2B demonstrate superiority of N′ NKG2D (i.e., NKG2D peptide(s) is located at the N terminus of Fc fragment) and two NKG2D homodimers in inducing ADCC signaling against NKG2D-L target cells based on a reporter gene assay. In FIG. 2A, JNL Reporter Gene Fluorescence was induced by NKG2D-Fc constructs, indicating cross-linking of the Fc receptor on JNL cells in co-culture with NKG2D-L+ target cells. FIG. 2B provides diagrams of NKG2D-Fc variants used in this experiment, with either N′ NKG2D (H1/H2) or C′ NKG2D (H3/H4) (i.e., NKG2D peptide(s) is located at the C terminus of Fc fragment) and either one NKG2D homodimer (H1/H3), or two NKG2D homodimers (H2/H4).

FIGS. 3A-3B demonstrates mouse NKG2D-Fc surrogate constructs bind to cells expressing mouse NKG2D ligands with an optimal linker length of 0 amino acids. The Y axis is the mean R-PE fluorescence. FIG. 3A depicts binding of constructs to cells overexpressing mouse RAE-1alpha. FIG. 3B depicts binding of constructs to cells expressing mouse RAE-1 epsilon.

FIGS. 4A-4D demonstrate that two NKG2D homodimers is the optimal valency, additional valency beyond two homodimers does not provide a significant advantage to killing activity based on NKG2D valency and orientation optimization. FIG. 4A depicts diagrams of constructs used in FIG. 4B. FIG. 4B depicts the results of ADCC Reporter Gene Assay against human MICA*008-expressing target cells. FIG. 4C depicts diagrams of constructs used in FIG. 4D. FIG. 4D depicts the results of ADCC Reporter Gene Assay against human MICA*008-expressing target cells.

FIGS. 5A-5D provides information on exemplary NKG2D-Fc variants. FIG. 5A provides diagrams of HH8 and HH2, two variants of NKG2D-Fc. HH8 differs from HH2 by the deletion of two FLNSLF sequences. FIG. 5B provides a diagram of the NKG2D-Fc structure. FIG. 5C provides the full sequence of HH2/HH8. The sequences in red are deleted from HH8. FIG. 5D provides production characteristics of HH2, HH7, and HH8, depicting Biacore sensograms of binding of HH2, HH7, and HH8 (right graph) and quantification summary (left table).

FIG. 6 provides the sequence of HH8. NKG2D is shown in black, the hinge region in green, CH2 in blue, CH3 in purple, N-glycosylation sites in red, isomerization sites in orange, and linker sequence in light green (SEQ ID NO: 29).

FIG. 7 provides the sequence of HH10. NKG2D is shown in black, the hinge region in green, CH2 in blue, CH3 in purple, N-glycosylation sites in red, isomerization sites in orange, and linker sequence in light green (SEQ ID NO: 31).

FIG. 8 provides the sequence of HH8-afuco. NKG2D is shown in black, the hinge region in green, CH2 in blue, CH3 in purple, N-glycosylation sites in red, isomerization sites in orange, and linker sequence in light green (SEQ ID NO: 29).

FIGS. 9A-9E demonstrate CHO production improves NKG2D-Fc ADCC activity. NKG2D-Fc ADCC activity was measured by reporter gene activity in co-culture with CT26.WT-huMICA*008 (FIG. 9A), CT26.WT-huMICA*001 (FIG. 9B), CT26.WT-huMICB*001 (FIG. 9C), and CT26.WT-cyMICB*05 (FIG. 9D). FIG. 9E depicts a summary of EC50 improvement of CHO-derived NKG2D-Fc compared to HEK-derived NKG2D-Fc.

FIG. 10 depicts identification by LC-MS of a clipping site in the second NKG2D fragment of HH2 protein produced in CHO MaKO cell line.

FIGS. 11A-11B demonstrate NKG2D-Fc specifically binds to all eight NKG2D-L with single-digit nM EC50. FIG. 11A provides an experimental diagram. FIG. 11B depicts binding of HH8 to cells overexpressing human NKG2D-L.

FIGS. 12A-12B demonstrate HH8 binds to all tested cyno NKG2D-L. FIG. 12A provides an experimental diagram. FIG. 12B depicts binding of HH8 to cells overexpressing cyno NKG2D-L.

FIGS. 13A-13B demonstrate NKG2D-Fc binds to cancer cell lines from many different indications, but not normal huPBMC. FIG. 13A depicts binding of NKG2D-Fc to human tumor cell lines. FIG. 13B depicts binding of NKG2D-Fc to normal human PBMCs.

FIGS. 14A-14B demonstrate NKG2D-Fc preserves NKG2D expression in vitro. FIG. 14A provides an experimental diagram. FIG. 14B demonstrates NKG2D expression on NK cells co-cultured with NKG2D-L+ target cells is restored by the addition of NKG2D-Fc.

FIG. 15 demonstrates enhanced ADCC variants have improved in vitro function, depicting the effect of various amino acid variants on ADCC activity in a reporter gene assay.

FIGS. 16A-16D demonstrates HH8, HH10, and HH8-AFUCO promote ADCC against NKG2D-L+ target cell lines. HH10 and HH8-AFUCO have improved activity in an NK3.3 killing assay. FIGS. 16A-16D depict Specific killing of NKG2D-L+ target cell lines.

FIGS. 17A-17E demonstrate NKG2D-Fc binds tumor cells directly and recruits immune cells to kill tumors through ADCC/ADCP, initiating the cancer immunity cycle. FIG. 17A demonstrate NKG2D-Fc, HH8, promotes killing of HCT-116 cells by primary human NK cells.

FIGS. 17B-17C demonstrate NKG2D-Fc promotes killing of OVCAR-3 cells by primary human NK cells. FIGS. 17D-17E demonstrate NKG2D-Fc, HH8, promotes prolonged activation of primary human NK cells in a 96 hour co-culture with HCT-116 cells as measured by CD69 expression, NKG2D expression, size, and granularity.

FIG. 18 demonstrates NKG2D-Fc, HH8, promotes killing of NKG2D-L+ tumors through ADCP with sub-nM EC50 in a co-culture with primary mouse macrophages and NKG2D-L expressing mouse tumor cells.

FIGS. 19A-19F demonstrate HH8 and HH10 promote in vitro ADCC against all tested NKG2D-L, including huMICA*008 (FIG. 19A), huMICB*001 (FIG. 19B), huULBP1 (FIG. 19C), huULBP2 (FIG. 19D), cyMICA*08 (FIG. 19E), and (FIG. 19F) cyMICB*05 (FIG. 19F).

FIGS. 20A-20B depict pharmacokinetics of NKG2D-Fc in mouse, demonstrating the impact of production cell line and Fc format on in vivo profile. FIG. 20A depicts in vivo pharmacokinetics of NKG2D-Fc variants produced in HEK or CHO, or with Fc variations. FIG. 20B depicts in vivo pharmacokinetics of NKG2D-Fc variants produced in CHO.

FIGS. 21A-21B depict pharmacokinetics of HH8 in cynomolgus monkeys, demonstrating antibody-like PK profile and retention of ability to bind target after in vivo circulation. FIG. 21A depicts HH8 concentration at the indicated number of hours post-dose, quantified as either as total or amount capable of binding to MICA ex vivo (target). FIG. 21B depicts HH8 serum concentration at the indicated number of hours post-dose along with a typical IgG simulation.

FIGS. 22A-22B demonstrate mouse tumors overexpressing NKG2D-L have poor CD8/NK infiltration, phenocopying human NKG2D-L+ tumors. NKG2D-Fc restores mouse CD8+ and NK cell tumor presence. FIG. 22A provides a diagram of study design. FIG. 22B depicts quantification of NK cell and CD8+ T cell infiltrate into the tumor.

FIGS. 23A-23B demonstrate tumor-infiltrating mouse CD8+ and NK cells from NKG2D-L+ tumors are desensitized. NKG2D-Fc restores activation of tumor-infiltrating NK and CD8+ T cells. FIG. 23A provides a diagram of the mouse study, indicating the treatment of mice with either isotype or NKG2D-Fc at 20 mg/kg on day 2 post-tumor implant. FIG. 23B depicts pharmacodynamic effects of NKG2D-Fc at 5 days post-dosing, demonstrating recovery of NKG2D expression and activation as measured by CD69.

FIGS. 24A-24B demonstrate HH8 and HH10 are efficacious in mouse models. FIG. 24A depicts EL4-huMICA*001 tumor growth in C57Bl/6 mice treated with either Human IgG1 isotype, HH8, or HH10 at 5 mg/kg on day 2 and 9. FIG. 24B depicts a Kaplan-Meier curve of mice in A.

### DETAILED DESCRIPTION

### Definitions

While various embodiments and aspects of the invention are shown and described herein, it will be obvious to those skilled in the art that such embodiments and aspects are provided by way of example only. Numerous variations, changes, and substitutions will now occur to those skilled in the art without departing from the invention. It should be understood that various alternatives to the embodiments of the invention described herein may be employed in practicing the invention.

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

The use of a singular indefinite or definite article (e.g., “a,” “an,” “the,” etc.) in this disclosure and in the following claims follows the traditional approach in patents of meaning “at least one” unless in a particular instance it is clear from context that the term is intended in that particular instance to mean specifically one and only one. Likewise, the term “comprising” is open ended, not excluding additional items, features, components, etc. References identified herein are expressly incorporated herein by reference in their entireties unless otherwise indicated.

The terms “comprise,” “include,” and “have,” and the derivatives thereof, are used herein interchangeably as comprehensive, open-ended terms. For example, use of “comprising,” “including,” or “having” means that whatever element is comprised, had, or included, is not the only element encompassed by the subject of the clause that contains the verb.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and 0-phosphoserine. Amino acid analogs refers to compounds that have the same basic chemical structure as a naturally occurring amino acid, i.e., an α carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. The terms “non-naturally occurring amino acid” and “unnatural amino acid” refer to amino acid analogs, synthetic amino acids, and amino acid mimetics which are not found in nature.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The terms “polypeptide,” “peptide” and “protein” are used interchangeably herein to refer to a polymer of amino acid residues, wherein the polymer may in embodiments be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers. A “fusion protein” refers to a chimeric protein encoding two or more separate protein sequences that are recombinantly expressed as a single moiety.

As may be used herein, the terms “nucleic acid,” “nucleic acid molecule,” “nucleic acid oligomer,” “oligonucleotide,” “nucleic acid sequence,” “nucleic acid fragment” and “polynucleotide” are used interchangeably and are intended to include, but are not limited to, a polymeric form of nucleotides covalently linked together that may have various lengths, either deoxyribonucleotides or ribonucleotides, or analogs, derivatives or modifications thereof. Different polynucleotides may have different three-dimensional structures, and may perform various functions, known or unknown. Non-limiting examples of polynucleotides include a gene, a gene fragment, an exon, an intron, intergenic DNA (including, without limitation, heterochromatic DNA), messenger RNA (mRNA), transfer RNA, ribosomal RNA, a ribozyme, cDNA, a recombinant polynucleotide, a branched polynucleotide, a plasmid, a vector, isolated DNA of a sequence, isolated RNA of a sequence, a nucleic acid probe, and a primer. Polynucleotides useful in the methods of the invention may comprise natural nucleic acid sequences and variants thereof, artificial nucleic acid sequences, or a combination of such sequences.

A polynucleotide is typically composed of a specific sequence of four nucleotide bases: adenine (A); cytosine (C); guanine (G); and thymine (T) (uracil (U) for thymine (T) when the polynucleotide is RNA). Thus, the term “polynucleotide sequence” is the alphabetical representation of a polynucleotide molecule; alternatively, the term may be applied to the polynucleotide molecule itself. This alphabetical representation can be input into databases in a computer having a central processing unit and used for bioinformatics applications such as functional genomics and homology searching. Polynucleotides may optionally include one or more non-standard nucleotide(s), nucleotide analog(s) and/or modified nucleotides.

“Conservatively modified variants” applies to both amino acid and nucleic acid sequences. With respect to particular nucleic acid sequences, “conservatively modified variants” refers to those nucleic acids that encode identical or essentially identical amino acid sequences. Because of the degeneracy of the genetic code, a number of nucleic acid sequences will encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are conservative substitutions for one another:

### 1) Alanine (A), Glycine (G);

2) Aspartic acid (D), Glutamic acid (E);

### 3) Asparagine (N), Glutamine (Q);

### 4) Arginine (R), Lysine (K);

### 5) Isoleucine (I), Leucine (L), Methionine (M), Valine (V);

### 6) Phenylalanine (F), Tyrosine (Y), Tryptophan (W);

### 7) Serine (S), Threonine (T); and

### 8) Cysteine (C), Methionine (M)

(see, e.g., Creighton, Proteins (1984)).

“Percentage of sequence identity” is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., NCBI web site http://www.ncbi.nlm.nih.gov/BLAST/ or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

An amino acid or nucleotide base “position” is denoted by a number that sequentially identifies each amino acid (or nucleotide base) in the reference sequence based on its position relative to the N-terminus (or 5′-end). Due to deletions, insertions, truncations, fusions, and the like that must be taken into account when determining an optimal alignment, in general the amino acid residue number in a test sequence determined by simply counting from the N-terminus will not necessarily be the same as the number of its corresponding position in the reference sequence. For example, in a case where a variant has a deletion relative to an aligned reference sequence, there will be no amino acid in the variant that corresponds to a position in the reference sequence at the site of deletion. Where there is an insertion in an aligned reference sequence, that insertion will not correspond to a numbered amino acid position in the reference sequence. In the case of truncations or fusions there can be stretches of amino acids in either the reference or aligned sequence that do not correspond to any amino acid in the corresponding sequence.

The terms “numbered with reference to” or “corresponding to,” when used in the context of the numbering of a given amino acid or polynucleotide sequence, refers to the numbering of the residues of a specified reference sequence when the given amino acid or polynucleotide sequence is compared to the reference sequence.

The term “probe” or “primer”, as used herein, is defined to be one or more nucleic acid fragments whose specific hybridization to a sample can be detected. A probe or primer can be of any length depending on the particular technique it will be used for. For example, PCR primers are generally between 10 and 40 nucleotides in length, while nucleic acid probes for, e.g., a Southern blot, can be more than a hundred nucleotides in length. The probe may be unlabeled or labeled as described below so that its binding to the target or sample can be detected. The probe can be produced from a source of nucleic acids from one or more particular (preselected) portions of a chromosome, e.g., one or more clones, an isolated whole chromosome or chromosome fragment, or a collection of polymerase chain reaction (PCR) amplification products. The length and complexity of the nucleic acid fixed onto the target element is not critical to the invention. One of skill can adjust these factors to provide optimum hybridization and signal production for a given hybridization procedure, and to provide the required resolution among different genes or genomic locations.

The probe may also be isolated nucleic acids immobilized on a solid surface (e.g., nitrocellulose, glass, quartz, fused silica slides), as in an array. In some embodiments, the probe may be a member of an array of nucleic acids as described, for instance, in WO 96/17958. Techniques capable of producing high density arrays can also be used for this purpose (see, e.g., Fodor (1991) Science 767-773; Johnston (1998) Curr. Biol. 8: R171-R174; Schummer (1997) Biotechniques 23: 1087-1092; Kern (1997) Biotechniques 23: 120-124; U.S. Pat. No. 5,143,854).

The phrase “selectively (or specifically) hybridizes to” refers to the binding, duplexing, or hybridizing of a molecule only to a particular nucleotide sequence with a higher affinity, e.g., under more stringent conditions, than to other nucleotide sequences (e.g., total cellular or library DNA or RNA).

The phrase “stringent hybridization conditions” refers to conditions under which a nucleic acid will hybridize to its target sequence, typically in a complex mixture of nucleic acids, but to no other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). Generally, stringent hybridization conditions are selected to be about 5-10° C. lower than the thermal melting point (Tm) for the specific sequence at a defined ionic strength pH. The Tm is the temperature (under defined ionic strength, pH, and nucleic concentration) at which 50% of the probes complementary to the target hybridize to the target sequence at equilibrium (as the target sequences are present in excess, at Tm, 50% of the probes are occupied at equilibrium). Stringent hybridization conditions may also be achieved with the addition of destabilizing agents such as formamide. For selective or specific hybridization, a positive signal is at least two times background, preferably 10 times background hybridization. Exemplary stringent hybridization conditions can be as following: 50% formamide, 5×SSC, and 1% SDS, incubating at 42° C., or, 5×SSC, 1% SDS, incubating at 65° C., with wash in 0.2×SSC, and 0.1% SDS at 65° C. Exemplary “moderately stringent hybridization conditions” include a hybridization in a buffer of 40% formamide, 1 M NaCl, 1% SDS at 37° C., and a wash in 1×SSC at 45° C. A positive hybridization is at least twice background. Those of ordinary skill will readily recognize that alternative hybridization and wash conditions can be utilized to provide conditions of similar stringency. Additional guidelines for determining hybridization parameters are provided in numerous reference, e.g., and Current Protocols in Molecular Biology, ed. Ausubel, et al., John Wiley & Sons.

As used herein, the term “conjugate” refers to the association between atoms or molecules. The association can be direct or indirect. For example, a conjugate between a first moiety (e.g., nucleic acid moiety) and a second moiety (peptide moiety) provided herein can be direct, e.g., by covalent bond, or indirect, e.g., by non-covalent bond (e.g. electrostatic interactions (e.g. ionic bond, hydrogen bond, halogen bond), van der Waals interactions (e.g. dipole-dipole, dipole-induced dipole, London dispersion), ring stacking (pi effects), hydrophobic interactions and the like). In some embodiments, conjugates are formed using conjugate chemistry including, but are not limited to nucleophilic substitutions (e.g., reactions of amines and alcohols with acyl halides, active esters), electrophilic substitutions (e.g., enamine reactions) and additions to carbon-carbon and carbon-heteroatom multiple bonds (e.g., Michael reaction, Diels-Alder addition). These and other useful reactions are discussed in, for example, March, ADVANCED ORGANIC CHEMISTRY, 3rd Ed., John Wiley & Sons, New York, 1985; Hermanson, BIOCONJUGATE TECHNIQUES, Academic Press, San Diego, 1996; and Feeney et al., MODIFICATION OF PROTEINS; Advances in Chemistry Series, Vol. 198, American Chemical Society, Washington, D.C., 1982.

For specific proteins described herein (e.g., NKG2D, MICA, MICB, ULBP1, ULBP2, ULBP3, ULBP4, ULBP5, ULBP6), the named protein includes any of the protein's naturally occurring forms, or variants that maintain the protein's expected activity (e.g., within at least 50%, 80%, 90%, 95%, 96%, 97%, 98%, 99% or 100% activity compared to the native protein). In some embodiments, variants have at least 85%, 86%, 87%, 88%, 89%, 90%, 91%, 92%, 93%, 94%, 95%, 96%, 97%, 98%, 99% or 100% amino acid sequence identity across the whole sequence or a portion of the sequence (e.g. a 50, 100, 150 or 200 continuous amino acid portion) compared to a naturally occurring form. In other embodiments, the protein is the protein as identified by its NCBI sequence reference. In other embodiments, the protein is the protein as identified by its NCBI sequence reference or functional fragment thereof.

Thus, NKG2D, also referred to as KLRK1; killer cell lectin-like receptor subfamily K, member 1; CD314; KLR; NKG2-D; FLJ17759; FLJ75772 or D12S2489E, a transmembrane protein belonging to the CD94/NKG2 family of C-type lectin-like receptors. In humans, it is expressed by NK cells, γδ T cells and CD8+αβ T cells. NKG2D recognizes induced-self proteins from MIC and RAET1/ULBP families which appear on the surface of stressed, malignant transformed, and infected cells. NKG2D described herein includes any of NKG2D's naturally occurring forms, or variants that maintain its protein activity (e.g., within at least 50%, 80%, 90%, 95%, 96%, 97%, 98%, 99% or 100% activity compared to the native NKG2D). In some embodiments, NKG2D variants or homologues have at least 85%, 86%, 87%, 88%, 89%, 90%, 91%, 92%, 93%, 94%, 95%, 96%, 97%, 98%, 99% or 100% amino acid sequence identity across the whole sequence or a portion of the sequence (e.g. a 50, 100, 150 or 200 continuous amino acid portion) compared to a naturally occurring NKG2D. In other embodiments, NKG2D is the protein as identified by its NCBI sequence reference NP_031386. In other embodiments, NKG2D is substantially identical (e.g., at least 85%, 86%, 87%, 88%, 89%, 90%, 91%, 92%, 93%, 94%, 95%, 96%, 97%, 98%, 99% or 100%) to the protein as identified by its NCBI sequence reference (NP_031386) or functional fragment thereof. More detailed descriptions of different types of NKG2D variants that can be used in the dimeric proteins described herein are provided below.

The term “MICA”, also referred as MEC class I polypeptide-related sequence A, MIC-A, PERB11.1, includes any of MICA's naturally occurring forms, or variants that maintain its protein activity (e.g., within at least 50%, 80%, 90%, 95%, 96%, 97%, 98%, 99% or 100% activity compared to the native MICA). In some embodiments, MICA variants or homologues have at least 85%, 86%, 87%, 88%, 89%, 90%, 91%, 92%, 93%, 94%, 95%, 96%, 97%, 98%, 99% or 100% amino acid sequence identity across the whole sequence or a portion of the sequence (e.g. a 50, 100, 150 or 200 continuous amino acid portion) compared to a naturally occurring MICA. In other embodiments, MICA is the protein as identified by its NCBI sequence reference NP_000238, NP_001170990, NP_001276081, NP_001276082, or NP_001276083. In other embodiments, MICA is substantially identical (e.g., at least 85%, 86%, 87%, 88%, 89%, 90%, 91%, 92%, 93%, 94%, 95%, 96%, 97%, 98%, 99% or 100%) to the protein as identified by its NCBI sequence reference (NP_000238, NP_001170990, NP_001276081, NP_001276082, or NP_001276083) or functional fragment thereof.

The term “MICB”, also referred as MEC class I polypeptide-related sequence B, MIC-B, PERB11.2, includes any of MICB's naturally occurring forms, or variants that maintain its protein activity (e.g., within at least 50%, 80%, 90%, 95%, 96%, 97%, 98%, 99% or 100% activity compared to the native MICB). In some embodiments, MICB variants or homologues have at least 85%, 86%, 87%, 88%, 89%, 90%, 91%, 92%, 93%, 94%, 95%, 96%, 97%, 98%, 99% or 100% amino acid sequence identity across the whole sequence or a portion of the sequence (e.g. a 50, 100, 150 or 200 continuous amino acid portion) compared to a naturally occurring MICB. In other embodiments, MICB is the protein as identified by its NCBI sequence reference NP_001276089, NP_001276090, or NP_005922. In other embodiments, MICB is substantially identical (e.g., at least 85%, 86%, 87%, 88%, 89%, 90%, 91%, 92%, 93%, 94%, 95%, 96%, 97%, 98%, 99% or 100%) to the protein as identified by its NCBI sequence reference (NP_001276089, NP_001276090, or NP_005922) or functional fragment thereof.

The term “ULBP1”, also referred as UL16 binding protein 1, RAET1I, N2DL-1, NKG2DL1, includes any of ULBP1's naturally occurring forms, or variants that maintain its protein activity (e.g., within at least 50%, 80%, 90%, 95%, 96%, 97%, 98%, 99% or 100% activity compared to the native ULBP1). In some embodiments, ULBP1 variants or homologues have at least 85%, 86%, 87%, 88%, 89%, 90%, 91%, 92%, 93%, 94%, 95%, 96%, 97%, 98%, 99% or 100% amino acid sequence identity across the whole sequence or a portion of the sequence (e.g. a 50, 100, 150 or 200 continuous amino acid portion) compared to a naturally occurring ULBP1. In other embodiments, ULBP1 is the protein as identified by its NCBI sequence reference NP_001304018 or NP_079494. In other embodiments, ULBP1 is substantially identical (e.g., at least 85%, 86%, 87%, 88%, 89%, 90%, 91%, 92%, 93%, 94%, 95%, 96%, 97%, 98%, 99% or 100%) to the protein as identified by its NCBI sequence reference (NP_001304018 or NP_079494) or functional fragment thereof.

The term “ULBP2”, also referred as UL16 binding protein 2, N2DL2, RAET1H, ALCAN-alpha, NKG2DL2, RAET1L, includes any of ULBP2's naturally occurring forms, or variants that maintain its protein activity (e.g., within at least 50%, 80%, 90%, 95%, 96%, 97%, 98%, 99% or 100% activity compared to the native ULBP2). In some embodiments, ULBP2 variants or homologues have at least 85%, 86%, 87%, 88%, 89%, 90%, 91%, 92%, 93%, 94%, 95%, 96%, 97%, 98%, 99% or 100% amino acid sequence identity across the whole sequence or a portion of the sequence (e.g. a 50, 100, 150 or 200 continuous amino acid portion) compared to a naturally occurring ULBP2. In other embodiments, ULBP2 is the protein as identified by its NCBI sequence reference NP_079493. In other embodiments, ULBP2 is substantially identical (e.g., at least 85%, 86%, 87%, 88%, 89%, 90%, 91%, 92%, 93%, 94%, 95%, 96%, 97%, 98%, 99% or 100%) to the protein as identified by its NCBI sequence reference (NP_079493) or functional fragment thereof.

The term “ULBP3”, also referred as UL16 binding protein 3, RAET1N, N2DL-3, NKG2DL3, includes any of ULBP3's naturally occurring forms, or variants that maintain its protein activity (e.g., within at least 50%, 80%, 90%, 95%, 96%, 97%, 98%, 99% or 100% activity compared to the native ULBP3). In some embodiments, ULBP3 variants or homologues have at least 85%, 86%, 87%, 88%, 89%, 90%, 91%, 92%, 93%, 94%, 95%, 96%, 97%, 98%, 99% or 100% amino acid sequence identity across the whole sequence or a portion of the sequence (e.g. a 50, 100, 150 or 200 continuous amino acid portion) compared to a naturally occurring ULBP3. In other embodiments, ULBP3 is the protein as identified by its NCBI sequence reference NP_078794. In other embodiments, ULBP3 is substantially identical (e.g., at least 85%, 86%, 87%, 88%, 89%, 90%, 91%, 92%, 93%, 94%, 95%, 96%, 97%, 98%, 99% or 100%) to the protein as identified by its NCBI sequence reference (NP_078794) or functional fragment thereof.

The term “ULBP4”, also referred as UL16 binding protein 4, RAET1E, LETAL, N2DL-4, NKG2DL4, RAET1E2, RL-4, bA350J20.7, retinoic acid early transcript 1E, includes any of ULBP4's naturally occurring forms, or variants that maintain its protein activity (e.g., within at least 50%, 80%, 90%, 95%, 96%, 97%, 98%, 99% or 100% activity compared to the native ULBP4). In some embodiments, ULBP4 variants or homologues have at least 85%, 86%, 87%, 88%, 89%, 90%, 91%, 92%, 93%, 94%, 95%, 96%, 97%, 98%, 99% or 100% amino acid sequence identity across the whole sequence or a portion of the sequence (e.g. a 50, 100, 150 or 200 continuous amino acid portion) compared to a naturally occurring ULBP4. In other embodiments, ULBP4 is the protein as identified by its NCBI sequence reference NP_001230254, NP_001230256, NP_001230257, or NP_631904. In other embodiments, ULBP4 is substantially identical (e.g., at least 85%, 86%, 87%, 88%, 89%, 90%, 91%, 92%, 93%, 94%, 95%, 96%, 97%, 98%, 99% or 100%) to the protein as identified by its NCBI sequence reference (NP_001230254, NP_001230256, NP_001230257, or NP_631904) or functional fragment thereof.

The term “ULBP5”, also referred as UL16 binding protein 5 or retinoic acid early transcript 1G (RAET1G), includes any of ULBP5's naturally occurring forms, or variants that maintain its protein activity (e.g., within at least 50%, 80%, 90%, 95%, 96%, 97%, 98%, 99% or 100% activity compared to the native ULBP5). In some embodiments, ULBP5 variants or homologues have at least 85%, 86%, 87%, 88%, 89%, 90%, 91%, 92%, 93%, 94%, 95%, 96%, 97%, 98%, 99% or 100% amino acid sequence identity across the whole sequence or a portion of the sequence (e.g. a 50, 100, 150 or 200 continuous amino acid portion) compared to a naturally occurring ULBP5. In other embodiments, ULBP5 is the protein as identified by its NCBI sequence reference NP_001001788.2. In other embodiments, ULBP5 is substantially identical (e.g., at least 85%, 86%, 87%, 88%, 89%, 90%, 91%, 92%, 93%, 94%, 95%, 96%, 97%, 98%, 99% or 100%) to the protein as identified by its NCBI sequence reference (NP_001001788.2) or functional fragment thereof.

The term “ULBP6”, also referred as UL16 binding protein 6 or Retinoic acid early transcript 1L (RAET1L), includes any of ULBP6's naturally occurring forms, or variants that maintain its protein activity (e.g., within at least 50%, 80%, 90%, 95%, 96%, 97%, 98%, 99% or 100% activity compared to the native ULBP6). In some embodiments, ULBP6 variants or homologues have at least 85%, 86%, 87%, 88%, 89%, 90%, 91%, 92%, 93%, 94%, 95%, 96%, 97%, 98%, 99% or 100% amino acid sequence identity across the whole sequence or a portion of the sequence (e.g. a 50, 100, 150 or 200 continuous amino acid portion) compared to a naturally occurring ULBP6. In other embodiments, ULBP6 is the protein as identified by its NCBI sequence reference NP_570970.2. In other embodiments, ULBP6 is substantially identical (e.g., at least 85%, 86%, 87%, 88%, 89%, 90%, 91%, 92%, 93%, 94%, 95%, 96%, 97%, 98%, 99% or 100%) to the protein as identified by its NCBI sequence reference (NP_570970.2) or functional fragment thereof.

The term “linker” or “bioconjugate linker” used herein refers to a molecule that results an association between atoms or molecules of bioconjugate reactive groups. The association can be direct or indirect. For example, a conjugate between a first bioconjugate reactive group (e.g., —NH2, —COOH, —N-hydroxysuccinimide, or -maleimide) and a second bioconjugate reactive group (e.g., sulfhydryl, sulfur-containing amino acid, amine, amine sidechain containing amino acid, or carboxylate) provided herein can be direct, e.g., by covalent bond, or indirect, e.g., by non-covalent bond (e.g. electrostatic interactions (e.g. ionic bond, hydrogen bond, halogen bond), van der Waals interactions (e.g. dipole-dipole, dipole-induced dipole, London dispersion), ring stacking (pi effects), hydrophobic interactions and the like). In some embodiments, bioconjugate linkers are formed using bioconjugate chemistry (i.e. the association of two bioconjugate reactive groups) including, but are not limited to nucleophilic substitutions (e.g., reactions of amines and alcohols with acyl halides, active esters), electrophilic substitutions (e.g., enamine reactions) and additions to carbon-carbon and carbon-heteroatom multiple bonds (e.g., Michael reaction, Diels-Alder addition). These and other useful reactions are discussed in, for example, March, ADVANCED ORGANIC CHEMISTRY, 3rd Ed., John Wiley & Sons, New York, 1985; Hermanson, BIOCONJUGATE TECHNIQUES, Academic Press, San Diego, 1996; and Feeney et al., MODIFICATION OF PROTEINS; Advances in Chemistry Series, Vol. 198, American Chemical Society, Washington, D.C., 1982. In some embodiments, the first bioconjugate reactive group (e.g., maleimide moiety) is covalently attached to the second bioconjugate reactive group (e.g. a sulfhydryl). In some embodiments, the first bioconjugate reactive group (e.g., haloacetyl moiety) is covalently attached to the second bioconjugate reactive group (e.g. a sulfhydryl). In some embodiments, the first bioconjugate reactive group (e.g., pyridyl moiety) is covalently attached to the second bioconjugate reactive group (e.g. a sulfhydryl). In some embodiments, the first bioconjugate reactive group (e.g., —N-hydroxysuccinimide moiety) is covalently attached to the second bioconjugate reactive group (e.g. an amine). In some embodiments, the first bioconjugate reactive group (e.g., maleimide moiety) is covalently attached to the second bioconjugate reactive group (e.g. a sulfhydryl). In some embodiments, the first bioconjugate reactive group (e.g., -sulfo-N-hydroxysuccinimide moiety) is covalently attached to the second bioconjugate reactive group (e.g. an amine).

In some embodiments, the linker is a peptide linker. In some embodiments, the linker is a flexible peptide linker. In some embodiments, a flexible peptide linker is about 20 or fewer amino acids in length. In some embodiments, a peptide linker contains about 12 or fewer amino acid residues, e.g., 3, 4, 5, 6, 7, 8, 9, 10, 11, and 12. In some embodiments, the peptide linker is 20 amino acids in length. In some embodiments, the peptide linker ranges from about 4 to about 16 amino acids in length. In some embodiments, the peptide linker is 2, 3, 4, 5, 6, 7, 8, 9, 10, 11, 12, 13, 14, 15, 16, 17, 18, 19, 20, 21, 22, 23, 24, or 25 amino acids in length. In some embodiments, the peptide linker ranges from about 2 to about 25 amino acids in length. In some embodiments, the peptide linker is longer than 25 amino acids in length. In some embodiments, a peptide linker comprises two or more of the following amino acids: glycine, serine, alanine, and proline. In some embodiments, the peptide linker is a glycine-serine linker. In some embodiments, the glycine-serine linker is represented by the formula (GS)n, wherein n is 1, 2, 3, 4, 5, 6, 7, 8, 9, 10, 11, or 12 (SEQ ID NO: 7). In some embodiments, a peptide linker is a G4S linker represented by the formula of (GGGGS)n, where n is 1, 2, 3, 4, or 5 (SEQ ID NO: 8). In some embodiments, a peptide linker is a G4A linker represented by the formula of (GGGGA)n, where n is 1, 2, 3, 4, or 5 (SEQ ID NO: 12). In some embodiments, a peptide linker is a G4P linker represented by the formula of (GGGGP)n, where n is 1, 2, 3, 4, or 5 (SEQ ID NO: 13). In some embodiments, a peptide linker is a G3A linker represented by the formula of (GGGA)n, where n is 1, 2, 3, 4, or 5 (SEQ ID NO: 14). In some embodiments, a peptide linker is represented by the formula of (GGGS)ₙ, where n is 1, 2, 3, 4, or 5 (SEQ ID No: 9). In some embodiments, the glycine-serine linker is represented by the formula GGGSGGGS (SEQ ID NO: 10).

In some embodiments, a peptide linker includes a protease-dependent cleavable site. Examples of protease-cleavable peptide linkers include, without limitation, the MMP sensitive linker GGPLGL W AGG (SEQ ID NO: 15) and the factor Xa-sensitive linker IEGR (SEQ ID NO: 16). It would be appreciated by a skilled artisan in the field that a variety of cleavable sequences may be employed for the linker provided herein.

In some embodiments, a linking molecule or linker may be a non-peptide linker. As used herein, a non-peptide linker is a biocompatible polymer including two or more repeating units linked to each other. Examples of the non-peptide polymer include but are not limited to: polyethylene glycol (PEG), polypropylene glycol (PPG), co-poly (ethylene/propylene) glycol, polyoxyethylene (POE), polyurethane, polyphosphazene, polysaccharides, dextran, polyvinyl alcohol, polyvinylpyrrolidones, polyvinyl ethyl ether, polyacryl amide, polyacrylate, polycyanoacrylates, lipid polymers, chitins, hyaluronic acid, and heparin. For more detailed descriptions of non-peptide linkers useful for Fc fusion molecules, see, for example, WO/2006/107124, which is incorporated by reference herein. In some embodiments, such linkers will have a range of molecular weight of from about 1 kDa to 50 kDa, depending upon a particular linker. In some embodiments, a typical PEG has a molecular weight of about 1 to 5 kDa, and polyethylene glycol has a molecular weight of about 5 kDa to 50 kDa, and more preferably about 10 kDa to 40 kDa.

As used herein, the term “vector” refers to a nucleic acid molecule capable of transporting another nucleic acid to which it has been linked. In some embodiments, a “vector” is any genetic element (e.g., DNA, RNA, or a mixture thereof) that contains a nucleic acid of interest that is capable of being expressed in a host cell, e.g., a nucleic acid of interest within a larger nucleic acid sequence or structure suitable for delivery to a cell, tissue, and/or organism, such as a plasmid, phage, transposon, cosmid, chromosome, virus, virion, etc. For instance, a vector may comprise an insert (e.g., a heterologous nucleic acid encoding a gene to be expressed or an open reading frame of that gene) and one or more additional elements, e.g., elements suitable for delivering or controlling expression of the insert. One type of vector is a “plasmid”, which refers to a linear or circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions. Additionally, some viral vectors are capable of targeting a particular cells type either specifically or non-specifically. Replication-incompetent viral vectors or replication-defective viral vectors refer to viral vectors that are capable of infecting their target cells and delivering their viral payload, but then fail to continue the typical lytic pathway that leads to cell lysis and death.

In some embodiments, a “vector” described herein in particular refers to a polynucleotide capable of carrying at least one polynucleotide fragment. A vector functions like a molecular carrier, delivering polynucleotides into a host cell. An expression vector may comprise at least one expression cassette comprising regulatory sequences for properly expressing a polynucleotide incorporated therein. Polynucleotides (e.g. encoding the polypeptide of interest or a selectable marker) to be introduced into the cell may be inserted into the expression cassette(s) of the vector in order to be expressed therefrom. When introduced into a host cell, an expression cassette inter alia is capable of directing the cell's machinery to transcribe an incorporated polynucleotide encoding a polypeptide of interest into RNA, which is then usually further processed and finally translated into the polypeptide of interest. The vector may be present in circular or linear(ized) form. The term “vector” also comprises artificial chromosomes, viral vectors or similar respective polynucleotides allowing the transfer of foreign nucleic acid fragments.

The compositions described herein can be purified. Purified compositions are at least about 60% by weight (dry weight) the compound of interest. Preferably, the preparation is at least about 75%, more preferably at least about 90%, and most preferably at least about 99% or higher by weight the compound of interest. Purity is measured by any appropriate standard method, for example, by High-performance liquid chromatography, polyacrylamide gel electrophoresis.

The term “isolated”, when applied to a nucleic acid or protein, denotes that the nucleic acid or protein is essentially free of other cellular components with which it is associated in the natural state. It can be, for example, in a homogeneous state and may be in either a dry or aqueous solution. Purity and homogeneity are typically determined using analytical chemistry techniques such as polyacrylamide gel electrophoresis or high performance liquid chromatography. A protein that is the predominant species present in a preparation is substantially purified.

A “cell” as used herein, refers to a cell carrying out metabolic or other function sufficient to preserve or replicate its genomic DNA. A cell can be identified by well-known methods in the art including, for example, presence of an intact membrane, staining by a particular dye, ability to produce progeny or, in the case of a gamete, ability to combine with a second gamete to produce a viable offspring. Cells may include prokaryotic and eukaryotic cells. Prokaryotic cells include but are not limited to bacteria. Eukaryotic cells include but are not limited to yeast cells and cells derived from plants and animals, for example mammalian, insect (e.g., spodoptera) and human cells.

The terms “inhibitor,” “repressor” or “antagonist” or “downregulator” interchangeably refer to a substance that results in a detectably lower expression or activity level as compared to a control. The inhibited expression or activity can be 10%, 20%, 30%, 40%, 50%, 60%, 70%, 80%, 90% or less than that in a control. In certain instances, the inhibition is 1.5-fold, 2-fold, 3-fold, 4-fold, 5-fold, 10-fold, or more in comparison to a control. An “inhibitor” is a peptide, siRNA, (e.g., shRNA, miRNA, snoRNA), compound or small molecule that inhibits cellular function (e.g., replication) e.g., by binding, partially or totally blocking stimulation, decrease, prevent, or delay activation, or inactivate, desensitize, or down-regulate signal transduction, gene expression or enzymatic activity necessary for protein activity.

A “pharmaceutical composition” is a formulation containing the composition (e.g., a dimer protein described herein) described herein in a form suitable for administration to a subject. In some embodiments, the pharmaceutical composition is in bulk or in unit dosage form. The unit dosage form is any of a variety of forms, including, for example, a lyophilization formulation, a capsule, an IV bag, a tablet, a single pump on an aerosol inhaler or a vial. The quantity of active ingredient (e.g., a formulation of the disclosed nucleic acid) in a unit dose of composition is an effective amount and is varied according to the particular treatment involved. One skilled in the art will appreciate that it is sometimes necessary to make routine variations to the dosage depending on the age and condition of the patient. The dosage will also depend on the route of administration. A variety of routes are contemplated, including oral, pulmonary, rectal, parenteral, transdermal, subcutaneous, intravenous, intramuscular, intraperitoneal, inhalational, buccal, sublingual, intrapleural, intrathecal, intranasal, and the like. Dosage forms for the topical or transdermal administration of a compound of this invention include powders, sprays, ointments, pastes, creams, lotions, gels, solutions, patches and inhalants.

As used herein, the phrase “pharmaceutically acceptable” refers to those compounds, anions, cations, materials, compositions, carriers, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically acceptable excipient” means an excipient that is useful in preparing a pharmaceutical composition that is generally safe, non-toxic and neither biologically nor otherwise undesirable, and includes excipient that is acceptable for veterinary use as well as human pharmaceutical use. A “pharmaceutically acceptable excipient” as used in the specification and claims includes both one and more than one such excipient. A thorough discussion of pharmaceutically acceptable excipients is available in REMINGTON'S PHARMACEUTICAL SCIENCES (Mack Pub. Co., N.J. 1991). Pharmaceutically acceptable excipients in therapeutic compositions may contain liquids such as water, saline, glycerol and ethanol. Additionally, auxiliary substances, such as wetting or emulsifying agents, pH buffering substances, and the like, may be present in such vehicles.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), and transmucosal administration.

Formulations suitable for oral administration can consist of (a) liquid solutions, such as an effective amount of the packaged nucleic acid suspended in diluents, such as water, saline or PEG 400; (b) capsules, sachets or tablets, each containing a predetermined amount of the active ingredient, as liquids, solids, granules or gelatin; (c) suspensions in an appropriate liquid; and (d) suitable emulsions. Tablet forms can include one or more of lactose, sucrose, mannitol, sorbitol, calcium phosphates, corn starch, potato starch, microcrystalline cellulose, gelatin, colloidal silicon dioxide, talc, magnesium stearate, stearic acid, and other excipients, colorants, fillers, binders, diluents, buffering agents, moistening agents, preservatives, flavoring agents, dyes, disintegrating agents, and pharmaceutically compatible carriers. Lozenge forms can comprise the active ingredient in a flavor, e.g., sucrose, as well as pastilles comprising the active ingredient in an inert base, such as gelatin and glycerin or sucrose and acacia emulsions, gels, and the like containing, in addition to the active ingredient, carriers known in the art.

Pharmaceutical compositions can also include large, slowly metabolized macromolecules such as proteins, polysaccharides such as chitosan, polylactic acids, polyglycolic acids and copolymers (such as latex functionalized Sepharose™, agarose, cellulose, and the like), polymeric amino acids, amino acid copolymers, and lipid aggregates (such as oil droplets or liposomes). Additionally, these carriers can function as immunostimulating agents (i.e., adjuvants).

Suitable formulations for rectal administration include, for example, suppositories, which consist of the packaged nucleic acid with a suppository base. Suitable suppository bases include natural or synthetic triglycerides or paraffin hydrocarbons. In addition, it is also possible to use gelatin rectal capsules which consist of a combination of the compound of choice with a base, including, for example, liquid triglycerides, polyethylene glycols, and paraffin hydrocarbons.

Formulations suitable for parenteral administration, such as, for example, by intraarticular (in the joints), intravenous, intramuscular, intratumoral, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. In the practice of this invention, compositions can be administered, for example, by intravenous infusion, orally, topically, intraperitoneally, intravesically or intrathecally. Parenteral administration, oral administration, and intravenous administration are the preferred methods of administration. The formulations of compounds can be presented in unit-dose or multi-dose sealed containers, such as ampules and vials.

Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

A pharmaceutical composition of the invention can be administered to a subject in many of the well-known methods currently used for chemotherapeutic treatment. For example, for treatment of cancers, a composition of the invention may be injected directly into tumors, injected into the blood stream or body cavities or taken orally or applied through the skin with patches. The dose chosen should be sufficient to constitute effective treatment but not so high as to cause unacceptable side effects. The state of the disease condition (e.g., cancer, precancer, and the like) and the health of the patient should preferably be closely monitored during and for a reasonable period after treatment.

As used herein, “monotherapy” refers to the administration of a single active or therapeutic compound to a subject in need thereof. Preferably, monotherapy will involve administration of a therapeutically effective amount of an active composition (e.g., a TWIST peptide, a TWIST peptide bound to a delivery vehicle, a fusion protein including a TWIST peptide, or any composition described herein). For example, described herein can be a cancer monotherapy with one of compositions described herein administered to a subject in need of for treatment of cancer. Monotherapy may be contrasted with combination therapy, in which a combination of multiple active compositions (e.g., multiple compositions described herein) are administered, preferably with each component of the combination present in a therapeutically effective amount. Monotherapy with a composition described herein may be more effective than combination therapy in inducing a desired biological effect.

As used herein, “combination therapy” or “co-therapy” or “co-administration” includes the administration of a composition described herein and at least a second agent as part of a specific treatment regimen intended to provide the beneficial effect from the co-action of these therapeutic agents. The beneficial effect of the combination may include, but is not limited to, pharmacokinetic or pharmacodynamic co-action resulting from the combination of therapeutic agents. Administration of these therapeutic agents in combination typically is carried out over a defined time period (usually minutes, hours, days or weeks depending upon the combination selected). “Combination therapy” may be, but generally is not, intended to encompass the administration of two or more of these therapeutic agents as part of separate monotherapy regimens that incidentally and arbitrarily result in the combinations described herein.

In some embodiments, co-administration includes administering one active agent within 0.5, 1, 2, 4, 6, 8, 10, 12, 16, 20, or 24 hours of a second active agent. Co-administration includes administering two active agents simultaneously, approximately simultaneously (e.g., within about 1, 5, 10, 15, 20, or 30 minutes of each other), or sequentially in any order. In some embodiments, co-administration can be accomplished by co-formulation, i.e., preparing a single pharmaceutical composition including both active agents. In other embodiments, the active agents can be formulated separately. In another embodiment, the active and/or adjunctive agents may be linked or conjugated to one another. In some embodiments, the compounds described herein may be combined with treatments for cancer (e.g. prostate cancer, castration-resistant prostate cancer, breast cancer, triple negative breast cancer, glioblastoma, ovarian cancer, lung cancer, squamous cell carcinoma (e.g head, neck, or esophagus), colorectal cancer, leukemia, acute myeloid leukemia, lymphoma, B cell lymphoma, or multiple myeloma) such as surgery or with other treatments known to be useful in treating viral disease (e.g. herpesvirus infection associated disease or hepatitis virus infection associated disease or HIV infection associated disease).

“Combination therapy” is intended to embrace administration of these therapeutic agents in a sequential manner, wherein each therapeutic agent is administered at a different time, as well as administration of these therapeutic agents, or at least two of the therapeutic agents, in a substantially simultaneous manner. Substantially simultaneous administration can be accomplished, for example, by administering to the subject a single capsule having a fixed ratio of each therapeutic agent or in multiple, single capsules for each of the therapeutic agents. Sequential or substantially simultaneous administration of each therapeutic agent can be effected by any appropriate route including, but not limited to, oral routes, intravenous routes, intramuscular routes, and direct absorption through mucous membrane tissues. The therapeutic agents can be administered by the same route or by different routes. For example, a first therapeutic agent of the combination selected may be administered by intravenous injection while the other therapeutic agents of the combination may be administered orally. Alternatively, for example, all therapeutic agents may be administered orally or all therapeutic agents may be administered by intravenous injection. The sequence in which the therapeutic agents are administered is not narrowly critical.

“Combination therapy” also embraces the administration of the therapeutic agents as described above in further combination with other biologically active ingredients and non-drug therapies (e.g., surgery or radiation treatment). Where the combination therapy further comprises a non-drug treatment, the non-drug treatment may be conducted at any suitable time so long as a beneficial effect from the co-action of the combination of the therapeutic agents and non-drug treatment is achieved. For example, in appropriate cases, the beneficial effect is still achieved when the non-drug treatment is temporally removed from the administration of the therapeutic agents, perhaps by days or even weeks.

A composition described herein may be administered in combination with a second chemotherapeutic agent. The second chemotherapeutic agent (also referred to as an anti-neoplastic agent or anti-proliferative agent) can be an alkylating agent; an antibiotic; an anti-metabolite; a detoxifying agent; an interferon; a polyclonal or monoclonal antibody; an EGFR inhibitor; a HER2 inhibitor; a histone deacetylase inhibitor; a hormone; a mitotic inhibitor; an MTOR inhibitor; a multi-kinase inhibitor; a serine/threonine kinase inhibitor; a tyrosine kinase inhibitors; a VEGF/VEGFR inhibitor; a taxane or taxane derivative, an aromatase inhibitor, an anthracycline, a microtubule targeting drug, a topoisomerase poison drug, an inhibitor of a molecular target or enzyme (e.g., a kinase or a protein methyltransferase), a cytidine analogue drug or any chemotherapeutic, anti-neoplastic or anti-proliferative agent listed in www.cancer.org/docroot/cdg/cdg_0.asp.

“Anti-cancer therapy” is used in accordance with its plain ordinary meaning and refers to a therapy, optionally conforming to a regimen, having antineoplastic properties or the ability to inhibit the growth or proliferation of cells.

“Anti-cancer agent” is used in accordance with its plain ordinary meaning and refers to a composition (e.g. compound, drug, antagonist, inhibitor, modulator) having antineoplastic properties or the ability to inhibit the growth or proliferation of cells. In some embodiments, an anti-cancer agent is a chemotherapeutic. In some embodiments, an anti-cancer agent is an agent identified herein having utility in methods of treating cancer. In some embodiments, an anti-cancer agent is an agent approved by the FDA or similar regulatory agency of a country other than the USA, for treating cancer.

The anti-cancer agents set forth below are for illustrative purposes and not intended to be limiting. In some embodiments, at least one anti-cancer agent is selected from the lists below. In some embodiments, more than one anti-cancer agent, e.g., two, three, four, or five anti-cancer agents are selected in order to perform its intended function. In some embodiments, the anti-cancer agent is an IL-15 (e.g., GenBank AAX37025)/IL-15Ra (e.g., GenBank AAP69528.1) heterocomplex (e.g., IL-15/IL-Ra heterocomplexes disclosed in WO2007/001677 and WO20007/084342 and/or encoded by SEQ ID NO: 67). In some embodiments, the anti-cancer agent is an anti-PD-1 antibody or other PD-1 inhibitor. Antibodies, antibody fragments, and other inhibitors of PD-1, PD-L1 and PD-L2 are available in the art and may be used combination with a cars of the present invention described herein. For example, nivolumab (also referred to as BMS-936558 or MDX1106; Bristol-Myers Squibb) is a fully human IgG4 monoclonal antibody which specifically blocks PD-1. Nivolumab (clone 5C4) and other human monoclonal antibodies that specifically bind to PD-1 are disclosed in U.S. Pat. No. 8,008,449 and WO2006/121168. Pidilizumab (CT-011; Cure Tech) is a humanized IgG1k monoclonal antibody that binds to PD-1. Pidilizumab and other humanized anti-PD-1 monoclonal antibodies are disclosed in WO2009/101611. Pembrolizumab (formerly known as lambrolizumab, and also referred to as MK03475; Merck) is a humanized IgG4 monoclonal antibody that binds to PD-1. Pembrolizumab and other humanized anti-PD-1 antibodies are disclosed in U.S. Pat. No. 8,354,509 and WO2009/114335. MEDI4736 (Medimmune) is a human monoclonal antibody that binds to PDL1, and inhibits interaction of the ligand with PD1. MDPL3280A (Genentech/Roche) is a human Fc optimized IgG1 monoclonal antibody that binds to PD-L1. MDPL3280A and other human monoclonal antibodies to PD-L1 are disclosed in U.S. Pat. No. 7,943,743 and U.S. Publication No.: 20120039906. Other anti-PD-L1 binding agents include YW243.55.570 (heavy and light chain variable regions are shown in SEQ ID NOs 20 and 21 in WO2010/077634) and MDX-1 105 (also referred to as BMS-936559, and, e.g., anti-PD-L1 binding agents disclosed in WO2007/005874). AMP-224 (B7-DCIg; Amplimmune; e.g., disclosed in WO2010/027827 and WO2011/066342), is a PD-L2 Fc fusion soluble receptor that blocks the interaction between PD-1 and B7-H1. Other anti-PD-1 antibodies include AMP 514 (Amplimmune), among others, e.g., anti-PD-1 antibodies disclosed in U.S. Pat. No. 8,609,089, US 2010028330, and/or US 20120114649. In some embodiments, the anti-cancer agent is a radioligand, such as but not limited to ¹⁷⁷Lu-PSMA-617 (lutetium (117Lu) oxodotreotide). In some embodiments, the anti-cancer agent is a cancer vaccine.

In some embodiments, the anticancer agent is a compound that affects histone modifications, such as an MAC inhibitor. In some embodiments, an anticancer agent is selected from the group consisting of chemotherapeutics (such as 2CdA, 5-FU, 6-Mercaptopurine, 6-TG, Abraxane™, Accutane®, Actinomycin-D, Adriamycin®, Alimta®, all-trans retinoic acid, amethopterin, Ara-C, Azacitadine, BCNU, Blenoxane®, Camptosar®, CeeNU®, Clofarabine, Clolar™, Cytoxan®, daunorubicin hydrochloride, DaunoXome®, Dacogen®, DIC, Doxil®, Ellence®, Eloxatin®, Emcyt®, etoposide phosphate, Fludara®, FUDR®, Gemzar®, Gleevec®, hexamethylmelamine, Hycamtin®, Hydrea®, Idamycin®, Ifex®, ixabepilone, Ixempra®, L-asparaginase, Leukeran®, liposomal Ara-C, L-PAM, Lysodren, Matulane®, mithracin, Mitomycin-C, Myleran®, Navelbine®, Neutrexin®, nilotinib, Nipent®, Nitrogen Mustard, Novantrone®, Oncaspar®, Panretin®, Paraplatin®, Platinol®, prolifeprospan 20 with carmustine implant, Sandostatin®, Targretin®, Tasigna®, Taxotere®, Temodar®, TESPA, Trisenox®, Valstar®, Velban®, Vidaza™, vincristine sulfate, VM 26, Xeloda® and Zanosar®); biologics (such as Alpha Interferon, Bacillus Calmette-Guerin, Bexxar®, Campath®, Ergamisol®, Erlotinib, Herceptin®, Interleukin-2, Iressa®, lenalidomide, Mylotarg®, Ontak®, Pegasys®, Revlimid®, Rituxan®, Tarceva™, Thalomid®, Tykerb®, Velcade® and Zevalin™); corticosteroids, (such as dexamethasone sodium phosphate, DeltaSone® and Delta-Cortef®); hormonal therapies (such as Arimidex®, Aromasin®, Casodex®, Cytadren®, Eligard®, Eulexin®, Evista®, Faslodex®, Femara®, Halotestin®, Megace®, Nilandron®, Nolvadex®, Plenaxis™ and Zoladex®); and radiopharmaceuticals (such as Iodotope®, Metastron®, Phosphocol® and Samarium SM-153).

In some embodiments, the anti-cancer agent is a chemotherapeutic agent (also referred to as an anti-neoplastic agent or anti-proliferative agent), selected from the group including an alkylating agent; an antibiotic; an anti-metabolite; a detoxifying agent; an interferon; a polyclonal or monoclonal antibody; an EGFR inhibitor; a HER2 inhibitor; a histone deacetylase inhibitor; a hormone; a mitotic inhibitor; an MTOR inhibitor; a multi-kinase inhibitor; a serine/threonine kinase inhibitor; a tyrosine kinase inhibitors; a VEGF/VEGFR inhibitor; a taxane or taxane derivative, an aromatase inhibitor, an anthracycline, a microtubule targeting drug, a topoisomerase poison drug, an inhibitor of a molecular target or enzyme (e.g., a kinase or a protein methyltransferase), a cytidine analogue drug or any chemotherapeutic, anti-neoplastic or anti-proliferative agent listed in www.cancer.org/docroot/cdg/cdg_0.asp.

Exemplary alkylating agents include, but are not limited to, cyclophosphamide (Cytoxan; Neosar); chlorambucil (Leukeran); melphalan (Alkeran); carmustine (BiCNU); busulfan (Busulfex); lomustine (CeeNU); dacarbazine (DTIC-Dome); oxaliplatin (Eloxatin); carmustine (Gliadel); ifosfamide (Ifex); mechlorethamine (Mustargen); busulfan (Myleran); carboplatin (Paraplatin); cisplatin (CDDP; Platinol); temozolomide (Temodar); thiotepa (Thioplex); bendamustine (Treanda); or streptozocin (Zanosar).

Exemplary antibiotics include, but are not limited to, doxorubicin (Adriamycin); doxorubicin liposomal (Doxil); mitoxantrone (Novantrone); bleomycin (Blenoxane); daunorubicin (Cerubidine); daunorubicin liposomal (DaunoXome); dactinomycin (Cosmegen); epirubicin (Ellence); idarubicin (Idamycin); plicamycin (Mithracin); mitomycin (Mutamycin); pentostatin (Nipent); or valrubicin (Valstar).

Exemplary anti-metabolites include, but are not limited to, fluorouracil (Adrucil); capecitabine (Xeloda); hydroxyurea (Hydrea); mercaptopurine (Purinethol); pemetrexed (Alimta); fludarabine (Fludara); nelarabine (Arranon); cladribine (Cladribine Novaplus); clofarabine (Clolar); cytarabine (Cytosar-U); decitabine (Dacogen); cytarabine liposomal (DepoCyt); hydroxyurea (Droxia); pralatrexate (Folotyn); floxuridine (FUDR); gemcitabine (Gemzar); cladribine (Leustatin); fludarabine (Oforta); methotrexate (MTX; Rheumatrex); methotrexate (Trexall); thioguanine (Tabloid); TS-1 or cytarabine (Tarabine PFS).

Exemplary detoxifying agents include, but are not limited to, amifostine (Ethyol) or mesna (Mesnex).

Exemplary interferons include, but are not limited to, interferon alfa-2b (Intron A) or interferon alfa-2a (Roferon-A).

Exemplary polyclonal or monoclonal antibodies include, but are not limited to, trastuzumab (Herceptin); ofatumumab (Arzerra); bevacizumab (Avastin); rituximab (Rituxan); cetuximab (Erbitux); panitumumab (Vectibix); tositumomab/iodine131 tositumomab (Bexxar); alemtuzumab (Campath); ibritumomab (Zevalin; In-111; Y-90 Zevalin); gemtuzumab (Mylotarg); eculizumab (Soliris) ordenosumab.

Exemplary EGFR inhibitors include, but are not limited to, gefitinib (Iressa); lapatinib (Tykerb); cetuximab (Erbitux); erlotinib (Tarceva); panitumumab (Vectibix); PKI-166; canertinib (CI-1033); matuzumab (Emd7200) or EKB-569.

Exemplary HER2 inhibitors include, but are not limited to, trastuzumab (Herceptin); lapatinib (Tykerb) or AC-480.

Exemplary Histone Deacetylase Inhibitors include, but are not limited to, vorinostat (Zolinza).

Exemplary hormones include, but are not limited to, tamoxifen (Soltamox; Nolvadex); raloxifene (Evista); megestrol (Megace); leuprolide (Lupron; Lupron Depot; Eligard; Viadur); fulvestrant (Faslodex); letrozole (Femara); triptorelin (Trelstar LA; Trelstar Depot); exemestane (Aromasin); goserelin (Zoladex); bicalutamide (Casodex); anastrozole (Arimidex); fluoxymesterone (Androxy; Halotestin); medroxyprogesterone (Provera; Depo-Provera); estramustine (Emcyt); flutamide (Eulexin); toremifene (Fareston); degarelix (Firmagon); nilutamide (Nilandron); abarelix (Plenaxis); or testolactone (Teslac).

Exemplary mitotic inhibitors include, but are not limited to, paclitaxel (Taxol; Onxol; Abraxane); docetaxel (Taxotere); vincristine (Oncovin; Vincasar PFS); vinblastine (Velban); etoposide (Toposar; Etopophos; VePesid); teniposide (Vumon); ixabepilone (Ixempra); nocodazole; epothilone; vinorelbine (Navelbine); camptothecin (CPT); irinotecan (Camptosar); topotecan (Hycamtin); amsacrine or lamellarin D (LAM-D).

Exemplary MTOR inhibitors include, but are not limited to, everolimus (Afinitor) or temsirolimus Torisel); rapamune, ridaforolimus; or AP23573.

Exemplary multi-kinase inhibitors include, but are not limited to, sorafenib (Nexavar); sunitinib (Sutent); BIBW 2992; E7080; Zd6474; PKC-412; motesanib; or AP24534.

Exemplary serine/threonine kinase inhibitors include, but are not limited to, ruboxistaurin; eril/easudil hydrochloride; flavopiridol; Pkc412; bryostatin; KAI-9803; SF1126; or PD 332991.

Exemplary tyrosine kinase inhibitors include, but are not limited to, erlotinib (Tarceva); gefitinib (Iressa); imatinib (Gleevec); sorafenib (Nexavar); sunitinib (Sutent); trastuzumab (Herceptin); bevacizumab (Avastin); rituximab (Rituxan); lapatinib (Tykerb); cetuximab (Erbitux); panitumumab (Vectibix); everolimus (Afinitor); alemtuzumab (Campath); gemtuzumab (Mylotarg); temsirolimus (Torisel); pazopanib (Votrient); dasatinib (Sprycel); nilotinib (Tasigna); vatalanib (Ptk787; ZK222584); WHI-P154; WHI-P131; AC-220; or AMG888.

Exemplary VEGF/VEGFR inhibitors include, but are not limited to, bevacizumab (Avastin); sorafenib (Nexavar); sunitinib (Sutent); ranibizumab; pegaptanib; or vandetinib.

Exemplary microtubule targeting drugs include, but are not limited to, paclitaxel, docetaxel, vincristine, vinblastin, nocodazole, epothilones and navelbine.

Exemplary topoisomerase poison drugs include, but are not limited to, teniposide, etoposide, adriamycin, camptothecin, daunorubicin, dactinomycin, mitoxantrone, amsacrine, epirubicin and idarubicin.

Exemplary taxanes or taxane derivatives include, but are not limited to, paclitaxel and docetaxol.

Exemplary general chemotherapeutic, anti-neoplastic, anti-proliferative agents include, but are not limited to, altretamine (Hexalen); isotretinoin (Accutane; Amnesteem; Claravis; Sotret); tretinoin (Vesanoid); azacitidine (Vidaza); bortezomib (Velcade) asparaginase (Elspar); levamisole (Ergamisol); mitotane (Lysodren); procarbazine (Matulane); pegaspargase (Oncaspar); denileukin diftitox (Ontak); porfimer (Photofrin); aldesleukin (Proleukin); lenalidomide (Revlimid); bexarotene (Targretin); thalidomide (Thalomid); temsirolimus (Torisel); arsenic trioxide (Trisenox); verteporfin (Visudyne); mimosine (Leucenol); (1M tegafur-0.4 M 5-chloro-2,4-dihydroxypyrimidine-1 M potassium oxonate), or lovastatin.

In some embodiments, the anti-cancer agent is a chemotherapeutic agent or a cytokine such as G-CSF (granulocyte colony stimulating factor).

In some embodiments, the anti-cancer agents can be standard chemotherapy combinations such as, but not restricted to, CMF (cyclophosphamide, methotrexate and 5-fluorouracil), CAF (cyclophosphamide, adriamycin and 5-fluorouracil), AC (adriamycin and cyclophosphamide), FEC (5-fluorouracil, epirubicin, and cyclophosphamide), ACT or ATC (adriamycin, cyclophosphamide, and paclitaxel), rituximab, Xeloda (capecitabine), Cisplatin (CDDP), Carboplatin, TS-1 (tegafur, gimestat and otastat potassium at a molar ratio of 1:0.4:1), Camptothecin-11 (CPT-11, Irinotecan or Camptosar™), CHOP (cyclophosphamide, hydroxydaunorubicin, oncovin, and prednisone or prednisolone), R-CHOP (rituximab, cyclophosphamide, hydroxydaunorubicin, oncovin, prednisone or prednisolone), or CMFP (cyclophosphamide, methotrexate, 5-fluorouracil and prednisone).

In some embodiments, the anti-cancer agents can be an inhibitor of an enzyme, such as a receptor or non-receptor kinase. Receptor and non-receptor kinases are, for example, tyrosine kinases or serine/threonine kinases. Kinase inhibitors described herein are small molecules, polynucleic acids, polypeptides, or antibodies. Exemplary kinase inhibitors include, but are not limited to, Bevacizumab (targets VEGF), BIBW 2992 (targets EGFR and Erb2), Cetuximab/Erbitux (targets Erb1), Imatinib/Gleevic (targets Bcr-Abl), Trastuzumab (targets Erb2), Gefitinib/Iressa (targets EGFR), Ranibizumab (targets VEGF), Pegaptanib (targets VEGF), Erlotinib/Tarceva (targets Erb1), Nilotinib (targets Bcr-Abl), Lapatinib (targets Erb1 and Erb2/Her2), GW-572016/lapatinib ditosylate (targets HER2/Erb2), Panitumumab/Vectibix (targets EGFR), Vandetinib (targets RET/VEGFR), E7080 (multiple targets including RET and VEGFR), Herceptin (targets HER2/Erb2), PM-166 (targets EGFR), Canertinib/CI-1033 (targets EGFR), Sunitinib/SU-11464/Sutent (targets EGFR and FLT3), Matuzumab/Emd7200 (targets EGFR), EKB-569 (targets EGFR), Zd6474 (targets EGFR and VEGFR), PKC-412 (targets VEGR and FLT3), Vatalanib/Ptk787/ZK222584 (targets VEGR), CEP-701 (targets FLT3), SU5614 (targets FLT3), MLN518 (targets FLT3), XL999 (targets FLT3), VX-322 (targets FLT3), Azd0530 (targets SRC), BMS-354825 (targets SRC), SKI-606 (targets SRC), CP-690 (targets JAK), AG-490 (targets JAK), WHI-P154 (targets JAK), WHI-P131 (targets JAK), sorafenib/Nexavar (targets RAF kinase, VEGFR-1, VEGFR-2, VEGFR-3, PDGFR-ß, KIT, FLT-3, and RET), Dasatinib/Sprycel (BCR/ABL and Src), AC-220 (targets Flt3), AC-480 (targets all HER proteins, “panHER”), Motesanib diphosphate (targets VEGF1-3, PDGFR, and c-kit), Denosumab (targets RANKL, inhibits SRC), AMG888 (targets HER3), and AP24534 (multiple targets including Flt3).

Exemplary serine/threonine kinase inhibitors include, but are not limited to, Rapamune (targets mTOR/FRAP1), Deforolimus (targets mTOR), Certican/Everolimus (targets mTOR/FRAP1), AP23573 (targets mTOR/FRAP1), Eril/Fasudil hydrochloride (targets RHO), Flavopiridol (targets CDK), Seliciclib/CYC202/Roscovitrine (targets CDK), SNS-032/BMS-387032 (targets CDK), Ruboxistaurin (targets PKC), Pkc412 (targets PKC), Bryostatin (targets PKC), KAI-9803 (targets PKC), SF1126 (targets PI3K), VX-680 (targets Aurora kinase), Azd1152 (targets Aurora kinase), Arry-142886/AZD-6244 (targets MAP/MEK), SCIO-469 (targets MAP/MEK), GW681323 (targets MAP/MEK), CC-401 (targets JNK), CEP-1347 (targets JNK), and PD 332991 (targets CDK).

Additionally, the peptide composition described herein can be co-administered with conventional immunotherapeutic agents including, but not limited to, immunostimulants (e.g., Bacillus Calmette-Guérin (BCG), levamisole, interleukin-2, alpha-interferon, etc.), monoclonal antibodies (e.g., anti-CD20, anti-HER2, anti-CD52, anti-HLA-DR, and anti-VEGF monoclonal antibodies), immunotoxins (e.g., anti-CD33 monoclonal antibody-calicheamicin conjugate, anti-CD22 monoclonal antibody-pseudomonas exotoxin conjugate, etc.), and radioimmunotherapy (e.g., anti-CD20 monoclonal antibody conjugated to ¹¹¹In, ⁹⁰Y, or ¹³¹I etc.).

In some embodiments, the peptide composition described herein can be co-administered with conventional radiotherapeutic agents including, but not limited to, radionuclides such as ⁴⁷Sc, ⁶⁴Cu, ⁶⁷Cu, ⁸⁹Sr, ⁸⁶Y, ⁸⁷Y, ⁹⁰Y, ¹⁰⁵Rh, ¹¹¹Ag, ¹¹¹In, ^{117m}Sn, ¹⁴⁹Pm, ¹⁵³Sm, ¹⁶⁶Ho, ¹⁷⁷Lu, ¹⁸⁶Re, ¹⁸⁸Re, ²¹¹At, and ²¹²Bi, optionally conjugated to antibodies directed against tumor antigens.

In some embodiments, the anti-cancer agent used herein refers to Doxorubicin, Cisplatin, Carboplatin, Taxanes, Camptothecin or any combination thereof.

A “subject” or a “patient” includes a mammal. The mammal can be e.g., a human or appropriate non-human mammal, such as primate, mouse, rat, dog, cat, cow, horse, goat, camel, sheep or a pig. The subject can also be a bird or fowl. In some embodiments, the mammal is a human. Thus the methods are applicable to both human therapy and veterinary applications. In some embodiments, a subject or a subject in need thereof is a subject having an NKG2D ligand expressing disease. An “NKG2D ligand expressing disease” is a disease or a condition where NKG2D ligand (e.g., MICA, MICB, ULBP1, ULBP2, ULBP3, ULBP4, ULBP5, and/or ULBP6) expression is abnormal (e.g., higher or lower) compared to that of a subject without such disease or condition. Exemplary NKG2D ligand expressing disease includes, but is not limited to, cancer, autoimmune disease. In some embodiments, a subject or a subject in need thereof is a subject having cancer or a subject having a precancerous condition. In some embodiments, a subject in need thereof has cancer. In some embodiments, a subject or a subject in need thereof is a subject having an autoimmune disease.

The term “autoimmune disease” refers to disorders wherein the immune system of a mammal mounts a humoral or cellular immune response to the mammal's own tissue or to antigens that are not intrinsically harmful to the mammal, thereby producing tissue injury in such a mammal. The symptoms and degree of severity vary from patient to patient. Further, the clinical features of the disease in a patient vary dramatically with time.

Examples of autoimmune disorders include, but are not limited to, systemic lupus erythematosus, rheumatoid arthritis and type I diabetes. Autoimmune diseases also include acute glomerulonephritis, Addison's disease, adult onset idiopathic hypoparathyroidism (AOIH), alopecia totalis, amyotrophic lateral sclerosis, ankylosing spondylitis, autoimmune aplastic anemia, autoimmune hemolytic anemia, Behcet's disease, Celiac disease, chronic active hepatitis, CREST syndrome, Crohn's disease, dermatomyositis, dilated cardiomyopathy, eosinophilia-myalgia syndrome, epidermolisis bullosa acquisita (EBA), giant cell arteritis, Goodpasture's syndrome, Graves' disease, Guillain-Barre syndrome, hemochromatosis, Henoch-Schonlein purpura, idiopathic IgA nephropathy, insulin-dependent diabetes mellitus (IDDM), juvenile rheumatoid arthritis, Lambert-Eaton syndrome, linear IgA dermatosis, lupus erythematosus, multiple sclerosis, myasthenia gravis, myocarditis, narcolepsy, necrotizing vasculitis, neonatal lupus syndrome (NLE), nephrotic syndrome, pemphigoid, phemphigus, polymyositis, primary sclerosing cholangitis, psoriasis, rapidly progressive glomerulonephritis (RPGN), Reiter's syndrome, rheumatoid arthritis, scleroderma, Sjogren's syndrome, stiff-man syndrome, thyroiditis, and ulcerative colitis. This is not intended to be an exhaustive list but rather, it is intended to demonstrate that autoimmunity is a wide ranging clinical phenomenon. Exemplary diseases in this field such as systemic lupus erythematosus, rheumatoid arthritis, mixed connective tissue disease and APLS are discussed in further detail herein below, with a view to providing some understanding of the problems involved in clinical diagnoses and treatment of these debilitating and potentially fatal disorders

In some embodiments, an autoimmune disease is rheumatoid arthritis, colitis, celiac disease, multiple sclerosis, alopecia areata, type 1 diabetes, chronic obstructive pulmonary disease, atherosclerosis, or metabolic syndrome associated with type 2 diabetes.

As used herein, the term “cancer” refers to all types of cancer, neoplasm or malignant tumors found in mammals, including leukemias, lymphomas, melanomas, neuroendocrine tumors, carcinomas and sarcomas. Exemplary cancers that may be treated with a composition, pharmaceutical composition, or method provided herein include lymphoma, sarcoma, bladder cancer, bone cancer, brain tumor, cervical cancer, colon cancer, esophageal cancer, gastric cancer, head and neck cancer, kidney cancer, myeloma, thyroid cancer, leukemia, prostate cancer, breast cancer (e.g. triple negative, ER positive, ER negative, chemotherapy resistant, herceptin resistant, HER2 positive, doxorubicin resistant, tamoxifen resistant, ductal carcinoma, lobular carcinoma, primary, metastatic), ovarian cancer, pancreatic cancer, liver cancer (e.g., hepatocellular carcinoma), lung cancer (e.g. non-small cell lung carcinoma, squamous cell lung carcinoma, adenocarcinoma, large cell lung carcinoma, small cell lung carcinoma, carcinoid, sarcoma), glioblastoma multiforme, glioma, melanoma, prostate cancer, castration-resistant prostate cancer, breast cancer, triple negative breast cancer, glioblastoma, ovarian cancer, lung cancer, squamous cell carcinoma (e.g., head, neck, or esophagus), colorectal cancer, leukemia, acute myeloid leukemia, lymphoma, B cell lymphoma, or multiple myeloma. Additional examples include, cancer of the thyroid, endocrine system, brain, breast, cervix, colon, head & neck, esophagus, liver, kidney, lung, non-small cell lung, melanoma, mesothelioma, ovary, sarcoma, stomach, uterus or Medulloblastoma, Hodgkin's Disease, Non-Hodgkin's Lymphoma, multiple myeloma, neuroblastoma, glioma, glioblastoma multiforme, ovarian cancer, rhabdomyosarcoma, primary thrombocytosis, primary macroglobulinemia, primary brain tumors, cancer, malignant pancreatic insulanoma, malignant carcinoid, urinary bladder cancer, premalignant skin lesions, testicular cancer, lymphomas, thyroid cancer, neuroblastoma, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, Paget's Disease of the Nipple, Phyllodes Tumors, Lobular Carcinoma, Ductal Carcinoma, cancer of the pancreatic stellate cells, cancer of the hepatic stellate cells, or prostate cancer. In some embodiments, an NKG2D ligand expressing cancer is lung cancer, ovarian cancer, colon cancer, pancreatic cancer, bladder cancer, breast cancer, hepatocellular carcinoma (HCC), renal cancer, nasopharyngeal carcinoma (NPC), prostate cancer, melanoma, plasma cell cancer, leukemia, lymphoma, glioma, or neuroblastoma.

The term “leukemia” refers broadly to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease-acute or chronic; (2) the type of cell involved; myeloid (myelogenous), lymphoid (lymphogenous), or monocytic; and (3) the increase or non-increase in the number abnormal cells in the blood-leukemic or aleukemic (subleukemic). Exemplary leukemias that may be treated with a composition, pharmaceutical composition, or method provided herein include, for example, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, multiple myeloma, plasmacytic leukemia, promyelocytic leukemia, Rieder cell leukemia, Schilling's leukemia, stem cell leukemia, subleukemic leukemia, or undifferentiated cell leukemia.

The term “sarcoma” generally refers to a tumor which is made up of a substance like the embryonic connective tissue and is generally composed of closely packed cells embedded in a fibrillar or homogeneous substance. Sarcomas that may be treated with a composition, pharmaceutical composition, or method provided herein include a chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, or telangiectaltic sarcoma.

The term “melanoma” is taken to mean a tumor arising from the melanocytic system of the skin and other organs. Melanomas that may be treated with a composition, pharmaceutical composition, or method provided herein include, for example, acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma, subungal melanoma, or superficial spreading melanoma.

“Breast cancer” includes all forms of cancer of the breast. Breast cancer can include primary epithelial breast cancers. Breast cancer can include cancers in which the breast is involved by other tumors such as lymphoma, sarcoma or melanoma. Breast cancer can include carcinoma of the breast, ductal carcinoma of the breast, lobular carcinoma of the breast, undifferentiated carcinoma of the breast, cystosarcoma phyllodes of the breast, angiosarcoma of the breast, and primary lymphoma of the breast. Breast cancer can include Stage I, II, IIIA, IIIB, IIIC and IV breast cancer. Ductal carcinoma of the breast can include invasive carcinoma, invasive carcinoma in situ with predominant intraductal component, inflammatory breast cancer, and a ductal carcinoma of the breast with a histologic type selected from the group consisting of comedo, mucinous (colloid), medullary, medullary with lymphcytic infiltrate, papillary, scirrhous, and tubular. Lobular carcinoma of the breast can include invasive lobular carcinoma with predominant in situ component, invasive lobular carcinoma, and infiltrating lobular carcinoma. Breast cancer can include Paget's disease, Paget's disease with intraductal carcinoma, and Paget's disease with invasive ductal carcinoma. Breast cancer can include breast neoplasms having histologic and ultrastructual heterogeneity (e.g., mixed cell types).

A breast cancer that is to be treated can include familial breast cancer. A breast cancer that is to be treated can include sporadic breast cancer. A breast cancer that is to be treated can arise in a male subject. A breast cancer that is to be treated can arise in a female subject. A breast cancer that is to be treated can arise in a premenopausal female subject or a postmenopausal female subject. A breast cancer that is to be treated can arise in a subject equal to or older than 30 years old, or a subject younger than 30 years old. A breast cancer that is to be treated has arisen in a subject equal to or older than 50 years old, or a subject younger than 50 years old. A breast cancer that is to be treated can arise in a subject equal to or older than 70 years old, or a subject younger than 70 years old.

A breast cancer that is to be treated can be typed to identify a familial or spontaneous mutation in BRCA1, BRCA2, or p53. A breast cancer that is to be treated can be typed as having a HER2/neu gene amplification, as overexpressing HER2/neu, or as having a low, intermediate or high level of HER2/neu expression. A breast cancer that is to be treated can be typed for a marker selected from the group consisting of estrogen receptor (ER), progesterone receptor (PR), human epidermal growth factor receptor-2, Ki-67, CA15-3, CA 27-29, and c-Met. A breast cancer that is to be treated can be typed as ER-unknown, ER-rich or ER-poor. A breast cancer that is to be treated can be typed as ER-negative or ER-positive. ER-typing of a breast cancer may be performed by any reproducible means. ER-typing of a breast cancer may be performed as set forth in Onkologie 27: 175-179 (2004). A breast cancer that is to be treated can be typed as PR-unknown, PR-rich, or PR-poor. A breast cancer that is to be treated can be typed as PR-negative or PR-positive. A breast cancer that is to be treated can be typed as receptor positive or receptor negative. A breast cancer that is to be treated can be typed as being associated with elevated blood levels of CA 15-3, or CA 27-29, or both. A breast cancer that is to be treated can be “triple-negative breast cancer” (TNBC) (estrogen receptor [ER]-negative, progesterone receptor [PR]-negative, and human epidermal growth factor receptor 2 [HER2]-negative).

A breast cancer that is to be treated can include a localized tumor of the breast. A breast cancer that is to be treated can include a tumor of the breast that is associated with a negative sentinel lymph node (SLN) biopsy. A breast cancer that is to be treated can include a tumor of the breast that is associated with a positive sentinel lymph node (SLN) biopsy. A breast cancer that is to be treated can include a tumor of the breast that is associated with one or more positive axillary lymph nodes, where the axillary lymph nodes have been staged by any applicable method. A breast cancer that is to be treated can include a tumor of the breast that has been typed as having nodal negative status (e.g., node-negative) or nodal positive status (e.g., node-positive). A breast cancer that is to be treated can include a tumor of the breast that has metastasized to other locations in the body. A breast cancer that is to be treated can be classified as having metastasized to a location selected from the group consisting of bone, lung, liver, or brain. A breast cancer that is to be treated can be classified according to a characteristic selected from the group consisting of metastatic, localized, regional, local-regional, locally advanced, distant, multicentric, bilateral, ipsilateral, contralateral, newly diagnosed, recurrent, and inoperable.

The term “carcinoma” refers to a malignant new growth made up of epithelial cells tending to infiltrate the surrounding tissues and give rise to metastases. Exemplary carcinomas that may be treated with a composition, pharmaceutical composition, or method provided herein include, for example, medullary thyroid carcinoma, familial medullary thyroid carcinoma, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, ductal carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatiniforni carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypernephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lobular carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tubular carcinoma, tuberous carcinoma, verrucous carcinoma, or carcinoma villosum.

As used herein, the terms “metastasis,” “metastatic,” and “metastatic cancer” can be used interchangeably and refer to the spread of a proliferative disease or disorder, e.g., cancer, from one organ or another non-adjacent organ or body part. Cancer occurs at an originating site, e.g., breast, which site is referred to as a primary tumor, e.g., primary breast cancer. Some cancer cells in the primary tumor or originating site acquire the ability to penetrate and infiltrate surrounding normal tissue in the local area and/or the ability to penetrate the walls of the lymphatic system or vascular system circulating through the system to other sites and tissues in the body. A second clinically detectable tumor formed from cancer cells of a primary tumor is referred to as a metastatic or secondary tumor. When cancer cells metastasize, the metastatic tumor and its cells are presumed to be similar to those of the original tumor. Thus, if lung cancer metastasizes to the breast, the secondary tumor at the site of the breast consists of abnormal lung cells and not abnormal breast cells. The secondary tumor in the breast is referred to a metastatic lung cancer. Thus, the phrase metastatic cancer refers to a disease in which a subject has or had a primary tumor and has one or more secondary tumors. The phrases non-metastatic cancer or subjects with cancer that is not metastatic refers to diseases in which subjects have a primary tumor but not one or more secondary tumors. For example, metastatic lung cancer refers to a disease in a subject with or with a history of a primary lung tumor and with one or more secondary tumors at a second location or multiple locations, e.g., in the breast.

A cancer that is to be treated can be staged according to the American Joint Committee on Cancer (AJCC) TNM classification system, where the tumor (T) has been assigned a stage of TX, T1, T1mic, T1a, T1b, T1c, T2, T3, T4, T4a, T4b, T4c, or T4d; and where the regional lymph nodes (N) have been assigned a stage of NX, N0, N1, N2, N2a, N2b, N3, N3a, N3b, or N3c; and where distant metastasis (M) can be assigned a stage of MX, M0, or M1. A cancer that is to be treated can be staged according to an American Joint Committee on Cancer (AJCC) classification as Stage I, Stage IIA, Stage IIB, Stage IIIA, Stage IIIB, Stage IIIC, or Stage IV. A cancer that is to be treated can be assigned a grade according to an AJCC classification as Grade GX (e.g., grade cannot be assessed), Grade 1, Grade 2, Grade 3 or Grade 4. A cancer that is to be treated can be staged according to an AJCC pathologic classification (pN) of pNX, pN0, PN0 (I−), PN0 (I+), PN0 (mol−), PN0 (mol+), PN1, PN1 (mi), PN1a, PN1b, PN1c, pN2, pN2a, pN2b, pN3, pN3a, pN3b, or pN3c.

A cancer that is to be treated can include a tumor that has been determined to be less than or equal to about 2 centimeters in diameter. A cancer that is to be treated can include a tumor that has been determined to be from about 2 to about 5 centimeters in diameter. A cancer that is to be treated can include a tumor that has been determined to be greater than or equal to about 3 centimeters in diameter. A cancer that is to be treated can include a tumor that has been determined to be greater than 5 centimeters in diameter. A cancer that is to be treated can be classified by microscopic appearance as well differentiated, moderately differentiated, poorly differentiated, or undifferentiated. A cancer that is to be treated can be classified by microscopic appearance with respect to mitosis count (e.g., amount of cell division) or nuclear pleiomorphism (e.g., change in cells). A cancer that is to be treated can be classified by microscopic appearance as being associated with areas of necrosis (e.g., areas of dying or degenerating cells). A cancer that is to be treated can be classified as having an abnormal karyotype, having an abnormal number of chromosomes, or having one or more chromosomes that are abnormal in appearance. A cancer that is to be treated can be classified as being aneuploid, triploid, tetraploid, or as having an altered ploidy. A cancer that is to be treated can be classified as having a chromosomal translocation, or a deletion or duplication of an entire chromosome, or a region of deletion, duplication or amplification of a portion of a chromosome.

A cancer that is to be treated can be evaluated by DNA cytometry, flow cytometry, or image cytometry. A cancer that is to be treated can be typed as having about 10%, 20%, 30%, 40%, 50%, 60%, 70%, 80%, or 90% of cells in the synthesis stage of cell division (e.g., in S phase of cell division). A cancer that is to be treated can be typed as having a low S-phase fraction or a high S-phase fraction.

An “effective amount” or “a therapeutically effective amount” as provided herein is an amount sufficient to accomplish a stated purpose (e.g. achieve the effect for which it is administered, treat a disease, reduce enzyme activity, increase enzyme activity, reduce transcriptional activity, increase transcriptional activity, reduce one or more symptoms of a disease or condition). An example of an “effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. An “activity decreasing amount,” as used herein, refers to an amount of antagonist (inhibitor) required to decrease the activity of an enzyme or protein (e.g. transcription factor) relative to the absence of the antagonist. An “activity increasing amount,” as used herein, refers to an amount of agonist (activator) required to increase the activity of an enzyme or protein (e.g. transcription factor) relative to the absence of the agonist. A “function disrupting amount,” as used herein, refers to the amount of antagonist (inhibitor) required to disrupt the function of an enzyme or protein (e.g. transcription factor) relative to the absence of the antagonist. A “function increasing amount,” as used herein, refers to the amount of agonist (activator) required to increase the function of an enzyme or protein (e.g. transcription factor) relative to the absence of the agonist. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins). In some embodiments, the disease or condition to be treated is cancer.

As used herein, “treating” or “treat” describes the management and care of a patient for the purpose of combating a disease, condition, or disorder and includes the administration of a composition described herein to alleviate the symptoms or complications of a disease, condition or disorder, or to eliminate the disease, condition or disorder. The term “treat” can also include treatment of a cell in vitro or an animal model.

As used herein, the term “alleviate” is meant to describe a process by which the severity of a sign or symptom of a disorder is decreased. Importantly, a sign or symptom can be alleviated without being eliminated. The administration of compositions or pharmaceutical compositions of the invention may or can lead to the elimination of a sign or symptom, however, elimination is not required. Effective dosages should be expected to decrease the severity of a sign or symptom. For instance, a sign or symptom of a disorder such as cancer, which can occur in multiple locations, is alleviated if the severity of the cancer is decreased within at least one of multiple locations.

As used herein, the term “severity” is meant to describe the potential of cancer to transform from a precancerous, or benign, state into a malignant state. Alternatively, or in addition, severity is meant to describe a cancer stage, for example, according to the TNM system (accepted by the International Union Against Cancer (UICC) and the American Joint Committee on Cancer (AJCC)) or by other art-recognized methods. Cancer stage refers to the extent or severity of the cancer, based on factors such as the location of the primary tumor, tumor size, number of tumors, and lymph node involvement (spread of cancer into lymph nodes). Alternatively, or in addition, severity is meant to describe the tumor grade by art-recognized methods (see, National Cancer Institute, www.cancer.gov). Tumor grade is a system used to classify cancer cells in terms of how abnormal they look under a microscope and how quickly the tumor is likely to grow and spread. Many factors are considered when determining tumor grade, including the structure and growth pattern of the cells. The specific factors used to determine tumor grade vary with each type of cancer. Severity also describes a histologic grade, also called differentiation, which refers to how much the tumor cells resemble normal cells of the same tissue type (see, National Cancer Institute, www.cancer.gov). Furthermore, severity describes a nuclear grade, which refers to the size and shape of the nucleus in tumor cells and the percentage of tumor cells that are dividing (see, National Cancer Institute, www.cancer.gov).

Severity can also describe the degree to which a tumor has secreted growth factors, degraded the extracellular matrix, become vascularized, lost adhesion to juxtaposed tissues, or metastasized. Moreover, severity can describe the number of locations to which a primary tumor has metastasized. Finally, severity can include the difficulty of treating tumors of varying types and locations. For example, inoperable tumors, those cancers which have greater access to multiple body systems (hematological and immunological tumors), and those which are the most resistant to traditional treatments are considered most severe. In these situations, prolonging the life expectancy of the subject and/or reducing pain, decreasing the proportion of cancerous cells or restricting cells to one system, and improving cancer stage/tumor grade/histological grade/nuclear grade are considered alleviating a sign or symptom of the cancer.

As used herein the term “symptom” is defined as an indication of disease, illness, injury, or that something is not right in the body. Symptoms are felt or noticed by the individual experiencing the symptom, but may not easily be noticed by others. Others are defined as non-health-care professionals.

As used herein the term “sign” is also defined as an indication that something is not right in the body. But signs are defined as things that can be seen by a doctor, nurse, or other health care professional.

Cancer is a group of diseases that may cause almost any sign or symptom. The signs and symptoms will depend on where the cancer is, the size of the cancer, and how much it affects the nearby organs or structures. If a cancer spreads (metastasizes), then symptoms may appear in different parts of the body. For example, a cancer may also cause symptoms such as fever, fatigue, or weight loss. Pain may be an early symptom with some cancers such as bone cancers or testicular cancer. But most often pain is a symptom of advanced disease. Along with cancers of the skin, some internal cancers can cause skin signs that can be seen. These changes include the skin looking darker (hyperpigmentation), yellow (jaundice), or red (erythema); itching; or excessive hair growth.

Alternatively, or in addition, cancer subtypes present specific signs or symptoms. Changes in bowel habits or bladder function could indicate cancer. Long-term constipation, diarrhea, or a change in the size of the stool may be a sign of colon cancer. Pain with urination, blood in the urine, or a change in bladder function (such as more frequent or less frequent urination) could be related to bladder or prostate cancer.

Changes in skin condition or appearance of a new skin condition could indicate cancer. Skin cancers may bleed and look like sores that do not heal. A long-lasting sore in the mouth could be an oral cancer, especially in patients who smoke, chew tobacco, or frequently drink alcohol. Sores on the penis or vagina may either be signs of infection or an early cancer.

Unusual bleeding or discharge could indicate cancer. Unusual bleeding can happen in either early or advanced cancer. Blood in the sputum (phlegm) may be a sign of lung cancer. Blood in the stool (or a dark or black stool) could be a sign of colon or rectal cancer. Cancer of the cervix or the endometrium (lining of the uterus) can cause vaginal bleeding. Blood in the urine may be a sign of bladder or kidney cancer. A bloody discharge from the nipple may be a sign of breast cancer.

A thickening or lump in the breast or in other parts of the body could indicate the presence of a cancer. Many cancers can be felt through the skin, mostly in the breast, testicle, lymph nodes (glands), and the soft tissues of the body. A lump or thickening may be an early or late sign of cancer. Any lump or thickening could be indicative of cancer, especially if the formation is new or has grown in size.

Indigestion or trouble swallowing could indicate cancer. While these symptoms commonly have other causes, indigestion or swallowing problems may be a sign of cancer of the esophagus, stomach, or pharynx (throat).

Recent changes in a wart or mole could be indicative of cancer. Any wart, mole, or freckle that changes in color, size, or shape, or loses its definite borders indicates the potential development of cancer. For example, the skin lesion may be a melanoma.

A persistent cough or hoarseness could be indicative of cancer. A cough that does not go away may be a sign of lung cancer. Hoarseness can be a sign of cancer of the larynx (voice box) or thyroid.

While the signs and symptoms listed above are the more common ones seen with cancer, there are many others that are less common and are not listed here.

Treating cancer may result in or can result in a reduction in size of a tumor. A reduction in size of a tumor may also be referred to as “tumor regression”. Preferably, after treatment, tumor size would be reduced by about 5% or greater relative to its size prior to treatment; more preferably, tumor size is reduced by about 10% or greater; more preferably, reduced by about 20% or greater; more preferably, reduced by about 30% or greater; more preferably, reduced by about 40% or greater; even more preferably, reduced by about 50% or greater; and most preferably, reduced by greater than about 75% or greater. Size of a tumor may be measured by any reproducible means of measurement. The size of a tumor may be measured as a diameter of the tumor.

Treating cancer may result in or can result in a reduction in tumor volume. Preferably, after treatment, tumor volume would be reduced by about 5% or greater relative to its size prior to treatment; more preferably, tumor volume is reduced by about 10% or greater; more preferably, reduced by about 20% or greater; more preferably, reduced by about 30% or greater; more preferably, reduced by about 40% or greater; even more preferably, reduced by about 50% or greater; and most preferably, reduced by greater than about 75% or greater. Tumor volume may be measured by any reproducible means of measurement.

Treating cancer may result in or can result in a decrease in number of tumors. Preferably, after treatment, tumor number would be reduced by about 5% or greater relative to number prior to treatment; more preferably, tumor number is reduced by about 10% or greater; more preferably, reduced by about 20% or greater; more preferably, reduced by about 30% or greater; more preferably, reduced by about 40% or greater; even more preferably, reduced by about 50% or greater; and most preferably, reduced by greater than about 75%. Number of tumors may be measured by any reproducible means of measurement. The number of tumors may be measured by counting tumors visible to the naked eye or at a specified magnification. Preferably, the specified magnification is 2×, 3×, 4×, 5×, 10×, or 50×.

Treating cancer may result in or can result in a decrease in number of metastatic lesions in other tissues or organs distant from the primary tumor site. Preferably, after treatment, the number of metastatic lesions would be reduced by about 5% or greater relative to number prior to treatment; more preferably, the number of metastatic lesions is reduced by about 10% or greater; more preferably, reduced by about 20% or greater; more preferably, reduced by about 30% or greater; more preferably, reduced by about 40% or greater; even more preferably, reduced by about 50% or greater; and most preferably, reduced by greater than about 75%. The number of metastatic lesions may be measured by any reproducible means of measurement. The number of metastatic lesions may be measured by counting metastatic lesions visible to the naked eye or at a specified magnification. Preferably, the specified magnification is 2×, 3×, 4×, 5×, 10×, or 50×.

Treating cancer may result in or can result in an increase in average survival time of a population of treated subjects in comparison to a population receiving carrier alone. Preferably, the average survival time would be increased by more than 30 days; more preferably, by more than 60 days; more preferably, by more than 90 days; and most preferably, by more than 120 days. An increase in average survival time of a population may be measured by any reproducible means. An increase in average survival time of a population may be measured, for example, by calculating for a population the average length of survival following initiation of treatment with an active composition. An increase in average survival time of a population may also be measured, for example, by calculating for a population the average length of survival following completion of a first round of treatment with an active composition.

Treating cancer may result in or can result in an increase in average survival time of a population of treated subjects in comparison to a population of untreated subjects. Preferably, the average survival time would be increased by more than 30 days; more preferably, by more than 60 days; more preferably, by more than 90 days; and most preferably, by more than 120 days. An increase in average survival time of a population may be measured by any reproducible means. An increase in average survival time of a population may be measured, for example, by calculating for a population the average length of survival following initiation of treatment with an active composition. An increase in average survival time of a population may also be measured, for example, by calculating for a population the average length of survival following completion of a first round of treatment with an active composition.

Treating cancer may result in or can result in increase in average survival time of a population of treated subjects in comparison to a population receiving monotherapy with a drug that is not a composition described herein. Preferably, the average survival time would be increased by more than 30 days; more preferably, by more than 60 days; more preferably, by more than 90 days; and most preferably, by more than 120 days. An increase in average survival time of a population may be measured by any reproducible means. An increase in average survival time of a population may be measured, for example, by calculating for a population the average length of survival following initiation of treatment with an active composition. An increase in average survival time of a population may also be measured, for example, by calculating for a population the average length of survival following completion of a first round of treatment with an active composition.

Treating cancer may result in or can result in a decrease in the mortality rate of a population of treated subjects in comparison to a population receiving carrier alone. Treating cancer may result in or can result in a decrease in the mortality rate of a population of treated subjects in comparison to an untreated population. Treating cancer may result in or can result in a decrease in the mortality rate of a population of treated subjects in comparison to a population receiving monotherapy with a drug that is not a composition described herein, or a pharmaceutically acceptable salt, prodrug, metabolite, analog or derivative thereof. Preferably, the mortality rate would be decreased by more than 2%; more preferably, by more than 5%; more preferably, by more than 10%; and most preferably, by more than 25%. A decrease in the mortality rate of a population of treated subjects may be measured by any reproducible means. A decrease in the mortality rate of a population may be measured, for example, by calculating for a population the average number of disease-related deaths per unit time following initiation of treatment with an active composition. A decrease in the mortality rate of a population may also be measured, for example, by calculating for a population the average number of disease-related deaths per unit time following completion of a first round of treatment with an active composition.

Treating cancer may result in or can result in a decrease in tumor growth rate. Preferably, after treatment, tumor growth rate would be reduced by at least 5% relative to number prior to treatment; more preferably, tumor growth rate would be reduced by at least 10%; more preferably, reduced by at least 20%; more preferably, reduced by at least 30%; more preferably, reduced by at least 40%; more preferably, reduced by at least 50%; even more preferably, reduced by at least 50%; and most preferably, reduced by at least 75%. Tumor growth rate may be measured by any reproducible means of measurement. Tumor growth rate can be measured according to a change in tumor diameter per unit time.

Treating cancer may result in or can result in a decrease in tumor regrowth. Preferably, after treatment, tumor regrowth would be less than 5%; more preferably, tumor regrowth would be less than 10%; more preferably, less than 20%; more preferably, less than 30%; more preferably, less than 40%; more preferably, less than 50%; even more preferably, less than 50%; and most preferably, less than 75%. Tumor regrowth may be measured by any reproducible means of measurement. Tumor regrowth is measured, for example, by measuring an increase in the diameter of a tumor after a prior tumor shrinkage that followed treatment. A decrease in tumor regrowth is indicated by failure of tumors to reoccur after treatment has stopped.

“Contacting” is used in accordance with its plain ordinary meaning and refers to the process of allowing at least two distinct species (e.g. chemical compounds including biomolecules or cells) to become sufficiently proximal to react, interact or physically touch. It should be appreciated, however, that the resulting reaction product can be produced directly from a reaction between the added reagents or from an intermediate from one or more of the added reagents which can be produced in the reaction mixture. In some embodiments contacting includes, for example, allowing a ribonucleic acid as described herein to interact with an endonuclease and an enhancer element.

A “control” sample or value refers to a sample that serves as a reference, usually a known reference, for comparison to a test sample. For example, a test sample can be taken from a test condition, e.g., in the presence of a test compound, and compared to samples from known conditions, e.g., in the absence of the test compound (negative control), or in the presence of a known compound (positive control). A control can also represent an average value gathered from a number of tests or results. One of skill in the art will recognize that controls can be designed for assessment of any number of parameters. For example, a control can be devised to compare therapeutic benefit based on pharmacological data (e.g., half-life) or therapeutic measures (e.g., comparison of side effects). One of skill in the art will understand which standard controls are most appropriate in a given situation and be able to analyze data based on comparisons to standard control values. Standard controls are also valuable for determining the significance (e.g. statistical significance) of data. For example, if values for a given parameter are widely variant in standard controls, variation in test samples will not be considered as significant.

As defined herein, the term “inhibition”, “inhibit”, “inhibiting” or the like means negatively affecting (e.g. decreasing) the activity or function of a biomolecule or biological system relative to the activity or function in the absence of the inhibitor. In some embodiments inhibition refers to reduction of a disease or symptoms of disease.

As defined herein, the term “activation”, “activate”, “activating” or the like in reference to a activator (e.g. agonist) interaction means positively affecting (e.g. increasing) the activity or function of a biomolecule or biological system relative to the activity or function in the absence of the activator

As used herein, the term “administering” means oral administration, administration as a suppository, topical contact, intravenous, parenteral, intraperitoneal, intramuscular, intralesional, intrathecal, intracranial, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). In some embodiments, administration includes direct administration to a tumor. Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. By “co-administer” it is meant that a composition described herein is administered at the same time, just prior to, or just after the administration of one or more additional therapies (e.g. anti-cancer agent or chemotherapeutic). The composition of the invention can be administered alone or can be co-administered to the patient. Co-administration is meant to include simultaneous or sequential administration of the compound individually or in combination (more than one compound or agent). Thus, the preparations can also be combined, when desired, with other active substances (e.g. to reduce metabolic degradation). The compositions described herein can be delivered by transdermally, by a topical route, formulated as applicator sticks, solutions, suspensions, emulsions, gels, creams, ointments, pastes, jellies, paints, powders, and aerosols. Oral preparations include tablets, pills, powder, dragees, capsules, liquids, lozenges, cachets, gels, syrups, slurries, suspensions, etc., suitable for ingestion by the patient. Solid form preparations include powders, tablets, pills, capsules, cachets, suppositories, and dispersible granules. Liquid form preparations include solutions, suspensions, and emulsions, for example, water or water/propylene glycol solutions. The compositions described herein may additionally include components to provide sustained release and/or comfort. Such components include high molecular weight, anionic mucomimetic polymers, gelling polysaccharides and finely-divided drug carrier substrates. These components are discussed in greater detail in U.S. Pat. Nos. 4,911,920; 5,403,841; 5,212,162; and 4,861,760. The entire contents of these patents are incorporated herein by reference in their entirety for all purposes. The compositions described herein can also be delivered as microspheres for slow release in the body. For example, microspheres can be administered via intradermal injection of drug-containing microspheres, which slowly release subcutaneously (see Rao, J. Biomater Sci. Polym. Ed. 7:623-645, 1995; as biodegradable and injectable gel formulations (see, e.g., Gao Pharm. Res. 12:857-863, 1995); or, as microspheres for oral administration (see, e.g., Eyles, J. Pharm. Pharmacol. 49:669-674, 1997). In another embodiment, the formulations of the compositions described herein can be delivered by the use of liposomes which fuse with the cellular membrane or are endocytosed, i.e., by employing receptor ligands attached to the liposome, that bind to surface membrane protein receptors of the cell resulting in endocytosis. By using liposomes, particularly where the liposome surface carries receptor ligands specific for target cells, or are otherwise preferentially directed to a specific organ, one can focus the delivery of the compositions described herein into the target cells in vivo. (See, e.g., Al-Muhammed, J Microencapsul. 13:293-306, 1996; Chonn, Curr. Opin. Biotechnol. 6:698-708, 1995; Ostro, Am. J. Hosp. Pharm. 46:1576-1587, 1989). The compositions described herein can also be delivered as nanoparticles.

The dosage and frequency (single or multiple doses) administered to a mammal can vary depending upon a variety of factors, for example, whether the mammal suffers from another disease, and its route of administration; size, age, sex, health, body weight, body mass index, and diet of the recipient; nature and extent of symptoms of the disease being treated (e.g. symptoms of cancer (e.g. prostate cancer, castration-resistant prostate cancer, breast cancer, triple negative breast cancer, glioblastoma, ovarian cancer, lung cancer, squamous cell carcinoma (e.g head, neck, or esophagus), colorectal cancer, leukemia, acute myeloid leukemia, lymphoma, B cell lymphoma, or multiple myeloma)), kind of concurrent treatment, complications from the disease being treated or other health-related problems. Other therapeutic regimens or agents can be used in conjunction with the methods and compounds of Applicants' invention. Adjustment and manipulation of established dosages (e.g., frequency and duration) are well within the ability of those skilled in the art.

For compounds or compositions described herein, the therapeutically effective amount can be initially determined from cell culture assays. Target concentrations will be those concentrations of active compound(s) that are capable of achieving the methods described herein, as measured using the methods described herein or known in the art.

As is well known in the art, therapeutically effective amounts for use in humans can also be determined from animal models. For example, a dose for humans can be formulated to achieve a concentration that has been found to be effective in animals. The dosage in humans can be adjusted by monitoring compounds effectiveness and adjusting the dosage upwards or downwards, as described above. Adjusting the dose to achieve maximal efficacy in humans based on the methods described above and other methods is well within the capabilities of the ordinarily skilled artisan.

Dosages may be varied depending upon the requirements of the patient and the compound being employed. The dose administered to a patient should be sufficient to effect a beneficial therapeutic response in the patient over time. The size of the dose also will be determined by the existence, nature, and extent of any adverse side-effects. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached.

Dosage amounts and intervals can be adjusted individually to provide levels of the administered compound effective for the particular clinical indication being treated. This will provide a therapeutic regimen that is commensurate with the severity of the individual's disease state.

Utilizing the teachings provided herein, an effective prophylactic or therapeutic treatment regimen can be planned that does not cause substantial toxicity and yet is effective to treat the clinical symptoms demonstrated by the particular patient. This planning should involve the careful choice of active compound by considering factors such as compound potency, relative bioavailability, patient body weight, presence and severity of adverse side effects, preferred mode of administration and the toxicity profile of the selected agent.

As used herein, the term “about” means a range of values including the specified value, which a person of ordinary skill in the art would consider reasonably similar to the specified value. In some embodiments, the term “about” means within a standard deviation using measurements generally acceptable in the art. In some embodiments, about means a range extending to +/−10% of the specified value. In some embodiments, about means the specified value.

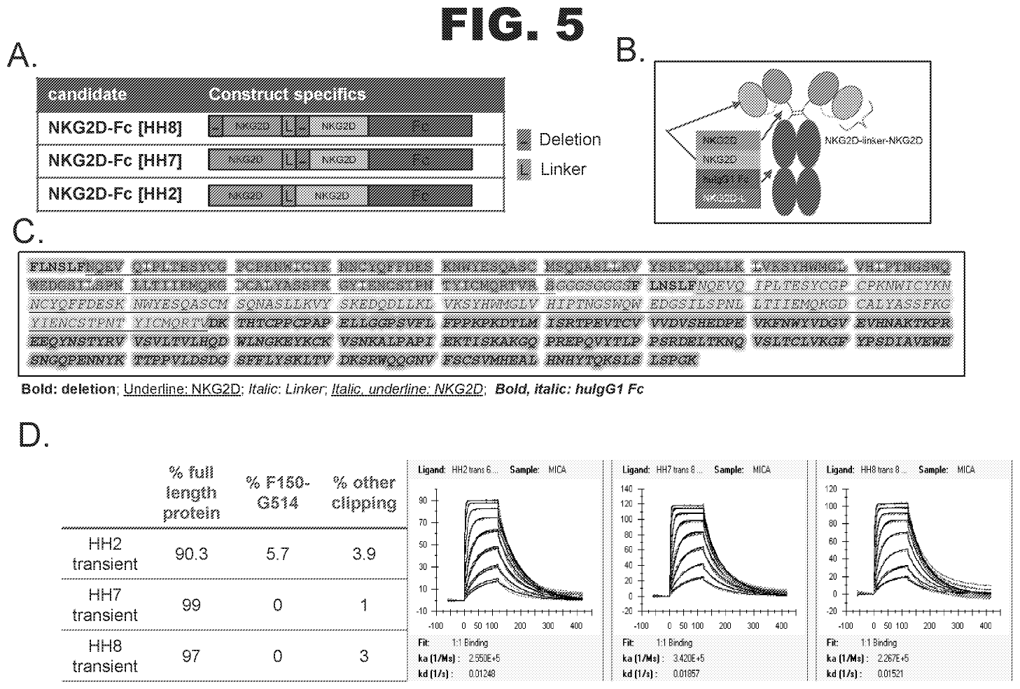

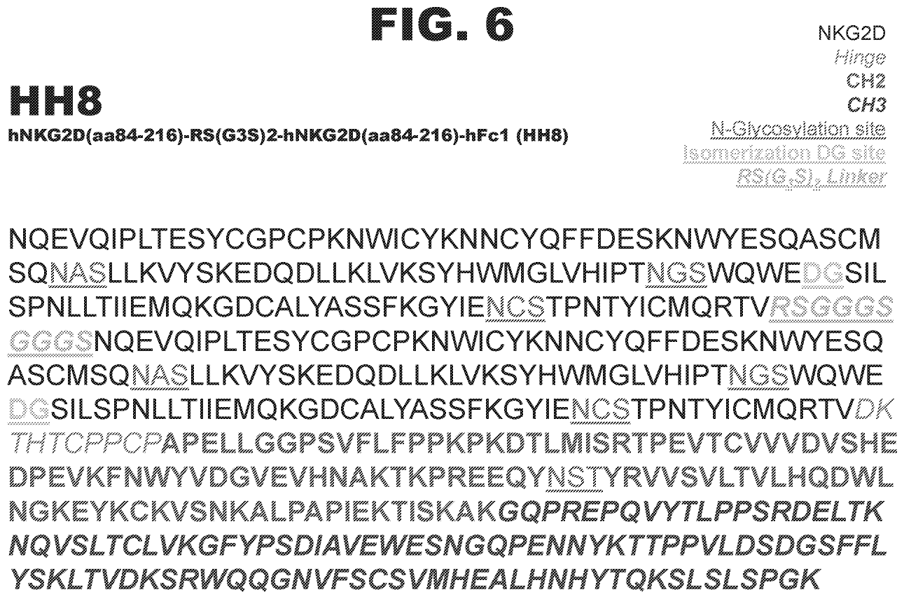

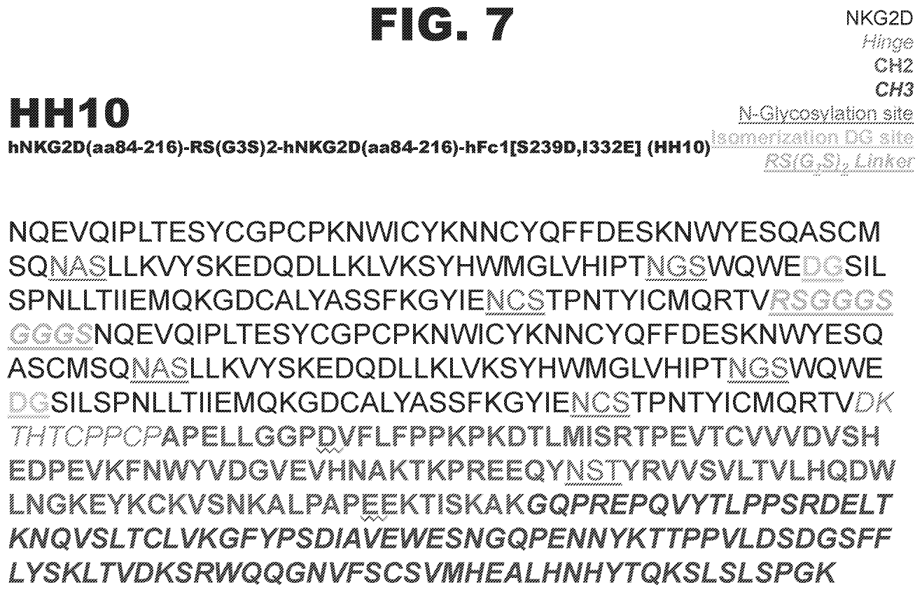

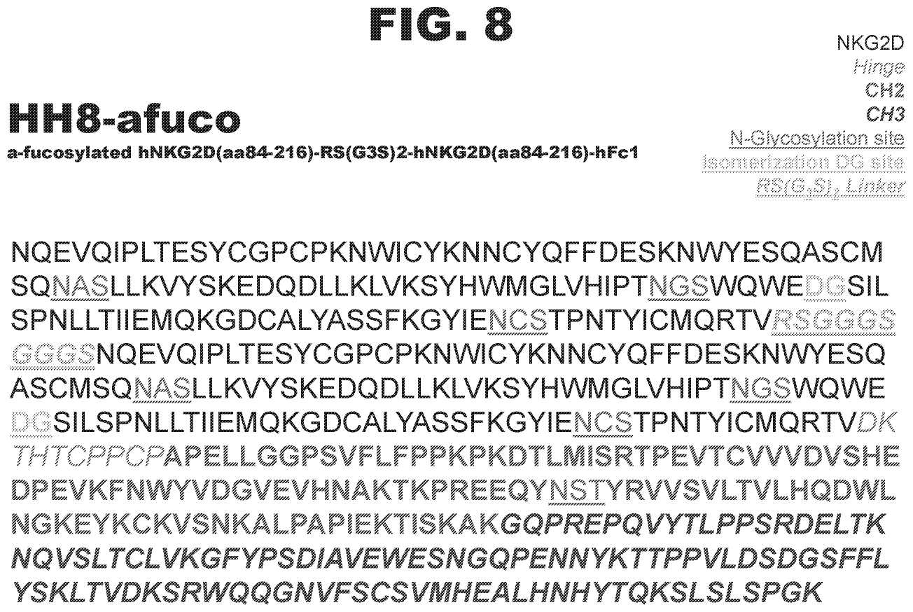

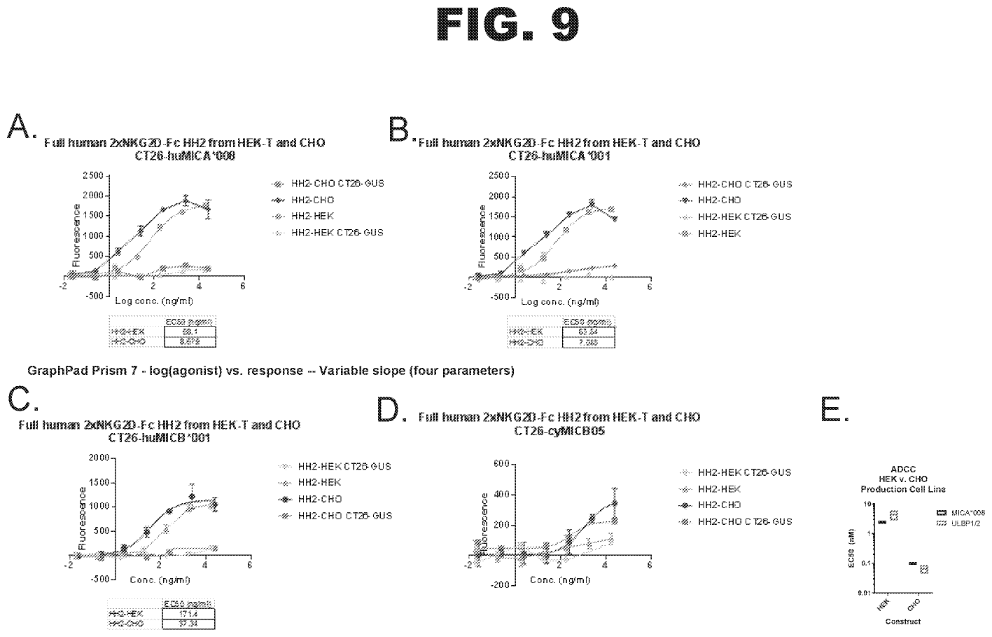

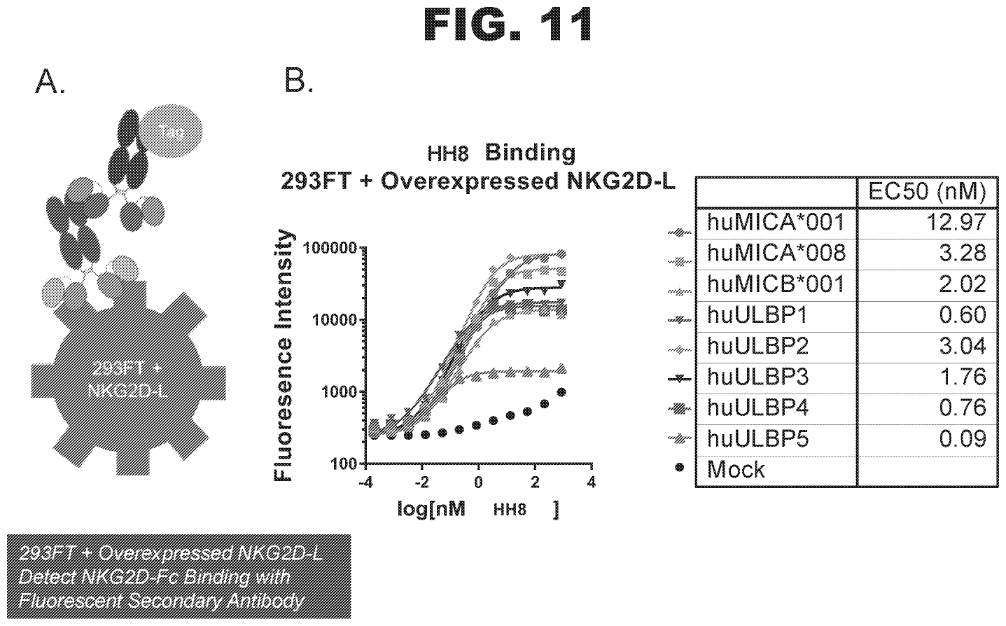

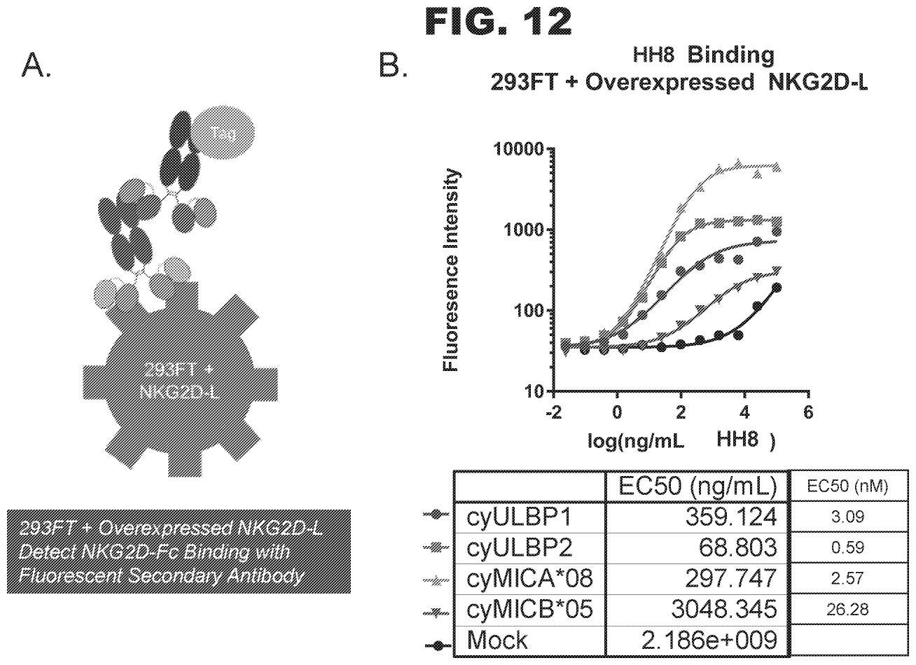

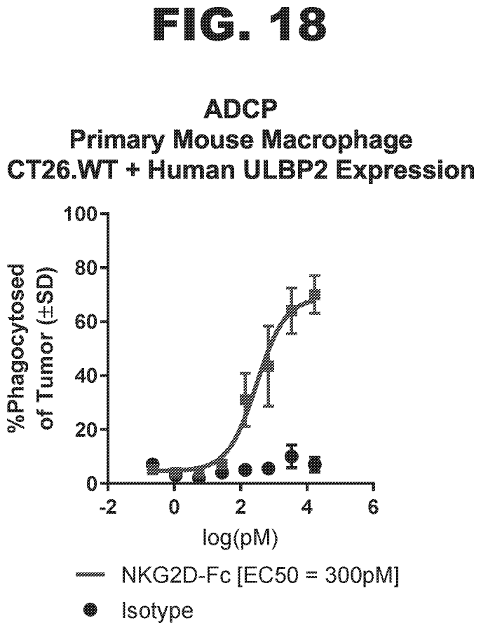

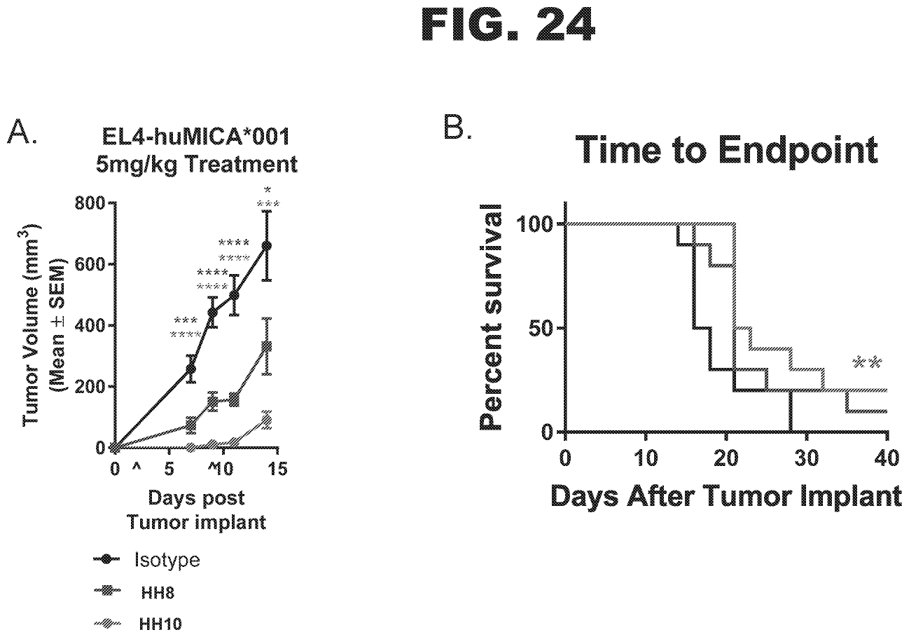

As described herein, the objective technical problem to be solved in light of the prior art is the provision of improved compositions and methods for preventing and treating NKG2D ligand-expressing (NKG2D-L+) diseases and conditions. In some embodiments, such NKG2D ligand-expressing diseases and conditions include cancer and autoimmune diseases. Provided herein is a solution to this problem for the first time. Clipping of recombinant proteins is a major issue in cell cultures. Proteins produced in bacterial and mammalian host cells by recombinant DNA technology often are fully or partially degraded in culture. This poses a major challenge to biotech industry, where efficiency and high productivity are often required at lower costs. Hence, mitigating the risk of clipping or minimizing the clipping level is critical for recombinant protein-based therapeutics. In some embodiments, the compositions described herein have demonstrated unexpected, excellent and higher stability (e.g., lower clipping) inside the cells (see, e.g., FIGS. 5A-5C) compared to the prior art construct(s). In some embodiments, the combination of the compositions described herein and the use of a modified CHO cell line (e.g., CHO cells comprising an inactivated or knock-out matriptase gene (CHO-Mako)) mitigates the risk of clipping to a minimum level. In addition, the compositions described herein have shown superior in vitro and in vivo therapeutic effects. In some embodiments, the compositions described herein have demonstrated excellent effect in improving ADCC activity (see, e.g., FIGS. 9A-9E). In some embodiments, the compositions described herein have shown excellent binding specificities to all NKG2D ligands, with a single digit nM EC50 (see, e.g., FIGS. 11A-11B, FIGS. 12A-12B). In some embodiments, the compositions described herein have demonstrated superior in vivo efficacies in killing of NKG2D-L+ tumors (see, e.g., FIGS. 18, 24A-24B). Equally importantly, the compositions described herein have demonstrated excellent developability with low risk and medium technical effort based on their developability assessments.

Compositions

Accordingly, provided herein is a dimeric protein that includes two monomers, where each monomer includes (1) a first NKG2D peptide or variant thereof; (2) a second NKG2D peptide or variant thereof; (3) a first peptide linker connecting said first NKG2D peptide or variant thereof and said second NKG2D peptide or variant thereof; and (4) a fragment crystallizable region (Fc region) of an immunoglobulin (Ig).

In some embodiments, the two monomers have identical amino acid sequences.

In some embodiments, the two monomers have different amino acid sequences.

In some embodiments, the first NKG2D peptide or variant thereof and the second NKG2D peptide or variant thereof have identical amino acid sequence.

In some embodiments, the first NKG2D peptide or variant thereof and the second NKG2D peptide or variant thereof have different amino acid sequences.

In some embodiments, each NKG2D peptide or variant thereof of the dimeric protein comprises the extracellular portion of the NKG2D sequence, e.g., amino acid residues 78-216 of human NKG2D; 78-232, 94-232 or 92-232 of murine NKG2D. In some embodiments, a dimeric protein comprises a portion of the extracellular domain of NKG2D. In some embodiments, the extracellular domain of NKG2D of the dimeric protein may be shortened at the N-terminus, at the C-terminus, or both. In some embodiments, the N-terminus of the extracellular domain used to generate a dimeric protein may be shortened by one or more amino acid residues, e.g., 1, 2, 3, 4, 5, 6, 7, 8, 9, 10, 11, 12, 13, 14, 15, 16, 17, 18, 19, 20, about 30, about 40, about 50, about 60, and so forth, relative to the full extracellular portion of the polypeptide. In some embodiments, the C-terminus of the extracellular domain used to generate a dimeric protein may be shortened by one or more amino acid residues, e.g., 1, 2, 3, 4, 5, 6, 7, 8, 9, 10, 11, 12, 13, 14, 15, 16, 17, 18, 19, 20, about 30, about 40, about 50, about 60, and so forth, relative to the full extracellular portion of the polypeptide. Using a human NKG2D as an example, the dimeric protein may contain a fragment of the extracellular domain of human NKG2D, where the N-terminus of the domain begins at amino acid residue 79, 80, 81, 90, 91, 92, 93, 94, 95, 96, 97, 98, 99, 100, about 110, about 120, about 130, about 140 or about 150. In some embodiments, the dimeric protein may contain a fragment of the extracellular domain of NKG2D, where the C-terminus of the domain ends at amino acid residue 231, 230, 229, 228, 227, 226, 225, 224, 223, 222, 221, 220, 219, 218, 217, 216, 215, 214, 213, 212, 211, 210, 209, 208, 207, 206, 205, and so forth. Such deletions at each end of the extracellular domain of the NKG2D sequence may be combined.

In some embodiments, the first NKG2D peptide or variant thereof comprises, or alternatively, consists essentially of or consists of, comprises, or alternatively, consists essentially of or consists of, an amino acid sequence having at least 85% (e.g., 85%, 86%, 87%, 88%, 89%, 90%, 91%, 92%, 93%, 94%, 95%, 96%, 97%, 98%, 99%, 100%) identity to SEQ ID NO: 1, 2, 3, or 4.

In some embodiments, the first NKG2D peptide or variant thereof comprises, or alternatively, consists essentially of or consists of, comprises, or alternatively, consists essentially of or consists of, an amino acid sequence having at least 85% (e.g., 85%, 86%, 87%, 88%, 89%, 90%, 91%, 92%, 93%, 94%, 95%, 96%, 97%, 98%, 99%, 100%) identity to SEQ ID NO: 1.

In some embodiments, the first NKG2D peptide or variant thereof comprises, or alternatively, consists essentially of or consists of, comprises, or alternatively, consists essentially of or consists of, an amino acid sequence having at least 85% (e.g., 85%, 86%, 87%, 88%, 89%, 90%, 91%, 92%, 93%, 94%, 95%, 96%, 97%, 98%, 99%, 100%) identity to SEQ ID NO: 2.

In some embodiments, the first NKG2D peptide or variant thereof comprises, or alternatively, consists essentially of or consists of, comprises, or alternatively, consists essentially of or consists of, an amino acid sequence having at least 85% (e.g., 85%, 86%, 87%, 88%, 89%, 90%, 91%, 92%, 93%, 94%, 95%, 96%, 97%, 98%, 99%, 100%) identity to SEQ ID NO: 3.

In some embodiments, the first NKG2D peptide or variant thereof comprises, or alternatively, consists essentially of or consists of, an amino acid sequence having at least 85% (e.g., 85%, 86%, 87%, 88%, 89%, 90%, 91%, 92%, 93%, 94%, 95%, 96%, 97%, 98%, 99%, 100%) identity to SEQ ID NO: 4.

In some embodiments, the first NKG2D peptide or variant thereof comprises, or alternatively, consists essentially of or consists of, an amino acid sequence of SEQ ID NO: 1, 2, 3, or 4.

In some embodiments, the first NKG2D peptide or variant thereof comprises, or alternatively, consists essentially of or consists of, an amino acid sequence of SEQ ID NO: 1.

In some embodiments, the first NKG2D peptide or variant thereof comprises, or alternatively, consists essentially of or consists of, an amino acid sequence of SEQ ID NO: 2.

In some embodiments, the first NKG2D peptide or variant thereof comprises, or alternatively, consists essentially of or consists of, an amino acid sequence of SEQ ID NO: 3.

In some embodiments, the first NKG2D peptide or variant thereof comprises, or alternatively, consists essentially of or consists of, an amino acid sequence of SEQ ID NO: 4.

In some embodiments, the second NKG2D peptide or variant thereof comprises, or alternatively, consists essentially of or consists of, an amino acid sequence having at least 85% (e.g., 85%, 86%, 87%, 88%, 89%, 90%, 91%, 92%, 93%, 94%, 95%, 96%, 97%, 98%, 99%, 100%) identity to SEQ ID NO: 1, 2, 3 or 4.

In some embodiments, the second NKG2D peptide or variant thereof comprises, or alternatively, consists essentially of or consists of, an amino acid sequence having at least 85% (e.g., 85%, 86%, 87%, 88%, 89%, 90%, 91%, 92%, 93%, 94%, 95%, 96%, 97%, 98%, 99%, 100%) identity to SEQ ID NO: 1.

In some embodiments, the second NKG2D peptide or variant thereof comprises, or alternatively, consists essentially of or consists of, an amino acid sequence having at least 85% (e.g., 85%, 86%, 87%, 88%, 89%, 90%, 91%, 92%, 93%, 94%, 95%, 96%, 97%, 98%, 99%, 100%) identity to SEQ ID NO: 2.

In some embodiments, the second NKG2D peptide or variant thereof comprises, or alternatively, consists essentially of or consists of, an amino acid sequence having at least 85% (e.g., 85%, 86%, 87%, 88%, 89%, 90%, 91%, 92%, 93%, 94%, 95%, 96%, 97%, 98%, 99%, 100%) identity to SEQ ID NO: 3.

In some embodiments, the second NKG2D peptide or variant thereof comprises, or alternatively, consists essentially of or consists of, an amino acid sequence having at least 85% (e.g., 85%, 86%, 87%, 88%, 89%, 90%, 91%, 92%, 93%, 94%, 95%, 96%, 97%, 98%, 99%, 100%) identity to SEQ ID NO: 4.

In some embodiments, the second NKG2D peptide or variant thereof comprises, or alternatively, consists essentially of or consists of, an amino acid sequence of SEQ ID NO: 1, 2, 3, or 4.

In some embodiments, the second NKG2D peptide or variant thereof comprises, or alternatively, consists essentially of or consists of, an amino acid sequence of SEQ ID NO: 1.

In some embodiments, the second NKG2D peptide or variant thereof comprises, or alternatively, consists essentially of or consists of, an amino acid sequence of SEQ ID NO: 2.

In some embodiments, the second NKG2D peptide or variant thereof comprises, or alternatively, consists essentially of or consists of, an amino acid sequence of SEQ ID NO: 3.

In some embodiments, the second NKG2D peptide or variant thereof comprises, or alternatively, consists essentially of or consists of, an amino acid sequence of SEQ ID NO: 4.

In some embodiments, the Fc region comprises an Fc region of human immunoglobulin G (IgG). In some embodiments, the Fc region comprises an Fc region of human IgG1. In some embodiments, the Fc region comprises an Fc region of human IgG2. In some embodiments, the Fc region comprises an Fc region of human IgG3. In some embodiments, the Fc region comprises an Fc region of human IgG4. In some embodiments, the Fc region comprises an Fc region of mouse IgG2a. In some embodiments, the Fc region comprises an Fc region of human IgM. In some embodiments, the Fc region comprises an Fc region of human IgA1. In some embodiments, the Fc region comprises an Fc region of human IgA2. In some embodiments, the Fc region comprises an Fc region of human IgD. In some embodiments, the Fc region comprises an Fc region of human IgE.

The Fc region of immunoglobulins plays a significant role in mediating immune defense. FcyRs are widely expressed as transmembrane glycoproteins on a number of cell types, including macrophages, NK cells, dendritic cells, B cells, neutrophils and mast cells. Fc-mediated activities include recruitment of effector cells via Fc-FcyR interactions. There are two classes of Fc receptors that can be distinguished functionally: the activating Fc receptor class and the inhibitory Fc receptor class. Activating Fc receptors include human FcyRIA, FcyRIIA and FcyRIIIA, as well as their murine orthologues, i.e., FcyRI, FcyRIII FcyRIV. Activating FcyRs mediate ADCC and ADCP, induce endocytosis of immune complexes leading to antigen presentation, and contribute to the production and release of cytokines and proinflammatory factors. For general review of the IgG structure and mechanisms of action, see Liu et al. (2008; Immunological Reviews, 222: 9-27). In some embodiments, the Fc portion of dimeric protein described herein is a domain that binds an activating Fc receptor, and in some embodiments an activating Fc Ig domain and includes the hinge region that allows for dimerization.

The Fc portion of the dimeric protein useful for this disclosure can be readily adapted to render it species-specific. In some embodiments for use in a murine system, e.g., cells derived from a mouse, the Fc fragment used to generate dimeric protein is that of a murine origin. In some embodiments, an Fc fragment of the murine IgG2a is used. In some embodiments for use in a human subject, the Fc fragment used to generate dimeric protein is that of a human origin. In some embodiments, each monomer of the dimeric protein comprises an activating Fc Ig domain. In some embodiments, the Fc comprises a fragment crystallizable region (Fc) of a human immunoglobulin (IgG). In some embodiments, the human immunoglobulin is IgG1.

In some embodiments, a mutant or an allotype of an Fc fragment is used in the dimeric protein described herein. A number of useful mutations within an Fc domain have been described, which can affect the interaction of an Fc and its receptor, the effector function of the Fc, as well as the half-life of the Fc-containing molecule. These include specific amino acid substitutions and/or modifications to carbohydrate moieties in the Fc. For review, see, for example, Liu et al., 2008, Immunological Reviews, 222:9-27; Nimmerjahn & Ravetch, 2007, Curr. Opin. Immunol., 19(2): 239-45. In some embodiments, a mutant Fc used herein exhibits a higher affinity for FcyRIIIA but not for FcyRIIB, thereby promoting ADCC. In some embodiments, a mutant Fc used herein exhibits a higher affinity for FcyRIIIA and a reduced binding to FcyRIIB In some embodiments, a mutant Fc used herein includes mutations Ser298Ala/Glu333Ala/Lys334Ala. In some embodiments, a mutant Fc used herein includes mutations Ser239Asp/Ile332Glu, which is expected to exert improved ADCC. Other mutations for enhancing ADCC include, without limitation, Ser239Asp/Ala330Leu/Ile332Glu, Ser239Asp/Ser298Ala/Ile332Ala, and Phe243Leu/Arg292Pro/Tyr300LeuNa1305Ile/Pro396Leu. In some embodiments, a mutant Fc used herein is “Fc silent,” i.e. has minimal interaction with effector cells. Silenced effector functions can be obtained by mutation in the Fc region of the antibodies and have been described in the art: LALA and N297A (Strohl, W., 2009, Curr. Opin. Biotechnol. vol. 20(6):685-691); and D265A (Baudino et al., 2008, J. Immunol. 181: 6664-69) see also Heusser et al., WO2012065950. Examples of Fc silencing mutations include the LALA mutant comprising L234A and L235A mutation in the IgG1 Fc amino acid sequence, DAPA (D265A, P329A) (see, e.g., U.S. Pat. No. 6,737,056), N297A, DANAPA (D265A, N297A, and P329A), and/or LALADANAPS (L234A, L235A, D265A, N297A and P331S).

In some embodiments, the Fc region comprises, or alternatively, consists essentially of or consists of, an amino acid sequence having at least 85% (e.g., 85%, 86%, 87%, 88%, 89%, 90%, 91%, 92%, 93%, 94%, 95%, 96%, 97%, 98%, 99%, 100%) identity to SEQ ID NO: 5, 6, 63, 64, 65, or 68.

In some embodiments, the Fc region comprises, or alternatively, consists essentially of or consists of, an amino acid sequence having at least 85% (e.g., 85%, 86%, 87%, 88%, 89%, 90%, 91%, 92%, 93%, 94%, 95%, 96%, 97%, 98%, 99%, 100%) identity to SEQ ID NO: 5.

In some embodiments, the Fc region comprises, or alternatively, consists essentially of or consists of, an amino acid sequence having at least 85% (e.g., 85%, 86%, 87%, 88%, 89%, 90%, 91%, 92%, 93%, 94%, 95%, 96%, 97%, 98%, 99%, 100%) identity to SEQ ID NO: 6.

In some embodiments, the Fc region comprises, or alternatively, consists essentially of or consists of, an amino acid sequence having at least 85% (e.g., 85%, 86%, 87%, 88%, 89%, 90%, 91%, 92%, 93%, 94%, 95%, 96%, 97%, 98%, 99%, 100%) identity to SEQ ID NO: 63.

In some embodiments, the Fc region comprises, or alternatively, consists essentially of or consists of, an amino acid sequence having at least 85% (e.g., 85%, 86%, 87%, 88%, 89%, 90%, 91%, 92%, 93%, 94%, 95%, 96%, 97%, 98%, 99%, 100%) identity to SEQ ID NO: 64.

In some embodiments, the Fc region comprises, or alternatively, consists essentially of or consists of, an amino acid sequence having at least 85% (e.g., 85%, 86%, 87%, 88%, 89%, 90%, 91%, 92%, 93%, 94%, 95%, 96%, 97%, 98%, 99%, 100%) identity to SEQ ID NO: 65.

In some embodiments, the Fc region comprises, or alternatively, consists essentially of or consists of, an amino acid sequence having at least 85% (e.g., 85%, 86%, 87%, 88%, 89%, 90%, 91%, 92%, 93%, 94%, 95%, 96%, 97%, 98%, 99%, 100%) identity to SEQ ID NO: 68.

In some embodiments, the Fc region comprises, or alternatively, consists essentially of or consists of, an amino acid sequence of SEQ ID NO: 5, 6, 63, 64, 65, or 68.

In some embodiments, the Fc region comprises, or alternatively, consists essentially of or consists of, an amino acid sequence of SEQ ID NO: 5.

In some embodiments, the Fc region comprises, or alternatively, consists essentially of or consists of, an amino acid sequence of SEQ ID NO: 6.

In some embodiments, the Fc region comprises, or alternatively, consists essentially of or consists of, an amino acid sequence of SEQ ID NO: 63.

In some embodiments, the Fc region comprises, or alternatively, consists essentially of or consists of, an amino acid sequence of SEQ ID NO: 64.

In some embodiments, the Fc region comprises, or alternatively, consists essentially of or consists of, an amino acid sequence of SEQ ID NO: 65.

In some embodiments, the Fc region comprises, or alternatively, consists essentially of or consists of, an amino acid sequence of SEQ ID NO: 68.

In some embodiments, one or both of the two monomers include, from N-terminus to C-terminus, the first NKG2D peptide or variant thereof, the first peptide linker, the second NKG2D peptide or variant thereof and the Fc region. In some embodiments, the second NKG2D peptide or variant thereof is directly fused with the Fc region without a peptide linker. In some embodiments, the second NKG2D peptide or variant thereof is linked with the Fc region via a second peptide linker.

In some embodiments, the dimer includes two identical monomers, where each monomer includes, from N-terminus to C-terminus, the first NKG2D peptide or variant thereof, the first peptide linker, the second NKG2D peptide or variant thereof and the Fc region. In some embodiments, the second NKG2D peptide or variant thereof is directly fused with the Fc region without a peptide linker. In some embodiments, the second NKG2D peptide or variant thereof is linked with the Fc region via a second peptide linker.

In some embodiments, one or both of the two monomers include, from N-terminus to C-terminus, the Fc region, the first NKG2D peptide or variant thereof, the first peptide linker, and the second NKG2D peptide or variant region. In some embodiments, the Fc region is directly fused with the first NKG2D peptide or variant thereof without a peptide linker. In some embodiments, the Fc region is linked with the first NKG2D peptide or variant thereof via a second peptide linker.

In some embodiments, the dimer includes two identical monomers, where each monomer includes, where each monomer includes, from N-terminus to C-terminus, the Fc region, the first NKG2D peptide or variant thereof, the first peptide linker, and the second NKG2D peptide or variant region. In some embodiments, the Fc region is directly fused with the first NKG2D peptide or variant thereof without a peptide linker. In some embodiments, the Fc region is linked with the first NKG2D peptide or variant thereof via a second peptide linker.

In some embodiments, one or both of the two monomers further comprising one or two additional NKG2D peptides or variants thereof.

In some embodiments, each of the additional one or two NKG2D peptides or variants thereof comprises, or alternatively, consists essentially of or consists of, an amino acid sequence having at least 85% (e.g., 85%, 86%, 87%, 88%, 89%, 90%, 91%, 92%, 93%, 94%, 95%, 96%, 97%, 98%, 99%, 100%) identity to SEQ ID NO: 1, 2, 3, or 4.

In some embodiments, each of the additional one or two NKG2D peptides or variants thereof comprises, or alternatively, consists essentially of or consists of, an amino acid sequence of SEQ ID NO: 1, 2, 3, or 4.

In some embodiments, the linker used herein is a peptide linker. In some embodiments, the linker is a flexible peptide linker. In some embodiments, a flexible peptide linker (e.g., the first peptide linker or the second peptide linker) is about 20 or fewer (e.g., 2, 3, 4, 5, 6, 7, 8, 9, 10, 11, 12, 13, 14, 15, 16, 17, 18, 19, or 20) amino acids in length. In some embodiments, a peptide linker contains about 12 or fewer amino acid residues, e.g., 2, 3, 4, 5, 6, 7, 8, 9, 10, 11, and 12. In some embodiments, the peptide linker (e.g., the first peptide linker or the second peptide linker) is about 20 amino acids in length. In some embodiments, the peptide linker (e.g., the first peptide linker or the second peptide linker) ranges from about 4 to about 16 (e.g., 4, 5, 6, 7, 8, 9, 10, 11, 12, 13, 14, 15, 16) amino acids in length. In some embodiments, the peptide linker (e.g., the first peptide linker or the second peptide linker) is 2, 3, 4, 5, 6, 7, 8, 9, 10, 11, 12, 13, 14, 15, 16, 17, 18, 19, 20, 21, 22, 23, 24, or 25 amino acids in length. In some embodiments, the peptide linker (e.g., the first peptide linker or the second peptide linker) is longer than 25 amino acids in length.

In some embodiments, a peptide linker (e.g., the first peptide linker or the second peptide linker) comprises two or more of the following amino acids: glycine, serine, alanine, and proline. In some embodiments, the peptide linker is a glycine-serine linker. In some embodiments, the glycine-serine linker is represented by the formula (GS)n, wherein n is 1, 2, 3, 4, 5, 6, 7, 8, 9, 10, 11, or 12 (SEQ ID NO: 7). In some embodiments, a peptide linker is a G4S linker represented by the formula of (GGGGS)n, where n is 1, 2, 3, 4, or 5 (SEQ ID NO: 8). In some embodiments, a peptide linker is a G4A linker represented by the formula of (GGGGA)n, where n is 1, 2, 3, 4, or 5 (SEQ ID NO: 12). In some embodiments, a peptide linker is a G4P linker represented by the formula of (GGGGP)n, where n is 1, 2, 3, 4, or 5 (SEQ ID NO: 13). In some embodiments, a peptide linker is a G3A linker represented by the formula of (GGGA)n, where n is 1, 2, 3, 4, or 5 (SEQ ID NO: 14). In some embodiments, a peptide linker is represented by the formula of (GGGS)ₙ, where n is 1, 2, 3, 4, or 5 (SEQ ID No: 9). In some embodiments, the glycine-serine linker comprises an amino acid sequence of GGGSGGGS (SEQ ID NO: 10). In some embodiments, the glycine-serine linker comprises an amino acid sequence of GGGGS (SEQ ID NO: 11). In some embodiments, the glycine-serine linker comprises an amino acid sequence of GGGS (SEQ ID NO: 66).

In some embodiments, the first peptide linker is about 20 or fewer amino acids in length. In some embodiments, the first peptide linker contains about 12 or fewer amino acid residues, e.g., 3, 4, 5, 6, 7, 8, 9, 10, 11, and 12. In some embodiments, the first peptide linker is 20 amino acids in length. In some embodiments, the first peptide linker ranges from about 4 to about 16 amino acids in length. In some embodiments, the first peptide linker is 2, 3, 4, 5, 6, 7, 8, 9, 10, 11, 12, 13, 14, 15, 16, 17, 18, 19, 20, 21, 22, 23, 24, or 25 amino acids in length. In some embodiments, the first peptide linker ranges from about 2 to about 25 amino acids in length. In some embodiments, the first peptide linker is longer than 25 amino acids in length. In some embodiments, the first peptide linker comprises two or more of the following amino acids: glycine, serine, alanine, and proline. In some embodiments, the first peptide linker is a glycine-serine linker. In some embodiments, the first peptide linker is represented by the formula (GS)n, wherein n is 1, 2, 3, 4, 5, 6, 7, 8, 9, 10, 11, or 12 (SEQ ID NO: 7). In some embodiments, the first peptide linker is a G4S linker represented by the formula of (GGGGS)n, where n is 1, 2, 3, 4, or 5 (SEQ ID NO: 8). In some embodiments, the first peptide linker is a G4A linker represented by the formula of (GGGGA)n, where n is 1, 2, 3, 4, or 5 (SEQ ID NO: 12). In some embodiments, the first peptide linker is a G4P linker represented by the formula of (GGGGP)n, where n is 1, 2, 3, 4, or 5 (SEQ ID NO: 13). In some embodiments, the first peptide linker is a G3A linker represented by the formula of (GGGA)n, where n is 1, 2, 3, 4, or 5 (SEQ ID NO: 14). In some embodiments, the first peptide linker is represented by the formula of (GGGS)ₙ, where n is 1, 2, 3, 4, or 5 (SEQ ID No: 9). In some embodiments, the first peptide linker comprises an amino acid sequence of GGGSGGGS (SEQ ID NO: 10). In some embodiments, the first peptide linker comprises an amino acid sequence of GGGGS (SEQ ID NO: 11). In some embodiments, the first peptide linker comprises an amino acid sequence of GGGS (SEQ ID NO: 66).

In some embodiments, the second peptide linker is about 20 or fewer amino acids in length. In some embodiments, the second peptide linker contains about 12 or fewer amino acid residues, e.g., 3, 4, 5, 6, 7, 8, 9, 10, 11, and 12. In some embodiments, the second peptide linker is 20 amino acids in length. In some embodiments, the second peptide linker ranges from about 4 to about 16 amino acids in length. In some embodiments, the second peptide linker is 2, 3, 4, 5, 6, 7, 8, 9, 10, 11, 12, 13, 14, 15, 16, 17, 18, 19, 20, 21, 22, 23, 24, or 25 amino acids in length. In some embodiments, the second peptide linker ranges from about 2 to about 25 amino acids in length. In some embodiments, the second peptide linker is longer than 25 amino acids in length. In some embodiments, the second peptide linker comprises two or more of the following amino acids: glycine, serine, alanine, and proline. In some embodiments, the second peptide linker is a glycine-serine linker. In some embodiments, the second peptide linker is represented by the formula (GS)n, wherein n is 1, 2, 3, 4, 5, 6, 7, 8, 9, 10, 11, or 12 (SEQ ID NO: 7). In some embodiments, the second peptide linker is a G4S linker represented by the formula of (GGGGS)n, where n is 1, 2, 3, 4, or 5 (SEQ ID NO: 8). In some embodiments, the second peptide linker is a G4A linker represented by the formula of (GGGGA)n, where n is 1, 2, 3, 4, or 5 (SEQ ID NO: 12). In some embodiments, the second peptide linker is a G4P linker represented by the formula of (GGGGP)n, where n is 1, 2, 3, 4, or 5 (SEQ ID NO: 13). In some embodiments, the second peptide linker is a G3A linker represented by the formula of (GGGA)n, where n is 1, 2, 3, 4, or 5 (SEQ ID NO: 14). In some embodiments, the second peptide linker is represented by the formula of (GGGS)ₙ, where n is 1, 2, 3, 4, or 5 (SEQ ID No: 9). In some embodiments, the second peptide linker comprises an amino acid sequence of GGGSGGGS (SEQ ID NO: 10). In some embodiments, the second peptide linker comprises an amino acid sequence of GGGGS (SEQ ID NO: 11). In some embodiments, the second peptide linker comprises an amino acid sequence of GGGS (SEQ ID NO: 66).

In some embodiments, a peptide linker (e.g., the first peptide linker and/or the second peptide linker) includes a protease-dependent cleavable site. Examples of protease-cleavable peptide linkers include, without limitation, the MMP sensitive linker GGPLGL W AGG (SEQ ID NO: 15) and the factor Xa-sensitive linker IEGR (SEQ ID NO: 16). It would be appreciated by a skilled artisan in the field that a variety of cleavable sequences may be employed for the linker provided herein.

In some embodiments, a linking molecule or linker may be a non-peptide linker. As used herein, a non-peptide linker is a biocompatible polymer including two or more repeating units linked to each other. Examples of the non-peptide polymer include but are not limited to: polyethylene glycol (PEG), polypropylene glycol (PPG), co-poly (ethylene/propylene) glycol, polyoxyethylene (POE), polyurethane, polyphosphazene, polysaccharides, dextran, polyvinyl alcohol, polyvinylpyrrolidones, polyvinyl ethyl ether, polyacryl amide, polyacrylate, polycyanoacrylates, lipid polymers, chitins, hyaluronic acid, and heparin. For more detailed descriptions of non-peptide linkers useful for Fc fusion molecules, see, for example, WO/2006/107124, which is incorporated by reference herein. In some embodiments, such linkers will have a range of molecular weight of from about 1 kDa to 50 kDa, depending upon a particular linker. In some embodiments, a typical PEG has a molecular weight of about 1 to 5 kDa, and polyethylene glycol has a molecular weight of about 5 kDa to 50 kDa, and more preferably about 10 kDa to 40 kDa.

In some embodiments, the monomer comprises, or alternatively, consists essentially of or consists of, an amino acid sequence having at least 85% (e.g., 85%, 86%, 87%, 88%, 89%, 90%, 91%, 92%, 93%, 94%, 95%, 96%, 97%, 98%, 99%, 100%) identity to SEQ ID NO: 17, 19, 21, 23, 25, 27, 29, 31, 33, 35, 37, 39, 41, 43, 45, 47, 49, 51, 53, 55, 57, 59, 61, or 69.

In some embodiments, the monomer comprises, or alternatively, consists essentially of or consists of, an amino acid sequence having at least 85% (e.g., 85%, 86%, 87%, 88%, 89%, 90%, 91%, 92%, 93%, 94%, 95%, 96%, 97%, 98%, 99%, 100%) identity to SEQ ID NO: 17.

In some embodiments, the monomer comprises, or alternatively, consists essentially of or consists of, an amino acid sequence having at least 85% (e.g., 85%, 86%, 87%, 88%, 89%, 90%, 91%, 92%, 93%, 94%, 95%, 96%, 97%, 98%, 99%, 100%) identity to SEQ ID NO: 19.

In some embodiments, the monomer comprises, or alternatively, consists essentially of or consists of, an amino acid sequence having at least 85% (e.g., 85%, 86%, 87%, 88%, 89%, 90%, 91%, 92%, 93%, 94%, 95%, 96%, 97%, 98%, 99%, 100%) identity to SEQ ID NO: 21.

In some embodiments, the monomer comprises, or alternatively, consists essentially of or consists of, an amino acid sequence having at least 85% (e.g., 85%, 86%, 87%, 88%, 89%, 90%, 91%, 92%, 93%, 94%, 95%, 96%, 97%, 98%, 99%, 100%) identity to SEQ ID NO: 23.

In some embodiments, the monomer comprises, or alternatively, consists essentially of or consists of, an amino acid sequence having at least 85% (e.g., 85%, 86%, 87%, 88%, 89%, 90%, 91%, 92%, 93%, 94%, 95%, 96%, 97%, 98%, 99%, 100%) identity to SEQ ID NO: 25.

In some embodiments, the monomer comprises, or alternatively, consists essentially of or consists of, an amino acid sequence having at least 85% (e.g., 85%, 86%, 87%, 88%, 89%, 90%, 91%, 92%, 93%, 94%, 95%, 96%, 97%, 98%, 99%, 100%) identity to SEQ ID NO: 27.

In some embodiments, the monomer comprises, or alternatively, consists essentially of or consists of, an amino acid sequence having at least 85% (e.g., 85%, 86%, 87%, 88%, 89%, 90%, 91%, 92%, 93%, 94%, 95%, 96%, 97%, 98%, 99%, 100%) identity to SEQ ID NO: 29.

In some embodiments, the monomer comprises, or alternatively, consists essentially of or consists of, an amino acid sequence having at least 85% (e.g., 85%, 86%, 87%, 88%, 89%, 90%, 91%, 92%, 93%, 94%, 95%, 96%, 97%, 98%, 99%, 100%) identity to SEQ ID NO: 31.

In some embodiments, the monomer comprises, or alternatively, consists essentially of or consists of, an amino acid sequence having at least 85% (e.g., 85%, 86%, 87%, 88%, 89%, 90%, 91%, 92%, 93%, 94%, 95%, 96%, 97%, 98%, 99%, 100%) identity to SEQ ID NO: 33.

In some embodiments, the monomer comprises, or alternatively, consists essentially of or consists of, an amino acid sequence having at least 85% (e.g., 85%, 86%, 87%, 88%, 89%, 90%, 91%, 92%, 93%, 94%, 95%, 96%, 97%, 98%, 99%, 100%) identity to SEQ ID NO: 35.

In some embodiments, the monomer comprises, or alternatively, consists essentially of or consists of, an amino acid sequence having at least 85% (e.g., 85%, 86%, 87%, 88%, 89%, 90%, 91%, 92%, 93%, 94%, 95%, 96%, 97%, 98%, 99%, 100%) identity to SEQ ID NO: 37.

In some embodiments, the monomer comprises, or alternatively, consists essentially of or consists of, an amino acid sequence having at least 85% (e.g., 85%, 86%, 87%, 88%, 89%, 90%, 91%, 92%, 93%, 94%, 95%, 96%, 97%, 98%, 99%, 100%) identity to SEQ ID NO: 39.

In some embodiments, the monomer comprises, or alternatively, consists essentially of or consists of, an amino acid sequence having at least 85% (e.g., 85%, 86%, 87%, 88%, 89%, 90%, 91%, 92%, 93%, 94%, 95%, 96%, 97%, 98%, 99%, 100%) identity to SEQ ID NO: 41.

In some embodiments, the monomer comprises, or alternatively, consists essentially of or consists of, an amino acid sequence having at least 85% (e.g., 85%, 86%, 87%, 88%, 89%, 90%, 91%, 92%, 93%, 94%, 95%, 96%, 97%, 98%, 99%, 100%) identity to SEQ ID NO: 43.

In some embodiments, the monomer comprises, or alternatively, consists essentially of or consists of, an amino acid sequence having at least 85% (e.g., 85%, 86%, 87%, 88%, 89%, 90%, 91%, 92%, 93%, 94%, 95%, 96%, 97%, 98%, 99%, 100%) identity to SEQ ID NO: 45.

In some embodiments, the monomer comprises, or alternatively, consists essentially of or consists of, an amino acid sequence having at least 85% (e.g., 85%, 86%, 87%, 88%, 89%, 90%, 91%, 92%, 93%, 94%, 95%, 96%, 97%, 98%, 99%, 100%) identity to SEQ ID NO: 47.

In some embodiments, the monomer comprises, or alternatively, consists essentially of or consists of, an amino acid sequence having at least 85% (e.g., 85%, 86%, 87%, 88%, 89%, 90%, 91%, 92%, 93%, 94%, 95%, 96%, 97%, 98%, 99%, 100%) identity to SEQ ID NO: 49.

In some embodiments, the monomer comprises, or alternatively, consists essentially of or consists of, an amino acid sequence having at least 85% (e.g., 85%, 86%, 87%, 88%, 89%, 90%, 91%, 92%, 93%, 94%, 95%, 96%, 97%, 98%, 99%, 100%) identity to SEQ ID NO: 51.

In some embodiments, the monomer comprises, or alternatively, consists essentially of or consists of, an amino acid sequence having at least 85% (e.g., 85%, 86%, 87%, 88%, 89%, 90%, 91%, 92%, 93%, 94%, 95%, 96%, 97%, 98%, 99%, 100%) identity to SEQ ID NO: 53.

In some embodiments, the monomer comprises, or alternatively, consists essentially of or consists of, an amino acid sequence having at least 85% (e.g., 85%, 86%, 87%, 88%, 89%, 90%, 91%, 92%, 93%, 94%, 95%, 96%, 97%, 98%, 99%, 100%) identity to SEQ ID NO: 55.

In some embodiments, the monomer comprises, or alternatively, consists essentially of or consists of, an amino acid sequence having at least 85% (e.g., 85%, 86%, 87%, 88%, 89%, 90%, 91%, 92%, 93%, 94%, 95%, 96%, 97%, 98%, 99%, 100%) identity to SEQ ID NO: 57.

In some embodiments, the monomer comprises, or alternatively, consists essentially of or consists of, an amino acid sequence having at least 85% (e.g., 85%, 86%, 87%, 88%, 89%, 90%, 91%, 92%, 93%, 94%, 95%, 96%, 97%, 98%, 99%, 100%) identity to SEQ ID NO: 59.

In some embodiments, the monomer comprises, or alternatively, consists essentially of or consists of, an amino acid sequence having at least 85% (e.g., 85%, 86%, 87%, 88%, 89%, 90%, 91%, 92%, 93%, 94%, 95%, 96%, 97%, 98%, 99%, 100%) identity to SEQ ID NO: 61.

In some embodiments, the monomer comprises, or alternatively, consists essentially of or consists of, an amino acid sequence having at least 85% (e.g., 85%, 86%, 87%, 88%, 89%, 90%, 91%, 92%, 93%, 94%, 95%, 96%, 97%, 98%, 99%, 100%) identity to SEQ ID NO: 69.

In some embodiments, the dimer includes two monomers, where each monomer comprises, or alternatively, consists essentially of or consists of, an amino acid sequence having at least 85% (e.g., 85%, 86%, 87%, 88%, 89%, 90%, 91%, 92%, 93%, 94%, 95%, 96%, 97%, 98%, 99%, 100%) identity to SEQ ID NO: 17, 19, 21, 23, 25, 27, 29, 31, 33, 35, 37, 39, 41, 43, 45, 47, 49, 51, 53, 55, 57, 59, 61, or 69.

In some embodiments, the monomer comprises, or alternatively, consists essentially of or consists of, an amino acid sequence of SEQ ID NO: 17, 19, 21, 23, 25, 27, 29, 31, 33, 35, 37, 39, 41, 43, 45, 47, 49, 51, 53, 55, 57, 59, 61, or 69.

In some embodiments, the monomer comprises, or alternatively, consists essentially of or consists of, an amino acid sequence of SEQ ID NO: 17.

In some embodiments, the monomer comprises, or alternatively, consists essentially of or consists of, an amino acid sequence of SEQ ID NO: 19.

In some embodiments, the monomer comprises, or alternatively, consists essentially of or consists of, an amino acid sequence of SEQ ID NO: 21.

In some embodiments, the monomer comprises, or alternatively, consists essentially of or consists of, an amino acid sequence of SEQ ID NO: 23.

In some embodiments, the monomer comprises, or alternatively, consists essentially of or consists of, an amino acid sequence of SEQ ID NO: 25.

In some embodiments, the monomer comprises, or alternatively, consists essentially of or consists of, an amino acid sequence of SEQ ID NO: 27.

In some embodiments, the monomer comprises, or alternatively, consists essentially of or consists of, an amino acid sequence of SEQ ID NO: 29.

In some embodiments, the monomer comprises, or alternatively, consists essentially of or consists of, an amino acid sequence of SEQ ID NO: 31.

In some embodiments, the monomer comprises, or alternatively, consists essentially of or consists of, an amino acid sequence of SEQ ID NO: 33.

In some embodiments, the monomer comprises, or alternatively, consists essentially of or consists of, an amino acid sequence of SEQ ID NO: 35.

In some embodiments, the monomer comprises, or alternatively, consists essentially of or consists of, an amino acid sequence of SEQ ID NO: 37.

In some embodiments, the monomer comprises, or alternatively, consists essentially of or consists of, an amino acid sequence of SEQ ID NO: 39.

In some embodiments, the monomer comprises, or alternatively, consists essentially of or consists of, an amino acid sequence of SEQ ID NO: 41.

In some embodiments, the monomer comprises, or alternatively, consists essentially of or consists of, an amino acid sequence of SEQ ID NO: 43.

In some embodiments, the monomer comprises, or alternatively, consists essentially of or consists of, an amino acid sequence of SEQ ID NO: 45.

In some embodiments, the monomer comprises, or alternatively, consists essentially of or consists of, an amino acid sequence of SEQ ID NO: 47.

In some embodiments, the monomer comprises, or alternatively, consists essentially of or consists of, an amino acid sequence of SEQ ID NO: 49.

In some embodiments, the monomer comprises, or alternatively, consists essentially of or consists of, an amino acid sequence of SEQ ID NO: 51.

In some embodiments, the monomer comprises, or alternatively, consists essentially of or consists of, an amino acid sequence of SEQ ID NO: 53.

In some embodiments, the monomer comprises, or alternatively, consists essentially of or consists of, an amino acid sequence of SEQ ID NO: 55.

In some embodiments, the monomer comprises, or alternatively, consists essentially of or consists of, an amino acid sequence of SEQ ID NO: 57.

In some embodiments, the monomer comprises, or alternatively, consists essentially of or consists of, an amino acid sequence of SEQ ID NO: 59.

In some embodiments, the monomer comprises, or alternatively, consists essentially of or consists of, an amino acid sequence of SEQ ID NO: 61.

In some embodiments, the monomer comprises, or alternatively, consists essentially of or consists of, an amino acid sequence of SEQ ID NO: 69.

In some embodiments, the dimer includes two monomers, where each monomer comprises, or alternatively, consists essentially of or consists of, an amino acid sequence of SEQ ID NO: 17, 19, 21, 23, 25, 27, 29, 31, 33, 35, 37, 39, 41, 43, 45, 47, 49, 51, 53, 55, 57, 59, 61, or 69.

In some embodiments, the monomer is encoded by a nucleic acid sequence comprising or consisting of a nucleic acid sequence having at least 85% (e.g., 85%, 86%, 87%, 88%, 89%, 90%, 91%, 92%, 93%, 94%, 95%, 96%, 97%, 98%, 99%, 100%) identity to SEQ ID NO: 18, 20, 22, 24, 26, 28, 30, 32, 34, 36, 38, 40, 42, 44, 46, 48, 50, 52, 54, 56, 58, 60, 62, or 70.

In some embodiments, the monomer is encoded by a nucleic acid sequence comprising or consisting of a nucleic acid sequence having at least 85% (e.g., 85%, 86%, 87%, 88%, 89%, 90%, 91%, 92%, 93%, 94%, 95%, 96%, 97%, 98%, 99%, 100%) identity to SEQ ID NO: 18.

In some embodiments, the monomer is encoded by a nucleic acid sequence comprising or consisting of a nucleic acid sequence having at least 85% (e.g., 85%, 86%, 87%, 88%, 89%, 90%, 91%, 92%, 93%, 94%, 95%, 96%, 97%, 98%, 99%, 100%) identity to SEQ ID NO: 20.

In some embodiments, the monomer is encoded by a nucleic acid sequence comprising or consisting of a nucleic acid sequence having at least 85% (e.g., 85%, 86%, 87%, 88%, 89%, 90%, 91%, 92%, 93%, 94%, 95%, 96%, 97%, 98%, 99%, 100%) identity to SEQ ID NO: 22.

In some embodiments, the monomer is encoded by a nucleic acid sequence comprising or consisting of a nucleic acid sequence having at least 85% (e.g., 85%, 86%, 87%, 88%, 89%, 90%, 91%, 92%, 93%, 94%, 95%, 96%, 97%, 98%, 99%, 100%) identity to SEQ ID NO: 24.

In some embodiments, the monomer is encoded by a nucleic acid sequence comprising or consisting of a nucleic acid sequence having at least 85% (e.g., 85%, 86%, 87%, 88%, 89%, 90%, 91%, 92%, 93%, 94%, 95%, 96%, 97%, 98%, 99%, 100%) identity to SEQ ID NO: 26.

In some embodiments, the monomer is encoded by a nucleic acid sequence comprising or consisting of a nucleic acid sequence having at least 85% (e.g., 85%, 86%, 87%, 88%, 89%, 90%, 91%, 92%, 93%, 94%, 95%, 96%, 97%, 98%, 99%, 100%) identity to SEQ ID NO: 28.

In some embodiments, the monomer is encoded by a nucleic acid sequence comprising or consisting of a nucleic acid sequence having at least 85% (e.g., 85%, 86%, 87%, 88%, 89%, 90%, 91%, 92%, 93%, 94%, 95%, 96%, 97%, 98%, 99%, 100%) identity to SEQ ID NO: 30.

In some embodiments, the monomer is encoded by a nucleic acid sequence comprising or consisting of a nucleic acid sequence having at least 85% (e.g., 85%, 86%, 87%, 88%, 89%, 90%, 91%, 92%, 93%, 94%, 95%, 96%, 97%, 98%, 99%, 100%) identity to SEQ ID NO: 32.

In some embodiments, the monomer is encoded by a nucleic acid sequence comprising or consisting of a nucleic acid sequence having at least 85% (e.g., 85%, 86%, 87%, 88%, 89%, 90%, 91%, 92%, 93%, 94%, 95%, 96%, 97%, 98%, 99%, 100%) identity to SEQ ID NO: 34.

In some embodiments, the monomer is encoded by a nucleic acid sequence comprising or consisting of a nucleic acid sequence having at least 85% (e.g., 85%, 86%, 87%, 88%, 89%, 90%, 91%, 92%, 93%, 94%, 95%, 96%, 97%, 98%, 99%, 100%) identity to SEQ ID NO: 36.

In some embodiments, the monomer is encoded by a nucleic acid sequence comprising or consisting of a nucleic acid sequence having at least 85% (e.g., 85%, 86%, 87%, 88%, 89%, 90%, 91%, 92%, 93%, 94%, 95%, 96%, 97%, 98%, 99%, 100%) identity to SEQ ID NO: 38.

In some embodiments, the monomer is encoded by a nucleic acid sequence comprising or consisting of a nucleic acid sequence having at least 85% (e.g., 85%, 86%, 87%, 88%, 89%, 90%, 91%, 92%, 93%, 94%, 95%, 96%, 97%, 98%, 99%, 100%) identity to SEQ ID NO: 40.

In some embodiments, the monomer is encoded by a nucleic acid sequence comprising or consisting of a nucleic acid sequence having at least 85% (e.g., 85%, 86%, 87%, 88%, 89%, 90%, 91%, 92%, 93%, 94%, 95%, 96%, 97%, 98%, 99%, 100%) identity to SEQ ID NO: 42.

In some embodiments, the monomer is encoded by a nucleic acid sequence comprising or consisting of a nucleic acid sequence having at least 85% (e.g., 85%, 86%, 87%, 88%, 89%, 90%, 91%, 92%, 93%, 94%, 95%, 96%, 97%, 98%, 99%, 100%) identity to SEQ ID NO: 44.

In some embodiments, the monomer is encoded by a nucleic acid sequence comprising or consisting of a nucleic acid sequence having at least 85% (e.g., 85%, 86%, 87%, 88%, 89%, 90%, 91%, 92%, 93%, 94%, 95%, 96%, 97%, 98%, 99%, 100%) identity to SEQ ID NO: 46.

In some embodiments, the monomer is encoded by a nucleic acid sequence comprising or consisting of a nucleic acid sequence having at least 85% (e.g., 85%, 86%, 87%, 88%, 89%, 90%, 91%, 92%, 93%, 94%, 95%, 96%, 97%, 98%, 99%, 100%) identity to SEQ ID NO: 48.

In some embodiments, the monomer is encoded by a nucleic acid sequence comprising or consisting of a nucleic acid sequence having at least 85% (e.g., 85%, 86%, 87%, 88%, 89%, 90%, 91%, 92%, 93%, 94%, 95%, 96%, 97%, 98%, 99%, 100%) identity to SEQ ID NO: 50.

In some embodiments, the monomer is encoded by a nucleic acid sequence comprising or consisting of a nucleic acid sequence having at least 85% (e.g., 85%, 86%, 87%, 88%, 89%, 90%, 91%, 92%, 93%, 94%, 95%, 96%, 97%, 98%, 99%, 100%) identity to SEQ ID NO: 52.

In some embodiments, the monomer is encoded by a nucleic acid sequence comprising or consisting of a nucleic acid sequence having at least 85% (e.g., 85%, 86%, 87%, 88%, 89%, 90%, 91%, 92%, 93%, 94%, 95%, 96%, 97%, 98%, 99%, 100%) identity to SEQ ID NO: 54.

In some embodiments, the monomer is encoded by a nucleic acid sequence comprising or consisting of a nucleic acid sequence having at least 85% (e.g., 85%, 86%, 87%, 88%, 89%, 90%, 91%, 92%, 93%, 94%, 95%, 96%, 97%, 98%, 99%, 100%) identity to SEQ ID NO: 56.

In some embodiments, the monomer is encoded by a nucleic acid sequence comprising or consisting of a nucleic acid sequence having at least 85% (e.g., 85%, 86%, 87%, 88%, 89%, 90%, 91%, 92%, 93%, 94%, 95%, 96%, 97%, 98%, 99%, 100%) identity to SEQ ID NO: 58.

In some embodiments, the monomer is encoded by a nucleic acid sequence comprising or consisting of a nucleic acid sequence having at least 85% (e.g., 85%, 86%, 87%, 88%, 89%, 90%, 91%, 92%, 93%, 94%, 95%, 96%, 97%, 98%, 99%, 100%) identity to SEQ ID NO: 60.

In some embodiments, the monomer is encoded by a nucleic acid sequence comprising or consisting of a nucleic acid sequence having at least 85% (e.g., 85%, 86%, 87%, 88%, 89%, 90%, 91%, 92%, 93%, 94%, 95%, 96%, 97%, 98%, 99%, 100%) identity to SEQ ID NO: 62.

In some embodiments, the monomer is encoded by a nucleic acid sequence comprising or consisting of a nucleic acid sequence having at least 85% (e.g., 85%, 86%, 87%, 88%, 89%, 90%, 91%, 92%, 93%, 94%, 95%, 96%, 97%, 98%, 99%, 100%) identity to SEQ ID NO: 70.

In some embodiments, the dimer includes two monomers, where each monomer is encoded by a nucleic acid sequence comprising or consisting of a nucleic acid sequence having at least 85% (e.g., 85%, 86%, 87%, 88%, 89%, 90%, 91%, 92%, 93%, 94%, 95%, 96%, 97%, 98%, 99%, 100%) identity to SEQ ID NO: 18, 20, 22, 24, 26, 28, 30, 32, 34, 36, 38, 40, 42, 44, 46, 48, 50, 52, 54, 56, 58, 60, 62, or 70.

In some embodiments the monomer is encoded by a nucleic acid sequence comprising or consisting of SEQ ID NO: 18, 20, 22, 24, 26, 28, 30, 32, 34, 36, 38, 40, 42, 44, 46, 48, 50, 52, 54, 56, 58, 60, 62, or 70.

In some embodiments, the monomer is encoded by a nucleic acid sequence comprising or consisting of SEQ ID NO: 18.

In some embodiments, the monomer is encoded by a nucleic acid sequence comprising or consisting of SEQ ID NO: 20.

In some embodiments, the monomer is encoded by a nucleic acid sequence comprising or consisting of SEQ ID NO: 22.

In some embodiments, the monomer is encoded by a nucleic acid sequence comprising or consisting of SEQ ID NO: 24.

In some embodiments, the monomer is encoded by a nucleic acid sequence comprising or consisting of SEQ ID NO: 26.

In some embodiments, the monomer is encoded by a nucleic acid sequence comprising or consisting of SEQ ID NO: 28.

In some embodiments, the monomer is encoded by a nucleic acid sequence comprising or consisting of SEQ ID NO: 30.

In some embodiments, the monomer is encoded by a nucleic acid sequence comprising or consisting of SEQ ID NO: 32.

In some embodiments, the monomer is encoded by a nucleic acid sequence comprising or consisting of SEQ ID NO: 34.

In some embodiments, the monomer is encoded by a nucleic acid sequence comprising or consisting of SEQ ID NO: 36.

In some embodiments, the monomer is encoded by a nucleic acid sequence comprising or consisting of SEQ ID NO: 38.

In some embodiments, the monomer is encoded by a nucleic acid sequence comprising or consisting of SEQ ID NO: 40.

In some embodiments, the monomer is encoded by a nucleic acid sequence comprising or consisting of SEQ ID NO: 42.

In some embodiments, the monomer is encoded by a nucleic acid sequence comprising or consisting of SEQ ID NO: 44.

In some embodiments, the monomer is encoded by a nucleic acid sequence comprising or consisting of SEQ ID NO: 46.

In some embodiments, the monomer is encoded by a nucleic acid sequence comprising or consisting of SEQ ID NO: 48.

In some embodiments, the monomer is encoded by a nucleic acid sequence comprising or consisting of SEQ ID NO: 50.

In some embodiments, the monomer is encoded by a nucleic acid sequence comprising or consisting of SEQ ID NO: 52.

In some embodiments, the monomer is encoded by a nucleic acid sequence comprising or consisting of SEQ ID NO: 54.

In some embodiments, the monomer is encoded by a nucleic acid sequence comprising or consisting of SEQ ID NO: 56.

In some embodiments, the monomer is encoded by a nucleic acid sequence comprising or consisting of SEQ ID NO: 58.

In some embodiments, the monomer is encoded by a nucleic acid sequence comprising or consisting of SEQ ID NO: 60.

In some embodiments, the monomer is encoded by a nucleic acid sequence comprising or consisting of SEQ ID NO: 62.

In some embodiments, the monomer is encoded by a nucleic acid sequence comprising or consisting of SEQ ID NO: 70.

In some embodiments, the dimer includes two monomers, where each monomer is encoded by a nucleic acid sequence comprising or consisting of SEQ ID NO: 18, 20, 22, 24, 26, 28, 30, 32, 34, 36, 38, 40, 42, 44, 46, 48, 50, 52, 54, 56, 58, 60, 62, or 70.

In some embodiments, the two monomers are covalently linked. For example, a conjugate between a first bioconjugate reactive group (e.g., —NH2, —COOH, —N-hydroxysuccinimide, or -maleimide) of a monomer and a second bioconjugate reactive group (e.g., sulfhydryl, sulfur-containing amino acid, amine, amine sidechain containing amino acid, or carboxylate) of another monomer provided herein can be direct, e.g., by covalent bond or linker. In some embodiments, the monomers are linked through a Cys-Cys bridge.

In some embodiments, the dimeric protein further comprising a drug moiety.

As used herein, “drug moiety” refers to a therapeutic agent that is intended for delivery to a targeted cell (e.g., a cancer cell). Generally, a drug moiety is conjugated (e.g., directly or indirectly covalently bound) to the carboxy terminus of a monomer of the dimeric protein described herein. However, the skilled artisan recognizes that in some embodiments, a drug moiety is conjugated to the amino terminus of a monomer of the dimeric protein described herein. Examples of “drug moieties” include drugs (e.g., small molecules), toxins (e.g., molecules of the lymphotoxin family), radionuclides, enzymes, cytokines, chemokines, antibody single chain variable fragments directed against activating compounds or blocking angiogenesis, or essentially any anti-tumor compound. In some embodiments, the drug moiety is directly fused to the amino terminus and/or the carboxy terminus of a monomer of the dimeric protein described herein.

In some embodiments, the drug moiety comprises a cytokine or functional portion thereof. Cytokines are proteins and peptides that are capable of modulating immune cell function. A “functional portion” of a cytokine is a cytokine fragment that retains the ability to modulate immune cell function (e.g., bind to one or more cytokine receptors). Examples of cytokines include, but are not limited to transforming growth factor beta (TGFβ), interferon-alpha (IFN-a), interferon-beta (IFN-β), and interferon-gamma (IFN-γ), interleukins (e.g., IL-1 to IL-36, in particular, IL-2, IL-5, IL-6, IL-7, IL-10, IL-12, IL-15 and IL-18), tumor necrosis factors (e.g., TNF-alpha and TNF-beta), erythropoietin (EPO), MIP3a, monocyte chemotactic protein (MCP)-1, intracellular adhesion molecule (ICAM), macrophage colony stimulating factor (M-CSF), granulocyte colony stimulating factor (G-CSF) and granulocyte-macrophage colony stimulating factor (GM-CSF). In some embodiments, the drug moiety comprises a cytokine selected from the group consisting of: TGFβ, IL-2, IL-4, IL-12, IL-15, IL-18, IL-21 and IFN-a.

In some embodiments, a drug moiety comprises a cytokine/cytokine receptor heterocomplex. Cytokine/cytokine receptor heterocomplexes are known in the art and are described, for example in Rowley et al., Eur J Immunol. 2009 February; 39(2): 491-506. In some embodiments, a dimeric protein described herein includes a drug moiety comprising an IL-15 (e.g., GenBank AAX37025)/IL-15Ra (e.g., GenBank AAP69528.1) heterocomplex. In some embodiments, the drug moiety comprises amino acids 31-107 of the human IL-15 receptor alpha (hIL15Ra, GenBank AAP69528.1) fused to amino acids 22-135 of IL-15 (GenBank AAX37025). In some embodiments, the IL-15 and IL-15Ra are separated by a linker, for example, a 20-amino acid (G4S)4 (SEQ ID NO: 3) linker. A dimeric NKG2D-Fc chimera comprising an IL-15/IL-15Ra heterocomplex is further described in the Examples section. In some embodiments, a dimeric NKG2D-Fc chimera includes a drug moiety comprising a heterocomplex of IL-12p35 and IL-12p40. In some embodiments, the IL-12p35 and IL-12p40 are separated by a peptide linker described herein. In some embodiments, a dimeric protein described herein includes a drug moiety comprising a heterocomplex of IL-23p19 and IL-23p40. In some embodiments, the IL-23p19 and IL-23p40 are separated by a peptide linker described herein. In some embodiments, a dimeric protein described herein includes a drug moiety comprising a heterocomplex of IL-27p28 and EB 1. In some embodiments, the IL-27p28 and EB 1 are separated by a peptide linker described herein. In some embodiments, each subunit of a cytokine/cytokine receptor heterocomplex is on a different monomer of the dimeric protein described herein.

In some embodiments, the drug moiety is an antibody single chain variable fragment (ScFv). As used herein, an “antibody single chain variable fragment” refers to a fusion protein of the variable regions of the heavy (VH) and light chains (VL) of immunoglobulins, connected with a short linker peptide. ScFv proteins retain the specificity of the original immunoglobulin, despite removal of the constant regions and the introduction of the linker. In some embodiments, a ScFv binds to an immune checkpoint protein (e.g., PD1 or CTLA4). In some embodiments, an ScFv blocks angiogenesis (e.g., binds to a regulator of angiogenesis, such as VEGF).

In some embodiments, the drug moiety is a chemokine. As used herein, “chemokines” refers to low-molecular-weight proteins that stimulate recruitment of leukocytes. Generally, chemokines are secondary pro-inflammatory mediators that are induced by primary proinflammatory mediators such as interleukin-1 (IL-1) or tumor necrosis factor (TNF). Chemokines can be classified into four families: CC chemokines (e.g., CCL1 to CCL-28), CXC (e.g., CXCL1 to CXCL17), C (e.g., XCL1, XCL2), and CX3C (CX3CL1).

In some embodiments, the drug moiety is a small molecule. As used herein, “small molecule” refers to a non-peptidic, non-oligomeric organic compound either synthesized in the laboratory or found in nature. Non-limiting examples of small molecule drugs include small molecule kinase inhibitors (e.g., everolimus, gefitinib, imatinib, etc.), bromodomain inhibitors (e.g., JQ1, 1-BET 151, RVX-208, etc.), antibiotics (e.g., kanamycin, neomycin, ciprofloxacin, etc.), and antivirals (e.g., ribavirin, rimantadine, zidovudine, etc.). In some embodiments, the small molecule is an anti-tumor compound. Anti-tumor compounds are discussed in further detail elsewhere in this disclosure.

In some embodiments, the drug moiety is a radionuclide. As used herein, “radionuclide” refers to medically useful radionuclides. Examples of radionuclides include: ^{99m}Tc, ¹⁸⁸Re, ¹⁸⁶Re, ¹⁵³Sm, ¹⁶⁶Ho, ⁹⁰Y, ⁶⁷Ga, ⁶⁸Ga, ^{m}In, ¹⁸³Gd, ⁵⁹Fe, ²²⁵Ac, ²¹²Bi, ²¹¹At, ⁴⁵Ti, ⁶⁰Cu, and ⁶⁷Cu.

Also provided herein is an isolated polynucleotide comprising a nucleic acid sequence comprising or consisting of SEQ ID NO: 18, 20, 22, 24, 26, 28, 30, 32, 34, 36, 38, 40, 42, 44, 46, 48, 50, 52, 54, 56, 58, 60, 62, or 70.

Further provided herein is a vector comprising the isolated polynucleotide described herein.

In some embodiments, exemplary vectors used herein include, but are not limited to, those disclosed in EP3027646 B1 or WO2009/080720, the content of each of which is incorporated herein by reference.

Further provided herein is a composition comprising the dimeric protein described herein, the isolated polynucleotide described herein or the vector described herein.

In some embodiments, the composition further comprising a pharmaceutically acceptable carrier. “Pharmaceutically acceptable excipient” and “pharmaceutically acceptable carrier” refer to a substance that aids the administration of an active agent to and absorption by a subject and can be included in the compositions of the present disclosure without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the disclosure. One of skill in the art will recognize that other pharmaceutical excipients are useful in the present disclosure.

Also provided herein is a cell comprising the dimeric protein described herein, the isolated polynucleotide described herein, the vector described herein or the composition described herein.

In some embodiments, the cell is a Chinese hamster ovary (CHO) cell.

In some embodiments, the cell is a modified CHO cell.

In some embodiments, the modified CHO cell comprises an inactivated matriptase gene or a knock-out (deletion) of matriptase gene. In some embodiments, the modified CHO cells comprises the cells described in WO2015166427, the content of which is incorporated herein by reference. Suitable selection processes for production cell lines are disclosed in WO2010/022961 and WO2015/015419, the content of each of which is incorporated herein by reference.

In some embodiments, the dimeric protein described herein produced by a modified CHO cell is more potent than the ones produced by other types of cell lines (e.g., HEK cells) (see, e.g., FIG. 9). In some embodiments, the dimeric protein described herein produced by a modified CHO cell is at least 5%, 10%, 15%, 20%, 25%, 30%, 35%, 40%, 45%, 50%, 55%, 60%, 65%, 70%, 75%, 80%, 85%, 90%, 95%, 100% or more potent than the dimeric protein produced by other types of cell lines. In some embodiments, potency of the dimeric protein described herein can be measured via its ADCC activity according to any method known in the art. Accordingly, in some embodiments, the dimeric protein described herein produced by a modified CHO cell has an ADCC activity that is at least 5%, 10%, 15%, 20%, 25%, 30%, 35%, 40%, 45%, 50%, 55%, 60%, 65%, 70%, 75%, 80%, 85%, 90%, 95%, 100% or higher than the ADCC activity of the dimeric protein produced by other types of cell lines. In some embodiments, potency of the dimeric protein can be measured via kinetic binding affinity constants (K_{D}) according to any method known in the art. Accordingly, in some embodiments, the dimeric protein described herein produced by a modified CHO cell has a K_{D} for an NKG2D ligand that at least about 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, 10 times or lower (i.e., stronger) than the K_{D} of the dimeric protein produced by other types of cell lines.

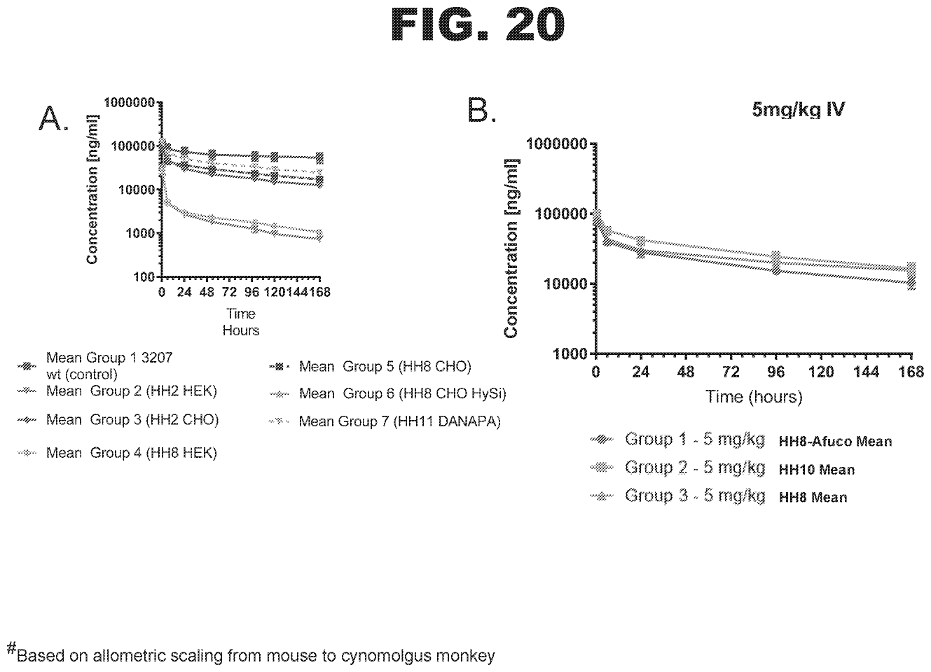

In some embodiments, the dimeric protein described herein produced by a modified CHO cell has much better in vivo pharmacokinetic (PK) properties than the ones produced by other types of cell lines (e.g., HEK cells) (see, e.g., FIG. 20). In some embodiments, the PK properties include, but are not limited to, the volume of distribution (V_{D}), the peak plasma concentration of a drug after administration (Cₘₐₓ), time to reach Cₘₐₓ (tₘₐₓ), the volume of plasma cleared of the drug per unit time (CL), the integral of the concentration-time curve (after a single dose or in steady state) (area under the curve, AUC), the time required for the concentration of the drug to reach half of its original value (t_{1/2}).

In some embodiments, the in vivo PK value of the dimeric protein described herein produced by a modified CHO cell is at least 5%, 10%, 15%, 20%, 25%, 30%, 35%, 40%, 45%, 50%, 55%, 60%, 65%, 70%, 75%, 80%, 85%, 90%, 95%, 100% better than the PK value of the dimeric protein produced by other types of cell lines. In some embodiments, the PK value may be determined based on the V_{D}, t_{1/2}, and/or AUC calculated according to methods known in the art.

Provided herein is a dimeric protein produced by a cell described herein. In some embodiments, the dimeric protein described herein or the dimeric protein produced by a cell described herein is glycosylated. In some embodiments, the Fc region of the dimeric protein described herein or the dimeric protein produced by a cell described herein is glycosylated. In some embodiments, the dimeric protein described herein or the dimeric protein produced by a cell described herein is afucosylated. In some embodiments, the Fc region of the dimeric protein described herein or the dimeric protein produced by a cell described herein is afucosylated.

In some embodiments, the dimeric protein described herein or the dimeric protein produced by a cell described herein includes at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 glycosylation sites.

In some embodiments, the dimeric protein described herein or the dimeric protein produced by a cell described herein includes at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 fucosylation sites.

In some embodiments, the glycosylation level of each monomer of the dimeric protein described herein or the dimeric protein produced by a cell described herein is at least 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%.

In some embodiments, the afucosylation level of each monomer of the dimeric protein described herein or the dimeric protein produced by a cell described herein is at least 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%.

In some embodiments, the glycosylation level of each Fc region of the dimeric protein described herein or the dimeric protein produced by a cell described herein is at least 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%.

In some embodiments, the afucosylation level of each Fc region of the dimeric protein described herein or the dimeric protein produced by a cell described herein is at least 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%.

In embodiments, a glycosylated dimeric protein described herein has higher (e.g., at least about 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, 10 times greater) binding affinity to an NKG2D ligand compared to a less or no glycosylated dimeric protein described herein.

In some embodiments, a glycosylated dimeric protein described herein has higher (e.g., at least about 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, 10 times or greater) ADCC activity compared to a less or no glycosylated dimeric protein described herein.

In some embodiments, a dimeric protein described herein with a higher afucosylation level has higher (e.g., at least about 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, 10, 20, 30, 40, 50, 60, 70, 80, 90, 100 times or greater) binding affinity to an Fc receptor (e.g. CD16) compared to a dimeric protein described herein with a lower afucosylation level.

In some embodiments, a dimeric protein described herein with a higher afucosylation level has higher (e.g., at least about 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, 10 times or greater) ADCC activity compared to a dimeric protein described herein with a lower afucosylation level.

Glycosylation level or afucosylation level can be determined by any method known in the art, for example, by LC-MS or 2AB-HILIC (2-aminobenzamide (2-AB)-labeled N-glycans). Further, methods of producing proteins with different afucosylation levels are known in the art. For example, proteins may be produced in a cell that either lacks the fucosylation enzyme, or by inclusion of RMD, which disrupts fucosylation.

In some embodiments, the dimeric protein described herein has a clipping level that is lower than about 20%, 19%, 18%, 17%, 16%, 15%, 14%, 13%, 12%, 11%, 10%, 9%, 8%, 7%, 6%, 5%, 4%, 3%, 2% or 1%. In some embodiments, the dimeric protein described herein has a clipping level that is lower than about 5%, 4%, 3%, 2% or 1%.

In some embodiments, the dimeric protein described herein has a clipping level that is at least 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% lower than the clipping level of a prior art dimeric construct.

In some embodiments, the dimeric protein produced in a modified CHO cell as described herein has a clipping level that is at least 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% lower than the clipping level of a dimeric protein produced in a different cell type.

Clipping level can be determined according to any method known in the art, for example, methods described in Example 4.

In any of the embodiments described in this disclosure, dimeric protein described herein is capable of binding the endogenous ligand of the NKG2D receptor. Known NKG2D-ligands in humans include MICA, MICB, ULBP1, ULBP2, ULBP3, ULBP4, ULBP5, and ULBP6. In some embodiments, the dimeric protein described herein is capable of binding more than one (e.g., 2, 3, 4, 5, 6, 7, or 8) type of NKG2D receptor ligand.

In some embodiments, the dimeric protein described herein binds to all eight ligands with a single-digit nM EC50 (see, e.g., FIG. 11). In some embodiments, the binding affinity (Ku) of the dimeric molecule for its ligands is about 10E-06 M to 10E-12 M. In some embodiments, the K_{D} of the dimeric molecule described herein is between about 0.01 nM to 50 nM—for example, about 0.06 to 12.97 nM (e.g., about 0.06 nM, 0.07 nM, 0.08 nM, 0.09 nM, 0.1 nM, 0.11 nM, 0.12 nM, 0.13 nM, 0.14 nM, 0.15 nM, 0.16 nM, 0.17 nM, 0.18 nM, 0.19 nM, 0.2 nM, 0.21 nM, 0.22 nM, 0.23 nM, 0.24 nM, 0.25 nM, 0.26 nM, 0.27 nM, 0.28 nM, 0.29 nM, 0.3 nM, 0.31 nM, 0.32 nM, 0.33 nM, 0.34 nM, 0.35 nM, 0.36 nM, 0.37 nM, 0.38 nM, 0.39 nM, 0.4 nM, 0.41 nM, 0.42 nM, 0.43 nM, 0.44 nM, 0.45 nM, 0.46 nM, 0.47 nM, 0.48 nM, 0.49 nM, 0.5 nM, 0.51 nM, 0.52 nM, 0.53 nM, 0.54 nM, 0.55 nM, 0.56 nM, 0.57 nM, 0.58 nM, 0.59 nM, 0.6 nM, 0.61 nM, 0.62 nM, 0.63 nM, 0.64 nM, 0.65 nM, 0.66 nM, 0.67 nM, 0.68 nM, 0.69 nM, 0.7 nM, 0.71 nM, 0.72 nM, 0.73 nM, 0.74 nM, 0.75 nM, 0.76 nM, 0.77 nM, 0.78 nM, 0.79 nM, 0.8 nM, 0.81 nM, 0.82 nM, 0.83 nM, 0.84 nM, 0.85 nM, 0.86 nM, 0.87 nM, 0.88 nM, 0.89 nM, 0.9 nM, 0.91 nM, 0.92 nM, 0.93 nM, 0.94 nM, 0.95 nM, 0.96 nM, 0.97 nM, 0.98 nM, 0.99 nM, 1 nM, 1.01 nM, 1.02 nM, 1.03 nM, 1.04 nM, 1.05 nM, 1.06 nM, 1.07 nM, 1.08 nM, 1.09 nM, 1.1 nM, 1.11 nM, 1.12 nM, 1.13 nM, 1.14 nM, 1.15 nM, 1.16 nM, 1.17 nM, 1.18 nM, 1.19 nM, 1.2 nM, 1.21 nM, 1.22 nM, 1.23 nM, 1.24 nM, 1.25 nM, 1.26 nM, 1.27 nM, 1.28 nM, 1.29 nM, 1.3 nM, 1.31 nM, 1.32 nM, 1.33 nM, 1.34 nM, 1.35 nM, 1.36 nM, 1.37 nM, 1.38 nM, 1.39 nM, 1.4 nM, 1.41 nM, 1.42 nM, 1.43 nM, 1.44 nM, 1.45 nM, 1.46 nM, 1.47 nM, 1.48 nM, 1.49 nM, 1.5 nM, 1.51 nM, 1.52 nM, 1.53 nM, 1.54 nM, 1.55 nM, 1.56 nM, 1.57 nM, 1.58 nM, 1.59 nM, 1.6 nM, 1.61 nM, 1.62 nM, 1.63 nM, 1.64 nM, 1.65 nM, 1.66 nM, 1.67 nM, 1.68 nM, 1.69 nM, 1.7 nM, 1.71 nM, 1.72 nM, 1.73 nM, 1.74 nM, 1.75 nM, 1.76 nM, 1.77 nM, 1.78 nM, 1.79 nM, 1.8 nM, 1.81 nM, 1.82 nM, 1.83 nM, 1.84 nM, 1.85 nM, 1.86 nM, 1.87 nM, 1.88 nM, 1.89 nM, 1.9 nM, 1.91 nM, 1.92 nM, 1.93 nM, 1.94 nM, 1.95 nM, 1.96 nM, 1.97 nM, 1.98 nM, 1.99 nM, 2 nM, 2.01 nM, 2.02 nM, 2.03 nM, 2.04 nM, 2.05 nM, 2.06 nM, 2.07 nM, 2.08 nM, 2.09 nM, 2.1 nM, 2.11 nM, 2.12 nM, 2.13 nM, 2.14 nM, 2.15 nM, 2.16 nM, 2.17 nM, 2.18 nM, 2.19 nM, 2.2 nM, 2.21 nM, 2.22 nM, 2.23 nM, 2.24 nM, 2.25 nM, 2.26 nM, 2.27 nM, 2.28 nM, 2.29 nM, 2.3 nM, 2.31 nM, 2.32 nM, 2.33 nM, 2.34 nM, 2.35 nM, 2.36 nM, 2.37 nM, 2.38 nM, 2.39 nM, 2.4 nM, 2.41 nM, 2.42 nM, 2.43 nM, 2.44 nM, 2.45 nM, 2.46 nM, 2.47 nM, 2.48 nM, 2.49 nM, 2.5 nM, 2.51 nM, 2.52 nM, 2.53 nM, 2.54 nM, 2.55 nM, 2.56 nM, 2.57 nM, 2.58 nM, 2.59 nM, 2.6 nM, 2.61 nM, 2.62 nM, 2.63 nM, 2.64 nM, 2.65 nM, 2.66 nM, 2.67 nM, 2.68 nM, 2.69 nM, 2.7 nM, 2.71 nM, 2.72 nM, 2.73 nM, 2.74 nM, 2.75 nM, 2.76 nM, 2.77 nM, 2.78 nM, 2.79 nM, 2.8 nM, 2.81 nM, 2.82 nM, 2.83 nM, 2.84 nM, 2.85 nM, 2.86 nM, 2.87 nM, 2.88 nM, 2.89 nM, 2.9 nM, 2.91 nM, 2.92 nM, 2.93 nM, 2.94 nM, 2.95 nM, 2.96 nM, 2.97 nM, 2.98 nM, 2.99 nM, 3 nM, 3.01 nM, 3.02 nM, 3.03 nM, 3.04 nM, 3.05 nM, 3.06 nM, 3.07 nM, 3.08 nM, 3.09 nM, 3.1 nM, 3.11 nM, 3.12 nM, 3.13 nM, 3.14 nM, 3.15 nM, 3.16 nM, 3.17 nM, 3.18 nM, 3.19 nM, 3.2 nM, 3.21 nM, 3.22 nM, 3.23 nM, 3.24 nM, 3.25 nM, 3.26 nM, 3.27 nM, 3.28 nM, 3.29 nM, 3.3 nM, 3.31 nM, 3.32 nM, 3.33 nM, 3.34 nM, 3.35 nM, 3.36 nM, 3.37 nM, 3.38 nM, 3.39 nM, 3.4 nM, 3.41 nM, 3.42 nM, 3.43 nM, 3.44 nM, 3.45 nM, 3.46 nM, 3.47 nM, 3.48 nM, 3.49 nM, 3.5 nM, 3.51 nM, 3.52 nM, 3.53 nM, 3.54 nM, 3.55 nM, 3.56 nM, 3.57 nM, 3.58 nM, 3.59 nM, 3.6 nM, 3.61 nM, 3.62 nM, 3.63 nM, 3.64 nM, 3.65 nM, 3.66 nM, 3.67 nM, 3.68 nM, 3.69 nM, 3.7 nM, 3.71 nM, 3.72 nM, 3.73 nM, 3.74 nM, 3.75 nM, 3.76 nM, 3.77 nM, 3.78 nM, 3.79 nM, 3.8 nM, 3.81 nM, 3.82 nM, 3.83 nM, 3.84 nM, 3.85 nM, 3.86 nM, 3.87 nM, 3.88 nM, 3.89 nM, 3.9 nM, 3.91 nM, 3.92 nM, 3.93 nM, 3.94 nM, 3.95 nM, 3.96 nM, 3.97 nM, 3.98 nM, 3.99 nM, 4 nM, 4.01 nM, 4.02 nM, 4.03 nM, 4.04 nM, 4.05 nM, 4.06 nM, 4.07 nM, 4.08 nM, 4.09 nM, 4.1 nM, 4.11 nM, 4.12 nM, 4.13 nM, 4.14 nM, 4.15 nM, 4.16 nM, 4.17 nM, 4.18 nM, 4.19 nM, 4.2 nM, 4.21 nM, 4.22 nM, 4.23 nM, 4.24 nM, 4.25 nM, 4.26 nM, 4.27 nM, 4.28 nM, 4.29 nM, 4.3 nM, 4.31 nM, 4.32 nM, 4.33 nM, 4.34 nM, 4.35 nM, 4.36 nM, 4.37 nM, 4.38 nM, 4.39 nM, 4.4 nM, 4.41 nM, 4.42 nM, 4.43 nM, 4.44 nM, 4.45 nM, 4.46 nM, 4.47 nM, 4.48 nM, 4.49 nM, 4.5 nM, 4.51 nM, 4.52 nM, 4.53 nM, 4.54 nM, 4.55 nM, 4.56 nM, 4.57 nM, 4.58 nM, 4.59 nM, 4.6 nM, 4.61 nM, 4.62 nM, 4.63 nM, 4.64 nM, 4.65 nM, 4.66 nM, 4.67 nM, 4.68 nM, 4.69 nM, 4.7 nM, 4.71 nM, 4.72 nM, 4.73 nM, 4.74 nM, 4.75 nM, 4.76 nM, 4.77 nM, 4.78 nM, 4.79 nM, 4.8 nM, 4.81 nM, 4.82 nM, 4.83 nM, 4.84 nM, 4.85 nM, 4.86 nM, 4.87 nM, 4.88 nM, 4.89 nM, 4.9 nM, 4.91 nM, 4.92 nM, 4.93 nM, 4.94 nM, 4.95 nM, 4.96 nM, 4.97 nM, 4.98 nM, 4.99 nM, 5 nM, 5.01 nM, 5.02 nM, 5.03 nM, 5.04 nM, 5.05 nM, 5.06 nM, 5.07 nM, 5.08 nM, 5.09 nM, 5.1 nM, 5.11 nM, 5.12 nM, 5.13 nM, 5.14 nM, 5.15 nM, 5.16 nM, 5.17 nM, 5.18 nM, 5.19 nM, 5.2 nM, 5.21 nM, 5.22 nM, 5.23 nM, 5.24 nM, 5.25 nM, 5.26 nM, 5.27 nM, 5.28 nM, 5.29 nM, 5.3 nM, 5.31 nM, 5.32 nM, 5.33 nM, 5.34 nM, 5.35 nM, 5.36 nM, 5.37 nM, 5.38 nM, 5.39 nM, 5.4 nM, 5.41 nM, 5.42 nM, 5.43 nM, 5.44 nM, 5.45 nM, 5.46 nM, 5.47 nM, 5.48 nM, 5.49 nM, 5.5 nM, 5.51 nM, 5.52 nM, 5.53 nM, 5.54 nM, 5.55 nM, 5.56 nM, 5.57 nM, 5.58 nM, 5.59 nM, 5.6 nM, 5.61 nM, 5.62 nM, 5.63 nM, 5.64 nM, 5.65 nM, 5.66 nM, 5.67 nM, 5.68 nM, 5.69 nM, 5.7 nM, 5.71 nM, 5.72 nM, 5.73 nM, 5.74 nM, 5.75 nM, 5.76 nM, 5.77 nM, 5.78 nM, 5.79 nM, 5.8 nM, 5.81 nM, 5.82 nM, 5.83 nM, 5.84 nM, 5.85 nM, 5.86 nM, 5.87 nM, 5.88 nM, 5.89 nM, 5.9 nM, 5.91 nM, 5.92 nM, 5.93 nM, 5.94 nM, 5.95 nM, 5.96 nM, 5.97 nM, 5.98 nM, 5.99 nM, 6 nM, 6.01 nM, 6.02 nM, 6.03 nM, 6.04 nM, 6.05 nM, 6.06 nM, 6.07 nM, 6.08 nM, 6.09 nM, 6.1 nM, 6.11 nM, 6.12 nM, 6.13 nM, 6.14 nM, 6.15 nM, 6.16 nM, 6.17 nM, 6.18 nM, 6.19 nM, 6.2 nM, 6.21 nM, 6.22 nM, 6.23 nM, 6.24 nM, 6.25 nM, 6.26 nM, 6.27 nM, 6.28 nM, 6.29 nM, 6.3 nM, 6.31 nM, 6.32 nM, 6.33 nM, 6.34 nM, 6.35 nM, 6.36 nM, 6.37 nM, 6.38 nM, 6.39 nM, 6.4 nM, 6.41 nM, 6.42 nM, 6.43 nM, 6.44 nM, 6.45 nM, 6.46 nM, 6.47 nM, 6.48 nM, 6.49 nM, 6.5 nM, 6.51 nM, 6.52 nM, 6.53 nM, 6.54 nM, 6.55 nM, 6.56 nM, 6.57 nM, 6.58 nM, 6.59 nM, 6.6 nM, 6.61 nM, 6.62 nM, 6.63 nM, 6.64 nM, 6.65 nM, 6.66 nM, 6.67 nM, 6.68 nM, 6.69 nM, 6.7 nM, 6.71 nM, 6.72 nM, 6.73 nM, 6.74 nM, 6.75 nM, 6.76 nM, 6.77 nM, 6.78 nM, 6.79 nM, 6.8 nM, 6.81 nM, 6.82 nM, 6.83 nM, 6.84 nM, 6.85 nM, 6.86 nM, 6.87 nM, 6.88 nM, 6.89 nM, 6.9 nM, 6.91 nM, 6.92 nM, 6.93 nM, 6.94 nM, 6.95 nM, 6.96 nM, 6.97 nM, 6.98 nM, 6.99 nM, 7 nM, 7.01 nM, 7.02 nM, 7.03 nM, 7.04 nM, 7.05 nM, 7.06 nM, 7.07 nM, 7.08 nM, 7.09 nM, 7.1 nM, 7.11 nM, 7.12 nM, 7.13 nM, 7.14 nM, 7.15 nM, 7.16 nM, 7.17 nM, 7.18 nM, 7.19 nM, 7.2 nM, 7.21 nM, 7.22 nM, 7.23 nM, 7.24 nM, 7.25 nM, 7.26 nM, 7.27 nM, 7.28 nM, 7.29 nM, 7.3 nM, 7.31 nM, 7.32 nM, 7.33 nM, 7.34 nM, 7.35 nM, 7.36 nM, 7.37 nM, 7.38 nM, 7.39 nM, 7.4 nM, 7.41 nM, 7.42 nM, 7.43 nM, 7.44 nM, 7.45 nM, 7.46 nM, 7.47 nM, 7.48 nM, 7.49 nM, 7.5 nM, 7.51 nM, 7.52 nM, 7.53 nM, 7.54 nM, 7.55 nM, 7.56 nM, 7.57 nM, 7.58 nM, 7.59 nM, 7.6 nM, 7.61 nM, 7.62 nM, 7.63 nM, 7.64 nM, 7.65 nM, 7.66 nM, 7.67 nM, 7.68 nM, 7.69 nM, 7.7 nM, 7.71 nM, 7.72 nM, 7.73 nM, 7.74 nM, 7.75 nM, 7.76 nM, 7.77 nM, 7.78 nM, 7.79 nM, 7.8 nM, 7.81 nM, 7.82 nM, 7.83 nM, 7.84 nM, 7.85 nM, 7.86 nM, 7.87 nM, 7.88 nM, 7.89 nM, 7.9 nM, 7.91 nM, 7.92 nM, 7.93 nM, 7.94 nM, 7.95 nM, 7.96 nM, 7.97 nM, 7.98 nM, 7.99 nM, 8 nM, 8.01 nM, 8.02 nM, 8.03 nM, 8.04 nM, 8.05 nM, 8.06 nM, 8.07 nM, 8.08 nM, 8.09 nM, 8.1 nM, 8.11 nM, 8.12 nM, 8.13 nM, 8.14 nM, 8.15 nM, 8.16 nM, 8.17 nM, 8.18 nM, 8.19 nM, 8.2 nM, 8.21 nM, 8.22 nM, 8.23 nM, 8.24 nM, 8.25 nM, 8.26 nM, 8.27 nM, 8.28 nM, 8.29 nM, 8.3 nM, 8.31 nM, 8.32 nM, 8.33 nM, 8.34 nM, 8.35 nM, 8.36 nM, 8.37 nM, 8.38 nM, 8.39 nM, 8.4 nM, 8.41 nM, 8.42 nM, 8.43 nM, 8.44 nM, 8.45 nM, 8.46 nM, 8.47 nM, 8.48 nM, 8.49 nM, 8.5 nM, 8.51 nM, 8.52 nM, 8.53 nM, 8.54 nM, 8.55 nM, 8.56 nM, 8.57 nM, 8.58 nM, 8.59 nM, 8.6 nM, 8.61 nM, 8.62 nM, 8.63 nM, 8.64 nM, 8.65 nM, 8.66 nM, 8.67 nM, 8.68 nM, 8.69 nM, 8.7 nM, 8.71 nM, 8.72 nM, 8.73 nM, 8.74 nM, 8.75 nM, 8.76 nM, 8.77 nM, 8.78 nM, 8.79 nM, 8.8 nM, 8.81 nM, 8.82 nM, 8.83 nM, 8.84 nM, 8.85 nM, 8.86 nM, 8.87 nM, 8.88 nM, 8.89 nM, 8.9 nM, 8.91 nM, 8.92 nM, 8.93 nM, 8.94 nM, 8.95 nM, 8.96 nM, 8.97 nM, 8.98 nM, 8.99 nM, 9 nM, 9.01 nM, 9.02 nM, 9.03 nM, 9.04 nM, 9.05 nM, 9.06 nM, 9.07 nM, 9.08 nM, 9.09 nM, 9.1 nM, 9.11 nM, 9.12 nM, 9.13 nM, 9.14 nM, 9.15 nM, 9.16 nM, 9.17 nM, 9.18 nM, 9.19 nM, 9.2 nM, 9.21 nM, 9.22 nM, 9.23 nM, 9.24 nM, 9.25 nM, 9.26 nM, 9.27 nM, 9.28 nM, 9.29 nM, 9.3 nM, 9.31 nM, 9.32 nM, 9.33 nM, 9.34 nM, 9.35 nM, 9.36 nM, 9.37 nM, 9.38 nM, 9.39 nM, 9.4 nM, 9.41 nM, 9.42 nM, 9.43 nM, 9.44 nM, 9.45 nM, 9.46 nM, 9.47 nM, 9.48 nM, 9.49 nM, 9.5 nM, 9.51 nM, 9.52 nM, 9.53 nM, 9.54 nM, 9.55 nM, 9.56 nM, 9.57 nM, 9.58 nM, 9.59 nM, 9.6 nM, 9.61 nM, 9.62 nM, 9.63 nM, 9.64 nM, 9.65 nM, 9.66 nM, 9.67 nM, 9.68 nM, 9.69 nM, 9.7 nM, 9.71 nM, 9.72 nM, 9.73 nM, 9.74 nM, 9.75 nM, 9.76 nM, 9.77 nM, 9.78 nM, 9.79 nM, 9.8 nM, 9.81 nM, 9.82 nM, 9.83 nM, 9.84 nM, 9.85 nM, 9.86 nM, 9.87 nM, 9.88 nM, 9.89 nM, 9.9 nM, 9.91 nM, 9.92 nM, 9.93 nM, 9.94 nM, 9.95 nM, 9.96 nM, 9.97 nM, 9.98 nM, 9.99 nM, 10 nM, 10.01 nM, 10.02 nM, 10.03 nM, 10.04 nM, 10.05 nM, 10.06 nM, 10.07 nM, 10.08 nM, 10.09 nM, 10.1 nM, 10.11 nM, 10.12 nM, 10.13 nM, 10.14 nM, 10.15 nM, 10.16 nM, 10.17 nM, 10.18 nM, 10.19 nM, 10.2 nM, 10.21 nM, 10.22 nM, 10.23 nM, 10.24 nM, 10.25 nM, 10.26 nM, 10.27 nM, 10.28 nM, 10.29 nM, 10.3 nM, 10.31 nM, 10.32 nM, 10.33 nM, 10.34 nM, 10.35 nM, 10.36 nM, 10.37 nM, 10.38 nM, 10.39 nM, 10.4 nM, 10.41 nM, 10.42 nM, 10.43 nM, 10.44 nM, 10.45 nM, 10.46 nM, 10.47 nM, 10.48 nM, 10.49 nM, 10.5 nM, 10.51 nM, 10.52 nM, 10.53 nM, 10.54 nM, 10.55 nM, 10.56 nM, 10.57 nM, 10.58 nM, 10.59 nM, 10.6 nM, 10.61 nM, 10.62 nM, 10.63 nM, 10.64 nM, 10.65 nM, 10.66 nM, 10.67 nM, 10.68 nM, 10.69 nM, 10.7 nM, 10.71 nM, 10.72 nM, 10.73 nM, 10.74 nM, 10.75 nM, 10.76 nM, 10.77 nM, 10.78 nM, 10.79 nM, 10.8 nM, 10.81 nM, 10.82 nM, 10.83 nM, 10.84 nM, 10.85 nM, 10.86 nM, 10.87 nM, 10.88 nM, 10.89 nM, 10.9 nM, 10.91 nM, 10.92 nM, 10.93 nM, 10.94 nM, 10.95 nM, 10.96 nM, 10.97 nM, 10.98 nM, 10.99 nM, 11 nM, 11.01 nM, 11.02 nM, 11.03 nM, 11.04 nM, 11.05 nM, 11.06 nM, 11.07 nM, 11.08 nM, 11.09 nM, 11.1 nM, 11.11 nM, 11.12 nM, 11.13 nM, 11.14 nM, 11.15 nM, 11.16 nM, 11.17 nM, 11.18 nM, 11.19 nM, 11.2 nM, 11.21 nM, 11.22 nM, 11.23 nM, 11.24 nM, 11.25 nM, 11.26 nM, 11.27 nM, 11.28 nM, 11.29 nM, 11.3 nM, 11.31 nM, 11.32 nM, 11.33 nM, 11.34 nM, 11.35 nM, 11.36 nM, 11.37 nM, 11.38 nM, 11.39 nM, 11.4 nM, 11.41 nM, 11.42 nM, 11.43 nM, 11.44 nM, 11.45 nM, 11.46 nM, 11.47 nM, 11.48 nM, 11.49 nM, 11.5 nM, 11.51 nM, 11.52 nM, 11.53 nM, 11.54 nM, 11.55 nM, 11.56 nM, 11.57 nM, 11.58 nM, 11.59 nM, 11.6 nM, 11.61 nM, 11.62 nM, 11.63 nM, 11.64 nM, 11.65 nM, 11.66 nM, 11.67 nM, 11.68 nM, 11.69 nM, 11.7 nM, 11.71 nM, 11.72 nM, 11.73 nM, 11.74 nM, 11.75 nM, 11.76 nM, 11.77 nM, 11.78 nM, 11.79 nM, 11.8 nM, 11.81 nM, 11.82 nM, 11.83 nM, 11.84 nM, 11.85 nM, 11.86 nM, 11.87 nM, 11.88 nM, 11.89 nM, 11.9 nM, 11.91 nM, 11.92 nM, 11.93 nM, 11.94 nM, 11.95 nM, 11.96 nM, 11.97 nM, 11.98 nM, 11.99 nM, 12 nM, 12.01 nM, 12.02 nM, 12.03 nM, 12.04 nM, 12.05 nM, 12.06 nM, 12.07 nM, 12.08 nM, 12.09 nM, 12.1 nM, 12.11 nM, 12.12 nM, 12.13 nM, 12.14 nM, 12.15 nM, 12.16 nM, 12.17 nM, 12.18 nM, 12.19 nM, 12.2 nM, 12.21 nM, 12.22 nM, 12.23 nM, 12.24 nM, 12.25 nM, 12.26 nM, 12.27 nM, 12.28 nM, 12.29 nM, 12.3 nM, 12.31 nM, 12.32 nM, 12.33 nM, 12.34 nM, 12.35 nM, 12.36 nM, 12.37 nM, 12.38 nM, 12.39 nM, 12.4 nM, 12.41 nM, 12.42 nM, 12.43 nM, 12.44 nM, 12.45 nM, 12.46 nM, 12.47 nM, 12.48 nM, 12.49 nM, 12.5 nM, 12.51 nM, 12.52 nM, 12.53 nM, 12.54 nM, 12.55 nM, 12.56 nM, 12.57 nM, 12.58 nM, 12.59 nM, 12.6 nM, 12.61 nM, 12.62 nM, 12.63 nM, 12.64 nM, 12.65 nM, 12.66 nM, 12.67 nM, 12.68 nM, 12.69 nM, 12.7 nM, 12.71 nM, 12.72 nM, 12.73 nM, 12.74 nM, 12.75 nM, 12.76 nM, 12.77 nM, 12.78 nM, 12.79 nM, 12.8 nM, 12.81 nM, 12.82 nM, 12.83 nM, 12.84 nM, 12.85 nM, 12.86 nM, 12.87 nM, 12.88 nM, 12.89 nM, 12.9 nM, 12.91 nM, 12.92 nM, 12.93 nM, 12.94 nM, 12.95 nM, 12.96 nM, or 12.97 nM).

In some embodiments, a dimeric protein described herein binds to (e.g., with higher affinity for) a subset of NKG2D receptor ligands. 3D structural data in combination with mutagenesis analyses have revealed that NKG2D is permissive in the recognition and binding of a diverse array of its endogenous ligands. A ligand for NKG2D may be expressed on a cell surface. In some embodiments, a ligand for NKG2D may be “shed” from the cell surface and is present as a soluble ligand. It has been known in certain cancers that NKG2D ligands such as MICA are over-expressed and in some cases released (e.g., shed) into the bloodstream or surrounding tissues in a soluble form, e.g., in sera. It is believed that this contributes, at least in part, to the pathogenesis and/or progression of cancer. Thus, the dimeric protein described herein is useful for binding such ligand, either present on cell surface or as a released form, in counterbalancing the expression of the ligands that are present at an abnormally elevated level by functioning as a neutralizing agent. Where an NKG2D ligand is expressed on the surface of cancer cells of a subject, dimeric protein described herein binds to the cell surface ligand when administered to the subject. The binding of the dimeric protein described herein to its ligand may prevent activation of endogenous NKG2D receptors present on NK cells. Where an NKG2D ligand is “shed” from cancer cells, e.g., released into the bloodstream of a subject, dimeric protein described herein binds to the soluble ligand, sequestering it from further action.

In some embodiments, the dimeric protein described herein has enhanced ADCC capability compared to wild type NKG2D receptor or other chimeric protein known in the art.

Pharmaceutical Compositions

Also provided herein are pharmaceutical compositions comprising a dimeric protein disclosed herein (e.g., a dimeric protein produced in a modified CHO cell) in combination with at least one pharmaceutically acceptable excipient or carrier.

A “pharmaceutical composition” is a formulation containing the dimeric protein disclosed herein in a form suitable for administration to a subject. In one embodiment, the pharmaceutical composition is in bulk or in unit dosage form. The unit dosage form is any of a variety of forms, including, for example, a lyophilization formulation, a capsule, an IV bag, a tablet, a single pump on an aerosol inhaler or a vial. The quantity of active ingredient (e.g., a formulation of the disclosed dimeric protein) in a unit dose of composition is an effective amount and is varied according to the particular treatment involved. One skilled in the art will appreciate that it is sometimes necessary to make routine variations to the dosage depending on the age and condition of the patient. The dosage will also depend on the route of administration. A variety of routes are contemplated, including oral, pulmonary, rectal, parenteral, transdermal, subcutaneous, intravenous, intramuscular, intraperitoneal, inhalational, buccal, sublingual, intrapleural, intrathecal, intranasal, and the like. Dosage forms for the topical or transdermal administration of a compound of this invention include powders, sprays, ointments, pastes, creams, lotions, gels, solutions, patches and inhalants. In one embodiment, the active compound is mixed under sterile conditions with a pharmaceutically acceptable carrier, and with any preservatives, buffers, or propellants that are required.

As used herein, the phrase “pharmaceutically acceptable” refers to those compounds, anions, cations, materials, compositions, carriers, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically acceptable excipient” means an excipient that is useful in preparing a pharmaceutical composition that is generally safe, non-toxic and neither biologically nor otherwise undesirable, and includes excipient that is acceptable for veterinary use as well as human pharmaceutical use. A “pharmaceutically acceptable excipient” as used in the specification and claims includes both one and more than one such excipient.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), and transmucosal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic. In some embodiments, the pH of the solutions or suspensions used for parenteral, intradermal, or subcutaneous application is about 6 to about 8 (e.g., about 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, or 8).

Methods and Uses

Provided herein is a method for treating a disease, the method comprising administering to a subject in need thereof a dimeric protein described herein or a composition described herein or a pharmaceutical composition described herein.

In some embodiments, the disease is an NKG2D ligand expressing disease. In some embodiments, the disease is cancer, autoimmune disease, liver disease, nerve injury, hereditary motor and sensory neuropathy, aging, or viral infection. In some embodiments, the disease is cancer or autoimmune disease. In some embodiments, the cancer is an NKG2D ligand expressing cancer. In some embodiments, the subject has an NKG2D ligand expressing cancer.

In some embodiments, the ligand expressing cancer is lung cancer, ovarian cancer, colon cancer, pancreatic cancer, bladder cancer, breast cancer, hepatocellular carcinoma (HCC), renal cancer, nasopharyngeal carcinoma (NPC), prostate cancer, melanoma, plasma cell cancer, leukemia, lymphoma, glioma, or neuroblastoma.

In some embodiments, the leukemia is acute myeloid leukemia (AML), chronic myeloid leukemia (CML) or chronic lymphocytic leukemia (CLL).

In some embodiments, the NKG2D ligand is MICA, MICB, ULBP1, ULBP2, ULBP3, ULBP4, ULBP5, or ULBP6.

In some embodiments, the autoimmune disease is rheumatoid arthritis, colitis, celiac disease, multiple sclerosis, alopecia areata, type 1 diabetes, chronic obstructive pulmonary disease, atherosclerosis, or metabolic syndrome associated with type 2 diabetes.

In some embodiments, the liver disease is liver fibrosis, portal vein hypertension, nonalcoholic steatohepatitis (NASH), fatty liver disease, and cirrhosis.

In some embodiments, the nerve injury is traumatic or toxic injuries to peripheral or cranial nerves, spinal cord or to the brain, cranial nerves, traumatic brain injury, stroke, cerebral aneurism, spinal cord injury, or injury related to a underlying genetic disease.

In some embodiments, the hereditary motor and sensory neuropathy is Charcot-Marie-Tooth (e.g. type 1A, type2, type3, with pyramidal features, or type 6) or refsum disease.

In some embodiments, the viral infection is hepatitis, Epstein-Barr, or cytomegalovirus.

Whether a particular subject (e.g., patient) should receive a cancer therapy comprising a dimeric protein described herein can be determined by testing for aberrant expression of one or more NKG2D ligands in the subject. “Aberrant expression of one or more NKG2D ligands” in the subject means over-expression of the ligand(s) in a biological sample obtained from the subject compared to a control level (e.g., an expression level from a healthy subject). In some embodiments, a biological sample may include a biopsy sample taken from a tissue of the subject suspected to be cancerous. In some embodiments, a biological sample is collected from a solid tumor to test for malignancy. In some embodiments, a biological sample may constitute a blood sample, e.g., serum, a stool sample, urine sample, etc. In some embodiments, a biological sample may be any cell or tissue sample that is collected from a subject for the purpose of testing for the diagnosis or progression of a disease, such as cancer or autoimmune disease.

One of ordinary skill in the art is familiar with a variety of laboratory techniques and protocols used to assay for the presence of and the levels of one or more markers present in a biological sample. To determine whether a subject has cancer that is associated with over-expression of NKG2D ligand(s), typically immunoaffinity assays are performed. In certain situations, depending on the type of biological samples that are available, immunohistological or immunocytochemical analyses may be carried out. A number of antibodies are commercially available for performing these analyses. Methods commonly employed for this purpose include, but are not limited to, ELISA, immunoblotting, and immunohistochemistry.

The composition described herein can be administered directly to a subject. The terms “administration” and “administer” refer to a means of providing a pharmaceutical agent to a subject such that the pharmaceutical agent is to contact its target cells, e.g., diseased cells, in vivo, i.e., in the body of the subject. In some embodiments, the composition comprising NKG2D-Fc is systematically administered to a subject. In some embodiments, a systematic administration is delivered via an intravenous injection. In some embodiments, the composition comprising dimeric protein is administered locally. In some embodiments, the composition may be delivered directly to or within close proximity of a solid tumor.

Any composition described herein can be administered to any part of the subject's body via various administration routes. The composition can be administered by intravenous, intraperitoneal, intramuscular, subcutaneous, intramuscular, intrarectal, intravaginal, intrathecal, intratracheal, intradermal, or transdermal injection, by oral or nasal administration, by inhalation, or by gradual perfusion over time. The composition can be delivered to specific tissue. For example, the composition can be delivered to, without limitation, the joints, nasal mucosa, blood, lungs, intestines, muscle tissues, skin, or peritoneal cavity of a mammal. In a further example, an aerosol preparation of a composition can be given to a subject by inhalation.

The dosage required depends on the route of administration, the nature of the formulation, the nature of the patient's illness, the subject's size, weight, surface area, age, and sex, other drugs being administered, and the judgment of the attending physician. Suitable dosages are typically in the range of about 0.01 g to about 5 g (e.g., about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, or 5 g). In some embodiments, suitable dosage is in the range of about 250 mg to about 1400 mg (e.g., about 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1010, 1020, 1030, 1040, 1050, 1060, 1070, 1080, 1090, 1100, 1110, 1120, 1130, 1140, 1150, 1160, 1170, 1180, 1190, 1200, 1210, 1220, 1230, 1240, 1250, 1260, 1270, 1280, 1290, 1300, 1310, 1320, 1330, 1340, 1350, 1360, 1370, 1380, 1390, or 1400). Wide variations in the needed dosage are to be expected in view of the variety of dimeric compositions available and the differing efficiencies of various routes of administration. Variations in these dosage levels can be adjusted using standard empirical routines for optimization as is well understood in the art. Administrations can be single or multiple (e.g., 2- or 3-, 4-, 6-, 8-, 10-, 20-, 50-, 100, 150-, or more fold). Encapsulation of the composition in a suitable delivery vehicle (e.g. polymeric microparticles or implantable devices) may increase the efficiency of delivery.

The duration of treatment with any composition provided herein can be any length of time from as short as one day to as long as the life span of the host (e.g., many years). For example, dimeric compositions can be administered once a month for three months, or once a year for a period of ten years. It is also noted that the frequency of treatment can be variable. In some embodiments, dimeric compositions can be administered once (or twice, three times, etc.) daily, weekly, monthly, or yearly. In some embodiments, dimeric compositions can be administered once every two week. Dimeric compositions can be administered together, e.g., at the same point in time or sequentially, with one or more other cancer therapies.

An effective amount of any composition described herein can be administered to a subject. The term “effective” as used herein refers to any amount that induces a desired therapeutic effect, such as an immune response, while not inducing significant toxicity in the subject. Such an amount can be determined by assessing a subject's biological reaction, e.g., immune response and improvement in a symptom, after administration of a known amount of a particular composition. In addition, the level of toxicity, if any, can be determined by assessing a subject's clinical symptoms before and after administering a known amount of a particular composition. It is noted that the effective amount of a particular composition administered to a subject can be adjusted according to a desired outcome as well as the host's response and level of toxicity. Significant toxicity can vary for each particular host and depends on multiple factors including, without limitation, the subject's disease state, age, and tolerance to pain.

In some embodiments, the method further comprising treating the subject with an additional anti-cancer therapy or an anti-cancer agent. In some embodiments, the additional anti-cancer therapy is surgery, radiation therapy, chemotherapy, gene therapy, DNA therapy, viral therapy, RNA therapy, adjuvant therapy, or immunotherapy. In some embodiments, the additional cancer therapy is a chemotherapy that damages DNA. In some embodiments, the anti-cancer agent is an IL-15 (e.g., GenBank AAX37025)/IL-15Ra (e.g., GenBank AAP69528.1) heterocomplex (e.g., IL-15/IL-Ra heterocomplexes disclosed in WO2007/001677 and WO20007/084342 and/or encoded by SEQ ID NO: 67). In some embodiments, the anti-cancer agent is an anti-PD-1 antibody or other PD-1 inhibitor. Antibodies, antibody fragments, and other inhibitors of PD-1, PD-L1 and PD-L2 are available in the art and may be used combination with a cars of the present invention described herein. For example, nivolumab (also referred to as BMS-936558 or MDX1106; Bristol-Myers Squibb) is a fully human IgG4 monoclonal antibody which specifically blocks PD-1. Nivolumab (clone 5C4) and other human monoclonal antibodies that specifically bind to PD-1 are disclosed in U.S. Pat. No. 8,008,449 and WO2006/121168. Pidilizumab (CT-011; Cure Tech) is a humanized IgG1k monoclonal antibody that binds to PD-1. Pidilizumab and other humanized anti-PD-1 monoclonal antibodies are disclosed in WO2009/101611. Pembrolizumab (formerly known as lambrolizumab, and also referred to as MK03475; Merck) is a humanized IgG4 monoclonal antibody that binds to PD-1. Pembrolizumab and other humanized anti-PD-1 antibodies are disclosed in U.S. Pat. No. 8,354,509 and WO2009/114335. MEDI4736 (Medimmune) is a human monoclonal antibody that binds to PDL1, and inhibits interaction of the ligand with PD1. MDPL3280A (Genentech/Roche) is a human Fc optimized IgG1 monoclonal antibody that binds to PD-L1. MDPL3280A and other human monoclonal antibodies to PD-L1 are disclosed in U.S. Pat. No. 7,943,743 and U.S. Publication No.: 20120039906. Other anti-PD-L1 binding agents include YW243.55.570 (heavy and light chain variable regions are shown in SEQ ID NOs 20 and 21 in WO2010/077634) and MDX-1 105 (also referred to as BMS-936559, and, e.g., anti-PD-L1 binding agents disclosed in WO2007/005874). AMP-224 (B7-DCIg; Amplimmune; e.g., disclosed in WO2010/027827 and WO2011/066342), is a PD-L2 Fc fusion soluble receptor that blocks the interaction between PD-1 and B7-H1. Other anti-PD-1 antibodies include AMP 514 (Amplimmune), among others, e.g., anti-PD-1 antibodies disclosed in U.S. Pat. No. 8,609,089, US 2010028330, and/or US 20120114649. In some embodiments, the anti-cancer agent is a radioligand, such as but not limited to ¹⁷⁷Lu-PSMA-617 (lutetium (117Lu) oxodotreotide). In some embodiments, the anti-cancer agent is a cancer vaccine.

In some embodiments, the additional cancer therapy includes a cytotoxic agent and/or non-cytotoxic agent. A “cytotoxic agent” refers to a substance that inhibits or prevents the function of cells and/or causes destruction of cells. The term is intended to include radioactive isotopes (e.g., 1311, 1251, 90Y and 186Re), chemotherapeutic agents, and toxins such as enzymatically active toxins of bacterial, fungal, plant or animal origin or synthetic toxins, or fragments thereof. A non-cytotoxic agent refers to a substance that does not inhibit or prevent the function of cells and/or does not cause destruction of cells. A “non-cytotoxic agent” may include an agent that can be activated to be cytotoxic. A non-cytotoxic agent may include a bead, liposome, matrix or particle (see, e.g., U. S. Patent Publications 2003/0028071 and 2003/0032995, which are incorporated by reference herein). Such agents may be conjugated, coupled, linked or associated with a dimeric composition described herein.

In some embodiments, conventional cancer medicaments are administered with the compositions described herein. In some embodiments, the subject in need of cancer treatment is treated with the dimeric composition described herein in conjunction with one or more additional agents directed to target cancer cells. Highly suitable agents include those agents that promote DNA-damage, e.g., double stranded breaks in cellular DNA, in cancer cells. Any form of DNA-damaging agent know to those of skill in the art can be used. DNA damage can typically be produced by radiation therapy and/or chemotherapy. DNA-damaging agents are also referred to as genotoxic agents. As used herein, “in conjunction with” shall mean that dimeric composition is administered to a subject concurrently with one or more additional therapies (either simultaneously or separately but in close proximity), prior to, or after administration of one or more additional therapies.

Examples of radiation therapy include, without limitation, external radiation therapy and internal radiation therapy (also called brachytherapy). Energy sources for external radiation therapy include x-rays, gamma rays and particle beams, energy sources used in internal radiation include radioactive iodine (iodine or iodine), strontium, or radioisotopes of phosphorous, palladium, cesium, indium, phosphate, or cobalt. Methods of administering radiation therapy are well known to those of skill in the art.

Examples of DNA-damaging chemotherapeutic agents that may be particularly useful include, without limitation: Busulfan (Myleran), Carboplatin (Paraplatin), Carmustme (BCNU), Chlorambucil (Leukeran), Cisplatin (Platmol), Cyclophosphamide (Cytoxan, Neosar), Dacarbazme (DTIC-Dome), Ifosfamide (Ifex), Lomustme (CCNU), Mechlorethamme (nitrogen mustard, Mustargen), Melphalan (Alkeran), and Procarbazine (Matulane).

A number of other chemotherapeutic agents may be also used for the method described herein, either alone or in combination. These include: methotrexate, vincristine, adriamycin, cisplatin, non-sugar containing chloroethylnitrosoureas, 5-fluorouracil, mitomycin C, bleomycin, doxorubicin, dacarbazine, taxol, fragyline, Meglamine GLA, valrubicin, carmustaine and poliferposan, MMI270, BAY 12-9566, RAS farnesyl transferase inhibitor, farnesyl transferase inhibitor, MMP, MTA/LY231514, LY264618/Lometexol, Glamolec, CI-994, TNP-470, Hycamtin/Topotecan, PKC412, Valspodar/PSC833, Novantrone/Mitroxantrone, Metaret/Suramin, Batimastat, E7070, BCH-4556, CS-682, 9-AC, AG3340, AG3433, Incel/VX-710, VX-853, ZD0101, ISI641, ODN 698, TA 2516/Marmistat, BB2516/Marmistat, CDP 845, D2163, PD183805, DX8951f, Lemonal DP 2202, FK 317, Picibanil/OK-432, AD 32Nalrubicin, Metastron/strontium derivative, Temodal/Temozolomide, Evacet/liposomal doxorubicin, Yewtaxan/Paclitaxel, Taxol/Paclitaxel, Xeload/Capecitabine, Furtulon/Doxifluridine, Cyclopax/oral paclitaxel, Oral Taxoid, SPU-077/Cisplatin, HMR 1275/Flavopiridol, CP-358 (774)/EGFR, CP-609 (754)/RAS oncogene inhibitor, BMS-182751/oral platinum, UFT (Tegafur/Uracil), Ergamisol/Levamisole, Eniluracil/776C85/5FU enhancer, Campto/Levamisole, Camptosar/Irinotecan, Tumodex/Ralitrexed, Leustatin/Cladribine, Paxex/Paclitaxel, Doxil/liposomal doxorubicin, Caelyx/liposomal doxorubicin, Fludara/Fludarabine, Pharmarubicin/Epirubicin, DepoCyt, ZD 1839, LU 79553/Bis-Naphtalimide, LU 103793/Dolastain, Caetyx/liposomal doxorubicin, Gemzar/Gemcitabine, ZD 0473/Anormed, YM 116, Iodine seeds, CDK4 and CDK2 inhibitors, PARP inhibitors, D4809/Dexifosamide, Ifes/Mesnex/Ifosamide, Vumon/Teniposide, Paraplatin/Carboplatin, Plantinol/cisplatin, Vepeside/Etoposide, ZD 9331, Taxotere/Docetaxel, prodrug of guanine arabinoside, Taxane Analog, nitrosoureas, alkylating agents such as melphelan and cyclophosphamide, Aminoglutethimide, Asparaginase, Busulfan, Carboplatin, Chlorombucil, cisplatin, Cytarabine HCl, Dactinomycin, Daunorubicin HCl, Estramustine phosphate sodium, Etoposide (VP16-213), Floxuridine, Fluorouracil (5-FU), Flutamide, Hydroxyurea (hydroxycarbamide), Ifosfamide, Interferon Alfa-2a, Alfa-2b, Leuprolide acetate (LHRH-releasing factor analog), Lomustine (CCNU), Mechlorethamine HCl (nitrogen mustard), Mercaptopurine, Mesna, Mitotane (o.p′-DDD), Mitoxantrone HCl, Octreotide, Plicamycin, Procarbazine HCl, Streptozocin, Tamoxifen citrate, Thioguanine, Thiotepa, Vinblastine sulfate, Amsacrine (m-AMSA), Azacitidine, Erthropoietin, Hexamethylmelamine (HMM), Interleukin 2, Mitoguazone (methyl-GAG; methyl glyoxal bis-guanylhydrazone; MGBG), Pentostatin (2′deoxycoformycin), Semustine (methyl-CCNU), Teniposide (VM-26), and Vindesine sulfate, but it is not so limited.

In addition, the following agents may be also useful for the instant invention: alkylating agents, such as carboplatin and cisplatin, nitrogen mustard alkylating agents, nitrosourea alkylating agents, such as carmustine (BCNU), antimetabolites, such as methotrexate, folinic acid, purine analog antimetabolites, mercaptopurine, pyrimidine analog antimetabolites, such as fluorouracil (5-FU) and gemcitabine (Gemzar®), hormonal antineoplastics, such as goserelin, leuprolide, and tamoxifen, natural antineoplastics, such as aldesleukin, interleukin-2, docetaxel, etoposide (VP-16), interferon alfa, paclitaxel (Taxol®), and tretinoin (ATRA), antibiotic natural antineoplastics, such as bleomycin, dactmomycin, daunorubicin, doxorubicin, daunomycin and mitomycins including mitomycin C, and vinca alkaloid natural antineoplastics, such as vinblastine, vincristine, vindesine, hydroxyurea, acetone, adriamycin, ifosfamide, enocitabine, epitiostanol, aclarubicin, ancitabine, nimustine, procarbazine hydrochloride, carboquone, carboplatin, carmofur, chromomycin A3, antitumor polysaccharides, antitumor platelet factors, cyclophosphamide (Cytoxan®), Schizophyllan, cytarabine (cytosine arabinoside), dacarbazine, thiomosine, thiotepa, tegafur, dolastatins, dolastatin analogs such as auristatin, CPT-11 (irinotecan), mitozantrone, vinorelbine, teniposide, aminopterin, carbomycin, esperamicins (See, e.g., U.S. Pat. No. 4,675,187, which is incorporated by reference herein), neocarzinostatin, OK 432, bleomycin, furtulon, broxundine, busulfan, honvan, peplomycin, bestatin (Ubenimex®), interferon-β, mepitiostane, mitobromtol, melphalan, laminin peptides, lentinan, Coriolus versicolor extract, tegafur/uracil, estramustine (estrogen/mechlorethamine), thalidomide, and lenalidomide (Revlmid®).

Other suitable chemo therapeutics include proteasome inhibiting agents. Proteasome inhibitors block the action of proteasomes, cellular complexes that degrade proteins, particularly those short-lived proteins that are involved in cell maintenance, growth, division, and cell death. Examples of proteasome inhibitors include bortezomib (Velcade®), lactacystin (AG Scientific, Inc, San Diego, Calif.), MG1 32 (Biomol International, Plymouth Meeting, Pa.) PS-519, eponemycin, epoxomycin, aclacinomycin A, the dipeptide benzamide, CVT-63417, and vinyl sulfone tripeptide proteasome inhibitors.

In some embodiments, the methods described herein are used in conjunction with one or more other cancer treatments, including cancer immunotherapy. Cancer immunotherapy is the use of the immune system to reject cancer. The main premise is stimulating the subject's immune system to attack the tumor cells that are responsible for the disease. This can be either through immunization of the subject, in which case the subject's own immune system is rendered to recognize tumor cells as targets to be destroyed, or through the administration of therapeutics, such as antibodies, as drugs, in which case the subject's immune system is recruited to destroy tumor cells by the therapeutic agents. Cancer immunotherapy includes an antibody-based therapy and cytokine-based therapy.

A number of therapeutic monoclonal antibodies have been approved by the FDA for use in humans, and more are underway. The FDA-approved monoclonal antibodies for cancer immunotherapy include antibodies against CD52, CD33, CD20, ErbB2, vascular endothelial growth factor and epidermal growth factor receptor. These and other antibodies targeting one or more cancer-associated antigen are thus suitable for use in a combination therapy to be administered in conjunction with dimeric composition described herein. Examples of monoclonal antibodies approved by the FDA for cancer therapy include, without limitation: Rituximab (available as Rituxan™), Trastuzumab (available as Herceptin™), Alemtuzumab (available as Campath-IH™) Cetuximab (available as Erbitux™), Bevacizumab (available as Avastin™), Panitumumab (available as Vectibix™), Gemtuzumab ozogamicin (available as Mylotarg™), Ibritumomab tiuxetan (available as Zevalin™) and Tositumomab (available as Bexxar™). Examples of monoclonal antibodies currently undergoing human clinical testing for cancer therapy in the United States include, without limitation: WX-G250 (available as Rencarex™), Ipilimumab (available as MDX-010), Zanolimumab (available as HuMax-CD4), Ofatunumab (available as HuMax-CD20), ch14.18, Zalutumumab (available as HuMax-EGFr), Oregovomab (available as B43.13, OvalRex™) Edrecolomab (available as IGN-101, Panorex™), 131I-chTNT-I/B (available as Cotara™) Pemtumomab (available as R-1549, Theragyn™), Lintuzumab (available as SGN-33), Labetuzumab (available as hMN14, CEAcide™), Catumaxomab (available as Removab™), CNTO 328 (available as cCLB8), 3F8, 177Lu-J591, Nimotuzumab, SGN-30, Ticilimumab (available as CP-675206), Daclizumab (available as Zenapax™), Epratuzumab (available as hLL2, LymphoCide™), 90Y-Epratuzumab, Galiximab (available as IDEC-114), MDX-060, CT-011, CS-1008, SGN-40, Mapatumumab (available as TRM-I), Apolizumab (available as HuIDlO, Remitogen™) and Volociximab (available as M200).

Cancer immunotherapy also includes a cytokine-based therapy. The cytokine-based cancer therapy utilizes one or more cytokines that modulate a subject's immune response. Non-limiting examples of cytokines useful in cancer treatment include interferon-a (IFN-a), interleukin-2 (IL-2), Granulocyte-macrophage colony-stimulating factor (GM-CSF) and interleukin-12 (IL-12).

The entire contents of all of the references (including literature references, issued patents, published patent applications, and co pending patent applications) cited throughout this application are hereby expressly incorporated by reference.

Also provided herein is a method for diagnosing an NKG2D ligand expressing disease, the method comprising (1) obtaining a biological sample from a subject having an NKG2D ligand expressing disease; (2) contacting the biological sample with a binding reagent comprising a dimeric protein described herein or a composition described herein; and (3) detecting a binding of the NKG2D ligand to the binding reagent; where a binding of the NKG2D ligand to the binding reagent indicates a diagnosis of the NKG2D ligand expressing disease in the subject.

In some embodiments, the NKG2D ligand is MICA, MICB, ULBP1, ULBP2, ULBP3, ULBP4, ULBP5, or ULBP6.

Provided herein is a dimeric protein described herein, a polynucleotide described herein, a vector described herein, or a composition described herein, for use as a medicament

Provided herein is a dimeric protein described herein, a polynucleotide described herein, a vector described herein, or a composition described herein, for use in the treatment of a disease.

Provided herein is a dimeric protein described herein, a polynucleotide described herein, a vector described herein, or a composition described herein, for use in the treatment of a disease, wherein said disease is an NKG2D ligand expressing disease.

Provided herein is a dimeric protein described herein, a polynucleotide described herein, a vector described herein, or a composition described herein, for use in the treatment of a disease, wherein said disease is cancer or autoimmune disease.

Preparation of Dimeric NKG2D-Fc Proteins

In some embodiments, dimeric NKG2D-Fc chimeric constructs are produced by conventional recombinatory DNA methods. In some embodiments, a dimeric NKG2D protein described herein is produced as a single (e.g., contiguous) recombinant polypeptide. In other embodiments, two or more portions of dimeric NKG2D protein are produced as separate fragments and are subsequently linked together to yield a dimeric NKG2D-Fc molecule. In some embodiments, each NKG2D portion of the dimeric protein and an Fc portion of the dimeric protein are each produced as separate recombinant polypeptides then fused together by a chemical linking means to yield fused NKG2D-Fc. This production methodology may be preferred particularly in situations where a non-peptide linking molecule is employed. Similarly, this production methodology may be also preferred if a dimeric NKG2D-Fc protein does not fold correctly (e.g., does not properly bind a ligand) when made as a single contiguous polypeptide. In some embodiments, each monomer of the dimeric protein (e.g., the NKG2D portion and the Fc portion) is encoded by a single continuous nucleotide sequence, and produced in the cells as a singled recombinant polypeptide. In some embodiments, a dimeric NKG2D molecule is generated by protein-protein interaction, e.g., by Fc-fragment.

For the production of recombinant polypeptides, a variety of host organisms may be used. Suitable hosts include, but are not limited to: bacteria such as E. coli, yeast cells, insect cells, plant cells, and mammalian cells. Choice of a suitable host organism will depend on the particular application of the dimeric NKG2D-Fc protein. The skilled artisan will understand how to take into consideration certain criteria in selecting a suitable host for producing the recombinant polypeptide. Factors affecting selection of a suitable host include, for example, post-translational modifications, such as phosphorylation and glycosylation patterns, as well as technical factors, such as the general expected yield and the ease of purification. Host-specific post-translational modifications of a dimeric NKG2D-Fc, which is to be used in vivo, should be carefully considered because certain post-specific modifications are known to be highly immunogenic (antigenic).

Once produced, dimeric NKG2D-Fc can be purified by any suitable means, such as chromatographic methods known to those of skill in the art. Examples of chromatographic methods include gel filtration chromatography. See, for example, Caine et al., Protein Expr. Purif., 1996, 8: 159-66. In some embodiments, dimeric NKG2D-Fc is purified by Protein A immunoaffinity chromatography.

As will be recognized by one of ordinary skill in the art, dimeric NKG2D chimera portions also can be prepared and isolated separately, and joined by chemical synthesis.

### EXAMPLES

### Example 1. Constructs Design

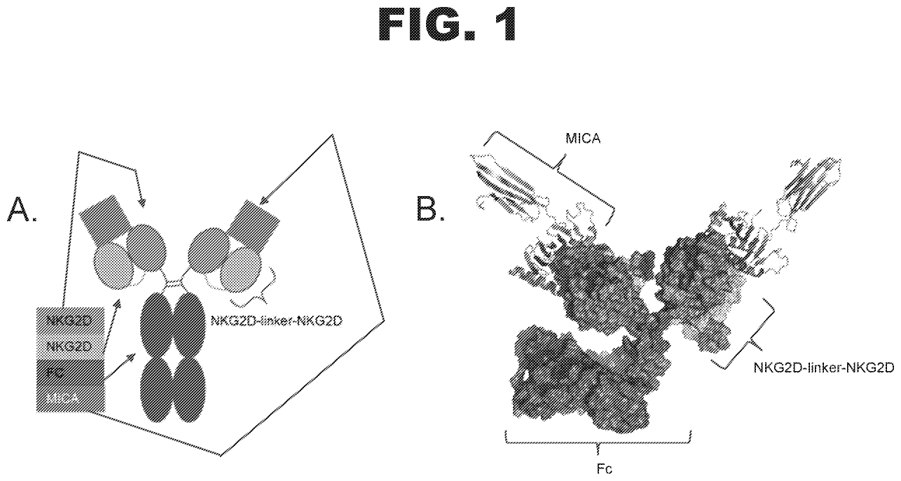

A model of the NKG2D-Fc variant was built based on structural analysis of full length human IgG1 antibody (PDB 1HZH) and complex NKG2D/MICA (PDB IHYR), using MOE (Chemical Computing Group ULA). FIG. 1A shows a schematic representation of a NKG2D-Fc variant. The model was visualized in PyMol Molecular Graphics System (Schroedinger LLC). In the model, the regions in blue represent the Fc portion of the NKG2D-Fc variant, the regions in green represent the NKG2D portions of the NKG2D-Fc variant, and the regions in orange represent bound MICA protein (FIG. 1B).

### Example 2. Engineering and Sequences

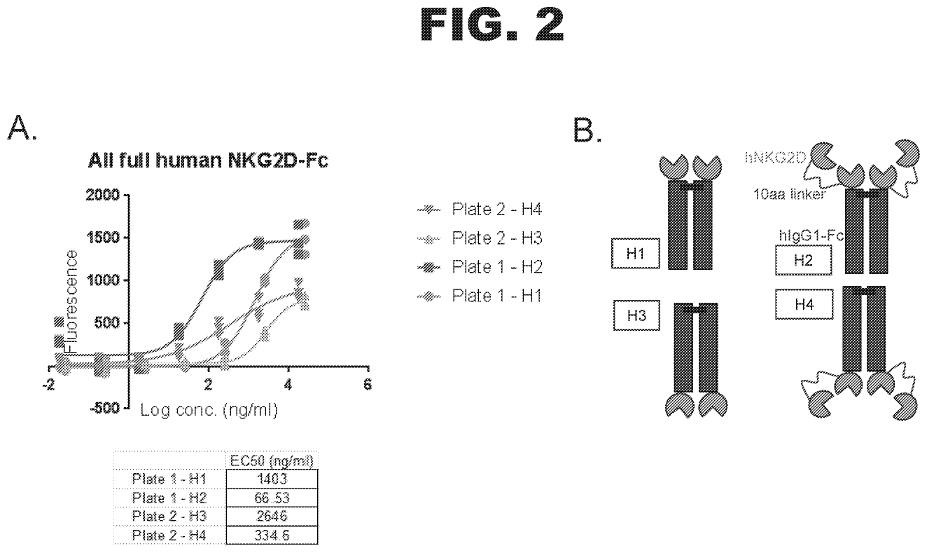

### Example 2.1 (FIGS. 2A-2B)

NKG2D binds to its ligands as a dimer. When NKG2D is fused to an Fc portion, the NKG2D dimer can be formed either by the dimerization of the Fc fragment, or, two NKG2D fragments can be linked by a peptide linker and fused to Fc fragment, resulting in a bivalent homo-dimeric protein with two NKG2D dimers.

The ability to induce ADCC signaling of both previously described variants as well as N-terminal and C-terminal Fc fusions were tested in a reporter gene assay (RGA).

The four human dimeric NKG2D-Fc variants were first transiently expressed into HEK293T cells and purified by Protein A immunoaffinity chromatography. 1.5e4 engineered stable CT26.WT cells over-expressing recombinant human MICA*008 (target cells) were re-suspended in RPMI-Glutamax medium (Gibco) supplemented with 10% FBS (assay medium) and plated in the wells of a white 96-well plate. The cells were incubated with the different molar concentrations of the dimeric NKG2D-Fc variants diluted in assay medium. After one hour incubation, 9e4 FcγRIIIa V158 NFAT luciferase Jurkat (JNL) cells (effector cells) were added to the experimental wells resulting in a target:effector cell ratio of 1:6. Assay plate was incubated for four hours at 37° C. and 5% CO2. Concentration-dependent activation of the FcγRIIIa receptor was revealed by the addition of 60 ul Bright-Glo™ (Promega) per well and incubation for 3 minutes at room temperature protected from light. The resulting bioluminescence was measured using a Synergy HT plate reader (Biotek). Half maximal effective concentration (EC50) in ng/ml was determined with background subtracted fluorescence signals (see, e.g., FIGS. 2A-2B).

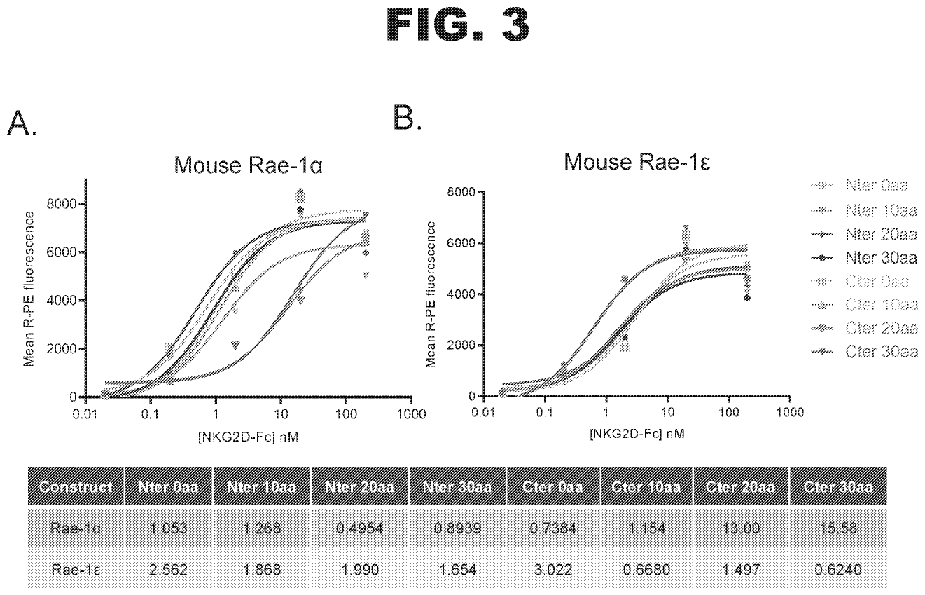

### Example 2.2 (FIGS. 3A-3B)

A Flow-activated cell sorting (FACS) based binding assay against mouse NKG2D-ligands expressed at the cell surface was performed to characterize the binding of the dimeric mouse NKG2D-Fc variants fused either to N-terminus or to C-terminus of the Fc region with no peptide linker or with different peptide linker lengths (10, 20 or 30 amino acids).

Half maximal effective concentration (EC50) in nM was determined on two different engineered stable CT26.WT cell lines, over-expressing at their surface the recombinant ligands mouse Rae-1α and mouse Rae-1E. The cells were incubated with the different concentrations of protein in DPBS supplemented with 5% FCS and 0.04% NaN₃ (FACS buffer). The bound proteins were detected with an anti-mouse Fc antibody labelled with R-Phycoerythrin (R-PE) (Jackson ImmunoResearch, ref. 115-115-164). For each sample, the mean of the resulting fluorescence of alive gated cells was extracted using the Cell Quest Pro software (BD Biosciences).

All variants bind similar to Rae-1e, independent on linker length and orientation. In contrast, C-terminal variant with a linker length of 20 or 30 aa show much decreased binding to Rae-1a. All other variants show comparable binding to this ligand.

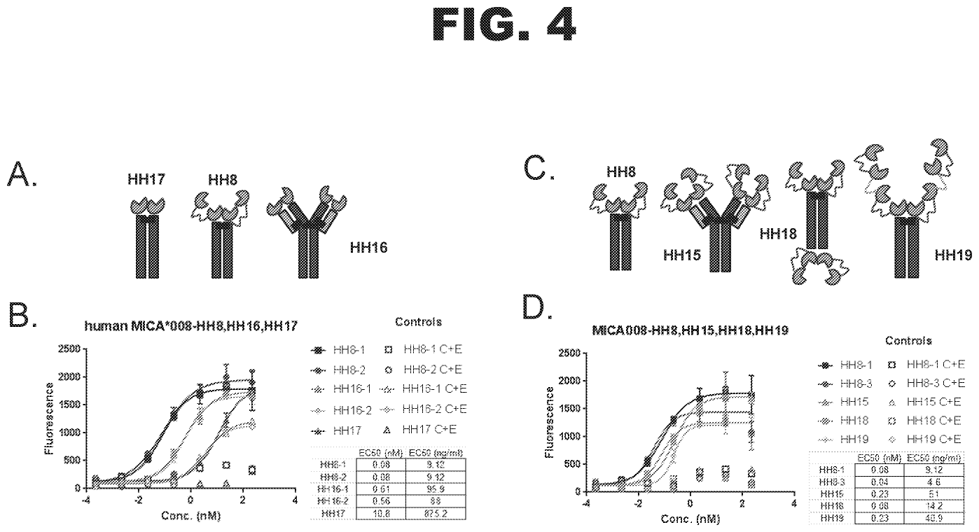

### Example 2.3 (FIGS. 4A-4D)

The ability to induce ADCC signaling of dimeric NKG2D-Fc variants with different architecture and NKG2D valences was tested in a reporter gene assay.

The variants were first transiently expressed into HEK293T cells and purified by Protein A immunoaffinity chromatography.

1.5e4 engineered stable CT26.WT cells over-expressing recombinant human MICA*008 (target cells) were re-suspended in RPMI-Glutamax medium (Gibco) supplemented with 10% FBS (assay medium) and plated in the wells of a white 96-well plate. The cells were incubated with the different molar concentrations of the dimeric NKG2D-Fc variants diluted in assay medium. After one hour incubation, 9e4 FcγRIIIa V158 NFAT luciferase Jurkat (JNL) cells (effector cells) previously blocked for 1-2 hours with an anti-ULBP2, ULBP5, ULBP6 goat antibody (Thermo Scientific, ref. PAS-47118) were added to the experimental wells resulting in a target:effector cell ratio of 1:6. As control, reactivity of the NKG2D-Fc variants with effector cells was assessed by incubating NKG2D-Fc proteins with effector cells in absence of target cells (C+E wells). Assay plate was incubated for four hours at 37° C. and 5% CO2. Concentration-dependent activation of the FcγRIIIa receptor was revealed by the addition of 60 ul One-Glo™ (Promega) per well and incubation for 3 minutes at room temperature protected from light. The resulting bioluminescence was measured with a Synergy HT plate reader (Biotek). Half maximal effective concentration (EC50) in ng/ml was determined with background subtracted fluorescence signals (see, e.g., FIGS. 4A-4D).

Valency and architecture play a role in ability to target a cell and induce cell killing. FIG. 4B shows increase in EC50 of at least one log from monovalent (HH17) to a bivalent (HH16) binding. Surprisingly, architecture of molecules, here HH16 vs. HH8 has at least one log difference in cell killing (EC50). A further increase of valency from 2 to 4 or another architecture (FIGS. 4C-4D) does not improve cell killing.

### Example 2.4 (FIGS. 5A-5D)

An analysis of protease cleavage and degradation in a relevant expression host is an important parameter for further development in this expression host.

The variants were transiently expressed into CHO MaKO cells by flow electroporation (Maxcyte®) and purified by Protein A immunoaffinity chromatography.

The level of clipped proteins for each variant was assessed by LC-MS after full denaturation reduction and de-glycosylation of the protein.

A direct binding assay against recombinant human NKG2D ligand was performed to determine if the five amino acid deletion affects the binding of dimeric NKG2D-Fc to its ligands.

Kinetic binding affinity constants (K_{D}) were measured on protein-A captured NKG2D-Fc variants using recombinant human MICA*001 ligand as analyte. Measurements were conducted on a BIAcore® T200 (GE Healthcare, Glattbrugg, Switzerland) at room temperature. The NKG2D-Fc variants were captured on the flow cell of a Protein A research grade sensor chip (GE Healthcare, ref 29127555). All proteins were diluted in 0.01 M HEPES pH 7.4, 0.15 M NaCl, 3 mM EDTA, 0.005% v/v Surfactant P20. To serve as reference, one flow cell was left blank without any captured protein. Binding data were acquired by subsequent injection of analyte dilutions series on the reference and measuring flow cell. Zero concentration samples (running buffer only) were included to allow double referencing during data evaluation. For data evaluation, doubled referenced sensorgrams were used and dissociation constants (Kb) analyzed.

As specific protease cleavage site could be identified by LC-MS in second NKG2D domain (FIGS. 5A-5C). By deletion of this site a significant reduction of degradation products could be achieved (FIG. 5D). As expected, clipping at aa position 149 contributes to only to 6% reduction of monomeric fraction the binding affinities are comparable of tested samples (FIG. 5D).

### Example 3. Construct Format

Material from cell line close to developmental production was tested to confirm binding and cell killing activity.

1.5e4 engineered stable CT26.WT cells (target cells) over-expressing recombinant human MICA*008, human MICA*001, human MICB*001 or cynomolgus MICB05 ligands and GUS, an irrelevant target, were resuspended in RPMI-Glutamax medium (Gibco) supplemented with 10% FBS (assay medium) and plated in the wells of a white 96-well plate. The cells were incubated with the different molar concentrations of the dimeric NKG2D-Fc variants diluted in assay medium. After one hour incubation, 9e4 FcγRIIIa V158 NFAT luciferase Jurkat (JNL) cells (effector cells) previously blocked for 1-2 hours with an anti-ULBP2, ULBP5, ULBP6 goat antibody (Thermo Scientific, ref. PAS-47118) were added to the experimental wells resulting in a target:effector cell ratio of 1:6. Assay plate was incubated for four hours at 37° C. and 5% CO2. Concentration-dependent activation of the FcγRIIIa receptor was revealed by the addition per well of 60 ul One-Glo™ (Promega) and incubation for 3 minutes at room temperature protected from light. The resulting bioluminescence was measured with a Synergy HT plate reader (Biotek). Half maximal effective concentration (EC50) in ng/ml was determined with background subtracted fluorescence signals.

Results demonstrate, surprisingly, an increase in activity of CHO derived material versus HEK derived material from a genetically identical construct by at least a factor of 10. Findings are similar for at least 2 relevant human targets (FIG. 9E).

### Example 4. Production and Clipping

Level of protease cleavage, in particular for position 149/150 was analysed for material derived from different expression hosts, but genetically identical.

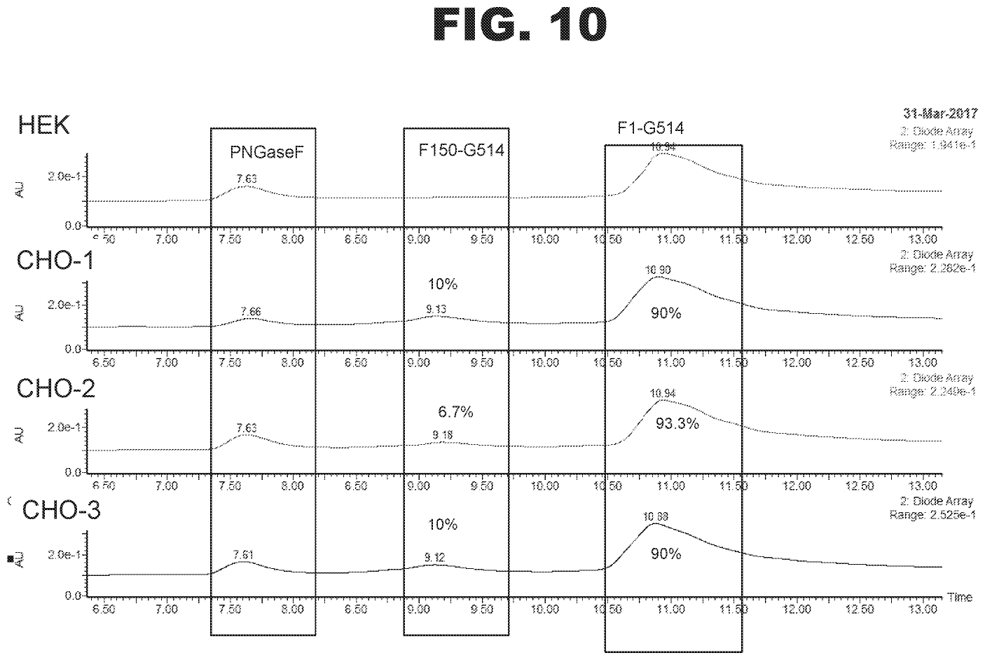

Each protein batch was denatured reduced and deglycosylated by dissolving 25 micrograms in 8M Urea, 0.4M NH₄CO₃, 0.17M DTT and heating for 30 minutes at 50° C., followed by a 1-hour incubation at 37° C. with 1 microgram of PNGase F after addition of 40 microliter of milliQ water. The reduced denatured deglycosylated protein was injected in Liquid-chromatography-mass spectroscopy (LC-MS) (BEH C4 1 mm*150 mm with UV at 214 nm, Waters QTOF premier) (see, e.g., FIG. 10).

2 mgs of HH2 reference sample (produced in HEK-6E) were spiked into 50 mL DM133 cell culture media (Irvine Scientific) inoculated with 2E5 cells/mL of CHO-C8TD, CHO-MaKo and CHO-HPT3. The shake flasks (Corning) were cultivated for 8 days at 36.5° C. in an orbital shaker (Infors HT) at 150 rpm and 10% CO₂ under monitoring of cell densities and viabilities using Beckman Coulter Vicell. All supernatants were harvested and frozen at <−60° C. until purification. After thawing at 30° C. in a water bath, the supernatants were sterile filtered prior to purification on a 1 ml MabSelect SuRe column (GE Healthcare), with step elution using 50 mM acetic acid, pH 3.0. All eluates were directly titrated to pH 5.0 with 1 M Tris. Aliquots for analytics were taken and immediately frozen at <−60° C. until analysis by Mass Spectrometry. 25 ug protein of each sample were denatured with Urea, reduced with DTT and deglycosylated with PNGaseF. All proteins were subsequently analyzed with a LC-MS QTOF premier system (Waters) on a BEH C4 RP column (Waters) using a water/acetonitrile gradient. The results are summarized in the Table 1 below.

TABLE 1
Results summary of CHO co-cultivation clipping study on HH2

| | Reference | Mako | C8TD | HPT3 |
|---|---|---|---|---|
| | (UV | (UV | (UV | (UV |
| RT(min) | %) | %) | %) | %) |
| 5.7 | 0.07 | 0.75 | 1 | 0.57 |
| | (G236- | (V230- | (V230- | (V230- |
| | G514) | G514) | G514) | G514) |
| | 0.13 | 0.25 | 0.5 | 0.13 |
| | (V230- | (L229- | (L229- | (L229- |
| | G514) | G514) | G514) | G514) |
| 7.22 | 1.9 | 5.8 | 10.8 | 4.4 |
| | (N156- | (F155- | (F155- | (F155- |
| | G514) | G514) | G514) | G514) |
| | 3.1 | | | |
| | (F155- | | | |
| | G514) | | | |
| | 0.8 | | | |
| | (S153- | | | |
| | G514) | | | |
| 7.97 | | | 4.6 | |
| | | | (S141- | |
| | | | G514) | |
| 9.41 | 93.9 | 93 | 82.9 | 94.7 |
| | (F1- | (F1- | (F1- | (F1- |
| | G514) | G514) | G514) | G514) |

Further productions of HH2, HH8, HH8-afuco and HH10 in stable CHO-MaKO cells confirmed initial results of the co-cultivation assay. Only minor amounts of clipped products (low levels of 1% or below) were detected by RP-MS analyses for all submitted samples.

Method 1 (reduced and alkylated RP-MS): 400 ug of protein were diluted to a total volume of 356 ul with Milli-Q water and pH was adjusted adding 20 uL of 1M Tris, pH 8.0. Deglycosylation was carried out adding 24 ul of PNGaseF (GNF, 1 mg/mL) and incubated over night at 37° C. 44 ul of the deglycosylated sample were mixed with 50 uL 8M Guanidine-HCl, 5 ul 1M Tris pH 8.0, 1 uL 1M DTT. After an incubation for 1 hour at 37° C., immediately 2 ul 1M IAM were added and incubated for another 1 hour at room temperature in darkness. 1 ul of 1M DTT were finally added to the sample to quench the alkylation and 3 ug of protein were subsequently analyzed with a LC-MS QTOF system (Waters) on a PLRP-S column (Agilent) at 60° C. using a water/isopropanol and acetonitrile gradient.

Method 2 (reduced RP-MS): 100 ug of protein were diluted 1:18 to a total volume of 9 ul with Milli-Q water and pH was adjusted adding 1.7 uL of 0.5M Tris, pH 7.5. Deglycosylation was carried out adding 6 ul of PNGaseF (GNF, 1 mg/mL) and 1 ul of EndoH (NEB, 125,000 U/mL). The samples were incubated over night at 37° C. 7.3 ul of the deglycosylated sample were mixed with 50 uL 8M Guanidine-HCl, 5 ul 1M Tris pH 8.0, 1 uL 1M DTT and incubated for 1 hour at 37° C. Immediately 1 ul of 10% TFA was added to the sample. 3 ug of protein were subsequently analyzed with a LC-MS QTOF system (Waters) on an Acquity CSH C18 (Waters) at 80° C. using a water/isopropanol and acetonitrile gradient.

### Example 5. In Vitro Binding

A direct binding assay was performed to characterize the binding of the ADCC enhanced and wild-type NKG2D-Fc variants against recombinant human MICA*001 and MICB*001 ligands. The NKG2D-Fc variants were first used as ligand to characterize the binding of each NKG2D dimer to MICA and MICB (monovalent binding setup) and secondly as analyte in order to determine the effect on the binding when having two homodimers (bivalent binding setup).

For the monovalent binding setup, kinetic binding affinity constants (K_{D}) were measured on protein-A captured NKG2D-Fc variants using recombinant human NKG2D ligands as analyte. Measurements were conducted on a BIAcore® T200 (GE Healthcare, Glattbrugg, Switzerland) at room temperature. The NKG2D-Fc variants were captured on the flow cell of a Protein A research grade sensor chip (GE Healthcare, ref 29127555). All proteins were diluted in 0.01 M HEPES pH 7.4, 0.15 M NaCl, 3 mM EDTA, 0.005% v/v Surfactant P20. To serve as reference, one flow cell was left blank without any captured protein. Binding data were acquired by subsequent injection of analyte dilutions series on the reference and measuring flow cell. Zero concentration samples (running buffer only) were included to allow double referencing during data evaluation. For data evaluation, doubled referenced sensograms were used and dissociation constants (K_{D}) analyzed.

For the bivalent binding setup, kinetic binding affinity constants (K_{D}) were measured on streptavidin captured recombinant human NKG2D ligands using NKG2D-Fc variants as analyte. Measurements were conducted on a BIAcore® T200 (GE Healthcare, Glattbrugg, Switzerland) at room temperature. All proteins were diluted in 0.01 M HEPES pH 7.4, 0.15 M NaCl, 3 mM EDTA, 0.005% v/v Surfactant P20. The biotinylated NKG2D ligands were covalently captured on the flow cells of a SA research grade sensor chip (GE Healthcare, ref 29104992) using standard procedure according to the manufacturer's recommendation (GE Healthcare). To serve as reference, one flow cell was blank immobilized. Binding data were acquired by subsequent injection of analyte dilutions series on the reference and measuring flow cell. Zero concentration samples (running buffer only) were included to allow double referencing during data evaluation. For data evaluation, doubled referenced sensograms were used and dissociation constants (K_{D}) analyzed.

In a bivalent binding setup, all 3 analyzed samples (HH8 with IgG1-Fc wt, HH10 with IgG1-Fc_SDIE, and HH8_afuc with IgG1-Fc wt with low content of fucose) show comparable binding in sub-nanomolar range for both ligands, MICA*001 and MICB*001. As expected, measured affinities are lower in a monovalent binding setup due to avidity effect. Here, binding of all samples to MICA*001 is in double digit nM range there as binding to MICB*001 is decreased by factor of 2.

TABLE 2
Bivalent and monovalent binding

| | | | | | Monovalent |
|---|---|---|---|---|---|
| | | Bivalent binding | | | binding |
| Sample | Ligand | ka (1/Ms) | kd (1/s) | KD (nM) | KD (nM) |
| HH8 | hMICA*001 | 4.83E+06 | 8.14E−04 | 0.79 | 60 |
| | hMICB*001 | 3.89E+06 | 0.002815 | 0.89 | 114 |
| HH10 | hMICA*001 | 1.11E+06 | 9.07E−04 | 0.82 | 77.5 |
| | hMICB*001 | 6.22E+06 | 0.003277 | 0.53 | 86 |
| HH8-AFUCO | hMICA*001 | 7.55E+05 | 0.001067 | 1.41 | 70 |
| | hMICB*001 | 3.93E+06 | 0.004109 | 1.05 | 104 |

6e6 293FT cells (Invitrogen) were plated into a 10 cm dish with 10 mL cD10, then 3 hours later media was changed to 5 mL pre-warmed Opti-MEM1. Cells were transfected 4 hours after OptiMem-1 addition using lipofectamine-2000 according to manufacturers instructions with DNA plasmids encoding for the indicated ligands. After overnight incubation, the cells were harvested with two sequential washes of 5 mL PBS+2 mM EDTA. Cells were counted on a Nexcelom Cell Counter, and 100e3 cells were resuspended in PBS+2 mM EDTA+2% Fetal Bovine Serum (FACS Buffer) with 10 ug/mL HH8 or 10 ug/mL Human IgG1 Isotype. Cells were stained for 15 minutes at 4 degrees, then washed once in 200 uL FACS Buffer, and stained with a secondary antibody, Alexa488-labeled goat anti-huIgG (A11013, Thermo Fisher) for 15 minutes at 4 degrees. Cells were then washed once in 200 uL FACS Buffer, resuspended in 100 uL FACS Buffer and fixed with an additional 100 uL of IC Fixation Buffer (eBioscience) before running on a BD Fortessa and analyzing in Flowjo (see, e.g., FIGS. 11A-11B).

6e6 293FT cells (Invitrogen) were plated into a 10 cm dish with 10 mL cD10, then 3 hours later media was changed to 5 mL pre-warmed Opti-MEM1. Cells were transfected 4 hours after OptiMem-1 addition using lipofectamine-2000 according to manufacturers instructions with DNA plasmids encoding for the indicated ligands. After overnight incubation, the cells were harvested with two sequential washes of 5 mL PBS+2 mM EDTA. Cells were counted on a Nexcelom Cell Counter, and 100e3 cells were resuspended in PBS+2 mM EDTA+2% Fetal Bovine Serum (FACS Buffer) with 10 ug/mL HH8 or 10 ug/mL Human IgG1 Isotype. Cells were stained for 15 minutes at 4 degrees, then washed once in 200 uL FACS Buffer, and stained with a secondary antibody, Alexa488-labeled goat anti-huIgG (A11013, Thermo Fisher) for 15 minutes at 4 degrees. Cells were then washed once in 200 uL FACS Buffer, resuspended in 100 uL FACS Buffer and fixed with an additional 100 uL of IC Fixation Buffer (eBioscience) before running on a BD Fortessa and analyzing in Flowjo (see, e.g., FIGS. 12A-12B).

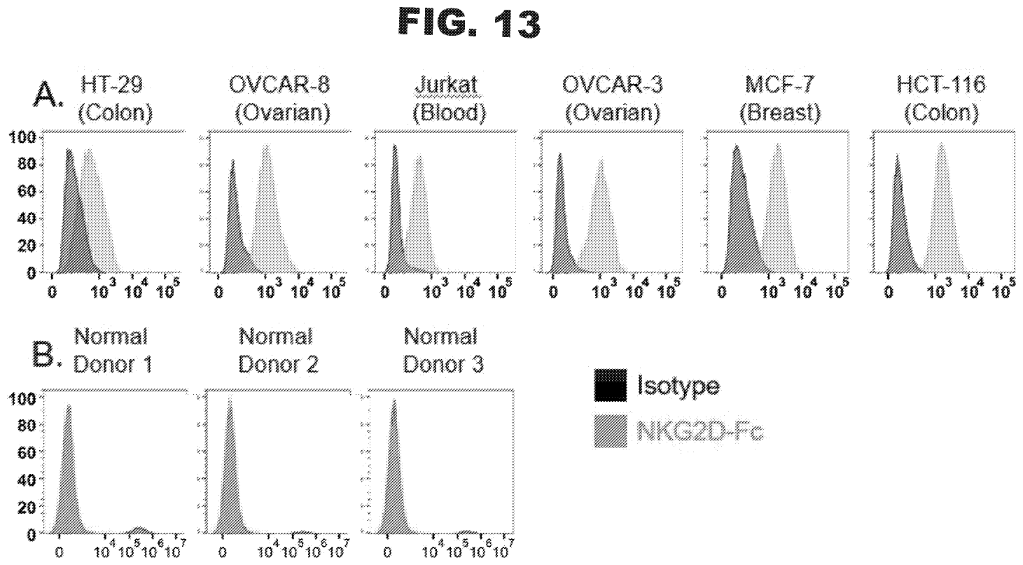

1e6-5e6 tumor cells were seeded directly into 25 mL DMEM+10% FBS in a T-75 flask and then cultured at 37 degrees in 5% CO2. 72 hours later, cells were harvested by pipetting in PBS+2 mM EDTA. Normal donor PBMCs were harvested from Leukopaks (HemaCare) and purified with Ficoll. Cells were counted on a Nexcelom Cell Counter, and 100e3 cells were resuspended in PBS+2 mM EDTA+2% Fetal Bovine Serum (FACS Buffer) with 5 uL TruStain FcX (eBioscience) for 15 minutes at 4 degrees Celsius. Cells were then centrifuged and resuspended in FACS Buffer+10 ug/mL HH8 or 10 ug/mL Human IgG1 Isotype. Cells were stained for 15 minutes at 4 degrees, then washed once in 200 uL FACS Buffer, and stained with a secondary antibody, Alexa488-labeled goat anti-huIgG (A11013, Thermo Fisher) for 15 minutes at 4 degrees. Cells were then washed once in 200 uL FACS Buffer, resuspended in 100 uL FACS Buffer and fixed with an additional 100 uL of IC Fixation Buffer (eBioscience) before running on a BD Fortessa and analyzing in Flowjo (see, e.g., FIGS. 13A-13B).

### Example 6. In Vitro Function

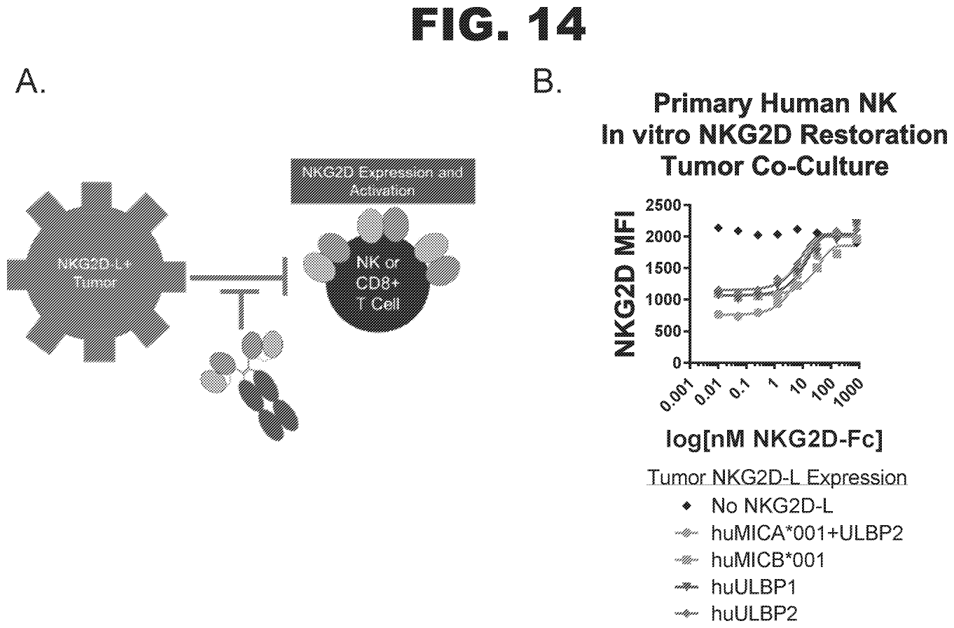

Primary Human NK cells were purified from Leukopaks and frozen in 90% Fetal Bovine Serum+10% DMSO. NK cells were thawed into phenol red-free RPMI+10% Heat-Inactivated FBS+50 uM beta-mercaptoethanol+50 IU/mL human IL-2 (Corning) (Assay media) on the day of the assay. CT26.WT cells engineered to overexpress the indicated NKG2D-L were cultured in assay media and then harvested with PBS+2 mM EDTA. 50,000 NK cells and 25,000 CT26.WT cells were added to each well, and dilutions of NKG2D-Fc antibodies were added as indicated. The assay was then incubated at 37 degrees Celsius+5% CO2 for 24 hours. Non-adherent cells were then harvested and stained with anti-CD45 APC-Cy7 (eBioscience HI-30) and anti-NKG2D APC (eBioscience 1D11) for 15 minutes, followed by a wash and fixation, and then analyzed on a BD Fortessa and Flowjo. NKG2D Median Fluorescence Intensity of CD45+ was normalized to isotype (100%) or bead-based downregulation (0%) (see, e.g., FIGS. 14A-14B).

### Example 7. In vivo Function-ADCC

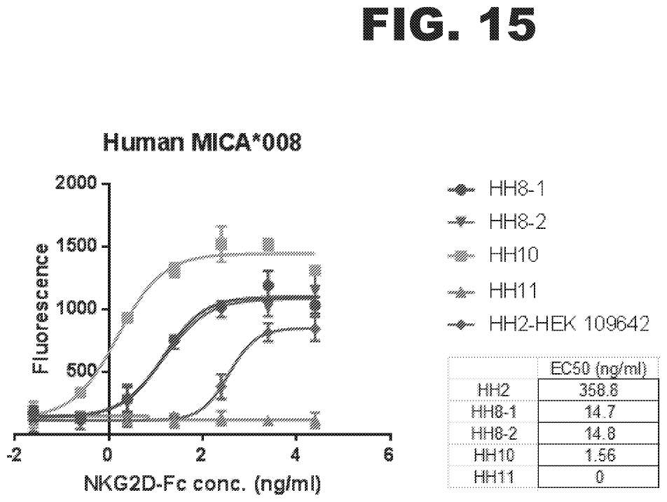

Enhanced Fc gamma receptor binding also results in an increase ADCC activity due to stronger activation of receptors on effector cells, which was tested in a setup using a cell line overexpressing human MICA*008 as target cell line in a cellular RGA (FIG. 15).

1.5e4 engineered stable CT26.WT cells over-expressing recombinant human MICA*008 ligand (target cells) were re-suspended in RPMI-Glutamax medium (Gibco) supplemented with 10% FBS (assay medium) and plated in the wells of a white 96-well plate. The cells were incubated with the different molar concentrations of the dimeric NKG2D-Fc variants diluted in assay medium. After one hour incubation, 9e4 FcγRIIIa V158 NFAT luciferase Jurkat (JNL) cells (effector cells) were added to the experimental wells resulting in a target:effector cell ratio of 1:6. Assay plate was incubated for four hours at 37° C. and 5% CO2. Concentration-dependent activation of the FcγRIIIa receptor was revealed by the addition per well of 60 ul One-Glo™ (Promega) and incubation for 3 minutes at room temperature protected from light. The resulting bioluminescence was measured with a Synergy HT plate reader (Biotek). Half maximal effective concentration (EC50) in ng/ml was determined with background subtracted fluorescence signals.

A NKG2D-Fc version (HH10) with mutations to enhance Fc_gamma receptor binding showed a 10 fold stronger cell killing activity than compared to a NKG2D-Fc version with wildtype IgG1-Fc (HH8). A version of NKG2D-Fc with mutations abrogating Fc_gamma receptor binding show no cell killing activity (FIG. 15). A sample of NKG2D-Fc with IgG1 wt, derived from HEK expression, showed at least a 10 fold lower cell killing activity as shown already in FIG. 9.

TABLE 3
Human CD16 RGA of NKG2D-Fc dimers
expressed in CHO Mako cells

| | |
|---|---|
| EC50 (ng/mL) | |
| Target cell | hRGA killing |

| line/Candidate | HH8 | HH10 | HH2 | HH5 |
|---|---|---|---|---|
| hULBP1 | 5.0 | 0.3 | 8.3 | 1.2 |
| hULBP2 | 10 | 1.2 | 15 | 0.9 |
| hMICA008 | 3.5 | 0.2 | 4.7 | 1 |
| hMICB001 | 35 | 9.5 | 91 | 16 |
| cyMICA08 | 2.1 | 0.3 | 7.3 | 0.2 |
| cyMICB05 | 194 | 145 | 1347 | 89 |
| GUS = negative control | / | / | / | / |

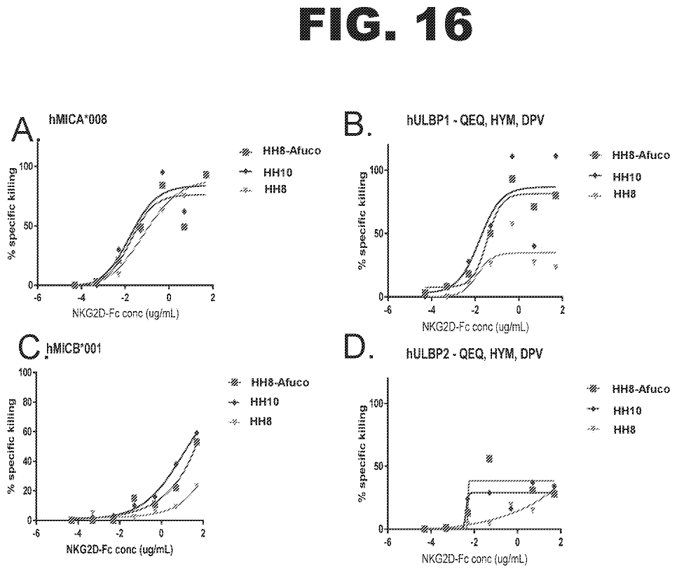

The ADCC activities of the ADCC enhanced (HH10 and HH8-AFUCO) and wild-type (HH8) NKG2D-Fc variants were determined by an in vitro ADCC assay (FIGS. 16A-16D), such as that described in U.S. Pat. No. 5,500,362 or 5,821,337 using a human CD16 expressing effector cell line. The biological ADCC activities of the NKG2D-Fc variants were measured based on their ability to trigger ADCC in the presence of NKG2D ligand-expressing target and NK effector cells. Calcein labeled CT26.WT cells stably transfected with human MICA*008, MICB*001, ULBP1 or ULBP2 were incubated with different concentrations of NKG2D-Fc variants and an excess of natural killer cells, here the NK3.3 cell line (Saint Louis University, ref SLU-1011). The cells were washed and re-suspended in RPMI Phenol free medium to reach a 1:20 target to effector cell ratio. The assay medium was supplemented with 1.5 mM of probenecid (Invitrogen, ref P36400) in order to reduce the spontaneous release of calcein of the target cells. Concentration-dependent killing of the CT26.WT target cells were analyzed after one hour and a half incubation at 37° C./5% CO2, by measuring the release of the calcein from the killed cells in each well. Each assay plate includes a combination of experimental and control wells. Several different types of control wells were used to account for (i) the spontaneous calcein release from target cells (Target Spontaneous) and (ii) the maximum calcein release from the target cells (Target Maximum). The percentage of specific killing was calculated as follow (Experimental-Target Spontaneous)/(Target Maximum−Target Spontaneous). Half maximal effective concentration (EC50) in ng/ml was determined for each target cell lines.

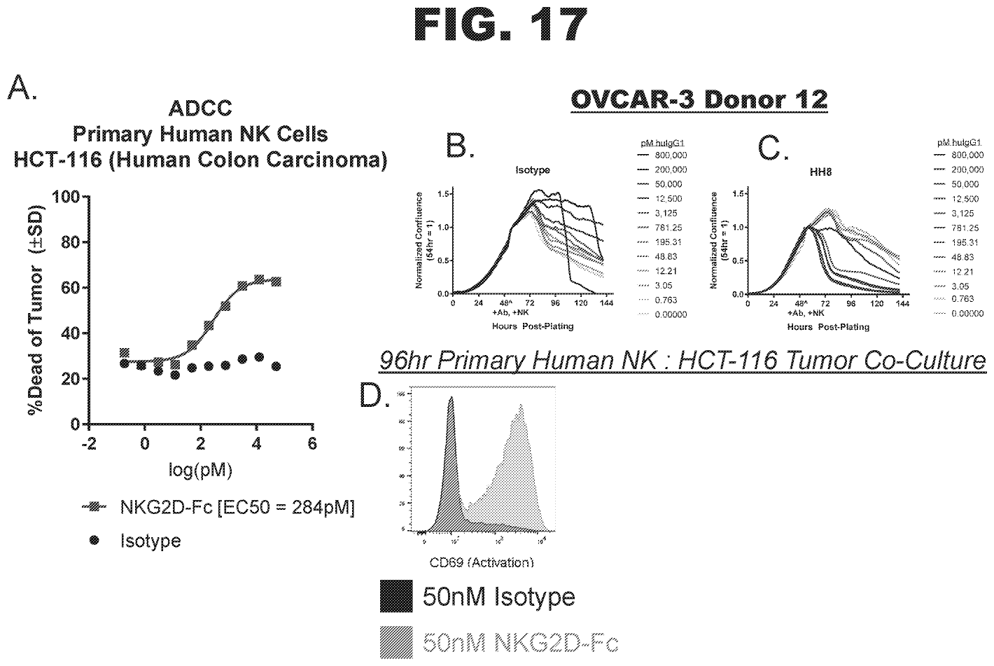

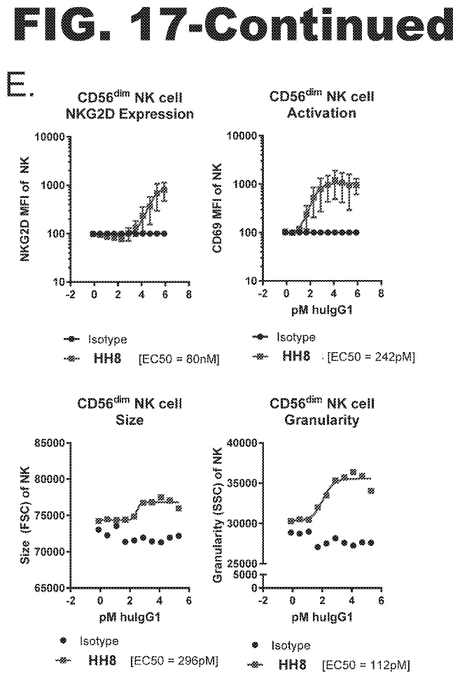

10,000 HCT-116 or OVCAR-3 cells were plated into a 96-well plate in 50 uL of Low IgG cR10+50 uM 2-ME+100 IU/mL human IL-2 and incubated for 51 hours at 37 degrees on an ACEA xCelligence. At 51 hours post-tumor plating, 100e3 NK cells and NKG2D-Fc at the indicated concentration were added in 50 uL for 100 uL assay media final volume. Killing was read at 72 hours post-tumor plating, or 21 hours after NK cell addition, and was normalized by setting the impedance before NK cell addition as 100% alive and the impedance before tumor cell addition as 0% alive. At 96 hours post-NK cell addition, the non-adherent fraction of the wells was isolated for flow cytometry and stained with anti-CD56 (eBioscience CMSSB PE-Cy7), anti-CD69 (eBioscience H1.2F3 FITC), anti-CD45 (eBioscience HI30 APC-Cy7), anti-NKG2D (Thermo Fisher 1D11 BV421), and Live/Dead Blue (Thermo Fisher L34962) and analyzed on a Becton Dickinson Fortessa and Flowjo (TreeStar) (see, e.g., FIGS. 17A-17E).

Primary Mouse Macrophages were generated from flushed mouse femur bone marrow cultured at 4e6/10 mL cR10+50 ng/mL M-CSF (Peprotech)/10 cm dish. Media was refreshed every other day until 2 weeks of total culture. After the first week of culture, no additional M-CSF was added. Macrophages were harvested after two weeks using a cell scraper, and labeled with CellTrace Violet (Life Technologies) at 10 uM in RPMI 1640+10% FBS. CT26.WT cells transduced to overexpress human ULBP2 were labeled with CFSE at 1 uM in RPMI 1640+10% FBS. 50,000 CT26.WT cells were plated into a 96-well v-bottom plate, and Mouse macrophages were added at an effector:target ratio of 2:1. NKG2D-Fc was then added at the indicated concentration. The cells were incubated for 2.5 hours at 37 degrees, and then spun down and stained with 1 ug/mL 7-AAD in 100 uL PBS+2% FBS+2 mM EDTA immediately before flow. Phagocytosed cells (CFSE+CellTraceViolet+) were quantified by flow cytometry on a BD Fortessa and Analyzed in Flowjo (see, e.g., FIG. 18).

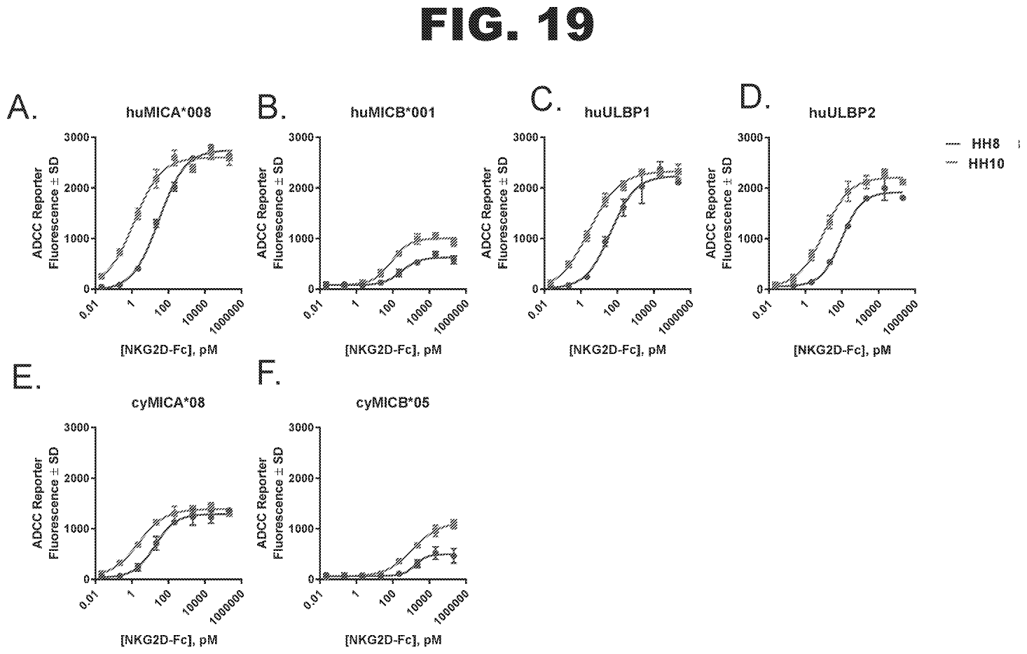

1.5e4 engineered stable CT26.WT cells over-expressing recombinant human and cynomolgus NKG2D ligands (target cells) were re-suspended in RPMI-Glutamax medium (Gibco) supplemented with 10% FBS (assay medium) and plated in the wells of a white 96-well plate. The cells were incubated with the different molar concentrations of the dimeric NKG2D-Fc variants diluted in assay medium. After one hour incubation, 9e4 FcγRIIIa V158 NFAT luciferase Jurkat (JNL) cells (effector cells) were added to the experimental wells resulting in a target:effector cell ratio of 1:6. Assay plate was incubated for four hours at 37° C. and 5% CO2. Concentration-dependent activation of the FcγRIIIa receptor was revealed by the addition per well of 60 ul One-Glo™ (Promega) and incubation for 3 minutes at room temperature protected from light. The resulting bioluminescence was measured with a Synergy HT plate reader (Biotek). Half maximal effective concentration (EC50) in ng/ml was determined with background subtracted fluorescence signals (see, e.g., FIGS. 19A-19F).

TABLE 4
ADCC EC50 values of HH8 molecule and HH10 molecule

| EC50 (pM) | HH8 | HH10 |
|---|---|---|
| huMICA*008 | 30 | 1.33 |
| huMICB*001 | 30 | 8.21 |
| huULBP1 | 43 | 2.71 |
| huULBP2 | 93 | 10.47 |
| cyMICA*08 | 18 | 2.30 |
| cyMICB*05 | 1,675 | 1,248 |

### Example 8. In Vivo PK

8-10 week old female C57Bl/6 mice were injected with 5 mg/kg of the indicated proteins. At the indicated timepoints, a volume of blood sufficient to generate 20-25 uL serum was withdrawn from the tail vein and centrifuged to isolate serum. Serum was then frozen, and then subsequently thawed. Human IgG1 was quantified using an MSD streptavidin plate coated with Biotin anti-human (Southern Biotech 2049-08), adding the mouse serum, and detecting with anti-human SulfoTAG (Southern Biotech 2049-01) and detecting signal according to manufacturer instructions. Samples were then read and quantified on an MSD instrument. Initial mouse PK on HH8 and SQX244 with a 5 mg/kg dose using material derived from HEK cells showed rapid elimination of NKG2D-Fc and a short serum half-life. In comparison to HEK, CHO cell derived material has significantly improved PK in mouse and NHP. HH8 and SQX244 from CHO had similar PK to IgG isotype with a slightly faster distribution phase. No rapid elimination due to membrane target-mediated drug disposition (TMDD) was observed at the tested doses and no Anti-Drug Antibodies (ADA) were detected in NHPs. Hyper sialylation from CHO-HySi had no additional effect on PK and therefore CHO-HySi was deselected for further characterization at lead development phase (see, e.g., FIGS. 20A-20B).

TABLE 5
PK parameters

| Mean Mouse PK | | | | | |
|---|---|---|---|---|---|
| parameters | | | | | |
| after 5 mg/kg | Tmax | Cmax | AUC | CL | Vz |
| dose of HH8 | (hr) | (μg/mL) | (μg*hr/mL) | (mL*hr/kg) | (mL/kg) |
| Mean | 0.5 | 97.3 | AUClast = | 0.684 | 147 |
| | | | 4180 | | |
| Cyno PK | 0.25 | 22.9 | AUC0-168 h = | | |
| parameters | | | 1320 | | |
| after 1 mg/kg | | | | | |
| dose of HH8 | | | | | |

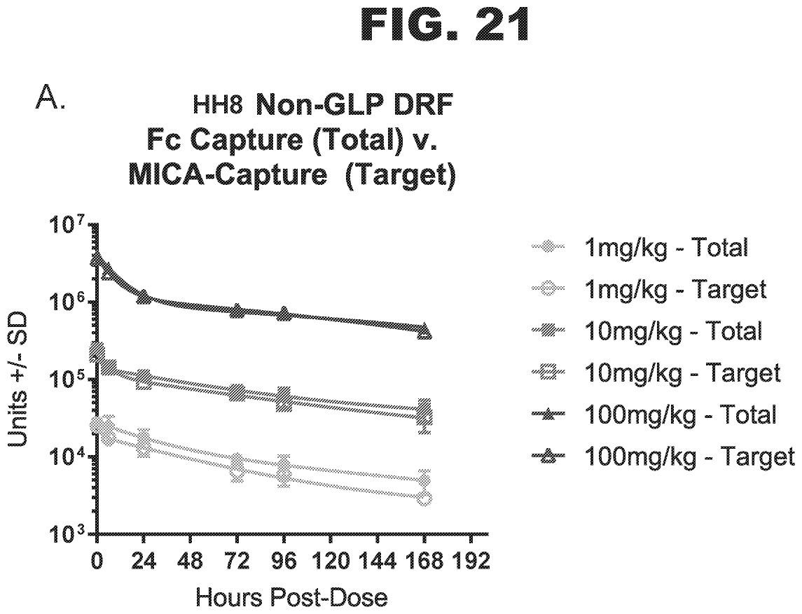

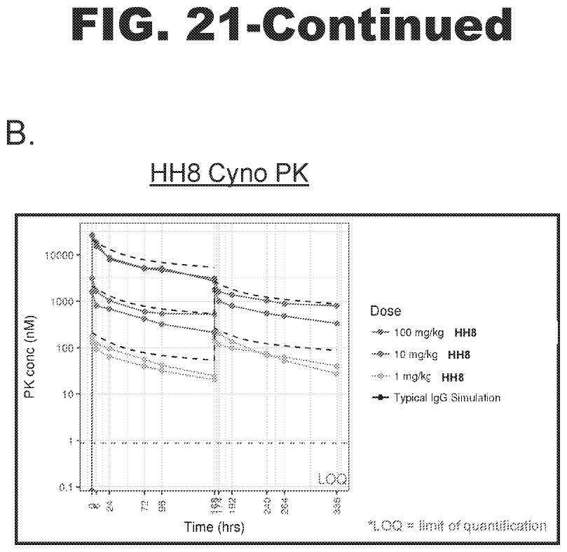

52-77 months old female Cynomolgus Monkeys were injected with 1 to 100 mg/kg of HH8. At the indicated timepoints, a volume of blood sufficient to generate 200-250 uL serum was withdrawn from the femoral vein and centrifuged to isolate serum. Serum was then frozen, and then subsequently thawed. Human IgG1 was quantified using Fc capture or target capture. For the Fc capture a streptavidin coated plate was coated with Biotin anti-human (Southern Biotech 2049-08) and for the target based capture a microtiter plate was directly coated with MICA. Then the cynomolgus monkey serum was added and detected with HRP conjugated anti-human (Southern Biotech 2049-05). The signal was measured using a colorimetric read out. The individual Cyno PK data from the Fc capture based PK assay was plotted along with a typical IgG PK prediction in Cynos. The typical IgG PK prediction in cynos was based on a two compartment PK model, with V1=41 mL/kg, V2=44 mL/kg, CL=6 mL/day/kg and Q=17 mL/day/kg, for a 3 kg monkey (Dostalek M et al., mAbs, 2017). HH8 PK was generally comparable to a typical IgG antibody PK, except for a slightly faster distribution and elimination phase (see, e.g., FIGS. 21A-21B).

### Example 9. In Vivo PD

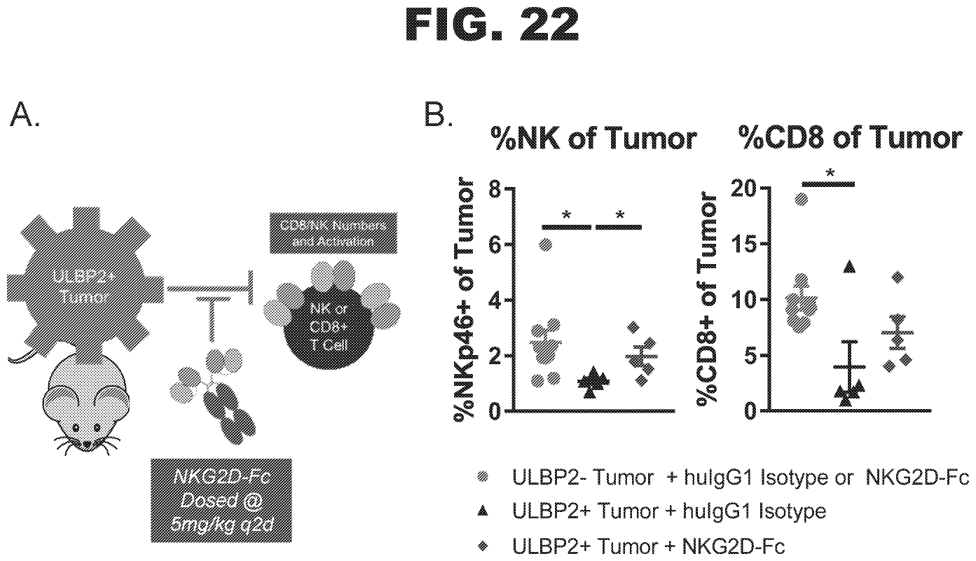

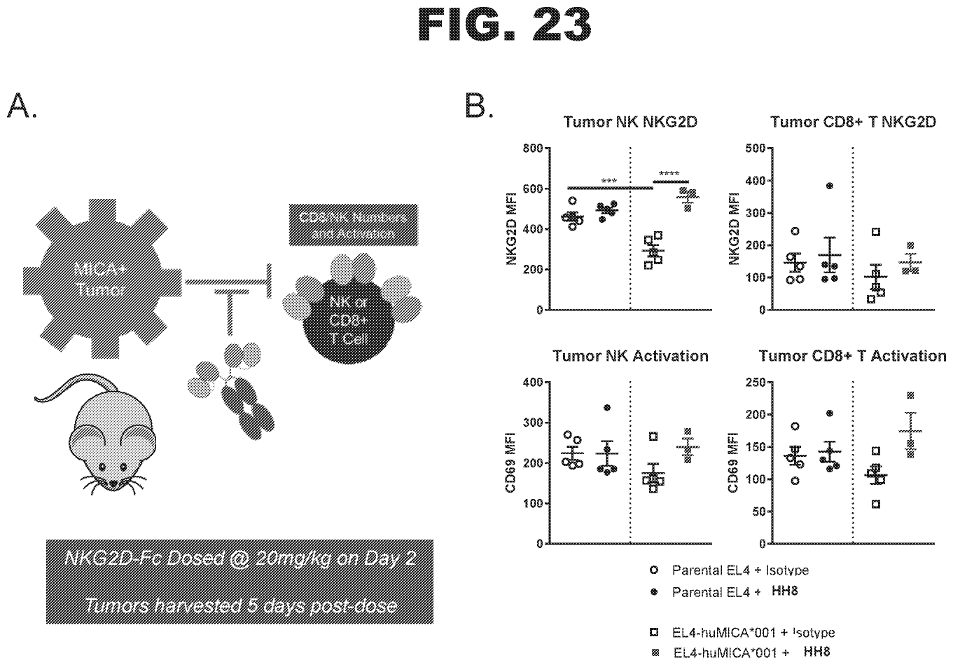

CT26.WT cells (ATCC) were engineered to overexpress human ULBP2 and were expanded in RPMI-1640+10% Heat-Inactivated FBS. 1 e6 cells were implanted subcutaneously into the upper left flank of 6-8 week old Balb/c mice (Jackson). Starting on Day 0, mice were injected with 5 mg/kg of Human IgG1 Isotype or HEK HH8 intraperitoneally. This injection was repeated every other day for 6 total doses. At 2 days post-6th dose, tumors were harvested and digested to single cell suspension, stained with anti-CD45, anti-NKp46 and anti-CD8, and analyzed on a BD Fortessa and Flowjo. NK cells were identified as NKp46+CD45+ cells. CD8+ T cells were identified as CD8+CD45+ cells, and the percent infiltrate into the tumor is quantified (see, e.g. FIGS. 22A-22B).

EL4 cells were engineered to overexpress Human MICA*001. These cells were expanded in DMEM+10% Heat-Inactivated FBS before implant of 500e3 cells subcutaneously into the upper right flank of 6-8 week old C57Bl/6 mice (Jackson). Mice were then dosed with 20 mg/kg HH8 or Human IgG1 isotype on days 2 post-tumor implant, and tumors were harvested by enzymatic digest 5 days later, stained with anti-CD69, anti-NKG2D, anti-CD45, anti-NKp46, and analyzed on a BD Fortessa and Flowjo. NK cells were identified as NKp46+CD45+ cells. CD69 and NKG2D Median fluorescence intensity were calculated and are presented here (see, e.g. FIGS. 23A-23B).

### Example 10. In Vivo Efficacy

EL4 cells were engineered to overexpress Human MICA*001. These cells were expanded in DMEM+10% Heat-Inactivated FBS before implant of 500e3 cells subcutaneously into the upper right flank of 6-8 week old C57Bl/6 mice (Jackson). Mice were then dosed with 5 mg/kg HH8, HH10, or Human IgG1 isotype on days 2, 9, and 16 post-tumor implant. Tumors were measured by caliper, and survival was calculated as time to reach 1,000 mm3 (see, e.g., FIGS. 24A-24B).

TABLE 6
Sequence Listing

| Name | Sequence | SEQ ID NO |
|---|---|---|
| hNKG2D(aa78-216) | FLNSLFNQEV QIPLTESYCG PCPKNWICYK | 1 |
| protein + RS | NNCYQFFDES KNWYESQASC MSQNASLLKV | |
| | YSKEDQDLLK LVKSYHWMGL VHIPTNGSWQ | |
| | WEDGSILSPN LLTIIEMQKG DCALYASSFK | |
| | GYIENCSTPN TYICMQRTVR S | |
| hNKG2D(aa84-216) | NQEV QIPLTESYCG PCPKNWICYK NNCYQFFDES | 2 |
| protein + RS | KNWYESQASC MSQNASLLKV YSKEDQDLLK | |
| | LVKSYHWMGL VHIPTNGSWQ WEDGSILSPN | |
| | LLTIIEMQKG DCALYASSFK GYIENCSTPN | |
| | TYICMQRTVR S | |
| mNKG2D (aa94-232) | FQPVLCNKEVPVSSREGYCGPCPNNWICHRNNCYQF | 3 |
| protein | FNEEKTWNQSQASCLSQNSSLLKIYSKEEQDFLKLV | |
| | KSYHWMGLVQIPANGSWQWEDGSSLSYNQLTLVEI | |
| | PKGSCAVYGSSFKAYTEDCANLNTYICMKRAV | |
| mNKG2D (full) | MALIRDRKSHHSEMSKCHNYDLKPAKWDTSQEQQ | 4 |
| protein | KQRLALTTSQPGENGIIRGRYPIEKLKISPMFVVRVL | |
| | AIALAIRFTLNTLMWLAIFKETFQPVLCNKEVPVSSR | |
| | EGYCGPCPNNWICHRNNCYQFFNEEKTWNQSQASC | |
| | LSQNSSLLKIYSKEEQDFLKLVKSYHWMGLVQIPAN | |
| | GSWQWEDGSSLSYNQLTLVEIPKGSCAVYGSSFKAY | |
| | TEDCANLNTYICMKRAV | |
| Fc of HH8 | DK THTCPPCPAP ELLGGPSVFL FPPKPKDTLM | 5 |
| protein | ISRTPEVTCV VVDVSHEDPE VKFNWYVDGV | |
| hFc1 | EVHNAKTKPR EEQYNSTYRV VSVLTVLHQD | |
| | WLNGKEYKCK VSNKALPAPI EKTISKAKGQ | |
| | PREPQVYTLP PSRDELTKNQ VSLTCLVKGF | |
| | YPSDIAVEWE SNGQPENNYK TTPPVLDSDG | |
| | SFFLYSKLTV DKSRWQQGNV FSCSVMHEAL | |
| | HNHYTQKSLS LSPGK | |
| Fc of HH10 | DK THTCPPCPAP ELLGGPDVFL FPPKPKDTLM | 6 |
| protein | ISRTPEVTCV VVDVSHEDPE VKFNWYVDGV | |
| hFc1 SDIE | EVHNAKTKPR EEQYNSTYRV VSVLTVLHQD | |
| | WLNGKEYKCK VSNKALPAPE EKTISKAKGQ | |
| | PREPQVYTLP PSRDELTKNQ VSLTCLVKGF | |
| | YPSDIAVEWE SNGQPENNYK TTPPVLDSDG | |
| | SFFLYSKLTV DKSRWQQGNV FSCSVMHEAL | |
| | HNHYTQKSLS LSPGK | |
| mFc2 | ISAMVRSPRGPTIKPCPPCKCPAPNLLGGPSVFIFPPKI | 63 |
| | KDVLMISLSPIVTCVVVDVSEDDPDVQISWFVNNVE | |
| | VHTAQTQTHREDYNSTLRVVSALPIQHQDWMSGKE | |
| | FKCKVNNKDLPAPIERTISKPKGSVRAPQVYVLPPPE | |
| | EEMTKKQVTLTCMVTDFMPEDIYVEWTNNGKTELN | |
| | YKNTEPVLDSDGSYFMYSKLRVEKKNWVERNSYSC | |
| | SVVHEGLHNHHTTKSFSRTPGK | |
| hLC kappa | RTVAAPSVFIFPPSDEQLKSGTASVVCLLNNFYPREA | 64 |
| | KVQWKVDNALQSGNSQESVTEQDSKDSTYSLSSTL | |
| | TLSKADYEKHKVYACEVTHQGLSSPVTKSFNRGEC | |
| CH-hFc1 | ASTKGPSVFPLAPSSKSTSGGTAALGCLVKDYFPEPV | 65 |
| | TVSWNSGALTSGVHTFPAVLQSSGLYSLSSVVTVPS | |
| | SSLGTQTYICNVNHKPSNTKVDKRVEPKSCDKTHTC | |
| | PPCPAPELLGGPSVFLFPPKPKDTLMISRTPEVTCVV | |
| | VDVSHEDPEVKFNWYVDGVEVHNAKTKPREEQYN | |
| | STYRVVSVLTVLHQDWLNGKEYKCKVSNKALPAPI | |
| | EKTISKAKGQPREPQVYTLPPSRDELTKNQVSLTCLV | |
| | KGFYPSDIAVEWESNGQPENNYKTTPPVLDSDGSFF | |
| | LYSKLTVDKSRWQQGNVFSCSVMHEALHNHYTQK | |
| | SLSLSPGK | |
| (GS)n | (GS)n, n = 1, 2, 3, 4, 5, 6, 7, 8, 9, 10, 11 or 12 | 7 |
| (G4S)n | (GGGGS)n, n = 1, 2, 3, 4 or 5 | 8 |
| (G3S)n | (GGGS)n, n = 1, 2, 3, 4 or 5 | 9 |
| (G3S)2 | GGGSGGGS | 10 |
| G4S | GGGGS | 11 |
| (G4A)n | (GGGGA)n, n = 1, 2, 3, 4 or 5 | 12 |
| (G4P)n | (GGGGP)n, n = 1, 2, 3, 4 or 5 | 13 |
| (G3A)n | (GGGA)n, n = 1, 2, 3, 4 or 5 | 14 |
| G3S | GGGS | 66 |
| GGPLGL W | GGPLGL W AGG | 15 |
| AGG | | |
| IEGR | IEGR | 16 |
| HH1, | FLNSLFNQEVQIPLTESYCGPCPKNWICYKNNCYQFF | 17 |
| hNKG2D(aa78-216)- | DESKNWYESQASCMSQNASLLKVYSKEDQDLLKLV | |
| hFc1 protein | KSYHWMGLVHIPTNGSWQWEDGSILSPNLLTIIEMQ | |
| | KGDCALYASSFKGYIENCSTPNTYICMQRTVDKTHT | |
| | CPPCPAPELLGGPSVFLFPPKPKDTLMISRTPEVTCVV | |
| | VDVSHEDPEVKFNWYVDGVEVHNAKTKPREEQYN | |
| | STYRVVSVLTVLHQDWLNGKEYKCKVSNKALPAPI | |
| | EKTISKAKGQPREPQVYTLPPSRDELTKNQVSLTCLV | |
| | KGFYPSDIAVEWESNGQPENNYKTTPPVLDSDGSFF | |
| | LYSKLTVDKSRWQQGNVFSCSVMHEALHNHYTQK | |
| | SLSLSPGK | |
| HH1, | ttcctgaactccctgttcaaccaggaagtgcagatccccctgaccgagagctactg | 18 |
| hNKG2D(aa78-216)- | cggcccctgccccaagaactggatctgctacaagaacaactgctaccagttcttcg | |
| hFc1 DNA | acgagagcaagaattggtacgagagccaggccagctgcatgagccagaacgcc | |
| | agcctgctgaaggtgtacagcaaagaggaccaggatctgctgaagctcgtgaagt | |
| | cctaccactggatgggcctggtgcacatccccaccaatggcagctggcagtggg | |
| | aggacggcagcatcctgagccccaacctgctgaccatcatcgagatgcagaagg | |
| | gcgactgcgccctgtacgccagcagcttcaagggctacatcgagaactgcagca | |
| | cccccaacacctacatctgtatgcagcggaccgtcgacaagacccacacctgccc | |
| | cccctgtcctgcccctgaactgctgggcggaccctccgtgttcctgttccccccaaa | |
| | gcccaaggacaccctgatgatcagccggacccccgaagtgacctgcgtggtggt | |
| | ggacgtgtcccacgaggaccctgaagtgaagttcaattggtacgtggacggcgtg | |
| | gaagtgcacaacgccaagaccaagcccagagaggaacagtacaacagcaccta | |
| | ccgggtggtgtccgtgctgaccgtgctgcaccaggactggctgaacggcaaaga | |
| | gtacaagtgcaaagtctccaacaaggccctgcctgcccccatcgagaaaaccatc | |
| | agcaaggccaagggccagccccgcgagccccaggtgtacacactgcccccca | |
| | gccgggacgagctgaccaagaaccaggtgtccctgacctgcctggtcaagggct | |
| | tctaccccagcgatatcgccgtggaatgggagagcaacggccagcccgagaac | |
| | aactacaagaccaccccccctgtgctggacagcgacggctcattcttcctgtacag | |
| | caagctgaccgtggacaagtcccggtggcagcagggcaacgtgttcagctgcag | |
| | cgtgatgcacgaggccctgcacaaccactacacccagaagtccctgagcctgag | |
| | ccccggcaaa | |
| HH2, | FLNSLFNQEVQIPLTESYCGPCPKNWICYKNNCYQFF | 19 |
| hNKG2D(aa78-216)- | DESKNWYESQASCMSQNASLLKVYSKEDQDLLKLV | |
| RSG3S)2- | KSYHWMGLVHIPTNGSWQWEDGSILSPNLLTIIEMQ | |
| hNKG2D(aa78-216)- | KGDCALYASSFKGYIENCSTPNTYICMQRTVRSGGG | |
| hFc1 protein | SGGGSFLNSLFNQEVQIPLTESYCGPCPKNWICYKNN | |
| | CYQFFDESKNWYESQASCMSQNASLLKVYSKEDQD | |
| | LLKLVKSYHWMGLVHIPTNGSWQWEDGSILSPNLL | |
| | TIIEMQKGDCALYASSFKGYIENCSTPNTYICMQRTV | |
| | DKTHTCPPCPAPELLGGPSVFLFPPKPKDTLMISRTPE | |
| | VTCVVVDVSHEDPEVKFNWYVDGVEVHNAKTKPR | |
| | EEQYNSTYRVVSVLTVLHQDWLNGKEYKCKVSNK | |
| | ALPAPIEKTISKAKGQPREPQVYTLPPSRDELTKNQV | |
| | SLTCLVKGFYPSDIAVEWESNGQPENNYKTTPPVLD | |
| | SDGSFFLYSKLTVDKSRWQQGNVFSCSVMHEALHN | |
| | HYTQKSLSLSPGK | |
| HH2, | ttcctgaactccctgttcaaccaggaagtgcagatccccctgaccgagagctactg | 20 |
| hNKG2D(aa78-216)- | cggcccctgccccaagaactggatctgctacaagaacaactgctaccagttcttcg | |
| RS(G3S)2- | acgagagcaagaattggtacgagagccaggccagctgcatgagccagaacgcc | |
| hNKG2D(aa78-216)- | agcctgctgaaggtgtacagcaaagaggaccaggatctgctgaagctcgtgaagt | |
| hFc1 DNA | cctaccactggatgggcctggtgcacatccccaccaatggcagctggcagtggg | |
| | aggacggcagcatcctgagccccaacctgctgaccatcatcgagatgcagaagg | |
| | gcgactgcgccctgtacgccagcagcttcaagggctacatcgagaactgcagca | |
| | cccccaacacctacatctgtatgcagcggaccgtcagaagtggaggcgggtctg | |
| | gaggcggatcctttctgaatagcctgtttaaccaggaagtgcagatccccctgacc | |
| | gagagctactgcggcccctgccccaagaactggatctgctacaagaacaactgct | |
| | accagttcttcgacgagagcaagaattggtacgagagccaggccagctgcatga | |
| | gccagaacgccagcctgctgaaggtgtacagcaaagaggaccaggatctgctga | |
| | agctcgtgaagtcctaccactggatgggcctggtgcacatccccaccaatggcag | |
| | ctggcagtgggaggacggcagcatcctgagccccaacctgctgaccatcatcga | |
| | gatgcagaagggcgactgcgccctgtacgccagcagcttcaagggctacatcga | |
| | gaactgcagcacccccaacacctacatttgcatgcagcgtacggttgacaagacc | |
| | cacacctgccccccctgtcctgcccctgaactgctgggcggaccctccgtgttcct | |
| | gttccccccaaagcccaaggacaccctgatgatcagccggacccccgaagtgac | |
| | ctgcgtggtggtggacgtgtcccacgaggaccctgaagtgaagttcaattggtacg | |
| | tggacggcgtggaagtgcacaacgccaagaccaagcccagagaggaacagtac | |
| | aacagcacctaccgggtggtgtccgtgctgaccgtgctgcaccaggactggctga | |
| | acggcaaagagtacaagtgcaaagtctccaacaaggccctgcctgcccccatcg | |
| | agaaaaccatcagcaaggccaagggccagccccgcgagccccaggtgtacaca | |
| | ctgccccccagccgggacgagctgaccaagaaccaggtgtccctgacctgcctg | |
| | gtcaagggcttctaccccagcgatatcgccgtggaatgggagagcaacggccag | |
| | cccgagaacaactacaagaccaccccccctgtgctggacagcgacggctcattct | |
| | tcctgtacagcaagctgaccgtggacaagtcccggtggcagcagggcaacgtgtt | |
| | cagctgcagcgtgatgcacgaggccctgcacaaccactacacccagaagtccct | |
| | gagcctgagccccggcaaa | |
| HH3, hFc1- | DKTHTCPPCPAPELLGGPSVFLFPPKPKDTLMISRTPE | 21 |
| hNKG2D(aa78-216) | VTCVVVDVSHEDPEVKFNWYVDGVEVHNAKTKPR | |
| protein | EEQYNSTYRVVSVLTVLHQDWLNGKEYKCKVSNK | |
| | ALPAPIEKTISKAKGQPREPQVYTLPPSREEMTKNQV | |
| | SLTCLVKGFYPSDIAVEWESNGQPENNYKTTPPVLD | |
| | SDGSFFLYSKLTVDKSRWQQGNVFSCSVMHEALHN | |
| | HYTQKSLSLSPGFLNSLFNQEVQIPLTESYCGPCPKN | |
| | WICYKNNCYQFFDESKNWYESQASCMSQNASLLKV | |
| | YSKEDQDLLKLVKSYHWMGLVHIPTNGSWQWEDG | |
| | SILSPNLLTIIEMQKGDCALYASSFKGYIENCSTPNTY | |
| | ICMQRTV | |
| HH3, hFc1- | gacaagacccacacctgtcctccctgtcctgcccctgaactgctgggcggaccta | 22 |
| hNKG2D(aa78-216) | gcgtgttcctgttccccccaaagcccaaggacaccctgatgatcagccggacccc | |
| DNA | cgaagtgacctgcgtggtggtggatgtgtcccacgaggaccctgaagtgaagttc | |
| | aattggtacgtggacggcgtggaagtgcacaacgccaagaccaagcccagaga | |
| | ggaacagtacaacagcacctaccgggtggtgtccgtgctgaccgtgctgcacca | |
| | ggactggctgaacggcaaagagtacaagtgcaaggtgtccaacaaggccctgcc | |
| | agcccccatcgagaaaaccatcagcaaggccaagggccagccccgcgaaccc | |
| | caggtgtacacactgccccctagccgggaagagatgaccaagaaccaggtgtcc | |
| | ctgacctgtctcgtgaagggcttctacccctccgatatcgccgtggaatgggagag | |
| | caacggccagcccgagaacaactacaagaccaccccccctgtgctggacagcg | |
| | acggctcattcttcctgtacagcaagctgacagtggacaagagccggtggcagca | |
| | gggcaacgtgttcagctgcagcgtgatgcacgaggccctgcacaaccactacac | |
| | ccagaagagcttaagcctgagccctggcttcctgaactccctgttcaaccaggaag | |
| | tgcagatccccctgaccgagagctactgcggcccctgccccaagaactggatctg | |
| | ctacaagaacaactgctaccagttcttcgacgagagcaagaattggtacgagagc | |
| | caggccagctgcatgagccagaacgccagcctgctgaaggtgtacagcaaaga | |
| | ggaccaggatctgctgaagctcgtgaagtcctaccactggatgggcctggtgcac | |
| | atccccaccaatggcagctggcagtgggaggacggcagcatcctgagccccaa | |
| | cctgctgaccatcatcgagatgcagaagggcgactgcgccctgtacgccagcag | |
| | cttcaagggctacatcgagaactgcagcacccccaacacctacatctgtatgcagc | |
| | ggaccgtc | |
| HH4, hFc1- | DKTHTCPPCPAPELLGGPSVFLFPPKPKDTLMISRTPE | 23 |
| hNKG2D(aa78-216)- | VTCVVVDVSHEDPEVKFNWYVDGVEVHNAKTKPR | |
| RS(G3S)2- | EEQYNSTYRVVSVLTVLHQDWLNGKEYKCKVSNK | |
| hNKG2D(aa78-216) | ALPAPIEKTISKAKGQPREPQVYTLPPSREEMTKNQV | |
| protein | SLTCLVKGFYPSDIAVEWESNGQPENNYKTTPPVLD | |
| | SDGSFFLYSKLTVDKSRWQQGNVFSCSVMHEALHN | |
| | HYTQKSLSLSPGFLNSLFNQEVQIPLTESYCGPCPKN | |
| | WICYKNNCYQFFDESKNWYESQASCMSQNASLLKV | |
| | YSKEDQDLLKLVKSYHWMGLVHIPTNGSWQWEDG | |
| | SILSPNLLTIIEMQKGDCALYASSFKGYIENCSTPNTY | |
| | ICMQRTVRSGGGSGGGSFLNSLFNQEVQIPLTESYCG | |
| | PCPKNWICYKNNCYQFFDESKNWYESQASCMSQNA | |
| | SLLKVYSKEDQDLLKLVKSYHWMGLVHIPTNGSWQ | |
| | WEDGSILSPNLLTIIEMQKGDCALYASSFKGYIENCS | |
| | TPNTYICMQRTV | |
| HH4, hFc1- | gacaagacccacacctgtcctccctgtcctgcccctgaactgctgggcggaccta | 24 |
| hNKG2D(aa78-216)- | gcgtgttcctgttccccccaaagcccaaggacaccctgatgatcagccggacccc | |
| RS(G3S)2- | cgaagtgacctgcgtggtggtggatgtgtcccacgaggaccctgaagtgaagttc | |
| hNKG2D(aa78-216) | aattggtacgtggacggcgtggaagtgcacaacgccaagaccaagcccagaga | |
| DNA | ggaacagtacaacagcacctaccgggtggtgtccgtgctgaccgtgctgcacca | |
| | ggactggctgaacggcaaagagtacaagtgcaaggtgtccaacaaggccctgcc | |
| | agcccccatcgagaaaaccatcagcaaggccaagggccagccccgcgaaccc | |
| | caggtgtacacactgccccctagccgggaagagatgaccaagaaccaggtgtcc | |
| | ctgacctgtctcgtgaagggcttctacccctccgatatcgccgtggaatgggagag | |
| | caacggccagcccgagaacaactacaagaccaccccccctgtgctggacagcg | |
| | acggctcattcttcctgtacagcaagctgacagtggacaagagccggtggcagca | |
| | gggcaacgtgttcagctgcagcgtgatgcacgaggccctgcacaaccactacac | |
| | ccagaagagcttaagcctgagccctggcttcctgaactccctgttcaaccaggaag | |
| | tgcagatccccctgaccgagagctactgcggcccctgccccaagaactggatctg | |
| | ctacaagaacaactgctaccagttcttcgacgagagcaagaattggtacgagagc | |
| | caggccagctgcatgagccagaacgccagcctgctgaaggtgtacagcaaaga | |
| | ggaccaggatctgctgaagctcgtgaagtcctaccactggatgggcctggtgcac | |
| | atccccaccaatggcagctggcagtgggaggacggcagcatcctgagccccaa | |
| | cctgctgaccatcatcgagatgcagaagggcgactgcgccctgtacgccagcag | |
| | cttcaagggctacatcgagaactgcagcacccccaacacctacatctgtatgcagc | |
| | ggaccgtcagaagtggaggcgggtctggaggcggatcctttctgaatagcctgttt | |
| | aaccaggaagtgcagatccccctgaccgagagctactgcggcccctgccccaag | |
| | aactggatctgctacaagaacaactgctaccagttcttcgacgagagcaagaattg | |
| | gtacgagagccaggccagctgcatgagccagaacgccagcctgctgaaggtgta | |
| | cagcaaagaggaccaggatctgctgaagctcgtgaagtcctaccactggatggg | |
| | cctggtgcacatccccaccaatggcagctggcagtgggaggacggcagcatcct | |
| | gagccccaacctgctgaccatcatcgagatgcagaagggcgactgcgccctgta | |
| | cgccagcagcttcaagggctacatcgagaactgcagcacccccaacacctacatt | |
| | tgcatgcagcgtacggtt | |
| HH5, | FLNSLFNQEVQIPLTESYCGPCPKNWICYKNNCYQFF | 25 |
| hNKG2D(aa78-216)- | DESKNWYESQASCMSQNASLLKVYSKEDQDLLKLV | |
| RS(G3S)2- | KSYHWMGLVEOPTNGSWQWEDGSILSPNLLTIIEMQ | |
| hNKG2D(aa78-216)- | KGDCALYASSFKGYIENCSTPNTYICMQRTVRSGGG | |
| hFc1[S239D, | SGGGSFLNSLFNQEVQIPLTESYCGPCPKNWICYKNN | |
| I332E] protein | CYQFFDESKNWYESQASCMSQNASLLKVYSKEDQD | |
| | LLKLVKSYHWMGLVHIPTNGSWQWEDGSILSPNLL | |
| | TIIEMQKGDCALYASSFKGYIENCSTPNTYICMQRTV | |
| | DKTHTCPPCPAPELLGGPDVFLFPPKPKDTLMISRTP | |
| | EVTCVVVDVSHEDPEVKFNWYVDGVEVHNAKTKP | |
| | REEQYNSTYRVVSVLTVLHQDWLNGKEYKCKVSN | |
| | KALPAPEEKTISKAKGQPREPQVYTLPPSRDELTKNQ | |
| | VSLTCLVKGFYPSDIAVEWESNGQPENNYKTTPPVL | |
| | DSDGSFFLYSKLTVDKSRWQQGNVFSCSVMHEALH | |
| | NHYTQKSLSLSPGK | |
| HH5, | ttcctgaactccctgttcaaccaggaagtgcagatccccctgaccgagagctactg | 26 |
| hNKG2D(aa78-216)- | cggcccctgccccaagaactggatctgctacaagaacaactgctaccagttcttcg | |
| RS(G3S)2- | acgagagcaagaattggtacgagagccaggccagctgcatgagccagaacgcc | |
| hNKG2D(aa78-216)- | agcctgctgaaggtgtacagcaaagaggaccaggatctgctgaagctcgtgaagt | |
| hFc1[S239D, | cctaccactggatgggcctggtgcacatccccaccaatggcagctggcagtggg | |
| I332E] DNA | aggacggcagcatcctgagccccaacctgctgaccatcatcgagatgcagaagg | |
| | gcgactgcgccctgtacgccagcagcttcaagggctacatcgagaactgcagca | |
| | cccccaacacctacatctgtatgcagcggaccgtcagaagtggaggcgggtctg | |
| | gaggcggatcctttctgaatagcctgtttaaccaggaagtgcagatccccctgacc | |
| | gagagctactgcggcccctgccccaagaactggatctgctacaagaacaactgct | |
| | accagttcttcgacgagagcaagaattggtacgagagccaggccagctgcatga | |
| | gccagaacgccagcctgctgaaggtgtacagcaaagaggaccaggatctgctga | |
| | agctcgtgaagtcctaccactggatgggcctggtgcacatccccaccaatggcag | |
| | ctggcagtgggaggacggcagcatcctgagccccaacctgctgaccatcatcga | |
| | gatgcagaagggcgactgcgccctgtacgccagcagcttcaagggctacatcga | |
| | gaactgcagcacccccaacacctacatttgcatgcagcgtacggttgacaagacc | |
| | cacacctgccccccctgtcctgcccctgaactgctgggcggacccgacgtgttcct | |
| | gttccccccaaagcccaaggacaccctgatgatcagccggacccccgaagtgac | |
| | ctgcgtggtggtggacgtgtcccacgaggaccctgaagtgaagttcaattggtacg | |
| | tggacggcgtggaagtgcacaacgccaagaccaagcccagagaggaacagtac | |
| | aacagcacctaccgggtggtgtccgtgctgaccgtgctgcaccaggactggctga | |
| | acggcaaagagtacaagtgcaaagtctccaacaaggccctgcctgcccccgaag | |
| | agaaaaccatcagcaaggccaagggccagccccgcgagccccaggtgtacaca | |
| | ctgccccccagccgggacgagctgaccaagaaccaggtgtccctgacctgcctg | |
| | gtcaagggcttctaccccagcgatatcgccgtggaatgggagagcaacggccag | |
| | cccgagaacaactacaagaccaccccccctgtgctggacagcgacggctcattct | |
| | tcctgtacagcaagctgaccgtggacaagtcccggtggcagcagggcaacgtgtt | |
| | cagctgcagcgtgatgcacgaggccctgcacaaccactacacccagaagtccct | |
| | gagcctgagccccggcaaa | |
| HH7, | FLNSLFNQEVQIPLTESYCGPCPKNWICYKNNCYQFF | 27 |
| hNKG2D(aa78-216)- | DESKNWYESQASCMSQNASLLKVYSKEDQDLLKLV | |
| RS(G3S)2- | KSYHWMGLVEOPTNGSWQWEDGSILSPNLLTIIEMQ | |
| hNKG2D(aa84-216)- | KGDCALYASSFKGYIENCSTPNTYICMQRTVRSGGG | |
| hFc1 protein | SGGGSNQEVQIPLTESYCGPCPKNWICYKNNCYQFF | |
| | DESKNWYESQASCMSQNASLLKVYSKEDQDLLKLV | |
| | KSYHWMGLVEOPTNGSWQWEDGSILSPNLLTIIEMQ | |
| | KGDCALYASSFKGYIENCSTPNTYICMQRTVDKTHT | |
| | CPPCPAPELLGGPSVFLFPPKPKDTLMISRTPEVTCVV | |
| | VDVSHEDPEVKFNWYVDGVEVHNAKTKPREEQYN | |
| | STYRVVSVLTVLHQDWLNGKEYKCKVSNKALPAPI | |
| | EKTISKAKGQPREPQVYTLPPSRDELTKNQVSLTCLV | |
| | KGFYPSDIAVEWESNGQPENNYKTTPPVLDSDGSFF | |
| | LYSKLTVDKSRWQQGNVFSCSVMHEALHNHYTQK | |
| | SLSLSPGK | |
| HH7, | ttcctgaactccctgttcaaccaggaagtgcagatccccctgaccgagagctactg | 28 |
| hNKG2D(aa78-216)- | cggcccctgccccaagaactggatctgctacaagaacaactgctaccagttcttcg | |
| RS(G3S)2- | acgagagcaagaattggtacgagagccaggccagctgcatgagccagaacgcc | |
| hNKG2D(aa84-216)- | agcctgctgaaggtgtacagcaaagaggaccaggatctgctgaagctcgtgaagt | |
| hFc1 DNA | cctaccactggatgggcctggtgcacatccccaccaatggcagctggcagtggg | |
| | aggacggcagcatcctgagccccaacctgctgaccatcatcgagatgcagaagg | |
| | gcgactgcgccctgtacgccagcagcttcaagggctacatcgagaactgcagca | |
| | cccccaacacctacatctgtatgcagcggaccgtcagaagtggaggcgggtctg | |
| | gaggcggatccaaccaggaagtgcagatccccctgaccgagagctactgcggc | |
| | ccctgccccaagaactggatctgctacaagaacaactgctaccagttcttcgacga | |
| | gagcaagaattggtacgagagccaggccagctgcatgagccagaacgccagcc | |
| | tgctgaaggtgtacagcaaagaggaccaggatctgctgaagctcgtgaagtccta | |
| | ccactggatgggcctggtgcacatccccaccaatggcagctggcagtgggagga | |
| | cggcagcatcctgagccccaacctgctgaccatcatcgagatgcagaagggcga | |
| | ctgcgccctgtacgccagcagcttcaagggctacatcgagaactgcagcaccccc | |
| | aacacctacatttgcatgcagcgtacggttgacaagacccacacctgccccccctg | |
| | tcctgcccctgaactgctgggcggaccctccgtgttcctgttccccccaaagccca | |
| | aggacaccctgatgatcagccggacccccgaagtgacctgcgtggtggtggacg | |
| | tgtcccacgaggaccctgaagtgaagttcaattggtacgtggacggcgtggaagt | |
| | gcacaacgccaagaccaagcccagagaggaacagtacaacagcacctaccgg | |
| | gtggtgtccgtgctgaccgtgctgcaccaggactggctgaacggcaaagagtaca | |
| | agtgcaaagtctccaacaaggccctgcctgcccccatcgagaaaaccatcagcaa | |
| | ggccaagggccagccccgcgagccccaggtgtacacactgccccccagccgg | |
| | gacgagctgaccaagaaccaggtgtccctgacctgcctggtcaagggcttctacc | |
| | ccagcgatatcgccgtggaatgggagagcaacggccagcccgagaacaactac | |
| | aagaccaccccccctgtgctggacagcgacggctcattcttcctgtacagcaagct | |
| | gaccgtggacaagtcccggtggcagcagggcaacgtgttcagctgcagcgtgat | |
| | gcacgaggccctgcacaaccactacacccagaagtccctgagcctgagccccg | |
| | gcaaa | |
| HH8, | NQEVQIPLTESYCGPCPKNWICYKNNCYQFFDESKN | 29 |
| hNKG2D(aa84-216)- | WYESQASCMSQNASLLKVYSKEDQDLLKLVKSYH | |
| RS(G3S)2- | WMGLVHIPTNGSWQWEDGSILSPNLLTIIEMQKGDC | |
| hNKG2D(aa84-216)- | ALYASSFKGYIENCSTPNTYICMQRTVRSGGGSGGG | |
| hFc1 protein | SNQEVQIPLTESYCGPCPKNWICYKNNCYQFFDESK | |
| | NWYESQASCMSQNASLLKVYSKEDQDLLKLVKSYH | |
| | WMGLVHIPTNGSWQWEDGSILSPNLLTIIEMQKGDC | |
| | ALYASSFKGYIENCSTPNTYICMQRTVDKTHTCPPCP | |
| | APELLGGPSVFLFPPKPKDTLMISRTPEVTCVVVDVS | |
| | HEDPEVKFNWYVDGVEVHNAKTKPREEQYNSTYR | |
| | VVSVLTVLHQDWLNGKEYKCKVSNKALPAPIEKTIS | |
| | KAKGQPREPQVYTLPPSRDELTKNQVSLTCLVKGFY | |
| | PSDIAVEWESNGQPENNYKTTPPVLDSDGSFFLYSKL | |
| | TVDKSRWQQGNVFSCSVMHEALHNHYTQKSLSLSP | |
| | GK | |
| HH8, | aaccaggaagtgcagatccccctgaccgagagctactgcggcccctgccccaag | 30 |
| hNKG2D(aa84-216)- | aactggatctgctacaagaacaactgctaccagttcttcgacgagagcaagaattg | |
| RS(G3S)2- | gtacgagagccaggccagctgcatgagccagaacgccagcctgctgaaggtgta | |
| hNKG2D(aa84-216)- | cagcaaagaggaccaggatctgctgaagctcgtgaagtcctaccactggatggg | |
| hFc1 DNA | cctggtgcacatccccaccaatggcagctggcagtgggaggacggcagcatcct | |
| | gagccccaacctgctgaccatcatcgagatgcagaagggcgactgcgccctgta | |
| | cgccagcagcttcaagggctacatcgagaactgtagcacccccaacacctacatc | |
| | tgtatgcagcggaccgtcagaagtggaggcgggtctggaggcggatccaacca | |
| | ggaagtgcagatccccctgaccgagagctactgcggcccctgccccaagaactg | |
| | gatctgctacaagaacaactgctaccagttcttcgacgagagcaagaattggtacg | |
| | agagccaggccagctgcatgagccagaacgccagcctgctgaaggtgtacagc | |
| | aaagaggaccaggatctgctgaagctcgtgaagtcctaccactggatgggcctgg | |
| | tgcacatccccaccaatggcagctggcagtgggaggacggcagcatcctgagcc | |
| | ccaacctgctgaccatcatcgagatgcagaagggcgactgcgccctgtacgcca | |
| | gcagcttcaagggctacatcgagaactgcagcacccccaacacctacatttgcatg | |
| | cagcgtacggttgacaagacccacacctgccccccctgtcctgcccctgaactgct | |
| | gggcggaccctccgtgttcctgttccccccaaagcccaaggacaccctgatgatc | |
| | agccggacccccgaagtgacctgcgtggtggtggacgtgtcccacgaggaccct | |
| | gaagtgaagttcaattggtacgtggacggcgtggaagtgcacaacgccaagacc | |
| | aagcccagagaggaacagtacaacagcacctaccgggtggtgtccgtgctgacc | |
| | gtgctgcaccaggactggctgaacggcaaagagtacaagtgcaaagtctccaac | |
| | aaggccctgcctgcccccatcgagaaaaccatcagcaaggccaagggccagcc | |
| | ccgcgagccccaggtgtacacactgccccccagccgggacgagctgaccaaga | |
| | accaggtgtccctgacctgcctggtcaagggcttctaccccagcgatatcgccgtg | |
| | gaatgggagagcaacggccagcccgagaacaactacaagaccaccccccctgt | |
| | gctggacagcgacggctcattcttcctgtacagcaagctgaccgtggacaagtcc | |
| | cggtggcagcagggcaacgtgttcagctgcagcgtgatgcacgaggccctgcac | |
| | aaccactacacccagaagtccctgagcctgagccccggcaaa | |
| HH10, | NQEVQIPLTESYCGPCPKNWICYKNNCYQFFDESKN | 31 |
| hNKG2D(aa84-216)- | WYESQASCMSQNASLLKVYSKEDQDLLKLVKSYH | |
| RS(G3S)2- | WMGLVHIPTNGSWQWEDGSILSPNLLTIIEMQKGDC | |
| hNKG2D(aa84-216)- | ALYASSFKGYIENCSTPNTYICMQRTVRSGGGSGGG | |
| hFc1[S239D, I332E] | SNQEVQIPLTESYCGPCPKNWICYKNNCYQFFDESK | |
| protein | NWYESQASCMSQNASLLKVYSKEDQDLLKLVKSYH | |
| | WMGLVHIPTNGSWQWEDGSILSPNLLTIIEMQKGDC | |
| | ALYASSFKGYIENCSTPNTYICMQRTVDKTHTCPPCP | |
| | APELLGGPDVFLFPPKPKDTLMISRTPEVTCVVVDVS | |
| | HEDPEVKFNWYVDGVEVHNAKTKPREEQYNSTYR | |
| | VVSVLTVLHQDWLNGKEYKCKVSNKALPAPEEKTI | |
| | SKAKGQPREPQVYTLPPSRDELTKNQVSLTCLVKGF | |
| | YPSDIAVEWESNGQPENNYKTTPPVLDSDGSFFLYS | |
| | KLTVDKSRWQQGNVFSCSVMHEALHNHYTQKSLSL | |
| | SPGK | |
| HH10, | aaccaggaagtgcagatccccctgaccgagagctactgcggcccctgccccaag | 32 |
| hNKG2D(aa84-216)- | aactggatctgctacaagaacaactgctaccagttcttcgacgagagcaagaattg | |
| RS(G3S)2- | gtacgagagccaggccagctgcatgagccagaacgccagcctgctgaaggtgta | |
| hNKG2D(aa84-216)- | cagcaaagaggaccaggatctgctgaagctcgtgaagtcctaccactggatggg | |
| hFc1[S239D, I332E] | cctggtgcacatccccaccaatggcagctggcagtgggaggacggcagcatcct | |
| DNA | gagccccaacctgctgaccatcatcgagatgcagaagggcgactgcgccctgta | |
| | cgccagcagcttcaagggctacatcgagaactgcagcacccccaacacctacatc | |
| | tgtatgcagcggaccgtcagaagtggaggcgggtctggaggcggatccaacca | |
| | ggaagtgcagatccccctgaccgagagctactgcggcccctgccccaagaactg | |
| | gatctgctacaagaacaactgctaccagttcttcgacgagagcaagaattggtacg | |
| | agagccaggccagctgcatgagccagaacgccagcctgctgaaggtgtacagc | |
| | aaagaggaccaggatctgctgaagctcgtgaagtcctaccactggatgggcctgg | |
| | tgcacatccccaccaatggcagctggcagtgggaggacggcagcatcctgagcc | |
| | ccaacctgctgaccatcatcgagatgcagaagggcgactgcgccctgtacgcca | |
| | gcagcttcaagggctacatcgagaactgcagcacccccaacacctacatttgcatg | |
| | cagcgtacggttgacaagacccacacctgccccccctgtcctgcccctgaactgct | |
| | gggcggacccgacgtgttcctgttccccccaaagcccaaggacaccctgatgatc | |
| | agccggacccccgaagtgacctgcgtggtggtggacgtgtcccacgaggaccct | |
| | gaagtgaagttcaattggtacgtggacggcgtggaagtgcacaacgccaagacc | |
| | aagcccagagaggaacagtacaacagcacctaccgggtggtgtccgtgctgacc | |
| | gtgctgcaccaggactggctgaacggcaaagagtacaagtgcaaagtctccaac | |
| | aaggccctgcctgcccccgaagagaaaaccatcagcaaggccaagggccagcc | |
| | ccgcgagccccaggtgtacacactgccccccagccgggacgagctgaccaaga | |
| | accaggtgtccctgacctgcctggtcaagggcttctaccccagcgatatcgccgtg | |
| | gaatgggagagcaacggccagcccgagaacaactacaagaccaccccccctgt | |
| | gctggacagcgacggctcattcttcctgtacagcaagctgaccgtggacaagtcc | |
| | cggtggcagcagggcaacgtgttcagctgcagcgtgatgcacgaggccctgcac | |
| | aaccactacacccagaagtccctgagcctgagccccggcaaa | |
| HH15_1 | NQEVQIPLTESYCGPCPKNWICYKNNCYQFFDESKN | 33 |
| hNKG2D(aa84-216)- | WYESQASCMSQNASLLKVYSKEDQDLLKLVKSYH | |
| RS(G3S)2- | WMGLVHIPTNGSWQWEDGSILSPNLLTIIEMQKGDC | |
| hNKG2D(aa84-216)- | ALYASSFKGYIENCSTPNTYICMQRTVRSGGGSGGG | |
| CH1-hFc1 | SNQEVQIPLTESYCGPCPKNWICYKNNCYQFFDESK | |
| protein | NWYESQASCMSQNASLLKVYSKEDQDLLKLVKSYH | |
| | WMGLVHIPTNGSWQWEDGSILSPNLLT | |
| | IIEMQKGDCALYASSFKGYIENCSTPNTYICMQRTVA | |
| | STKGPSVFPLAPSSKSTSGGTAALGCLVKDYFPEPVT | |
| | VSWNSGALTSGVHTFPAVLQSSGLYSLSSVVTVPSSS | |
| | LGTQTYICNVNHKPSNTKVDKRVEPKSCDKTHTCPP | |
| | CPAPELLGGPSVFLFPPKPKDTLMISRTPEVTCVVVD | |
| | VSHEDPEVKFNWYVDGVEVHNAKTKPREEQYNSTY | |
| | RVVSVLTVLHQDWLNGKEYKCKVSNKALPAPIEKTI | |
| | SKAKGQPREPQVYTLPPSRDELTKNQVSLTCLVKGF | |
| | YPSDIAVEWESNGQPENNYKTTPPVLDSDGSFFLYS | |
| | KLTVDKSRWQQGNVFSCSVMHEALHNHYTQKSLSL | |
| | SPGK | |
| HH15_1 | aaccaggaagtgcagatccccctgaccgagagctactgcggcccctgccccaag | 34 |
| hNKG2D(aa84-216)- | aactggatctgctacaagaacaactgctaccagttcttcgacgagagcaagaattg | |
| RS(G3S)2- | gtacgagagccaggccagctgcatgagccagaacgccagcctgctgaaggtgta | |
| hNKG2D(aa84-216)- | cagcaaagaggaccaggatctgctgaagctcgtgaagtcctaccactggatggg | |
| CH1-hFc1 | cctggtgcacatccccaccaatggcagctggcagtgggaggacggcagcatcct | |
| DNA | gagccccaacctgctgaccatcatcgagatgcagaagggcgactgcgccctgta | |
| | cgccagcagcttcaagggctacatcgagaactgtagcacccccaacacctacatc | |
| | tgtatgcagcggaccgtcagaagtggaggcgggtctggaggcggatccaacca | |
| | ggaagtgcagatccccctgaccgagagctactgcggcccctgccccaagaactg | |
| | gatctgctacaagaacaactgctaccagttcttcgacgagagcaagaattggtacg | |
| | agagccaggccagctgcatgagccagaacgccagcctgctgaaggtgtacagc | |
| | aaagaggaccaggatctgctgaagctcgtgaagtcctaccactggatgggcctgg | |
| | tgcacatccccaccaatggcagctggcagtgggaggacggcagcatcctgagcc | |
| | ccaacctgctgaccatcatcgagatgcagaagggcgactgcgccctgtacgcca | |
| | gcagcttcaagggctacatcgagaactgcagcacccccaacacctacatttgcatg | |
| | cagcgtacggttgctagcaccaagggccccagcgtgttccccctggcgcccagc | |
| | agcaagagcaccagcggcggcacagccgccctgggctgcctggtgaaggacta | |
| | cttccccgagccagtgaccgtgtcctggaacagcggagccctgacctccggcgt | |
| | gcacaccttccccgccgtgctgcagagcagcggcctgtacagcctgagcagcgt | |
| | ggtgaccgtgcccagcagcagcctgggcacccagacctacatctgcaacgtgaa | |
| | ccacaagcccagcaacaccaaggtggacaagagagtggagcccaagagctgc | |
| | gacaagacccacacctgccccccctgtcctgcccctgaactgctgggcggaccct | |
| | ccgtgttcctgttccccccaaagcccaaggacaccctgatgatcagccggacccc | |
| | cgaagtgacctgcgtggtggtggacgtgtcccacgaggaccctgaagtgaagttc | |
| | aattggtacgtggacggcgtggaagtgcacaacgccaagaccaagcccagaga | |
| | ggaacagtacaacagcacctaccgggtggtgtccgtgctgaccgtgctgcacca | |
| | ggactggctgaacggcaaagagtacaagtgcaaagtctccaacaaggccctgcc | |
| | tgcccccatcgagaaaaccatcagcaaggccaagggccagccccgcgagcccc | |
| | aggtgtacacactgccccccagccgggacgagctgaccaagaaccaggtgtccc | |
| | tgacctgcctggtcaagggcttctaccccagcgatatcgccgtggaatgggagag | |
| | caacggccagcccgagaacaactacaagaccaccccccctgtgctggacagcg | |
| | acggctcattcttcctgtacagcaagctgaccgtggacaagtcccggtggcagca | |
| | gggcaacgtgttcagctgcagcgtgatgcacgaggccctgcacaaccactacac | |
| | ccagaagtccctgagcctgagccccggcaaa | |
| HH15_2 | NQEVQIPLTESYCGPCPKNWICYKNNCYQFFDESKN | 35 |
| hNKG2D(aa84-216)- | WYESQASCMSQNASLLKVYSKEDQDLLKLVKSYH | |
| RS(G3S)2- | WMGLVHIPTNGSWQWEDGSILSPNLLTIIEMQKGDC | |
| hNKG2D(aa84-216)- | ALYASSFKGYIENCSTPNTYICMQRTVRSGGGSGGG | |
| hLCkappa | SNQEVQIPLTESYCGPCPKNWICYKNNCYQFFDESK | |
| protein | NWYESQASCMSQNASLLKVYSKEDQDLLKLVKSYH | |
| | WMGLVHIPTNGSWQWEDGSILSPNLLTIIEMQKGDC | |
| | ALYASSFKGYIENCSTPNTYICMQRTVRTVAAPSVFI | |
| | FPPSDEQLKSGTASVVCLLNNFYPREAKVQWKVDN | |
| | ALQSGNSQESVTEQDSKDSTYSLSSTLTLSKADYEK | |
| | HKVYACEVTHQGLSSPVTKSFNRGEC | |
| HH152 | aaccaggaagtgcagatccccctgaccgagagctactgcggcccctgccccaag | 36 |
| hNKG2D(aa84-216)- | aactggatctgctacaagaacaactgctaccagttcttcgacgagagcaagaattg | |
| RS(G3S)2- | gtacgagagccaggccagctgcatgagccagaacgccagcctgctgaaggtgta | |
| hNKG2D(aa84-216)- | cagcaaagaggaccaggatctgctgaagctcgtgaagtcctaccactggatggg | |
| hLCkappa | cctggtgcacatccccaccaatggcagctggcagtgggaggacggcagcatcct | |
| DNA | gagccccaacctgctgaccatcatcgagatgcagaagggcgactgcgccctgta | |
| | cgccagcagcttcaagggctacatcgagaactgtagcacccccaacacctacatc | |
| | tgtatgcagcggaccgtcagaagtggaggcgggtctggaggcggatccaacca | |
| | ggaagtgcagatccccctgaccgagagctactgcggcccctgccccaagaactg | |
| | gatctgctacaagaacaactgctaccagttcttcgacgagagcaagaattggtacg | |
| | agagccaggccagctgcatgagccagaacgccagcctgctgaaggtgtacagc | |
| | aaagaggaccaggatctgctgaagctcgtgaagtcctaccactggatgggcctgg | |
| | tgcacatccccaccaatggcagctggcagtgggaggacggcagcatcctgagcc | |
| | ccaacctgctgaccatcatcgagatgcagaagggcgactgcgccctgtacgcca | |
| | gcagcttcaagggctacatcgagaactgcagcacccccaacacctacatttgcatg | |
| | cagcgtacggttcggacagtggccgctcctagcgtgttcatctttccaccaagcga | |
| | cgagcagctgaagtctggcacagcctctgtcgtgtgcctgctgaacaacttctacc | |
| | ccagagaagccaaggtgcagtggaaggtggacaatgccctgcagagcggcaat | |
| | agccaagagagcgtgaccgagcaggacagcaaggatagcacatacagcctgag | |
| | cagcaccctgactctgagcaaggccgactacgagaagcacaaagtgtacgcctg | |
| | cgaagtgacacaccagggcctgtctagccctgtgaccaagagcttcaacagagg | |
| | cgagtgc | |
| HH16_1, | NQEVQIPLTESYCGPCPKNWICYKNNCYQFFDESKN | 37 |
| hNKG2D(aa84-216)- | WYESQASCMSQNASLLKVYSKEDQDLLKLVKSYH | |
| CH1-hFc1 | WMGLVHIPTNGSWQWEDGSILSPNLLTIIEMQKGDC | |
| Protein | ALYASSFKGYIENCSTPNTYICMQRTVASTKGPSVFP | |
| | LAPSSKSTSGGTAALGCLVKDYFPEPVTVSWNSGAL | |
| | TSGVHTFPAVLQSSGLYSLSSVVTVPSSSLGTQTYIC | |
| | NVNHKPSNTKVDKRVEPKSCDKTHTCPPCPAPELLG | |
| | GPSVFLFPPKPKDTLMISRTPEVTCVVVDVSHEDPEV | |
| | KFNWYVDGVEVHNAKTKPREEQYNSTYRVVSVLT | |
| | VLHQDWLNGKEYKCKVSNKALPAPIEKTISKAKGQ | |
| | PREPQVYTLPPSRDELTKNQVSLTCLVKGFYPSDIAV | |
| | EWESNGQPENNYKTTPPVLDSDGSFFLYSKLTVDKS | |
| | RWQQGNVFSCSVMELEALHNEIYTQKSLSLSPGK | |
| HH16_1, | aaccaggaagtgcagatccccctgaccgagagctactgcggcccctgccccaag | 38 |
| hNKG2D(aa84- | aactggatctgctacaagaacaactgctaccagttcttcgacgagagcaagaattg | |
| 216)-CH1-hFc1 | gtacgagagccaggccagctgcatgagccagaacgccagcctgctgaaggtgta | |
| DNA | cagcaaagaggaccaggatctgctgaagctcgtgaagtcctaccactggatggg | |
| | cctggtgcacatccccaccaatggcagctggcagtgggaggacggcagcatcct | |
| | gagccccaacctgctgaccatcatcgagatgcagaagggcgactgcgccctgta | |
| | cgccagcagcttcaagggctacatcgagaactgcagcacccccaacacctacatt | |
| | tgcatgcagcgtacggttgctagcaccaagggccccagcgtgttccccctggcgc | |
| | ccagcagcaagagcaccagcggcggcacagccgccctgggctgcctggtgaa | |
| | ggactacttccccgagccagtgaccgtgtcctggaacagcggagccctgacctcc | |
| | ggcgtgcacaccttccccgccgtgctgcagagcagcggcctgtacagcctgagc | |
| | agcgtggtgaccgtgcccagcagcagcctgggcacccagacctacatctgcaac | |
| | gtgaaccacaagcccagcaacaccaaggtggacaagagagtggagcccaaga | |
| | gctgcgacaagacccacacctgccccccctgtcctgcccctgaactgctgggcg | |
| | gaccctccgtgttcctgttccccccaaagcccaaggacaccctgatgatcagccg | |
| | gacccccgaagtgacctgcgtggtggtggacgtgtcccacgaggaccctgaagt | |
| | gaagttcaattggtacgtggacggcgtggaagtgcacaacgccaagaccaagcc | |
| | cagagaggaacagtacaacagcacctaccgggtggtgtccgtgctgaccgtgct | |
| | gcaccaggactggctgaacggcaaagagtacaagtgcaaagtctccaacaaggc | |
| | cctgcctgcccccatcgagaaaaccatcagcaaggccaagggccagccccgcg | |
| | agccccaggtgtacacactgccccccagccgggacgagctgaccaagaaccag | |
| | gtgtccctgacctgcctggtcaagggcttctaccccagcgatatcgccgtggaatg | |
| | ggagagcaacggccagcccgagaacaactacaagaccaccccccctgtgctgg | |
| | acagcgacggctcattcttcctgtacagcaagctgaccgtggacaagtcccggtg | |
| | gcagcagggcaacgtgttcagctgcagcgtgatgcacgaggccctgcacaacca | |
| | ctacacccagaagtccctgagcctgagccccggcaaa | |
| HH16_2 | NQEVQIPLTESYCGPCPKNWICYKNNCYQFFDESKN | 39 |
| hNKG2D(aa84-216)- | WYESQASCMSQNASLLKVYSKEDQDLLKLVKSYH | |
| hLCkappa | WMGLVHIPTNGSWQWEDGSILSPNLLTIIEMQKGDC | |
| protein | ALYASSFKGYIENCSTPNTYICMQRTVRTVAAPSVFI | |
| | FPPSDEQLKSGTASVVCLLNNFYPREAKVQWKVDN | |
| | ALQSGNSQESVTEQDSKDSTYSLSSTLTLSKADYEK | |
| | HKVYACEVTHQGLSSPVTKSFNRGEC | |
| HH16_2 | aaccaggaagtgcagatccccctgaccgagagctactgcggcccctgccccaag | 40 |
| hNKG2D(aa84-216)- | aactggatctgctacaagaacaactgctaccagttcttcgacgagagcaagaattg | |
| hLCkappa | gtacgagagccaggccagctgcatgagccagaacgccagcctgctgaaggtgta | |
| DNA | cagcaaagaggaccaggatctgctgaagctcgtgaagtcctaccactggatggg | |
| | cctggtgcacatccccaccaatggcagctggcagtgggaggacggcagcatcct | |
| | gagccccaacctgctgaccatcatcgagatgcagaagggcgactgcgccctgta | |
| | cgccagcagcttcaagggctacatcgagaactgcagcacccccaacacctacatt | |
| | tgcatgcagcgtacggttcggacagtggccgctcctagcgtgttcatctttccacca | |
| | agcgacgagcagctgaagtctggcacagcctctgtcgtgtgcctgctgaacaactt | |
| | ctaccccagagaagccaaggtgcagtggaaggtggacaatgccctgcagagcg | |
| | gcaatagccaagagagcgtgaccgagcaggacagcaaggatagcacatacagc | |
| | ctgagcagcaccctgactctgagcaaggccgactacgagaagcacaaagtgtac | |
| | gcctgcgaagtgacacaccagggcctgtctagccctgtgaccaagagcttcaaca | |
| | gaggcgagtgc | |
| HH17, | NQEVQIPLTESYCGPCPKNWICYKNNCYQFFDESKN | 41 |
| hNKG2D(aa84-216)- | WYESQASCMSQNASLLKVYSKEDQDLLKLVKSYH | |
| hFc1 protein | WMGLVHIPTNGSWQWEDGSILSPNLLTIIEMQKGDC | |
| | ALYASSFKGYIENCSTPNTYICMQRTVDKTHTCPPCP | |
| | APELLGGPSVFLFPPKPKDTLMISRTPEVTCVVVDVS | |
| | HEDPEVKFNWYVDGVEVHNAKTKPREEQYNSTYR | |
| | VVSVLTVLHQDWLNGKEYKCKVSNKALPAPIEKTIS | |
| | KAKGQPREPQVYTLPPSRDELTKNQVSLTCLVKGFY | |
| | PSDIAVEWESNGQPENNYKTTPPVLDSDGSFFLYSKL | |
| | TVDKSRWQQGNVFSCSVMHEALHNHYTQKSLSLSP | |
| | GK | |
| HH17, | aaccaggaagtgcagatccccctgaccgagagctactgcggcccctgccccaag | 42 |
| hNKG2D(aa84-216)- | aactggatctgctacaagaacaactgctaccagttcttcgacgagagcaagaattg | |
| hFc1 DNA | gtacgagagccaggccagctgcatgagccagaacgccagcctgctgaaggtgta | |
| | cagcaaagaggaccaggatctgctgaagctcgtgaagtcctaccactggatggg | |
| | cctggtgcacatccccaccaatggcagctggcagtgggaggacggcagcatcct | |
| | gagccccaacctgctgaccatcatcgagatgcagaagggcgactgcgccctgta | |
| | cgccagcagcttcaagggctacatcgagaactgcagcacccccaacacctacatc | |
| | tgtatgcagcggaccgtcgacaagacccacacctgccccccctgtcctgcccctg | |
| | aactgctgggcggaccctccgtgttcctgttccccccaaagcccaaggacaccct | |
| | gatgatcagccggacccccgaagtgacctgcgtggtggtggacgtgtcccacga | |
| | ggaccctgaagtgaagttcaattggtacgtggacggcgtggaagtgcacaacgcc | |
| | aagaccaagcccagagaggaacagtacaacagcacctaccgggtggtgtccgt | |
| | gctgaccgtgctgcaccaggactggctgaacggcaaagagtacaagtgcaaagt | |
| | ctccaacaaggccctgcctgcccccatcgagaaaaccatcagcaaggccaaggg | |
| | ccagccccgcgagccccaggtgtacacactgccccccagccgggacgagctga | |
| | ccaagaaccaggtgtccctgacctgcctggtcaagggcttctaccccagcgatatc | |
| | gccgtggaatgggagagcaacggccagcccgagaacaactacaagaccaccc | |
| | cccctgtgctggacagcgacggctcattcttcctgtacagcaagctgaccgtggac | |
| | aagtcccggtggcagcagggcaacgtgttcagctgcagcgtgatgcacgaggcc | |
| | ctgcacaaccactacacccagaagtccctgagcctgagccccggcaaa | |
| HH18, | NQEVQIPLTESYCGPCPKNWICYKNNCYQFFDESKN | 43 |
| hNKG2D(aa84-216)- | WYESQASCMSQNASLLKVYSKEDQDLLKLVKSYH | |
| RS(G3S)2- | WMGLVHIPTNGSWQWEDGSILSPNLLTIIEMQKGDC | |
| hNKG2D(aa84-216)- | ALYASSFKGYIENCSTPNTYICMQRTVRSGGGSGGG | |
| hFc1- | SNQEVQIPLTESYCGPCPKNWICYKNNCYQFFDESK | |
| hNKG2D(aa84-216)- | NWYESQASCMSQNASLLKVYSKEDQDLLKLVKSYH | |
| RS(G3S)2- | WMGLVHIPTNGSWQWEDGSILSPNLLTIIEMQKGDC | |
| hNKG2D(aa84-216) | ALYASSFKGYIENCSTPNTYICMQRTVDKTHTCPPCP | |
| protein | APELLGGPSVFLFPPKPKDTLMISRTPEVTCVVVDVS | |
| | HEDPEVKFNWYVDGVEVHNAKTKPREEQYNSTYR | |
| | VVSVLTVLHQDWLNGKEYKCKVSNKALPAPIEKTIS | |
| | KAKGQPREPQVYTLPPSRDELTKNQVSLTCLVKGFY | |
| | PSDIAVEWESNGQPENNYKTTPPVLDSDGSFFLYSKL | |
| | TVDKSRWQQGNVFSCSVMHEALHNHYTQKSLSLSP | |
| | GKNQEVQIPLTESYCGPCPKNWICYKNNCYQFFDES | |
| | KNWYESQASCMSQNASLLKVYSKEDQDLLKLVKSY | |
| | HWMGLVHIPTNGSWQWEDGSILSPNLLTIIEMQKGD | |
| | CALYASSFKGYIENCSTPNTYICMQRTVRSGGGSGG | |
| | GSNQEVQIPLTESYCGPCPKNWICYKNNCYQFFDES | |
| | KNWYESQASCMSQNASLLKVYSKEDQDLLKLVKSY | |
| | HWMGLVHIPTNGSWQWEDGSILSPNLLTIIEMQKGD | |
| | CALYASSFKGYIENCSTPNTYICMQRTV | |
| HH18, | aaccaggaagtgcagatccccctgaccgagagctactgcggcccctgccccaag | 44 |
| hNKG2D(aa84-216)- | aactggatctgctacaagaacaactgctaccagttcttcgacgagagcaagaattg | |
| RS(G3S)2- | gtacgagagccaggccagctgcatgagccagaacgccagcctgctgaaggtgta | |
| hNKG2D(aa84-216)- | cagcaaagaggaccaggatctgctgaagctcgtgaagtcctaccactggatggg | |
| hFc1- | cctggtgcacatccccaccaatggcagctggcagtgggaggacggcagcatcct | |
| hNKG2D(aa84-216)- | gagccccaacctgctgaccatcatcgagatgcagaagggcgactgcgccctgta | |
| RS(G3S)2- | cgccagcagcttcaagggctacatcgagaactgtagcacccccaacacctacatc | |
| hNKG2D(aa84-216) | tgtatgcagcggaccgtcagaagtggaggcgggtctggaggcggatccaacca | |
| DNA | ggaagtgcagatccccctgaccgagagctactgcggcccctgccccaagaactg | |
| | gatctgctacaagaacaactgctaccagttcttcgacgagagcaagaattggtacg | |
| | agagccaggccagctgcatgagccagaacgccagcctgctgaaggtgtacagc | |
| | aaagaggaccaggatctgctgaagctcgtgaagtcctaccactggatgggcctgg | |
| | tgcacatccccaccaatggcagctggcagtgggaggacggcagcatcctgagcc | |
| | ccaacctgctgaccatcatcgagatgcagaagggcgactgcgccctgtacgcca | |
| | gcagcttcaagggctacatcgagaactgcagcacccccaacacctacatttgcatg | |
| | cagcgtacggttgacaagacccacacctgccccccctgtcctgcccctgaactgct | |
| | gggcggaccctccgtgttcctgttccccccaaagcccaaggacaccctgatgatc | |
| | agccggacccccgaagtgacctgcgtggtggtggacgtgtcccacgaggaccct | |
| | gaagtgaagttcaattggtacgtggacggcgtggaagtgcacaacgccaagacc | |
| | aagcccagagaggaacagtacaacagcacctaccgggtggtgtccgtgctgacc | |
| | gtgctgcaccaggactggctgaacggcaaagagtacaagtgcaaagtctccaac | |
| | aaggccctgcctgcccccatcgagaaaaccatcagcaaggccaagggccagcc | |
| | ccgcgagccccaggtgtacacactgccccccagccgggacgagctgaccaaga | |
| | accaggtgtccctgacctgcctggtcaagggcttctaccccagcgatatcgccgtg | |
| | gaatgggagagcaacggccagcccgagaacaactacaagaccaccccccctgt | |
| | gctggacagcgacggctcattcttcctgtacagcaagctgaccgtggacaagtcc | |
| | cggtggcagcagggcaacgtgttcagctgcagcgtgatgcacgaggccctgcac | |
| | aaccactacacccagaagtccctgagcctgagccccggcaaaaaccaggaagtg | |
| | cagatccccctgaccgagagctactgcggcccctgccccaagaactggatctgct | |
| | acaagaacaactgctaccagttcttcgacgagagcaagaattggtacgagagcca | |
| | ggccagctgcatgagccagaacgccagcctgctgaaggtgtacagcaaagagg | |
| | accaggatctgctgaagctcgtgaagtcctaccactggatgggcctggtgcacatc | |
| | cccaccaatggcagctggcagtgggaggacggcagcatcctgagccccaacct | |
| | gctgaccatcatcgagatgcagaagggcgactgcgccctgtacgccagcagcttc | |
| | aagggctacatcgagaactgtagcacccccaacacctacatctgtatgcagcgga | |
| | ccgtcagaagtggaggcgggtctggaggcggatccaaccaggaagtgcagatc | |
| | cccctgaccgagagctactgcggcccctgccccaagaactggatctgctacaaga | |
| | acaactgctaccagttcttcgacgagagcaagaattggtacgagagccaggccag | |
| | ctgcatgagccagaacgccagcctgctgaaggtgtacagcaaagaggaccagg | |
| | atctgctgaagctcgtgaagtcctaccactggatgggcctggtgcacatccccacc | |
| | aatggcagctggcagtgggaggacggcagcatcctgagccccaacctgctgacc | |
| | atcatcgagatgcagaagggcgactgcgccctgtacgccagcagcttcaagggc | |
| | tacatcgagaactgcagcacccccaacacctacatttgcatgcagcgtacggtt | |
| HH19, | NQEVQIPLTESYCGPCPKNWICYKNNCYQFFDESKN | 45 |
| hNKG2D(aa84-216)- | WYESQASCMSQNASLLKVYSKEDQDLLKLVKSYH | |
| RS(G3S)2- | WMGLVHIPTNGSWQWEDGSILSPNLLTIIEMQKGDC | |
| hNKG2D(aa84-216- | ALYASSFKGYIENCSTPNTYICMQRTVRSGGGSGGG | |
| RSGGG- | SNQEVQIPLTESYCGPCPKNWICYKNNCYQFFDESK | |
| hNKG2D(aa84-216)- | NWYESQASCMSQNASLLKVYSKEDQDLLKLVKSYH | |
| RS(G3S)2- | WMGLVHIPTNGSWQWEDGSILSPNLLTIIEMQKGDC | |
| hNKG2D(aa84-216)- | ALYASSFKGYIENCSTPNTYICMQRTVRSGGGNQEV | |
| hFc1 protein | QIPLTESYCGPCPKNWICYKNNCYQFFDESKNWYES | |
| | QASCMSQNASLLKVYSKEDQDLLKLVKSYHWMGL | |
| | VHIPTNGSWQWEDGSILSPNLLTIIEMQKGDCALYAS | |
| | SFKGYIENCSTPNTYICMQRTVRSGGGSGGGSNQEV | |
| | QIPLTESYCGPCPKNWICYKNNCYQFFDESKNWYES | |
| | QASCMSQNASLLKVYSKEDQDLLKLVKSYHWMGL | |
| | VHIPTNGSWQWEDGSILSPNLLTIIEMQKGDCALYAS | |
| | SFKGYIENCSTPNTYICMQRTVDKTHTCPPCPAPELL | |
| | GGPSVFLFPPKPKDTLMISRTPEVTCVVVDVSHEDPE | |
| | VKFNWYVDGVEVHNAKTKPREEQYNSTYRVVSVL | |
| | TVLHQDWLNGKEYKCKVSNKALPAPIEKTISKAKG | |
| | QPREPQVYTLPPSRDELTKNQVSLTCLVKGFYPSDIA | |
| | VEWESNGQPENNYKTTPPVLDSDGSFFLYSKLTVDK | |
| | SRWQQGNVFSCSVMEIEALHNHYTQKSLSLSPGK | |
| HH19, | aaccaggaagtgcagatccccctgaccgagagctactgcggcccctgccccaag | 46 |
| hNKG2D(aa84-216)- | aactggatctgctacaagaacaactgctaccagttcttcgacgagagcaagaattg | |
| RS(G3S)2- | gtacgagagccaggccagctgcatgagccagaacgccagcctgctgaaggtgta | |
| hNKG2D(aa84-216- | cagcaaagaggaccaggatctgctgaagctcgtgaagtcctaccactggatggg | |
| RSGGG- | cctggtgcacatccccaccaatggcagctggcagtgggaggacggcagcatcct | |
| hNKG2D(aa84-216)- | gagccccaacctgctgaccatcatcgagatgcagaagggcgactgcgccctgta | |
| RS(G3S)2- | cgccagcagcttcaagggctacatcgagaactgtagcacccccaacacctacatc | |
| hNKG2D(aa84-216)- | tgtatgcagcggaccgtcagaagtggaggcgggtctggaggcggatccaacca | |
| hFc1 DNA | ggaagtgcagatccccctgaccgagagctactgcggcccctgccccaagaactg | |
| | gatctgctacaagaacaactgctaccagttcttcgacgagagcaagaattggtacg | |
| | agagccaggccagctgcatgagccagaacgccagcctgctgaaggtgtacagc | |
| | aaagaggaccaggatctgctgaagctcgtgaagtcctaccactggatgggcctgg | |
| | tgcacatccccaccaatggcagctggcagtgggaggacggcagcatcctgagcc | |
| | ccaacctgctgaccatcatcgagatgcagaagggcgactgcgccctgtacgcca | |
| | gcagcttcaagggctacatcgagaactgcagcacccccaacacctacatttgcatg | |
| | cagcgcacggttaggtccggcgggggaaaccaggaagtgcagatccccctgac | |
| | cgagagctactgcggcccctgccccaagaactggatctgctacaagaacaactgc | |
| | taccagttcttcgacgagagcaagaattggtacgagagccaggccagctgcatga | |
| | gccagaacgccagcctgctgaaggtgtacagcaaagaggaccaggatctgctga | |
| | agctcgtgaagtcctaccactggatgggcctggtgcacatccccaccaatggcag | |
| | ctggcagtgggaggacggcagcatcctgagccccaacctgctgaccatcatcga | |
| | gatgcagaagggcgactgcgccctgtacgccagcagcttcaagggctacatcga | |
| | gaactgtagcacccccaacacctacatctgtatgcagcggaccgtcagaagtgga | |
| | ggcgggtctggaggcggatccaaccaggaagtgcagatccccctgaccgagag | |
| | ctactgcggcccctgccccaagaactggatctgctacaagaacaactgctaccagt | |
| | tcttcgacgagagcaagaattggtacgagagccaggccagctgcatgagccaga | |
| | acgccagcctgctgaaggtgtacagcaaagaggaccaggatctgctgaagctcg | |
| | tgaagtcctaccactggatgggcctggtgcacatccccaccaatggcagctggca | |
| | gtgggaggacggcagcatcctgagccccaacctgctgaccatcatcgagatgca | |
| | gaagggcgactgcgccctgtacgccagcagcttcaagggctacatcgagaactg | |
| | cagcacccccaacacctacatttgcatgcagcgtacggttgacaagacccacacc | |
| | tgccccccctgtcctgcccctgaactgctgggcggaccctccgtgttcctgttcccc | |
| | ccaaagcccaaggacaccctgatgatcagccggacccccgaagtgacctgcgtg | |
| | gtggtggacgtgtcccacgaggaccctgaagtgaagttcaattggtacgtggacg | |
| | gcgtggaagtgcacaacgccaagaccaagcccagagaggaacagtacaacag | |
| | cacctaccgggtggtgtccgtgctgaccgtgctgcaccaggactggctgaacggc | |
| | aaagagtacaagtgcaaagtctccaacaaggccctgcctgcccccatcgagaaa | |
| | accatcagcaaggccaagggccagccccgcgagccccaggtgtacacactgcc | |
| | ccccagccgggacgagctgaccaagaaccaggtgtccctgacctgcctggtcaa | |
| | gggcttctaccccagcgatatcgccgtggaatgggagagcaacggccagcccga | |
| | gaacaactacaagaccaccccccctgtgctggacagcgacggctcattcttcctgt | |
| | acagcaagctgaccgtggacaagtcccggtggcagcagggcaacgtgttcagct | |
| | gcagcgtgatgcacgaggccctgcacaaccactacacccagaagtccctgagcc | |
| | tgagccccggcaaa | |
| MM1, mNKG2D | FQPVLCNKEVPVSSREGYCGPCPNNWICHRNNCYQF | 47 |
| (aa94-232)- | FNEEKTWNQSQASCLSQNSSLLKIYSKEEQDFLKLV | |
| RS(G3S)2- | KSYHWMGLVQIPANGSWQWEDGSSLSYNQLTLVEI | |
| mNKG2D (aa94-232)- | PKGSCAVYGSSFKAYTEDCANLNTYICMKRAVRSG | |
| mFc2 | GGSGGGSFQPVLCNKEVPVSSREGYCGPCPNNWICH | |
| protein | RNNCYQFFNEEKTWNQSQASCLSQNSSLLKIYSKEE | |
| | QDFLKLVKSYHWMGLVQIPANGSWQWEDGSSLSY | |
| | NQLTLVEIPKGSCAVYGSSFKAYTEDCANLNTYICM | |
| | KRAVISAMVRSPRGPTIKPCPPCKCPAPNLLGGPSVFI | |
| | FPPKIKDVLMISLSPIVTCVVVDVSEDDPDVQISWFV | |
| | NNVEVHTAQTQTHREDYNSTLRVVSALPIQHQDWM | |
| | SGKEFKCKVNNKDLPAPIERTISKPKGSVRAPQVYV | |
| | LPPPEEEMTKKQVTLTCMVTDFMPEDIYVEWTNNG | |
| | KTELNYKNTEPVLDSDGSYFMYSKLRVEKKNWVER | |
| | NSYSCSVVHEGLHNHHTTKSFSRTPGK | |
| MMI, mNKG2D | tttcaacctgtcctgtgtaacaaagaggttccagtgagttcaagagagggatactgc | 48 |
| (aa94-232)- | gggccatgtccgaataactggatctgccacaggaacaactgctatcagtttttcaac | |
| RS(G3S)2- | gaagaaaagacatggaaccagagtcaggcttcctgtttgtctcagaactccagtct | |
| mNKG2D (aa94-232)- | gctgaagatttactcaaaggaggaacaggacttcctcaaactggtaaagagctatc | |
| mFc2 DNA | attggatgggactggttcagatcccagccaatggctcttggcagtgggaggatgg | |
| | aagctctctcagctacaaccagcttaccctggtggaaattcccaaaggaagctgcg | |
| | cagtgtatggttcaagtttcaaagcctacacagaagattgcgctaacctgaatacat | |
| | acatttgcatgaaaagggctgtcagaagtggaggcgggtctggaggcgggtcttt | |
| | ccaacctgtcctgtgcaacaaagaagtgcccgtcagttccagagagggctactgt | |
| | gggccatgtcccaataattggatttgccacagaaacaactgctaccagttctttaatg | |
| | aggagaaaacttggaaccagtcacaagcctcttgtctgtcacaaaacagcagcttg | |
| | ctgaaaatctacagcaaggaggagcaggactttctgaagctggtcaaatcttatca | |
| | ctggatgggtctggtgcagatccccgccaatggatcttggcaatgggaggacggc | |
| | tcttctctctcctataatcagctgacactggtagaaatccccaaagggagctgtgca | |
| | gtgtacgggagcagttttaaggcctataccgaggattgcgcaaatttgaacacttac | |
| | atctgcatgaagagggccgtcatatcagctatggtccgctcaccaagaggaccaa | |
| | ccattaagccttgcccgccatgcaagtgccccgctccaaaccttctgggagggcc | |
| | gtctgtgttcatttttcctcccaaaatcaaagatgtgctgatgattagcctgtctccaat | |
| | cgtgacctgtgttgtcgtggacgtgtcagaggatgaccccgatgtgcagataagtt | |
| | ggtttgtaaacaatgtagaggtgcacacggcacagacacagacccatagggagg | |
| | attacaattctaccctgcgggtcgttagcgccctgccgatccagcaccaggactgg | |
| | atgtcaggtaaagaatttaagtgcaaggtgaataataaggatctgcccgctcctatt | |
| | gagagaacgattagcaagcccaagggctccgtgcgcgcaccccaagtctacgtt | |
| | cttcctcccccagaagaggagatgaccaaaaagcaggtcaccctgacgtgtatgg | |
| | tgactgattttatgcccgaagatatttacgtggaatggacaaacaacggtaaaacag | |
| | agctgaactacaagaatactgagcccgtgctggactccgacggctcttactttatgt | |
| | atagcaagctgagagttgagaagaaaaattgggtagagagaaattcttactcatgtt | |
| | ctgttgtacatgaaggactgcacaatcatcacaccacaaagagtttcagtagaactc | |
| | caggtaaa | |
| MM2, mNKG2D | FQPVLCNKEVPVSSREGYCGPCPNNWICHRNNCYQF | 49 |
| (aa94-232)- | FNEEKTWNQSQASCLSQNSSLLKIYSKEEQDFLKLV | |
| RS(G3S)2- | KSYHWMGLVQIPANGSWQWEDGSSLSYNQLTLVEI | |
| mNKG2D (aa94-232)- | PKGSCAVYGSSFKAYTEDCANLNTYICMKRAVRSG | |
| (G4S)2- | GGSGGGSFQPVLCNKEVPVSSREGYCGPCPNNWICH | |
| mFc2 protein | RNNCYQFFNEEKTWNQSQASCLSQNSSLLKIYSKEE | |
| | QDFLKLVKSYHWMGLVQIPANGSWQW | |
| | EDGSSLSYNQLTLVEIPKGSCAVYGSSFKAYTEDCA | |
| | NLNTYICMKRAVGGGGSGGGGSISAMVRSPRGPTIK | |
| | PCPPCKCPAPNLLGGPSVFIFPPKIKDVLMISLSPIVTC | |
| | VVVDVSEDDPDVQISWFVNNVEVHTAQTQTHREDY | |
| | NSTLRVVSALPIQHQDWMSGKEFKCKVNNKDLPAPI | |
| | ERTISKPKGSVRAPQVYVLPPPEEEMTKKQVTLTCM | |
| | VTDFMPEDIYVEWTNNGKTELN | |
| | YKNTEPVLDSDGSYFMYSKLRVEKKNWVERNSYSC | |
| | SVVHEGLHNHHTTKSFSRTPGK | |
| MM2, mNKG2D | tttcaacctgtcctgtgtaacaaagaggttccagtgagttcaagagagggatactgc | 50 |
| (aa94-232)- | gggccatgtccgaataactggatctgccacaggaacaactgctatcagtttttcaac | |
| RS(G3S)2- | gaagaaaagacatggaaccagagtcaggcttcctgtttgtctcagaactccagtct | |
| mNKG2D (aa94-232)- | gctgaagatttactcaaaggaggaacaggacttcctcaaactggtaaagagctatc | |
| (G4S)2- | attggatgggactggttcagatcccagccaatggctcttggcagtgggaggatgg | |
| mFc2 DNA | aagctctctcagctacaaccagcttaccctggtggaaattcccaaaggaagctgcg | |
| | cagtgtatggttcaagtttcaaagcctacacagaagattgcgctaacctgaatacat | |
| | acatttgcatgaaaagggctgtcagaagtggaggcgggtctggaggcgggtcttt | |
| | ccaacctgtcctgtgcaacaaagaagtgcccgtcagttccagagagggctactgt | |
| | gggccatgtcccaataattggatttgccacagaaacaactgctaccagttctttaatg | |
| | aggagaaaacttggaaccagtcacaagcctcttgtctgtcacaaaacagcagcttg | |
| | ctgaaaatctacagcaaggaggagcaggactttctgaagctggtcaaatcttatca | |
| | ctggatgggtctggtgcagatccccgccaatggatcttggcaatgggaggacggc | |
| | tcttctctctcctataatcagctgacactggtagaaatccccaaagggagctgtgca | |
| | gtgtacgggagcagttttaaggcctataccgaggattgcgcaaatttgaacacttac | |
| | atctgcatgaagagggccgtcggtggagggggctccggaggcggtgggagcat | |
| | atcagctatggtccgctcaccaagaggaccaaccattaagccttgcccgccatgca | |
| | agtgccccgctccaaaccttctgggagggccgtctgtgttcatttttcctcccaaaat | |
| | caaagatgtgctgatgattagcctgtctccaatcgtgacctgtgttgtcgtggacgtg | |
| | tcagaggatgaccccgatgtgcagataagttggtttgtaaacaatgtagaggtgca | |
| | cacggcacagacacagacccatagggaggattacaattctaccctgcgggtcgtt | |
| | agcgccctgccgatccagcaccaggactggatgtcaggtaaagaatttaagtgca | |
| | aggtgaataataaggatctgcccgctcctattgagagaacgattagcaagcccaag | |
| | ggctccgtgcgcgcaccccaagtctacgttcttcctcccccagaagaggagatga | |
| | ccaaaaagcaggtcaccctgacgtgtatggtgactgattttatgcccgaagatattta | |
| | cgtggaatggacaaacaacggtaaaacagagctgaactacaagaatactgagcc | |
| | cgtgctggactccgacggctcttactttatgtatagcaagctgagagttgagaagaa | |
| | aaattgggtagagagaaattcttactcatgttctgttgtacatgaaggactgcacaat | |
| | catcacaccacaaagagtttcagtagaactccaggtaaa | |
| MM3, mNKG2D | FQPVLCNKEVPVSSREGYCGPCPNNWICHRNNCYQF | 51 |
| (aa94-232)- | FNEEKTWNQSQASCLSQNSSLLKIYSKEEQDFLKLV | |
| RS(G3S)2- | KSYHWMGLVQIPANGSWQWEDGSSLSYNQLTLVEI | |
| mNKG2D (aa94-232)- | PKGSCAVYGSSFKAYTEDCANLNTYICMKRAVRSG | |
| (G4S)4- | GGSGGGSFQPVLCNKEVPVSSREGYCGPCPNNWICH | |
| mFc2 protein | RNNCYQFFNEEKTWNQSQASCLSQNSSLLKIYSKEE | |
| | QDFLKLVKSYHWMGLVQIPANGSWQW | |
| | EDGSSLSYNQLTLVEIPKGSCAVYGSSFKAYTEDCA | |
| | NLNTYICMKRAVGGGGSGGGGSGGGGSGGGGSISA | |
| | MVRSPRGPTIKPCPPCKCPAPNLLGGPSVFIFPPKIKD | |
| | VLMISLSPIVTCVVVDVSEDDPDVQISWFVNNVEVH | |
| | TAQTQTHREDYNSTLRVVSALPIQHQDWMSGKEFK | |
| | CKVNNKDLPAPIERnSKPKGSVRAPQVYVLPPPEEE | |
| | MTKKQVTLTCMVTDFMPEDIYVEWTNNGKTELNY | |
| | KNTEPVLDSDGSYFMYSKLRVEKKNWVERNSYSCS | |
| | VVHEGLHNHHTTKSFSRTPGK | |
| MM3, mNKG2D | tttcaacctgtcctgtgtaacaaagaggttccagtgagttcaagagagggatactgc | 52 |
| (aa94-232)- | gggccatgtccgaataactggatctgccacaggaacaactgctatcagtttttcaac | |
| RS(G3S)2- | gaagaaaagacatggaaccagagtcaggcttcctgtttgtctcagaactccagtct | |
| mNKG2D (aa94-232)- | gctgaagatttactcaaaggaggaacaggacttcctcaaactggtaaagagctatc | |
| (G4S)4- | attggatgggactggttcagatcccagccaatggctcttggcagtgggaggatgg | |
| mFc2 DNA | aagctctctcagctacaaccagcttaccctggtggaaattcccaaaggaagctgcg | |
| | cagtgtatggttcaagtttcaaagcctacacagaagattgcgctaacctgaatacat | |
| | acatttgcatgaaaagggctgtcagaagtggaggcgggtctggaggcgggtcttt | |
| | ccaacctgtcctgtgcaacaaagaagtgcccgtcagttccagagagggctactgt | |
| | gggccatgtcccaataattggatttgccacagaaacaactgctaccagttctttaatg | |
| | aggagaaaacttggaaccagtcacaagcctcttgtctgtcacaaaacagcagcttg | |
| | ctgaaaatctacagcaaggaggagcaggactttctgaagctggtcaaatcttatca | |
| | ctggatgggtctggtgcagatccccgccaatggatcttggcaatgggaggacggc | |
| | tcttctctctcctataatcagctgacactggtagaaatccccaaagggagctgtgca | |
| | gtgtacgggagcagttttaaggcctataccgaggattgcgcaaatttgaacacttac | |
| | atctgcatgaagagggccgtcggtggagggggcagcggaggtgggggcagcg | |
| | ggggaggtgggtccggaggcggtgggagcatatcagctatggtccgctcaccaa | |
| | gaggaccaaccattaagccttgcccgccatgcaagtgccccgctccaaaccttctg | |
| | ggagggccgtctgtgttcatttttcctcccaaaatcaaagatgtgctgatgattagcct | |
| | gtctccaatcgtgacctgtgttgtcgtggacgtgtcagaggatgaccccgatgtgca | |
| | gataagttggtttgtaaacaatgtagaggtgcacacggcacagacacagacccata | |
| | gggaggattacaattctaccctgcgggtcgttagcgccctgccgatccagcacca | |
| | ggactggatgtcaggtaaagaatttaagtgcaaggtgaataataaggatctgcccg | |
| | ctcctattgagagaacgattagcaagcccaagggctccgtgcgcgcaccccaagt | |
| | ctacgttcttcctcccccagaagaggagatgaccaaaaagcaggtcaccctgacg | |
| | tgtatggtgactgattttatgcccgaagatatttacgtggaatggacaaacaacggta | |
| | aaacagagctgaactacaagaatactgagcccgtgctggactccgacggctctta | |
| | ctttatgtatagcaagctgagagttgagaagaaaaattgggtagagagaaattctta | |
| | ctcatgttctgttgtacatgaaggactgcacaatcatcacaccacaaagagtttcagt | |
| | agaactccaggtaaa | |
| MM4, mNKG2D | FQPVLCNKEVPVSSREGYCGPCPNNWICHRNNCYQF | 53 |
| (aa94-232)- | FNEEKTWNQSQASCLSQNSSLLKIYSKEEQDFLKLV | |
| RS(G3S)2- | KSYHWMGLVQIPANGSWQWEDGSSLSYNQLTLVEI | |
| mNKG2D (aa94-232)- | PKGSCAVYGSSFKAYTEDCANLNTYICMKRAVRSG | |
| (G4S)6- | GGSGGGSFQPVLCNKEVPVSSREGYCGPCPNNWICH | |
| mFc2 protein | RNNCYQFFNEEKTWNQSQASCLSQNSSLLKIYSKEE | |
| | QDFLKLVKSYHWMGLVQIPANGSWQW | |
| | EDGSSLSYNQLTLVEIPKGSCAVYGSSFKAYTEDCA | |
| | NLNTYICMKRAVGGGGSGGGGSGGGGSGGGGSGG | |
| | GGSGGGGSISAMVRSPRGPTIKPCPPCKCPAPNLLGG | |
| | PSVFIFPPKIKDVLMISLSPIVTCVVVDVSEDDPDVQI | |
| | SWFVNNVEVHTAQTQTHREDYNSTLRVVSALPIQH | |
| | QDWMSGKEFKCKVNNKDLPAPIERTISKPKGSVRAP | |
| | QVYVLPPPEEEMTKKQVTLTCMVTDFMPEDIYVEW | |
| | TNNGKTELNYKNTEPVLDSDGSYFMYSKLRVEKKN | |
| | WVERNSYSCSVVHEGLHNHHTTKSFSRTPGK | |
| MM4, mNKG2D | tttcaacctgtcctgtgtaacaaagaggttccagtgagttcaagagagggatactgc | 54 |
| (aa94-232)- | gggccatgtccgaataactggatctgccacaggaacaactgctatcagtttttcaac | |
| RS(G3S)2- | gaagaaaagacatggaaccagagtcaggcttcctgtttgtctcagaactccagtct | |
| mNKG2D (aa94-232)- | gctgaagatttactcaaaggaggaacaggacttcctcaaactggtaaagagctatc | |
| (G4S)6- | attggatgggactggttcagatcccagccaatggctcttggcagtgggaggatgg | |
| mFc2 DNA | aagctctctcagctacaaccagcttaccctggtggaaattcccaaaggaagctgcg | |
| | cagtgtatggttcaagtttcaaagcctacacagaagattgcgctaacctgaatacat | |
| | acatttgcatgaaaagggctgtcagaagtggaggcgggtctggaggcgggtcttt | |
| | ccaacctgtcctgtgcaacaaagaagtgcccgtcagttccagagagggctactgt | |
| | gggccatgtcccaataattggatttgccacagaaacaactgctaccagttctttaatg | |
| | aggagaaaacttggaaccagtcacaagcctcttgtctgtcacaaaacagcagcttg | |
| | ctgaaaatctacagcaaggaggagcaggactttctgaagctggtcaaatcttatca | |
| | ctggatgggtctggtgcagatccccgccaatggatcttggcaatgggaggacggc | |
| | tcttctctctcctataatcagctgacactggtagaaatccccaaagggagctgtgca | |
| | gtgtacgggagcagttttaaggcctataccgaggattgcgcaaatttgaacacttac | |
| | atctgcatgaagagggccgtcggtggagggggctccggaggtgggggcagcg | |
| | ggggaggtgggtccggtgggggaggctctggcggaggtgggtctggaggcgg | |
| | tgggagcatatcagctatggtccgctcaccaagaggaccaaccattaagccttgcc | |
| | cgccatgcaagtgccccgctccaaaccttctgggagggccgtctgtgttcatttttc | |
| | ctcccaaaatcaaagatgtgctgatgattagcctgtctccaatcgtgacctgtgttgt | |
| | cgtggacgtgtcagaggatgaccccgatgtgcagataagttggtttgtaaacaatgt | |
| | agaggtgcacacggcacagacacagacccatagggaggattacaattctaccct | |
| | gcgggtcgttagcgccctgccgatccagcaccaggactggatgtcaggtaaaga | |
| | atttaagtgcaaggtgaataataaggatctgcccgctcctattgagagaacgattag | |
| | caagcccaagggctccgtgcgcgcaccccaagtctacgttcttcctcccccagaa | |
| | gaggagatgaccaaaaagcaggtcaccctgacgtgtatggtgactgattttatgcc | |
| | cgaagatatttacgtggaatggacaaacaacggtaaaacagagctgaactacaag | |
| | aatactgagcccgtgctggactccgacggctcttactttatgtatagcaagctgaga | |
| | gttgagaagaaaaattgggtagagagaaattcttactcatgttctgttgtacatgaag | |
| | gactgcacaatcatcacaccacaaagagtttcagtagaactccaggtaaa | |
| MM5, mFc2- | ISAMVRSPRGPTIKPCPPCKCPAPNLLGGPSVFIFPPKI | 55 |
| mNKG2D (aa94-232)- | KDVLMISLSPIVTCVVVDVSEDDPDVQISWFVNNVE | |
| RS(G3S)2- | VHTAQTQTHREDYNSTLRVVSALPIQHQDWMSGKE | |
| mNKG2D (aa94-232) | FKCKVNNKDLPAPIERTISKPKGSVRAPQVYVLPPPE | |
| protein | EEMTKKQVTLTCMVTDFMPEDIYVEWTNNGKTELN | |
| | YKNTEPVLDSDGSYFMYSKLRVEKKNWVERNSYSC | |
| | SVVHEGLHNHHTTKSFSRTPGKFQPVLCNKEVPVSS | |
| | REGYCGPCPNNWICHRNNCYQFFNEEKTWNQSQAS | |
| | CLSQNSSLLKIYSKEEQDFLKLVKSYHWMGLVQIPA | |
| | NGSWQWEDGSSLSYNQLTLVEIPKGSCAVYGSSFKA | |
| | YTEDCANLNTYICMKRAVRSGGGSGGGSFQPVLCN | |
| | KEVPVSSREGYCGPCPNNWICHRNNCYQFFNEEKT | |
| | WNQSQASCLSQNSSLLKIYSKEEQDFLKLVKSYHW | |
| | MGLVQIPANGSWQWEDGSSLSYNQLTLVEIPKGSCA | |
| | VYGSSFKAYTEDCANLNTYICMKRAV | |
| MM5, mFc2- | atatcagctatggtccgctcaccaagaggaccaaccattaagccttgcccgccatg | 56 |
| mNKG2D (aa94-232)- | caagtgccccgctccaaaccttctgggagggccgtctgtgttcatttttcctcccaaa | |
| RS(G3S)2- | atcaaagatgtgctgatgattagcctgtctccaatcgtgacctgtgttgtcgtggacg | |
| mNKG2D (aa94-232) | tgtcagaggatgaccccgatgtgcagataagttggtttgtaaacaatgtagaggtgc | |
| DNA | acacggcacagacacagacccatagggaggattacaattctaccctgcgggtcgt | |
| | tagcgccctgccgatccagcaccaggactggatgtcaggtaaagaatttaagtgc | |
| | aaggtgaataataaggatctgcccgctcctattgagagaacgattagcaagcccaa | |
| | gggctccgtgcgcgcaccccaagtctacgttcttcctcccccagaagaggagatg | |
| | accaaaaagcaggtcaccctgacgtgtatggtgactgattttatgcccgaagatattt | |
| | acgtggaatggacaaacaacggtaaaacagagctgaactacaagaatactgagc | |
| | ccgtgctggactccgacggctcttactttatgtatagcaagctgagagttgagaaga | |
| | aaaattgggtagagagaaattcttactcatgttctgttgtacatgaaggactgcacaa | |
| | tcatcacaccacaaagagtttcagtagaactccaggtaaatttcaacctgtcctgtgt | |
| | aacaaagaggttccagtgagttcaagagagggatactgcgggccatgtccgaata | |
| | actggatctgccacaggaacaactgctatcagtttttcaacgaagaaaagacatgg | |
| | aaccagagtcaggcttcctgtttgtctcagaactccagtctgctgaagatttactcaa | |
| | aggaggaacaggacttcctcaaactggtaaagagctatcattggatgggactggtt | |
| | cagatcccagccaatggctcttggcagtgggaggatggaagctctctcagctaca | |
| | accagcttaccctggtggaaattcccaaaggaagctgcgcagtgtatggttcaagtt | |
| | tcaaagcctacacagaagattgcgctaacctgaatacatacatttgcatgaaaagg | |
| | gctgtcagaagtggaggcgggtctggaggcgggtctttccaacctgtcctgtgca | |
| | acaaagaagtgcccgtcagttccagagagggctactgtgggccatgtcccaataa | |
| | ttggatttgccacagaaacaactgctaccagttctttaatgaggagaaaacttggaa | |
| | ccagtcacaagcctcttgtctgtcacaaaacagcagcttgctgaaaatctacagcaa | |
| | ggaggagcaggactttctgaagctggtcaaatcttatcactggatgggtctggtgc | |
| | agatccccgccaatggatcttggcaatgggaggacggctcttctctctcctataatc | |
| | agctgacactggtagaaatccccaaagggagctgtgcagtgtacgggagcagttt | |
| | taaggcctataccgaggattgcgcaaatttgaacacttacatctgcatgaagaggg | |
| | ccgtc | |
| MM6, mFc2- | ISAMVRSPRGPTIKPCPPCKCPAPNLLGGPSVFIFPPKI | 57 |
| (G4S)2- | KDVLMISLSPIVTCVVVDVSEDDPDVQISWFVNNVE | |
| mNKG2D (aa94-232)- | VHTAQTQTHREDYNSTLRVVSALPIQHQDWMSGKE | |
| RS(G3S)2- | FKCKVNNKDLPAPIERTISKPKGSVRAPQVYVLPPPE | |
| mNKG2D (aa94-232) | EEMTKKQVTLTCMVTDFMPEDIYVEWTNNGKTELN | |
| protein | YKNTEPVLDSDGSYFMYSKLRVEKKNWVERNSYSC | |
| | SVVHEGLHNHHTTKSFSRTPGKGGGGSGGGGSFQP | |
| | VLCNKEVPVS SREGYCGPCPNNWICHRNNC YQFFNE | |
| | EKTWNQSQASCLSQNSSLLKIYSKEEQDFLKLVKSY | |
| | HWMGLVQIPANGS WQWEDGS SLS YNQLTLVEIPKG | |
| | SCAVYGSSFKAYTEDCANLNTYICMKRAVRSGGGS | |
| | GGGS FQP VLCNKE VP VS S REGYCGPC PNNWICHRNN | |
| | CYQFFNEEKTWNQSQASCLSQNSSLLKIYSKEEQDF | |
| | LKLVKS YHWMGLVQIPANGS WQWEDGS SLS YNQL | |
| | TLVEIPKGSCAVYGSSFKAYTEDCANLNTYICMKRA | |
| | V | |
| MM6, mFc2- | atatcagctatggtccgctcaccaagaggaccaaccattaagccttgcccgccatg | 58 |
| (G4S)2- | caagtgccccgctccaaaccttctgggagggccgtctgtgttcatttttcctcccaaa | |
| mNKG2D (aa94-232)- | atcaaagatgtgctgatgattagcctgtctccaatcgtgacctgtgttgtcgtggacg | |
| RS(G3S)2- | tgtcagaggatgaccccgatgtgcagataagttggtttgtaaacaatgtagaggtgc | |
| mNKG2D (aa94-232) | acacggcacagacacagacccatagggaggattacaattctaccctgcgggtcgt | |
| DNA | tagcgccctgccgatccagcaccaggactggatgtcaggtaaagaatttaagtgc | |
| | aaggtgaataataaggatctgcccgctcctattgagagaacgattagcaagcccaa | |
| | gggctccgtgcgcgcaccccaagtctacgttcttcctcccccagaagaggagatg | |
| | accaaaaagcaggtcaccctgacgtgtatggtgactgattttatgcccgaagatattt | |
| | acgtggaatggacaaacaacggtaaaacagagctgaactacaagaatactgagc | |
| | ccgtgctggactccgacggctcttactttatgtatagcaagctgagagttgagaaga | |
| | aaaattgggtagagagaaattcttactcatgttctgttgtacatgaaggactgcacaa | |
| | tcatcacaccacaaagagtttcagtagaactccaggtaaaggtggagggggctcc | |
| | ggaggcggtgggagctttcaacctgtcctgtgtaacaaagaggttccagtgagttc | |
| | aagagagggatactgcgggccatgtccgaataactggatctgccacaggaacaa | |
| | ctgctatcagtttttcaacgaagaaaagacatggaaccagagtcaggcttcctgtttg | |
| | tctcagaactccagtctgctgaagatttactcaaaggaggaacaggacttcctcaaa | |
| | ctggtaaagagctatcattggatgggactggttcagatcccagccaatggctcttgg | |
| | cagtgggaggatggaagctctctcagctacaaccagcttaccctggtggaaattcc | |
| | caaaggaagctgcgcagtgtatggttcaagtttcaaagcctacacagaagattgcg | |
| | ctaacctgaatacatacatttgcatgaaaagggctgtcagaagtggaggcgggtct | |
| | ggaggcgggtctttccaacctgtcctgtgcaacaaagaagtgcccgtcagttccag | |
| | agagggctactgtgggccatgtcccaataattggatttgccacagaaacaactgct | |
| | accagttctttaatgaggagaaaacttggaaccagtcacaagcctcttgtctgtcac | |
| | aaaacagcagcttgctgaaaatctacagcaaggaggagcaggactttctgaagct | |
| | ggtcaaatcttatcactggatgggtctggtgcagatccccgccaatggatcttggca | |
| | atgggaggacggctcttctctctcctataatcagctgacactggtagaaatccccaa | |
| | agggagctgtgcagtgtacgggagcagttttaaggcctataccgaggattgcgca | |
| | aatttgaacacttacatctgcatgaagagggccgtc | |
| MM7, mFc2- | ISAMVRSPRGPTIKPCPPCKCPAPNLLGGPSVFIFPPKI | 59 |
| (G4S)4- | KDVLMISLSPIVTCVVVDVSEDDPDVQISWFVNNVE | |
| mNKG2D (aa94-232)- | VHTAQTQTHREDYNSTLRVVSALPIQHQDWMSGKE | |
| RS(G3S)2- | FKCKVNNKDLPAPIERTISKPKGSVRAPQVYVLPPPE | |
| mNKG2D (aa94-232) | EEMTKKQVTLTCMVTDFMPEDIYVEWTNNGKTELN | |
| protein | YKNTEPVLDSDGSYFMYSKLRVEKKNWVERNSYSC | |
| | SVVHEGLHNHHTTKSFSRTPGKGGGGSGGGGSGGG | |
| | GSGGGGSFQPVLCNKEVPVSSREGYCGPCPNNWICH | |
| | RNNCYQFFNEEKTWNQSQASCLSQNSSLLKIYSKEE | |
| | QDFLKLVKSYHWMGLVQIPANGSWQWEDGSSLSY | |
| | NQLTLVEIPKGSCAVYGSSFKAYTEDCANLNTYICM | |
| | KRAVRSGGGSGGGSFQPVLCNKEVPVSSREGYCGPC | |
| | PNNWICHRNNCYQFFNEEKTWNQSQASCLSQNSSLL | |
| | KIYSKEEQDFLKLVKSYHWMGLVQIPANGSWQWED | |
| | GSSLSYNQLTLVEIPKGSCAVYGSSFKAYTEDCANL | |
| | NTYICMKRAV | |
| MM7, mFc2- | atatcagctatggtccgctcaccaagaggaccaaccattaagccttgcccgccatg | 60 |
| (G4S)4- | caagtgccccgctccaaaccttctgggagggccgtctgtgttcatttttcctcccaaa | |
| mNKG2D (aa94-232)- | atcaaagatgtgctgatgattagcctgtctccaatcgtgacctgtgttgtcgtggacg | |
| RS(G3S)2- | tgtcagaggatgaccccgatgtgcagataagttggtttgtaaacaatgtagaggtgc | |
| mNKG2D (aa94-232) | acacggcacagacacagacccatagggaggattacaattctaccctgcgggtcgt | |
| DNA | tagcgccctgccgatccagcaccaggactggatgtcaggtaaagaatttaagtgc | |
| | aaggtgaataataaggatctgcccgctcctattgagagaacgattagcaagcccaa | |
| | gggctccgtgcgcgcaccccaagtctacgttcttcctcccccagaagaggagatg | |
| | accaaaaagcaggtcaccctgacgtgtatggtgactgattttatgcccgaagatattt | |
| | acgtggaatggacaaacaacggtaaaacagagctgaactacaagaatactgagc | |
| | ccgtgctggactccgacggctcttactttatgtatagcaagctgagagttgagaaga | |
| | aaaattgggtagagagaaattcttactcatgttctgttgtacatgaaggactgcacaa | |
| | tcatcacaccacaaagagtttcagtagaactccaggtaaaggtggagggggcagc | |
| | ggaggtgggggcagcgggggaggtgggtccggaggcggtgggagctttcaac | |
| | ctgtcctgtgtaacaaagaggttccagtgagttcaagagagggatactgcgggcc | |
| | atgtccgaataactggatctgccacaggaacaactgctatcagtttttcaacgaaga | |
| | aaagacatggaaccagagtcaggcttcctgtttgtctcagaactccagtctgctgaa | |
| | gatttactcaaaggaggaacaggacttcctcaaactggtaaagagctatcattggat | |
| | gggactggttcagatcccagccaatggctcttggcagtgggaggatggaagctct | |
| | ctcagctacaaccagcttaccctggtggaaattcccaaaggaagctgcgcagtgta | |
| | tggttcaagtttcaaagcctacacagaagattgcgctaacctgaatacatacatttgc | |
| | atgaaaagggctgtcagaagtggaggcgggtctggaggcgggtctttccaacctg | |
| | tcctgtgcaacaaagaagtgcccgtcagttccagagagggctactgtgggccatgt | |
| | cccaataattggatttgccacagaaacaactgctaccagttctttaatgaggagaaa | |
| | acttggaaccagtcacaagcctcttgtctgtcacaaaacagcagcttgctgaaaatc | |
| | tacagcaaggaggagcaggactttctgaagctggtcaaatcttatcactggatggg | |
| | tctggtgcagatccccgccaatggatcttggcaatgggaggacggctcttctctctc | |
| | ctataatcagctgacactggtagaaatccccaaagggagctgtgcagtgtacggg | |
| | agcagttttaaggcctataccgaggattgcgcaaatttgaacacttacatctgcatga | |
| | agagggccgtc | |
| MM8, mFc2- | ISAMVRSPRGPTIKPCPPCKCPAPNLLGGPSVFIFPPKI | 61 |
| (G4S)6- | KDVLMISLSPIVTCVVVDVSEDDPDVQISWFVNNVE | |
| mNKG2D (aa94-232)- | VHTAQTQTHREDYNSTLRVVSALPIQHQDWMSGKE | |
| RS(G3S)2- | FKCKVNNKDLPAPIERTISKPKGSVRAPQVYVLPPPE | |
| mNKG2D (aa94-232) | EEMTKKQVTLTCMVTDFMPEDIYVEWTNNGKTELN | |
| protein | YKNTEPVLDSDGSYFMYSKLRVEKKNWVERNSYSC | |
| | SVVHEGLHNHHTTKSFSRTPGKGGGGSGGGGSGGG | |
| | GSGGGGSGGGGSGGGGSFQPVLCNKEVPVSSREGY | |
| | CGPCPNNWICHRNNCYQFFNEEKTWNQSQASCLSQ | |
| | NSSLLKIYSKEEQDFLKLVKSYHWMGLVQIPANGS | |
| | WQWEDGSSLSYNQLTLVEIPKGSCAVYGSSFKAYTE | |
| | DCANLNTYICMKRAVRSGGGSGGGSFQPVLCNKEV | |
| | PVSSREGYCGPCPNNWICHRNNCYQFFNEEKTWNQ | |
| | SQASCLSQNSSLLKIYSKEEQDFLKLVKSYHWMGLV | |
| | QIPANGSWQWEDGSSLSYNQLTLVEIPKGSCAVYGS | |
| | SFKAYTEDCANLNTYICMKRAV | |
| MM8, mFc2- | atatcagctatggtccgctcaccaagaggaccaaccattaagccttgcccgccatg | 62 |
| (G4S)6- | caagtgccccgctccaaaccttctgggagggccgtctgtgttcatttttcctcccaaa | |
| mNKG2D (aa94-232)- | atcaaagatgtgctgatgattagcctgtctccaatcgtgacctgtgttgtcgtggacg | |
| RS(G3S)2- | tgtcagaggatgaccccgatgtgcagataagttggtttgtaaacaatgtagaggtgc | |
| mNKG2D (aa94-232) | acacggcacagacacagacccatagggaggattacaattctaccctgcgggtcgt | |
| DNA | tagcgccctgccgatccagcaccaggactggatgtcaggtaaagaatttaagtgc | |
| | aaggtgaataataaggatctgcccgctcctattgagagaacgattagcaagcccaa | |
| | gggctccgtgcgcgcaccccaagtctacgttcttcctcccccagaagaggagatg | |
| | accaaaaagcaggtcaccctgacgtgtatggtgactgattttatgcccgaagatattt | |
| | acgtggaatggacaaacaacggtaaaacagagctgaactacaagaatactgagc | |
| | ccgtgctggactccgacggctcttactttatgtatagcaagctgagagttgagaaga | |
| | aaaattgggtagagagaaattcttactcatgttctgttgtacatgaaggactgcacaa | |
| | tcatcacaccacaaagagtttcagtagaactccaggtaaaggtggagggggctcc | |
| | ggaggtgggggcagcgggggaggtgggtccggtgggggaggctctggcgga | |
| | ggtgggtctggaggcggtgggagctttcaacctgtcctgtgtaacaaagaggttcc | |
| | agtgagttcaagagagggatactgcgggccatgtccgaataactggatctgccac | |
| | aggaacaactgctatcagtttttcaacgaagaaaagacatggaaccagagtcagg | |
| | cttcctgtttgtctcagaactccagtctgctgaagatttactcaaaggaggaacagga | |
| | cttcctcaaactggtaaagagctatcattggatgggactggttcagatcccagccaa | |
| | tggctcttggcagtgggaggatggaagctctctcagctacaaccagcttaccctgg | |
| | tggaaattcccaaaggaagctgcgcagtgtatggttcaagtttcaaagcctacaca | |
| | gaagattgcgctaacctgaatacatacatttgcatgaaaagggctgtcagaagtgg | |
| | aggcgggtctggaggcgggtctttccaacctgtcctgtgcaacaaagaagtgccc | |
| | gtcagttccagagagggctactgtgggccatgtcccaataattggatttgccacag | |
| | aaacaactgctaccagttctttaatgaggagaaaacttggaaccagtcacaagcct | |
| | cttgtctgtcacaaaacagcagcttgctgaaaatctacagcaaggaggagcagga | |
| | ctttctgaagctggtcaaatcttatcactggatgggtctggtgcagatccccgccaat | |
| | ggatcttggcaatgggaggacggctcttctctctcctataatcagctgacactggta | |
| | gaaatccccaaagggagctgtgcagtgtacgggagcagttttaaggcctataccg | |
| | aggattgcgcaaatttgaacacttacatctgcatgaagagggccgtc | |
| Exemplary IL-15/ | NWVNVISDLKKIEDLIQSMHIDATLYTESDVHPSCK | 67 |
| IL-15Ra | VTAMKCFLLELQVISLESGDASIHDTVENLIILANNSL | |
| heterocomplex | SSNGNVTESGCKECEELEEKNIKEFLQSFVHIVQMFI | |
| | NTSITCPPPMSVEHADIWVKSYSLYSRERYICNSGFK | |
| | RKAGTSSLTECVLNKATNVAHWTTPSLKCIRDPALV | |
| | HQRPAPPSTVTTAGVTPQPESLSPSGKEPAASSPSSN | |
| | NTAATTAAIVPGSQLMPSKSPSTGTTEISSHESSHGTP | |
| | SQTTAKNWELTASASHQPPGVYPQG | |
| Fc of HH11 | DKTHTCPPCPAPELLGGPSVFLFPPKPKDTLMI | 68 |
| protein | SRTPEVTCVVVAVSHEDPEVKFNWYVDGVEVHNA | |
| hFc1 | KTKPREEQYASTYRVVSVLTVLHQDWLNGKEYKCK | |
| DANAPA | VSNKALAAPIEKTISKAKGQPREPQVYTLPPSRDELT | |
| | KNQVSLTCLVKGFYPSDIAVEWESNGQPENNYKTTP | |
| | PVLDSDGSFFLYSKLTVDKSRWQQGNVFSCSVMHE | |
| | ALHNHYTQKSLSLSPGK | |
| HH11, | NQEVQIPLTESYCGPCPKNWICYKNNCYQFF | 69 |
| hNKG2D(aa84-216)- | DESKNWYESQASCMSQNASLLKVYSKEDQDLLKLV | |
| RS(G3S)2- | KSYHWMGLVHIPTNGSWQWEDGSILSPNLLTIIEMQ | |
| hNKG2D(aa84-216)- | KGDCALYASSFKGYIENCSTPNTYICMQRTVRSGGG | |
| hFc1 _DANAPA | SGGGSNQEVQIPLTESYCGPCPKNWICYKNNCYQFF | |
| protein | DESKNWYESQASCMSQNASLLKVYSKEDQDLLKLV | |
| | KSYHWMGLVHIPTNGSWQWEDGSILSPNLLTIIEMQ | |
| | KGDCALYASSFKGYIENCSTPNTYICMQRTVDKTHT | |
| | CPPCPAPELLGGPSVFLFPPKPKDTLMISRTPEVTCVV | |
| | VAVSHEDPEVKFNWYVDGVEVHNAKTKPREEQYA | |
| | STYRVVSVLTVLHQDWLNGKEYKCKVSNKALAAPI | |
| | EKTISKAKGQPREPQVYTLPPSRDELTKNQVSLTCLV | |
| | KGFYPSDIAVEWESNGQPENNYKTTPPVLDSDGSFF | |
| | LYSKLTVDKSRWQQGNVFSCSVMHEALHNHYTQK | |
| | SLSLSPGK | |
| HH11, | aaccaggaagtgcagatccccctgaccgagagctactgcggcccctg | 70 |
| hNKG2D(aa84-216)- | ccccaagaactggatctgctacaagaacaactgctaccagttcttcgacgagagca | |
| RS(G3S)2- | agaattggtacgagagccaggccagctgcatgagccagaacgccagcctgctga | |
| hNKG2D(aa84-216)- | aggtgtacagcaaagaggaccaggatctgctgaagctcgtgaagtcctaccactg | |
| hFc1_DANAPA | gatgggcctggtgcacatccccaccaatggcagctggcagtgggaggacggca | |
| DNA | gcatcctgagccccaacctgctgaccatcatcgagatgcagaagggcgactgcg | |
| | ccctgtacgccagcagcttcaagggctacatcgagaactgtagcacccccaacac | |
| | ctacatctgtatgcagcggaccgtcagaagtggaggcgggtctggaggcggatc | |
| | caaccaggaagtgcagatccccctgaccgagagctactgcggcccctgccccaa | |
| | gaactggatctgctacaagaacaactgctaccagttcttcgacgagagcaagaatt | |
| | ggtacgagagccaggccagctgcatgagccagaacgccagcctgctgaaggtg | |
| | tacagcaaagaggaccaggatctgctgaagctcgtgaagtcctaccactggatgg | |
| | gcctggtgcacatccccaccaatggcagctggcagtgggaggacggcagcatcc | |
| | tgagccccaacctgctgaccatcatcgagatgcagaagggcgactgcgccctgta | |
| | cgccagcagcttcaagggctacatcgagaactgcagcacccccaacacctacatt | |
| | tgcatgcagcgtacggttgacaagacccacacctgccctccctgtccagcccctg | |
| | aactgctgggaggccctagcgtgttcctgttccccccaaagcccaaggacaccct | |
| | gatgatcagccggacccccgaagtgacctgtgtggtggtggccgtgtctcacgag | |
| | gaccctgaagtgaagtttaattggtacgtggacggcgtggaagtgcacaacgcca | |
| | agaccaagcccagagaggaacagtacgccagcacctaccgggtggtgtccgtg | |
| | ctgacagtgctgcaccaggactggctgaacggcaaagagtacaagtgcaaggtg | |
| | tccaacaaggccctggccgctcccatcgagaaaaccatcagcaaggccaaggg | |
| | ccagccccgcgaaccccaggtgtacacactgccccctagcagggacgagctga | |
| | ccaagaaccaggtgtccctgacctgcctcgtgaagggcttctacccctccgatatc | |
| | gccgtggaatgggagagcaacggccagcccgagaacaactacaagaccaccc | |
| | cccctgtgctggactccgacggctcattcttcctgtacagcaagctgaccgtggac | |
| | aagtcccggtggcagcagggcaacgtgttcagctgctccgtgatgcacgaggcc | |
| | ctgcacaaccactacacccagaagtccctgagcctgagccccggcaaa | |

### EQUIVALENTS

The disclosures of each and every patent, patent application, and publication cited herein are hereby incorporated herein by reference in their entirety. While this invention has been disclosed with reference to specific aspects, it is apparent that other aspects and variations of this invention may be devised by others skilled in the art without departing from the true spirit and scope of the invention. The appended claims are intended to be construed to include all such aspects and equivalent variations.

1. A dimeric protein comprising two monomers, wherein each monomer comprises:
(1) a first NKG2D peptide or variant thereof;
(2) a second NKG2D peptide or variant thereof;
(3) a first peptide linker connecting said first NKG2D peptide or variant thereof and said second NKG2D peptide or variant thereof; and
(4) a fragment crystallizable region (Fc region) of an immunoglobulin (Ig).

2. The dimeric protein of claim 1, wherein said first NKG2D peptide or variant thereof and said second NKG2D peptide or variant thereof have identical amino acid sequence.

3. The dimeric protein of claim 1, wherein said first NKG2D peptide or variant thereof and said second NKG2D peptide or variant thereof have different amino acid sequence.

4. The dimeric protein of claim 1, wherein said first or second NKG2D peptide or variant thereof comprises an amino acid sequence having at least 85% identity to SEQ ID NO: 1, 2, 3 or 4, optionally, wherein said first or second NKG2D peptide or variant thereof comprises an amino acid sequence of SEQ ID NO: 1, 2, 3, or 4.

5-7. (canceled)

8. The dimeric protein of claim 1, wherein said first peptide linker is a glycine-serine peptide linker, optionally, wherein said peptide linker is represented by the formula (GGGS)ₙ (SEQ ID NO: 9), wherein n is 1, 2, 3, 4, or 5.

9-10. (canceled)

11. The dimeric protein of claim 1, wherein said Fc region comprises an Fc region of human immunoglobulin G (IgG), optionally, (i) wherein said Fc region comprises an Fc region of human IgG1; (ii) wherein said Fc region comprises an amino acid sequence having at least 85% identity to SEQ ID NO: 5, 6, 63, 64, 65, or 68; and/or (iii) wherein said Fc region comprises an amino acid sequence of SEQ ID NO: 5, 6, 63, 64, 65, or 68.

12-14. (canceled)

15. The dimeric protein of claim 1, wherein said monomer comprises, from N-terminus to C-terminus, said first NKG2D peptide or variant thereof, said first peptide linker, said second NKG2D peptide or variant thereof and said Fc region, optionally, wherein (a) said second NKG2D peptide or variant thereof is directly fused with said Fc region without a peptide linker or (b) said second NKG2D peptide or variant thereof is linked with said Fc region via a second peptide linker.

16-17. (canceled)

18. The dimeric protein of claim 1, wherein said monomer comprises, from N-terminus to C-terminus, said Fc region, said first NKG2D peptide or variant thereof, said first peptide linker, and said second NKG2D peptide or variant thereof, optionally, wherein (a) said Fc region is directly fused with said first NKG2D peptide or variant thereof without a peptide linker or (b) said Fc region is linked with said first NKG2D peptide or variant thereof via a second peptide linker.

19-20. (canceled)

21. The dimeric protein of claim 1, wherein said monomer further comprising one or two additional NKG2D peptides or variants thereof.

22. The dimeric protein of claim 1, wherein said monomer comprises an amino acid sequence having at least 85% identity to any one of SEQ ID NOs: 17, 19, 21, 23, 25, 27, 29, 31, 33, 35, 37, 39, 41, 43, 45, 47, 49, 51, 53, 55, 57, 59, 61, and 69, optionally, wherein said monomer comprises an amino acid sequence of any one of SEQ ID NOs: 17, 19, 21, 23, 25, 27, 29, 31, 33, 35, 37, 39, 41, 43, 45, 47, 49, 51, 53, 55, 57, 59, 61, and 69.

23. (canceled)

24. The dimeric protein of claim 1, wherein said monomer is encoded by a nucleic acid sequence comprising or consisting of a nucleic acid sequence having at least 85% identity to any one of SEQ ID NOs: 18, 20, 22, 24, 26, 28, 30, 32, 34, 36, 38, 40, 42, 44, 46, 48, 50, 52, 54, 56, 58, 60, 62, and 70, optionally, wherein said monomer is encoded by a nucleic acid sequence comprising or consisting of any one of SEQ ID NOs: 18, 20, 22, 24, 26, 28, 30, 32, 34, 36, 38, 40, 42, 44, 46, 48, 50, 52, 54, 56, 58, 60, 62, and 70.

25. (canceled)

26. The dimeric protein of claim 1, (i) wherein said two monomers are covalently linked; (ii) wherein said two monomers are linked through a Cys-Cys bridge; and/or (iii) wherein the dimeric protein further comprises a drug moiety.

27-28. (canceled)

29. An isolated polynucleotide comprising a nucleic acid sequence comprising or consisting of any one of SEQ ID NOs: 18, 20, 22, 24, 26, 28, 30, 32, 34, 36, 38, 40, 42, 44, 46, 48, 50, 52, 54, 56, 58, 60, 62, and 70.

30. A vector comprising said isolated polynucleotide of claim 29.

31. A composition comprising a dimeric protein of claim 1; a polynucleotide encoding said dimeric protein; or a vector comprising said polynucleotide,
optionally, wherein said composition further comprises a pharmaceutically acceptable carrier.

32. (canceled)

33. A cell comprising a dimeric protein comprising two monomers, wherein each monomer comprises:
(1) a first NKG2D peptide or variant thereof;
(2) a second NKG2D peptide or variant thereof;
(3) a first peptide linker connecting said first NKG2D peptide or variant thereof and said second NKG2D peptide or variant thereof; and
(4) a fragment crystallizable region (Fc region) of an immunoglobulin (Ig);
a polynucleotide encoding said dimeric protein; a vector comprising said polynucleotide; or a composition comprising said dimeric protein, polynucleotide, or vector,
optionally, wherein said cell is a Chinese hamster ovary (CHO) cell.

34. (canceled)

35. The cell of claim 33, wherein said cell is a modified CHO cell, optionally, wherein said modified CHO cell comprises an inactivated matriptase gene or a knock-out of matriptase gene.

36. (canceled)

37. The dimeric protein by the cell of claim 33.

38. A dimeric protein of claim 1 produced by a CHO cell, optionally, a modified CHO cell, optionally, wherein said modified CHO cell comprises an inactivated matriptase gene or a knock-out of matriptase gene.

39-40. (canceled)

41. A method for treating a disease, the method comprising administering to a subject in need thereof a dimeric protein comprising two monomers, wherein each monomer comprises:
(1) a first NKG2D peptide or variant thereof;
(2) a second NKG2D peptide or variant thereof;
(3) a first peptide linker connecting said first NKG2D peptide or variant thereof and said second NKG2D peptide or variant thereof; and
(4) a fragment crystallizable region (Fc region) of an immunoglobulin (Ig),
or a composition comprising said dimeric protein, a polynucleotide encoding said dimeric protein, or vector comprising said polynucleotide,
optionally, (i) wherein said disease is an NKG2D ligand expressing disease; (ii) wherein said disease is cancer or autoimmune disease, optionally, an NKG2D ligand expressing cancer; (iii) wherein said subject has an NKG2D ligand expressing disease, optionally, cancer; and/or (iv) wherein said method further comprises treating the subject with an additional anti-cancer therapy or an anti-cancer agent, optionally, surgery, radiation therapy, gene therapy, DNA therapy, viral therapy, RNA therapy, adjuvant therapy, immunotherapy, or chemotherapy, optionally, chemotherapy that damages DNA.

42-45. (canceled)

46. The method of claim 41, wherein said NKG2D ligand expressing cancer is lung cancer, ovarian cancer, colon cancer, pancreatic cancer, bladder cancer, breast cancer, hepatocellular carcinoma (HCC), renal cancer, nasopharyngeal carcinoma (NPC), prostate cancer, melanoma, plasma cell cancer, lymphoma, glioma, neuroblastoma, or leukemia, optionally, wherein said leukemia is acute myeloid leukemia (AML), chronic myeloid leukemia (CML) or chronic lymphocytic leukemia (CLL).

47-50. (canceled)

51. The method of claim 41, wherein said NKG2D ligand is MICA, MICB, ULBP1, ULBP2, ULBP3, ULBP4, ULBP5, or ULBP6.

52. The method of claim 41, wherein said autoimmune disease is rheumatoid arthritis, colitis, celiac disease, multiple sclerosis, alopecia areata, type 1 diabetes, chronic obstructive pulmonary disease, atherosclerosis, or metabolic syndrome associated with type 2 diabetes.

53. A method for diagnosing an NKG2D ligand expressing disease, the method comprising
(1) obtaining a biological sample from a subject having an NKG2D ligand expressing disease;
(2) contacting said biological sample with a binding reagent comprising a dimeric protein comprising two monomers, wherein each monomer comprises: (i) a first NKG2D peptide or variant thereof; (ii) a second NKG2D peptide or variant thereof; (iii) a first peptide linker connecting said first NKG2D peptide or variant thereof and said second NKG2D peptide or variant thereof; and (iv) a fragment crystallizable region (Fc region) of an immunoglobulin (Ig); or a composition comprising said dimeric protein; and
(3) detecting a binding of said NKG2D ligand to said binding reagent; wherein a binding of said NKG2D ligand to said binding reagent indicates a diagnosis of said NKG2D ligand expressing disease in said subject,
optionally, wherein said NKG2D ligand is MICA, MICB, ULBP1, ULBP2, ULBP3, ULBP4, ULBP5, or ULBP6.

54-59. (canceled)


SEQUENCE LISTING (76 sequences)

SEQ ID NO: 1 - protein, 141 aa, Artificial Sequence
  source: /note=“Description of Artificial Sequence Synthetic polypeptide”
FLNSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVRS

SEQ ID NO: 2 - protein, 135 aa, Artificial Sequence
  source: /note=“Description of Artificial Sequence Synthetic polypeptide”
NQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVRS

SEQ ID NO: 3 - protein, 139 aa, Mus musculus
FQPVLCNKEVPVSSREGYCGPCPNNWICHRNNCYQFFNEEKTWNQSQASCLSQNSSLLKIYSKEEQDFLKLVKSYHWMGLVQIPANGSWQWEDGSSLSYNQLTLVEIPKGSCAVYGSSFKAYTEDCANLNTYICMKRAV

SEQ ID NO: 4 - protein, 232 aa, Mus musculus
MALIRDRKSHHSEMSKCHNYDLKPAKWDTSQEQQKQRLALTTSQPGENGIIRGRYPIEKLKISPMFVVRVLAIALAIRFTLNTLMWLAIFKETFQPVLCNKEVPVSSREGYCGPCPNNWICHRNNCYQFFNEEKTWNQSQASCLSQNSSLLKIYSKEEQDFLKLVKSYHWMGLVQIPANGSWQWEDGSSLSYNQLTLVEIPKGSCAVYGSSFKAYTEDCANLNTYICMKRAV

SEQ ID NO: 5 - protein, 227 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 227 aa, Artificial Sequence
  source: /note=“Description of Artificial Sequence Synthetic polypeptide”
DKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 24 aa, Artificial Sequence
  source: /note=“Description of Artificial Sequence Synthetic peptide”
  SITE (1)..(24): /note=“This sequence may encompass 1-12 ′Gly Ser′ repeating units”
GSGSGSGSGSGSGSGSGSGSGSGS

SEQ ID NO: 8 - protein, 25 aa, Artificial Sequence
  source: /note=“Description of Artificial Sequence Synthetic peptide”
  SITE (1)..(25): /note=“This sequence may encompass 1-5 ′Gly Gly Gly Gly Ser′ repeating units”
GGGGSGGGGSGGGGSGGGGSGGGGS

SEQ ID NO: 9 - protein, 20 aa, Artificial Sequence
  source: /note=“Description of Artificial Sequence Synthetic peptide”
  SITE (1)..(20): /note=“This sequence may encompass 1-5 ′Gly Gly Gly Ser′ repeating units”
GGGSGGGSGGGSGGGSGGGS

SEQ ID NO: 10 - protein, 8 aa, Artificial Sequence
  source: /note=“Description of Artificial Sequence Synthetic peptide”
GGGSGGGS

SEQ ID NO: 11 - protein, 5 aa, Artificial Sequence
  source: /note=“Description of Artificial Sequence Synthetic peptide”
GGGGS

SEQ ID NO: 12 - protein, 25 aa, Artificial Sequence
  source: /note=“Description of Artificial Sequence Synthetic peptide”
  SITE (1)..(25): /note=“This sequence may encompass 1-5 ′Gly Gly Gly Gly Ala′ repeating units”
GGGGAGGGGAGGGGAGGGGAGGGGA

SEQ ID NO: 13 - protein, 25 aa, Artificial Sequence
  source: /note=“Description of Artificial Sequence Synthetic peptide”
  SITE (1)..(25): /note=“This sequence may encompass 1-5 ′Gly Gly Gly Gly Pro′ repeating units”
GGGGPGGGGPGGGGPGGGGPGGGGP

SEQ ID NO: 14 - protein, 20 aa, Artificial Sequence
  source: /note=“Description of Artificial Sequence Synthetic peptide”
  SITE (1)..(20): /note=“This sequence may encompass 1-5 ′Gly Gly Gly Ala′ repeating units”
GGGAGGGAGGGAGGGAGGGA

SEQ ID NO: 15 - protein, 10 aa, Artificial Sequence
  source: /note=“Description of Artificial Sequence Synthetic peptide”
GGPLGLWAGG

SEQ ID NO: 16 - protein, 4 aa, Artificial Sequence
  source: /note=“Description of Artificial Sequence Synthetic peptide”
IEGR

SEQ ID NO: 17 - protein, 366 aa, Artificial Sequence
  source: /note=“Description of Artificial Sequence Synthetic polypeptide”
FLNSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 18 - DNA, 1098 bp, Artificial Sequence
  source: /note=“Description of Artificial Sequence Synthetic polynucleotide”
ttcctgaactccctgttcaaccaggaagtgcagatccccctgaccgagagctactgcggcccctgccccaagaactggatctgctacaagaacaactgctaccagttcttcgacgagagcaagaattggtacgagagccaggccagctgcatgagccagaacgccagcctgctgaaggtgtacagcaaagaggaccaggatctgctgaagctcgtgaagtcctaccactggatgggcctggtgcacatccccaccaatggcagctggcagtgggaggacggcagcatcctgagccccaacctgctgaccatcatcgagatgcagaagggcgactgcgccctgtacgccagcagcttcaagggctacatcgagaactgcagcacccccaacacctacatctgtatgcagcggaccgtcgacaagacccacacctgccccccctgtcctgcccctgaactgctgggcggaccctccgtgttcctgttccccccaaagcccaaggacaccctgatgatcagccggacccccgaagtgacctgcgtggtggtggacgtgtcccacgaggaccctgaagtgaagttcaattggtacgtggacggcgtggaagtgcacaacgccaagaccaagcccagagaggaacagtacaacagcacctaccgggtggtgtccgtgctgaccgtgctgcaccaggactggctgaacggcaaagagtacaagtgcaaagtctccaacaaggccctgcctgcccccatcgagaaaaccatcagcaaggccaagggccagccccgcgagccccaggtgtacacactgccccccagccgggacgagctgaccaagaaccaggtgtccctgacctgcctggtcaagggcttctaccccagcgatatcgccgtggaatgggagagcaacggccagcccgagaacaactacaagaccaccccccctgtgctggacagcgacggctcattcttcctgtacagcaagctgaccgtggacaagtcccggtggcagcagggcaacgtgttcagctgcagcgtgatgcacgaggccctgcacaaccactacacccagaagtccctgagcctgagccccggcaaa

SEQ ID NO: 19 - protein, 515 aa, Artificial Sequence
  source: /note=“Description of Artificial Sequence Synthetic polypeptide”
FLNSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVRSGGGSGGGSFLNSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 20 - DNA, 1545 bp, Artificial Sequence
  source: /note=“Description of Artificial Sequence Synthetic polynucleotide”
ttcctgaactccctgttcaaccaggaagtgcagatccccctgaccgagagctactgcggcccctgccccaagaactggatctgctacaagaacaactgctaccagttcttcgacgagagcaagaattggtacgagagccaggccagctgcatgagccagaacgccagcctgctgaaggtgtacagcaaagaggaccaggatctgctgaagctcgtgaagtcctaccactggatgggcctggtgcacatccccaccaatggcagctggcagtgggaggacggcagcatcctgagccccaacctgctgaccatcatcgagatgcagaagggcgactgcgccctgtacgccagcagcttcaagggctacatcgagaactgcagcacccccaacacctacatctgtatgcagcggaccgtcagaagtggaggcgggtctggaggcggatcctttctgaatagcctgtttaaccaggaagtgcagatccccctgaccgagagctactgcggcccctgccccaagaactggatctgctacaagaacaactgctaccagttcttcgacgagagcaagaattggtacgagagccaggccagctgcatgagccagaacgccagcctgctgaaggtgtacagcaaagaggaccaggatctgctgaagctcgtgaagtcctaccactggatgggcctggtgcacatccccaccaatggcagctggcagtgggaggacggcagcatcctgagccccaacctgctgaccatcatcgagatgcagaagggcgactgcgccctgtacgccagcagcttcaagggctacatcgagaactgcagcacccccaacacctacatttgcatgcagcgtacggttgacaagacccacacctgccccccctgtcctgcccctgaactgctgggcggaccctccgtgttcctgttccccccaaagcccaaggacaccctgatgatcagccggacccccgaagtgacctgcgtggtggtggacgtgtcccacgaggaccctgaagtgaagttcaattggtacgtggacggcgtggaagtgcacaacgccaagaccaagcccagagaggaacagtacaacagcacctaccgggtggtgtccgtgctgaccgtgctgcaccaggactggctgaacggcaaagagtacaagtgcaaagtctccaacaaggccctgcctgcccccatcgagaaaaccatcagcaaggccaagggccagccccgcgagccccaggtgtacacactgccccccagccgggacgagctgaccaagaaccaggtgtccctgacctgcctggtcaagggcttctaccccagcgatatcgccgtggaatgggagagcaacggccagcccgagaacaactacaagaccaccccccctgtgctggacagcgacggctcattcttcctgtacagcaagctgaccgtggacaagtcccggtggcagcagggcaacgtgttcagctgcagcgtgatgcacgaggccctgcacaaccactacacccagaagtccctgagcctgagccccggcaaa

SEQ ID NO: 21 - protein, 365 aa, Artificial Sequence
  source: /note=“Description of Artificial Sequence Synthetic polypeptide”
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGFLNSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTV

SEQ ID NO: 22 - DNA, 1095 bp, Artificial Sequence
  source: /note=“Description of Artificial Sequence Synthetic polynucleotide”
gacaagacccacacctgtcctccctgtcctgcccctgaactgctgggcggacctagcgtgttcctgttccccccaaagcccaaggacaccctgatgatcagccggacccccgaagtgacctgcgtggtggtggatgtgtcccacgaggaccctgaagtgaagttcaattggtacgtggacggcgtggaagtgcacaacgccaagaccaagcccagagaggaacagtacaacagcacctaccgggtggtgtccgtgctgaccgtgctgcaccaggactggctgaacggcaaagagtacaagtgcaaggtgtccaacaaggccctgccagcccccatcgagaaaaccatcagcaaggccaagggccagccccgcgaaccccaggtgtacacactgccccctagccgggaagagatgaccaagaaccaggtgtccctgacctgtctcgtgaagggcttctacccctccgatatcgccgtggaatgggagagcaacggccagcccgagaacaactacaagaccaccccccctgtgctggacagcgacggctcattcttcctgtacagcaagctgacagtggacaagagccggtggcagcagggcaacgtgttcagctgcagcgtgatgcacgaggccctgcacaaccactacacccagaagagcttaagcctgagccctggcttcctgaactccctgttcaaccaggaagtgcagatccccctgaccgagagctactgcggcccctgccccaagaactggatctgctacaagaacaactgctaccagttcttcgacgagagcaagaattggtacgagagccaggccagctgcatgagccagaacgccagcctgctgaaggtgtacagcaaagaggaccaggatctgctgaagctcgtgaagtcctaccactggatgggcctggtgcacatccccaccaatggcagctggcagtgggaggacggcagcatcctgagccccaacctgctgaccatcatcgagatgcagaagggcgactgcgccctgtacgccagcagcttcaagggctacatcgagaactgcagcacccccaacacctacatctgtatgcagcggaccgtc

SEQ ID NO: 23 - protein, 514 aa, Artificial Sequence
  source: /note=“Description of Artificial Sequence Synthetic polypeptide”
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGFLNSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVRSGGGSGGGSFLNSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTV

SEQ ID NO: 24 - DNA, 1542 bp, Artificial Sequence
  source: /note=“Description of Artificial Sequence Synthetic polynucleotide”
gacaagacccacacctgtcctccctgtcctgcccctgaactgctgggcggacctagcgtgttcctgttccccccaaagcccaaggacaccctgatgatcagccggacccccgaagtgacctgcgtggtggtggatgtgtcccacgaggaccctgaagtgaagttcaattggtacgtggacggcgtggaagtgcacaacgccaagaccaagcccagagaggaacagtacaacagcacctaccgggtggtgtccgtgctgaccgtgctgcaccaggactggctgaacggcaaagagtacaagtgcaaggtgtccaacaaggccctgccagcccccatcgagaaaaccatcagcaaggccaagggccagccccgcgaaccccaggtgtacacactgccccctagccgggaagagatgaccaagaaccaggtgtccctgacctgtctcgtgaagggcttctacccctccgatatcgccgtggaatgggagagcaacggccagcccgagaacaactacaagaccaccccccctgtgctggacagcgacggctcattcttcctgtacagcaagctgacagtggacaagagccggtggcagcagggcaacgtgttcagctgcagcgtgatgcacgaggccctgcacaaccactacacccagaagagcttaagcctgagccctggcttcctgaactccctgttcaaccaggaagtgcagatccccctgaccgagagctactgcggcccctgccccaagaactggatctgctacaagaacaactgctaccagttcttcgacgagagcaagaattggtacgagagccaggccagctgcatgagccagaacgccagcctgctgaaggtgtacagcaaagaggaccaggatctgctgaagctcgtgaagtcctaccactggatgggcctggtgcacatccccaccaatggcagctggcagtgggaggacggcagcatcctgagccccaacctgctgaccatcatcgagatgcagaagggcgactgcgccctgtacgccagcagcttcaagggctacatcgagaactgcagcacccccaacacctacatctgtatgcagcggaccgtcagaagtggaggcgggtctggaggcggatcctttctgaatagcctgtttaaccaggaagtgcagatccccctgaccgagagctactgcggcccctgccccaagaactggatctgctacaagaacaactgctaccagttcttcgacgagagcaagaattggtacgagagccaggccagctgcatgagccagaacgccagcctgctgaaggtgtacagcaaagaggaccaggatctgctgaagctcgtgaagtcctaccactggatgggcctggtgcacatccccaccaatggcagctggcagtgggaggacggcagcatcctgagccccaacctgctgaccatcatcgagatgcagaagggcgactgcgccctgtacgccagcagcttcaagggctacatcgagaactgcagcacccccaacacctacatttgcatgcagcgtacggtt

SEQ ID NO: 25 - protein, 515 aa, Artificial Sequence
  source: /note=“Description of Artificial Sequence Synthetic polypeptide”
FLNSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVRSGGGSGGGSFLNSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 26 - DNA, 1545 bp, Artificial Sequence
  source: /note=“Description of Artificial Sequence Synthetic polynucleotide”
ttcctgaactccctgttcaaccaggaagtgcagatccccctgaccgagagctactgcggcccctgccccaagaactggatctgctacaagaacaactgctaccagttcttcgacgagagcaagaattggtacgagagccaggccagctgcatgagccagaacgccagcctgctgaaggtgtacagcaaagaggaccaggatctgctgaagctcgtgaagtcctaccactggatgggcctggtgcacatccccaccaatggcagctggcagtgggaggacggcagcatcctgagccccaacctgctgaccatcatcgagatgcagaagggcgactgcgccctgtacgccagcagcttcaagggctacatcgagaactgcagcacccccaacacctacatctgtatgcagcggaccgtcagaagtggaggcgggtctggaggcggatcctttctgaatagcctgtttaaccaggaagtgcagatccccctgaccgagagctactgcggcccctgccccaagaactggatctgctacaagaacaactgctaccagttcttcgacgagagcaagaattggtacgagagccaggccagctgcatgagccagaacgccagcctgctgaaggtgtacagcaaagaggaccaggatctgctgaagctcgtgaagtcctaccactggatgggcctggtgcacatccccaccaatggcagctggcagtgggaggacggcagcatcctgagccccaacctgctgaccatcatcgagatgcagaagggcgactgcgccctgtacgccagcagcttcaagggctacatcgagaactgcagcacccccaacacctacatttgcatgcagcgtacggttgacaagacccacacctgccccccctgtcctgcccctgaactgctgggcggacccgacgtgttcctgttccccccaaagcccaaggacaccctgatgatcagccggacccccgaagtgacctgcgtggtggtggacgtgtcccacgaggaccctgaagtgaagttcaattggtacgtggacggcgtggaagtgcacaacgccaagaccaagcccagagaggaacagtacaacagcacctaccgggtggtgtccgtgctgaccgtgctgcaccaggactggctgaacggcaaagagtacaagtgcaaagtctccaacaaggccctgcctgcccccgaagagaaaaccatcagcaaggccaagggccagccccgcgagccccaggtgtacacactgccccccagccgggacgagctgaccaagaaccaggtgtccctgacctgcctggtcaagggcttctaccccagcgatatcgccgtggaatgggagagcaacggccagcccgagaacaactacaagaccaccccccctgtgctggacagcgacggctcattcttcctgtacagcaagctgaccgtggacaagtcccggtggcagcagggcaacgtgttcagctgcagcgtgatgcacgaggccctgcacaaccactacacccagaagtccctgagcctgagccccggcaaa

SEQ ID NO: 27 - protein, 509 aa, Artificial Sequence
  source: /note=“Description of Artificial Sequence Synthetic polypeptide”
FLNSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVRSGGGSGGGSNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 28 - DNA, 1527 bp, Artificial Sequence
  source: /note=“Description of Artificial Sequence Synthetic polynucleotide”
ttcctgaactccctgttcaaccaggaagtgcagatccccctgaccgagagctactgcggcccctgccccaagaactggatctgctacaagaacaactgctaccagttcttcgacgagagcaagaattggtacgagagccaggccagctgcatgagccagaacgccagcctgctgaaggtgtacagcaaagaggaccaggatctgctgaagctcgtgaagtcctaccactggatgggcctggtgcacatccccaccaatggcagctggcagtgggaggacggcagcatcctgagccccaacctgctgaccatcatcgagatgcagaagggcgactgcgccctgtacgccagcagcttcaagggctacatcgagaactgcagcacccccaacacctacatctgtatgcagcggaccgtcagaagtggaggcgggtctggaggcggatccaaccaggaagtgcagatccccctgaccgagagctactgcggcccctgccccaagaactggatctgctacaagaacaactgctaccagttcttcgacgagagcaagaattggtacgagagccaggccagctgcatgagccagaacgccagcctgctgaaggtgtacagcaaagaggaccaggatctgctgaagctcgtgaagtcctaccactggatgggcctggtgcacatccccaccaatggcagctggcagtgggaggacggcagcatcctgagccccaacctgctgaccatcatcgagatgcagaagggcgactgcgccctgtacgccagcagcttcaagggctacatcgagaactgcagcacccccaacacctacatttgcatgcagcgtacggttgacaagacccacacctgccccccctgtcctgcccctgaactgctgggcggaccctccgtgttcctgttccccccaaagcccaaggacaccctgatgatcagccggacccccgaagtgacctgcgtggtggtggacgtgtcccacgaggaccctgaagtgaagttcaattggtacgtggacggcgtggaagtgcacaacgccaagaccaagcccagagaggaacagtacaacagcacctaccgggtggtgtccgtgctgaccgtgctgcaccaggactggctgaacggcaaagagtacaagtgcaaagtctccaacaaggccctgcctgcccccatcgagaaaaccatcagcaaggccaagggccagccccgcgagccccaggtgtacacactgccccccagccgggacgagctgaccaagaaccaggtgtccctgacctgcctggtcaagggcttctaccccagcgatatcgccgtggaatgggagagcaacggccagcccgagaacaactacaagaccaccccccctgtgctggacagcgacggctcattcttcctgtacagcaagctgaccgtggacaagtcccggtggcagcagggcaacgtgttcagctgcagcgtgatgcacgaggccctgcacaaccactacacccagaagtccctgagcctgagccccggcaaa

SEQ ID NO: 29 - protein, 503 aa, Artificial Sequence
  source: /note=“Description of Artificial Sequence Synthetic polypeptide”
NQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVRSGGGSGGGSNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 30 - DNA, 1509 bp, Artificial Sequence
  source: /note=“Description of Artificial Sequence Synthetic polynucleotide”
aaccaggaagtgcagatccccctgaccgagagctactgcggcccctgccccaagaactggatctgctacaagaacaactgctaccagttcttcgacgagagcaagaattggtacgagagccaggccagctgcatgagccagaacgccagcctgctgaaggtgtacagcaaagaggaccaggatctgctgaagctcgtgaagtcctaccactggatgggcctggtgcacatccccaccaatggcagctggcagtgggaggacggcagcatcctgagccccaacctgctgaccatcatcgagatgcagaagggcgactgcgccctgtacgccagcagcttcaagggctacatcgagaactgtagcacccccaacacctacatctgtatgcagcggaccgtcagaagtggaggcgggtctggaggcggatccaaccaggaagtgcagatccccctgaccgagagctactgcggcccctgccccaagaactggatctgctacaagaacaactgctaccagttcttcgacgagagcaagaattggtacgagagccaggccagctgcatgagccagaacgccagcctgctgaaggtgtacagcaaagaggaccaggatctgctgaagctcgtgaagtcctaccactggatgggcctggtgcacatccccaccaatggcagctggcagtgggaggacggcagcatcctgagccccaacctgctgaccatcatcgagatgcagaagggcgactgcgccctgtacgccagcagcttcaagggctacatcgagaactgcagcacccccaacacctacatttgcatgcagcgtacggttgacaagacccacacctgccccccctgtcctgcccctgaactgctgggcggaccctccgtgttcctgttccccccaaagcccaaggacaccctgatgatcagccggacccccgaagtgacctgcgtggtggtggacgtgtcccacgaggaccctgaagtgaagttcaattggtacgtggacggcgtggaagtgcacaacgccaagaccaagcccagagaggaacagtacaacagcacctaccgggtggtgtccgtgctgaccgtgctgcaccaggactggctgaacggcaaagagtacaagtgcaaagtctccaacaaggccctgcctgcccccatcgagaaaaccatcagcaaggccaagggccagccccgcgagccccaggtgtacacactgccccccagccgggacgagctgaccaagaaccaggtgtccctgacctgcctggtcaagggcttctaccccagcgatatcgccgtggaatgggagagcaacggccagcccgagaacaactacaagaccaccccccctgtgctggacagcgacggctcattcttcctgtacagcaagctgaccgtggacaagtcccggtggcagcagggcaacgtgttcagctgcagcgtgatgcacgaggccctgcacaaccactacacccagaagtccctgagcctgagccccggcaaa

SEQ ID NO: 31 - protein, 503 aa, Artificial Sequence
  source: /note=“Description of Artificial Sequence Synthetic polypeptide”
NQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVRSGGGSGGGSNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 32 - DNA, 1509 bp, Artificial Sequence
  source: /note=“Description of Artificial Sequence Synthetic polynucleotide”
aaccaggaagtgcagatccccctgaccgagagctactgcggcccctgccccaagaactggatctgctacaagaacaactgctaccagttcttcgacgagagcaagaattggtacgagagccaggccagctgcatgagccagaacgccagcctgctgaaggtgtacagcaaagaggaccaggatctgctgaagctcgtgaagtcctaccactggatgggcctggtgcacatccccaccaatggcagctggcagtgggaggacggcagcatcctgagccccaacctgctgaccatcatcgagatgcagaagggcgactgcgccctgtacgccagcagcttcaagggctacatcgagaactgcagcacccccaacacctacatctgtatgcagcggaccgtcagaagtggaggcgggtctggaggcggatccaaccaggaagtgcagatccccctgaccgagagctactgcggcccctgccccaagaactggatctgctacaagaacaactgctaccagttcttcgacgagagcaagaattggtacgagagccaggccagctgcatgagccagaacgccagcctgctgaaggtgtacagcaaagaggaccaggatctgctgaagctcgtgaagtcctaccactggatgggcctggtgcacatccccaccaatggcagctggcagtgggaggacggcagcatcctgagccccaacctgctgaccatcatcgagatgcagaagggcgactgcgccctgtacgccagcagcttcaagggctacatcgagaactgcagcacccccaacacctacatttgcatgcagcgtacggttgacaagacccacacctgccccccctgtcctgcccctgaactgctgggcggacccgacgtgttcctgttccccccaaagcccaaggacaccctgatgatcagccggacccccgaagtgacctgcgtggtggtggacgtgtcccacgaggaccctgaagtgaagttcaattggtacgtggacggcgtggaagtgcacaacgccaagaccaagcccagagaggaacagtacaacagcacctaccgggtggtgtccgtgctgaccgtgctgcaccaggactggctgaacggcaaagagtacaagtgcaaagtctccaacaaggccctgcctgcccccgaagagaaaaccatcagcaaggccaagggccagccccgcgagccccaggtgtacacactgccccccagccgggacgagctgaccaagaaccaggtgtccctgacctgcctggtcaagggcttctaccccagcgatatcgccgtggaatgggagagcaacggccagcccgagaacaactacaagaccaccccccctgtgctggacagcgacggctcattcttcctgtacagcaagctgaccgtggacaagtcccggtggcagcagggcaacgtgttcagctgcagcgtgatgcacgaggccctgcacaaccactacacccagaagtccctgagcctgagccccggcaaa

SEQ ID NO: 33 - protein, 606 aa, Artificial Sequence
  source: /note=“Description of Artificial Sequence Synthetic polypeptide”
NQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVRSGGGSGGGSNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 34 - DNA, 1818 bp, Artificial Sequence
  source: /note=“Description of Artificial Sequence Synthetic polynucleotide”
aaccaggaagtgcagatccccctgaccgagagctactgcggcccctgccccaagaactggatctgctacaagaacaactgctaccagttcttcgacgagagcaagaattggtacgagagccaggccagctgcatgagccagaacgccagcctgctgaaggtgtacagcaaagaggaccaggatctgctgaagctcgtgaagtcctaccactggatgggcctggtgcacatccccaccaatggcagctggcagtgggaggacggcagcatcctgagccccaacctgctgaccatcatcgagatgcagaagggcgactgcgccctgtacgccagcagcttcaagggctacatcgagaactgtagcacccccaacacctacatctgtatgcagcggaccgtcagaagtggaggcgggtctggaggcggatccaaccaggaagtgcagatccccctgaccgagagctactgcggcccctgccccaagaactggatctgctacaagaacaactgctaccagttcttcgacgagagcaagaattggtacgagagccaggccagctgcatgagccagaacgccagcctgctgaaggtgtacagcaaagaggaccaggatctgctgaagctcgtgaagtcctaccactggatgggcctggtgcacatccccaccaatggcagctggcagtgggaggacggcagcatcctgagccccaacctgctgaccatcatcgagatgcagaagggcgactgcgccctgtacgccagcagcttcaagggctacatcgagaactgcagcacccccaacacctacatttgcatgcagcgtacggttgctagcaccaagggccccagcgtgttccccctggcgcccagcagcaagagcaccagcggcggcacagccgccctgggctgcctggtgaaggactacttccccgagccagtgaccgtgtcctggaacagcggagccctgacctccggcgtgcacaccttccccgccgtgctgcagagcagcggcctgtacagcctgagcagcgtggtgaccgtgcccagcagcagcctgggcacccagacctacatctgcaacgtgaaccacaagcccagcaacaccaaggtggacaagagagtggagcccaagagctgcgacaagacccacacctgccccccctgtcctgcccctgaactgctgggcggaccctccgtgttcctgttccccccaaagcccaaggacaccctgatgatcagccggacccccgaagtgacctgcgtggtggtggacgtgtcccacgaggaccctgaagtgaagttcaattggtacgtggacggcgtggaagtgcacaacgccaagaccaagcccagagaggaacagtacaacagcacctaccgggtggtgtccgtgctgaccgtgctgcaccaggactggctgaacggcaaagagtacaagtgcaaagtctccaacaaggccctgcctgcccccatcgagaaaaccatcagcaaggccaagggccagccccgcgagccccaggtgtacacactgccccccagccgggacgagctgaccaagaaccaggtgtccctgacctgcctggtcaagggcttctaccccagcgatatcgccgtggaatgggagagcaacggccagcccgagaacaactacaagaccaccccccctgtgctggacagcgacggctcattcttcctgtacagcaagctgaccgtggacaagtcccggtggcagcagggcaacgtgttcagctgcagcgtgatgcacgaggccctgcacaaccactacacccagaagtccctgagcctgagccccggcaaa

SEQ ID NO: 35 - protein, 383 aa, Artificial Sequence
  source: /note=“Description of Artificial Sequence Synthetic polypeptide”
NQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVRSGGGSGGGSNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 36 - DNA, 1149 bp, Artificial Sequence
  source: /note=“Description of Artificial Sequence Synthetic polynucleotide”
aaccaggaagtgcagatccccctgaccgagagctactgcggcccctgccccaagaactggatctgctacaagaacaactgctaccagttcttcgacgagagcaagaattggtacgagagccaggccagctgcatgagccagaacgccagcctgctgaaggtgtacagcaaagaggaccaggatctgctgaagctcgtgaagtcctaccactggatgggcctggtgcacatccccaccaatggcagctggcagtgggaggacggcagcatcctgagccccaacctgctgaccatcatcgagatgcagaagggcgactgcgccctgtacgccagcagcttcaagggctacatcgagaactgtagcacccccaacacctacatctgtatgcagcggaccgtcagaagtggaggcgggtctggaggcggatccaaccaggaagtgcagatccccctgaccgagagctactgcggcccctgccccaagaactggatctgctacaagaacaactgctaccagttcttcgacgagagcaagaattggtacgagagccaggccagctgcatgagccagaacgccagcctgctgaaggtgtacagcaaagaggaccaggatctgctgaagctcgtgaagtcctaccactggatgggcctggtgcacatccccaccaatggcagctggcagtgggaggacggcagcatcctgagccccaacctgctgaccatcatcgagatgcagaagggcgactgcgccctgtacgccagcagcttcaagggctacatcgagaactgcagcacccccaacacctacatttgcatgcagcgtacggttcggacagtggccgctcctagcgtgttcatctttccaccaagcgacgagcagctgaagtctggcacagcctctgtcgtgtgcctgctgaacaacttctaccccagagaagccaaggtgcagtggaaggtggacaatgccctgcagagcggcaatagccaagagagcgtgaccgagcaggacagcaaggatagcacatacagcctgagcagcaccctgactctgagcaaggccgactacgagaagcacaaagtgtacgcctgcgaagtgacacaccagggcctgtctagccctgtgaccaagagcttcaacagaggcgagtgc

SEQ ID NO: 37 - protein, 463 aa, Artificial Sequence
  source: /note=“Description of Artificial Sequence Synthetic polypeptide”
NQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 38 - DNA, 1389 bp, Artificial Sequence
  source: /note=“Description of Artificial Sequence Synthetic polynucleotide”
aaccaggaagtgcagatccccctgaccgagagctactgcggcccctgccccaagaactggatctgctacaagaacaactgctaccagttcttcgacgagagcaagaattggtacgagagccaggccagctgcatgagccagaacgccagcctgctgaaggtgtacagcaaagaggaccaggatctgctgaagctcgtgaagtcctaccactggatgggcctggtgcacatccccaccaatggcagctggcagtgggaggacggcagcatcctgagccccaacctgctgaccatcatcgagatgcagaagggcgactgcgccctgtacgccagcagcttcaagggctacatcgagaactgcagcacccccaacacctacatttgcatgcagcgtacggttgctagcaccaagggccccagcgtgttccccctggcgcccagcagcaagagcaccagcggcggcacagccgccctgggctgcctggtgaaggactacttccccgagccagtgaccgtgtcctggaacagcggagccctgacctccggcgtgcacaccttccccgccgtgctgcagagcagcggcctgtacagcctgagcagcgtggtgaccgtgcccagcagcagcctgggcacccagacctacatctgcaacgtgaaccacaagcccagcaacaccaaggtggacaagagagtggagcccaagagctgcgacaagacccacacctgccccccctgtcctgcccctgaactgctgggcggaccctccgtgttcctgttccccccaaagcccaaggacaccctgatgatcagccggacccccgaagtgacctgcgtggtggtggacgtgtcccacgaggaccctgaagtgaagttcaattggtacgtggacggcgtggaagtgcacaacgccaagaccaagcccagagaggaacagtacaacagcacctaccgggtggtgtccgtgctgaccgtgctgcaccaggactggctgaacggcaaagagtacaagtgcaaagtctccaacaaggccctgcctgcccccatcgagaaaaccatcagcaaggccaagggccagccccgcgagccccaggtgtacacactgccccccagccgggacgagctgaccaagaaccaggtgtccctgacctgcctggtcaagggcttctaccccagcgatatcgccgtggaatgggagagcaacggccagcccgagaacaactacaagaccaccccccctgtgctggacagcgacggctcattcttcctgtacagcaagctgaccgtggacaagtcccggtggcagcagggcaacgtgttcagctgcagcgtgatgcacgaggccctgcacaaccactacacccagaagtccctgagcctgagccccggcaaa

SEQ ID NO: 39 - protein, 240 aa, Artificial Sequence
  source: /note=“Description of Artificial Sequence Synthetic polypeptide”
NQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 40 - DNA, 720 bp, Artificial Sequence
  source: /note=“Description of Artificial Sequence Synthetic polynucleotide”
aaccaggaagtgcagatccccctgaccgagagctactgcggcccctgccccaagaactggatctgctacaagaacaactgctaccagttcttcgacgagagcaagaattggtacgagagccaggccagctgcatgagccagaacgccagcctgctgaaggtgtacagcaaagaggaccaggatctgctgaagctcgtgaagtcctaccactggatgggcctggtgcacatccccaccaatggcagctggcagtgggaggacggcagcatcctgagccccaacctgctgaccatcatcgagatgcagaagggcgactgcgccctgtacgccagcagcttcaagggctacatcgagaactgcagcacccccaacacctacatttgcatgcagcgtacggttcggacagtggccgctcctagcgtgttcatctttccaccaagcgacgagcagctgaagtctggcacagcctctgtcgtgtgcctgctgaacaacttctaccccagagaagccaaggtgcagtggaaggtggacaatgccctgcagagcggcaatagccaagagagcgtgaccgagcaggacagcaaggatagcacatacagcctgagcagcaccctgactctgagcaaggccgactacgagaagcacaaagtgtacgcctgcgaagtgacacaccagggcctgtctagccctgtgaccaagagcttcaacagaggcgagtgc

SEQ ID NO: 41 - protein, 360 aa, Artificial Sequence
  source: /note=“Description of Artificial Sequence Synthetic polypeptide”
NQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 42 - DNA, 1080 bp, Artificial Sequence
  source: /note=“Description of Artificial Sequence Synthetic polynucleotide”
aaccaggaagtgcagatccccctgaccgagagctactgcggcccctgccccaagaactggatctgctacaagaacaactgctaccagttcttcgacgagagcaagaattggtacgagagccaggccagctgcatgagccagaacgccagcctgctgaaggtgtacagcaaagaggaccaggatctgctgaagctcgtgaagtcctaccactggatgggcctggtgcacatccccaccaatggcagctggcagtgggaggacggcagcatcctgagccccaacctgctgaccatcatcgagatgcagaagggcgactgcgccctgtacgccagcagcttcaagggctacatcgagaactgcagcacccccaacacctacatctgtatgcagcggaccgtcgacaagacccacacctgccccccctgtcctgcccctgaactgctgggcggaccctccgtgttcctgttccccccaaagcccaaggacaccctgatgatcagccggacccccgaagtgacctgcgtggtggtggacgtgtcccacgaggaccctgaagtgaagttcaattggtacgtggacggcgtggaagtgcacaacgccaagaccaagcccagagaggaacagtacaacagcacctaccgggtggtgtccgtgctgaccgtgctgcaccaggactggctgaacggcaaagagtacaagtgcaaagtctccaacaaggccctgcctgcccccatcgagaaaaccatcagcaaggccaagggccagccccgcgagccccaggtgtacacactgccccccagccgggacgagctgaccaagaaccaggtgtccctgacctgcctggtcaagggcttctaccccagcgatatcgccgtggaatgggagagcaacggccagcccgagaacaactacaagaccaccccccctgtgctggacagcgacggctcattcttcctgtacagcaagctgaccgtggacaagtcccggtggcagcagggcaacgtgttcagctgcagcgtgatgcacgaggccctgcacaaccactacacccagaagtccctgagcctgagccccggcaaa

SEQ ID NO: 43 - protein, 779 aa, Artificial Sequence
  source: /note=“Description of Artificial Sequence Synthetic polypeptide”
NQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVRSGGGSGGGSNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVRSGGGSGGGSNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTV

SEQ ID NO: 44 - DNA, 2337 bp, Artificial Sequence
  source: /note=“Description of Artificial Sequence Synthetic polynucleotide”
aaccaggaagtgcagatccccctgaccgagagctactgcggcccctgccccaagaactggatctgctacaagaacaactgctaccagttcttcgacgagagcaagaattggtacgagagccaggccagctgcatgagccagaacgccagcctgctgaaggtgtacagcaaagaggaccaggatctgctgaagctcgtgaagtcctaccactggatgggcctggtgcacatccccaccaatggcagctggcagtgggaggacggcagcatcctgagccccaacctgctgaccatcatcgagatgcagaagggcgactgcgccctgtacgccagcagcttcaagggctacatcgagaactgtagcacccccaacacctacatctgtatgcagcggaccgtcagaagtggaggcgggtctggaggcggatccaaccaggaagtgcagatccccctgaccgagagctactgcggcccctgccccaagaactggatctgctacaagaacaactgctaccagttcttcgacgagagcaagaattggtacgagagccaggccagctgcatgagccagaacgccagcctgctgaaggtgtacagcaaagaggaccaggatctgctgaagctcgtgaagtcctaccactggatgggcctggtgcacatccccaccaatggcagctggcagtgggaggacggcagcatcctgagccccaacctgctgaccatcatcgagatgcagaagggcgactgcgccctgtacgccagcagcttcaagggctacatcgagaactgcagcacccccaacacctacatttgcatgcagcgtacggttgacaagacccacacctgccccccctgtcctgcccctgaactgctgggcggaccctccgtgttcctgttccccccaaagcccaaggacaccctgatgatcagccggacccccgaagtgacctgcgtggtggtggacgtgtcccacgaggaccctgaagtgaagttcaattggtacgtggacggcgtggaagtgcacaacgccaagaccaagcccagagaggaacagtacaacagcacctaccgggtggtgtccgtgctgaccgtgctgcaccaggactggctgaacggcaaagagtacaagtgcaaagtctccaacaaggccctgcctgcccccatcgagaaaaccatcagcaaggccaagggccagccccgcgagccccaggtgtacacactgccccccagccgggacgagctgaccaagaaccaggtgtccctgacctgcctggtcaagggcttctaccccagcgatatcgccgtggaatgggagagcaacggccagcccgagaacaactacaagaccaccccccctgtgctggacagcgacggctcattcttcctgtacagcaagctgaccgtggacaagtcccggtggcagcagggcaacgtgttcagctgcagcgtgatgcacgaggccctgcacaaccactacacccagaagtccctgagcctgagccccggcaaaaaccaggaagtgcagatccccctgaccgagagctactgcggcccctgccccaagaactggatctgctacaagaacaactgctaccagttcttcgacgagagcaagaattggtacgagagccaggccagctgcatgagccagaacgccagcctgctgaaggtgtacagcaaagaggaccaggatctgctgaagctcgtgaagtcctaccactggatgggcctggtgcacatccccaccaatggcagctggcagtgggaggacggcagcatcctgagccccaacctgctgaccatcatcgagatgcagaagggcgactgcgccctgtacgccagcagcttcaagggctacatcgagaactgtagcacccccaacacctacatctgtatgcagcggaccgtcagaagtggaggcgggtctggaggcggatccaaccaggaagtgcagatccccctgaccgagagctactgcggcccctgccccaagaactggatctgctacaagaacaactgctaccagttcttcgacgagagcaagaattggtacgagagccaggccagctgcatgagccagaacgccagcctgctgaaggtgtacagcaaagaggaccaggatctgctgaagctcgtgaagtcctaccactggatgggcctggtgcacatccccaccaatggcagctggcagtgggaggacggcagcatcctgagccccaacctgctgaccatcatcgagatgcagaagggcgactgcgccctgtacgccagcagcttcaagggctacatcgagaactgcagcacccccaacacctacatttgcatgcagcgtacggtt

SEQ ID NO: 45 - protein, 784 aa, Artificial Sequence
  source: /note=“Description of Artificial Sequence Synthetic polypeptide”
NQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVRSGGGSGGGSNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVRSGGGNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVRSGGGSGGGSNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 46 - DNA, 2352 bp, Artificial Sequence
  source: /note=“Description of Artificial Sequence Synthetic polynucleotide”
aaccaggaagtgcagatccccctgaccgagagctactgcggcccctgccccaagaactggatctgctacaagaacaactgctaccagttcttcgacgagagcaagaattggtacgagagccaggccagctgcatgagccagaacgccagcctgctgaaggtgtacagcaaagaggaccaggatctgctgaagctcgtgaagtcctaccactggatgggcctggtgcacatccccaccaatggcagctggcagtgggaggacggcagcatcctgagccccaacctgctgaccatcatcgagatgcagaagggcgactgcgccctgtacgccagcagcttcaagggctacatcgagaactgtagcacccccaacacctacatctgtatgcagcggaccgtcagaagtggaggcgggtctggaggcggatccaaccaggaagtgcagatccccctgaccgagagctactgcggcccctgccccaagaactggatctgctacaagaacaactgctaccagttcttcgacgagagcaagaattggtacgagagccaggccagctgcatgagccagaacgccagcctgctgaaggtgtacagcaaagaggaccaggatctgctgaagctcgtgaagtcctaccactggatgggcctggtgcacatccccaccaatggcagctggcagtgggaggacggcagcatcctgagccccaacctgctgaccatcatcgagatgcagaagggcgactgcgccctgtacgccagcagcttcaagggctacatcgagaactgcagcacccccaacacctacatttgcatgcagcgcacggttaggtccggcgggggaaaccaggaagtgcagatccccctgaccgagagctactgcggcccctgccccaagaactggatctgctacaagaacaactgctaccagttcttcgacgagagcaagaattggtacgagagccaggccagctgcatgagccagaacgccagcctgctgaaggtgtacagcaaagaggaccaggatctgctgaagctcgtgaagtcctaccactggatgggcctggtgcacatccccaccaatggcagctggcagtgggaggacggcagcatcctgagccccaacctgctgaccatcatcgagatgcagaagggcgactgcgccctgtacgccagcagcttcaagggctacatcgagaactgtagcacccccaacacctacatctgtatgcagcggaccgtcagaagtggaggcgggtctggaggcggatccaaccaggaagtgcagatccccctgaccgagagctactgcggcccctgccccaagaactggatctgctacaagaacaactgctaccagttcttcgacgagagcaagaattggtacgagagccaggccagctgcatgagccagaacgccagcctgctgaaggtgtacagcaaagaggaccaggatctgctgaagctcgtgaagtcctaccactggatgggcctggtgcacatccccaccaatggcagctggcagtgggaggacggcagcatcctgagccccaacctgctgaccatcatcgagatgcagaagggcgactgcgccctgtacgccagcagcttcaagggctacatcgagaactgcagcacccccaacacctacatttgcatgcagcgtacggttgacaagacccacacctgccccccctgtcctgcccctgaactgctgggcggaccctccgtgttcctgttccccccaaagcccaaggacaccctgatgatcagccggacccccgaagtgacctgcgtggtggtggacgtgtcccacgaggaccctgaagtgaagttcaattggtacgtggacggcgtggaagtgcacaacgccaagaccaagcccagagaggaacagtacaacagcacctaccgggtggtgtccgtgctgaccgtgctgcaccaggactggctgaacggcaaagagtacaagtgcaaagtctccaacaaggccctgcctgcccccatcgagaaaaccatcagcaaggccaagggccagccccgcgagccccaggtgtacacactgccccccagccgggacgagctgaccaagaaccaggtgtccctgacctgcctggtcaagggcttctaccccagcgatatcgccgtggaatgggagagcaacggccagcccgagaacaactacaagaccaccccccctgtgctggacagcgacggctcattcttcctgtacagcaagctgaccgtggacaagtcccggtggcagcagggcaacgtgttcagctgcagcgtgatgcacgaggccctgcacaaccactacacccagaagtccctgagcctgagccccggcaaa

SEQ ID NO: 47 - protein, 527 aa, Artificial Sequence
  source: /note=“Description of Artificial Sequence Synthetic polypeptide”
FQPVLCNKEVPVSSREGYCGPCPNNWICHRNNCYQFFNEEKTWNQSQASCLSQNSSLLKIYSKEEQDFLKLVKSYHWMGLVQIPANGSWQWEDGSSLSYNQLTLVEIPKGSCAVYGSSFKAYTEDCANLNTYICMKRAVRSGGGSGGGSFQPVLCNKEVPVSSREGYCGPCPNNWICHRNNCYQFFNEEKTWNQSQASCLSQNSSLLKIYSKEEQDFLKLVKSYHWMGLVQIPANGSWQWEDGSSLSYNQLTLVEIPKGSCAVYGSSFKAYTEDCANLNTYICMKRAVISAMVRSPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 48 - DNA, 1581 bp, Artificial Sequence
  source: /note=“Description of Artificial Sequence Synthetic polynucleotide”
tttcaacctgtcctgtgtaacaaagaggttccagtgagttcaagagagggatactgcgggccatgtccgaataactggatctgccacaggaacaactgctatcagtttttcaacgaagaaaagacatggaaccagagtcaggcttcctgtttgtctcagaactccagtctgctgaagatttactcaaaggaggaacaggacttcctcaaactggtaaagagctatcattggatgggactggttcagatcccagccaatggctcttggcagtgggaggatggaagctctctcagctacaaccagcttaccctggtggaaattcccaaaggaagctgcgcagtgtatggttcaagtttcaaagcctacacagaagattgcgctaacctgaatacatacatttgcatgaaaagggctgtcagaagtggaggcgggtctggaggcgggtctttccaacctgtcctgtgcaacaaagaagtgcccgtcagttccagagagggctactgtgggccatgtcccaataattggatttgccacagaaacaactgctaccagttctttaatgaggagaaaacttggaaccagtcacaagcctcttgtctgtcacaaaacagcagcttgctgaaaatctacagcaaggaggagcaggactttctgaagctggtcaaatcttatcactggatgggtctggtgcagatccccgccaatggatcttggcaatgggaggacggctcttctctctcctataatcagctgacactggtagaaatccccaaagggagctgtgcagtgtacgggagcagttttaaggcctataccgaggattgcgcaaatttgaacacttacatctgcatgaagagggccgtcatatcagctatggtccgctcaccaagaggaccaaccattaagccttgcccgccatgcaagtgccccgctccaaaccttctgggagggccgtctgtgttcatttttcctcccaaaatcaaagatgtgctgatgattagcctgtctccaatcgtgacctgtgttgtcgtggacgtgtcagaggatgaccccgatgtgcagataagttggtttgtaaacaatgtagaggtgcacacggcacagacacagacccatagggaggattacaattctaccctgcgggtcgttagcgccctgccgatccagcaccaggactggatgtcaggtaaagaatttaagtgcaaggtgaataataaggatctgcccgctcctattgagagaacgattagcaagcccaagggctccgtgcgcgcaccccaagtctacgttcttcctcccccagaagaggagatgaccaaaaagcaggtcaccctgacgtgtatggtgactgattttatgcccgaagatatttacgtggaatggacaaacaacggtaaaacagagctgaactacaagaatactgagcccgtgctggactccgacggctcttactttatgtatagcaagctgagagttgagaagaaaaattgggtagagagaaattcttactcatgttctgttgtacatgaaggactgcacaatcatcacaccacaaagagtttcagtagaactccaggtaaa

SEQ ID NO: 49 - protein, 537 aa, Artificial Sequence
  source: /note=“Description of Artificial Sequence Synthetic polypeptide”
FQPVLCNKEVPVSSREGYCGPCPNNWICHRNNCYQFFNEEKTWNQSQASCLSQNSSLLKIYSKEEQDFLKLVKSYHWMGLVQIPANGSWQWEDGSSLSYNQLTLVEIPKGSCAVYGSSFKAYTEDCANLNTYICMKRAVRSGGGSGGGSFQPVLCNKEVPVSSREGYCGPCPNNWICHRNNCYQFFNEEKTWNQSQASCLSQNSSLLKIYSKEEQDFLKLVKSYHWMGLVQIPANGSWQWEDGSSLSYNQLTLVEIPKGSCAVYGSSFKAYTEDCANLNTYICMKRAVGGGGSGGGGSISAMVRSPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 50 - DNA, 1611 bp, Artificial Sequence
  source: /note=“Description of Artificial Sequence Synthetic polynucleotide”
tttcaacctgtcctgtgtaacaaagaggttccagtgagttcaagagagggatactgcgggccatgtccgaataactggatctgccacaggaacaactgctatcagtttttcaacgaagaaaagacatggaaccagagtcaggcttcctgtttgtctcagaactccagtctgctgaagatttactcaaaggaggaacaggacttcctcaaactggtaaagagctatcattggatgggactggttcagatcccagccaatggctcttggcagtgggaggatggaagctctctcagctacaaccagcttaccctggtggaaattcccaaaggaagctgcgcagtgtatggttcaagtttcaaagcctacacagaagattgcgctaacctgaatacatacatttgcatgaaaagggctgtcagaagtggaggcgggtctggaggcgggtctttccaacctgtcctgtgcaacaaagaagtgcccgtcagttccagagagggctactgtgggccatgtcccaataattggatttgccacagaaacaactgctaccagttctttaatgaggagaaaacttggaaccagtcacaagcctcttgtctgtcacaaaacagcagcttgctgaaaatctacagcaaggaggagcaggactttctgaagctggtcaaatcttatcactggatgggtctggtgcagatccccgccaatggatcttggcaatgggaggacggctcttctctctcctataatcagctgacactggtagaaatccccaaagggagctgtgcagtgtacgggagcagttttaaggcctataccgaggattgcgcaaatttgaacacttacatctgcatgaagagggccgtcggtggagggggctccggaggcggtgggagcatatcagctatggtccgctcaccaagaggaccaaccattaagccttgcccgccatgcaagtgccccgctccaaaccttctgggagggccgtctgtgttcatttttcctcccaaaatcaaagatgtgctgatgattagcctgtctccaatcgtgacctgtgttgtcgtggacgtgtcagaggatgaccccgatgtgcagataagttggtttgtaaacaatgtagaggtgcacacggcacagacacagacccatagggaggattacaattctaccctgcgggtcgttagcgccctgccgatccagcaccaggactggatgtcaggtaaagaatttaagtgcaaggtgaataataaggatctgcccgctcctattgagagaacgattagcaagcccaagggctccgtgcgcgcaccccaagtctacgttcttcctcccccagaagaggagatgaccaaaaagcaggtcaccctgacgtgtatggtgactgattttatgcccgaagatatttacgtggaatggacaaacaacggtaaaacagagctgaactacaagaatactgagcccgtgctggactccgacggctcttactttatgtatagcaagctgagagttgagaagaaaaattgggtagagagaaattcttactcatgttctgttgtacatgaaggactgcacaatcatcacaccacaaagagtttcagtagaactccaggtaaa

SEQ ID NO: 51 - protein, 547 aa, Artificial Sequence
  source: /note=“Description of Artificial Sequence Synthetic polypeptide”
FQPVLCNKEVPVSSREGYCGPCPNNWICHRNNCYQFFNEEKTWNQSQASCLSQNSSLLKIYSKEEQDFLKLVKSYHWMGLVQIPANGSWQWEDGSSLSYNQLTLVEIPKGSCAVYGSSFKAYTEDCANLNTYICMKRAVRSGGGSGGGSFQPVLCNKEVPVSSREGYCGPCPNNWICHRNNCYQFFNEEKTWNQSQASCLSQNSSLLKIYSKEEQDFLKLVKSYHWMGLVQIPANGSWQWEDGSSLSYNQLTLVEIPKGSCAVYGSSFKAYTEDCANLNTYICMKRAVGGGGSGGGGSGGGGSGGGGSISAMVRSPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 52 - DNA, 1641 bp, Artificial Sequence
  source: /note=“Description of Artificial Sequence Synthetic polynucleotide”
tttcaacctgtcctgtgtaacaaagaggttccagtgagttcaagagagggatactgcgggccatgtccgaataactggatctgccacaggaacaactgctatcagtttttcaacgaagaaaagacatggaaccagagtcaggcttcctgtttgtctcagaactccagtctgctgaagatttactcaaaggaggaacaggacttcctcaaactggtaaagagctatcattggatgggactggttcagatcccagccaatggctcttggcagtgggaggatggaagctctctcagctacaaccagcttaccctggtggaaattcccaaaggaagctgcgcagtgtatggttcaagtttcaaagcctacacagaagattgcgctaacctgaatacatacatttgcatgaaaagggctgtcagaagtggaggcgggtctggaggcgggtctttccaacctgtcctgtgcaacaaagaagtgcccgtcagttccagagagggctactgtgggccatgtcccaataattggatttgccacagaaacaactgctaccagttctttaatgaggagaaaacttggaaccagtcacaagcctcttgtctgtcacaaaacagcagcttgctgaaaatctacagcaaggaggagcaggactttctgaagctggtcaaatcttatcactggatgggtctggtgcagatccccgccaatggatcttggcaatgggaggacggctcttctctctcctataatcagctgacactggtagaaatccccaaagggagctgtgcagtgtacgggagcagttttaaggcctataccgaggattgcgcaaatttgaacacttacatctgcatgaagagggccgtcggtggagggggcagcggaggtgggggcagcgggggaggtgggtccggaggcggtgggagcatatcagctatggtccgctcaccaagaggaccaaccattaagccttgcccgccatgcaagtgccccgctccaaaccttctgggagggccgtctgtgttcatttttcctcccaaaatcaaagatgtgctgatgattagcctgtctccaatcgtgacctgtgttgtcgtggacgtgtcagaggatgaccccgatgtgcagataagttggtttgtaaacaatgtagaggtgcacacggcacagacacagacccatagggaggattacaattctaccctgcgggtcgttagcgccctgccgatccagcaccaggactggatgtcaggtaaagaatttaagtgcaaggtgaataataaggatctgcccgctcctattgagagaacgattagcaagcccaagggctccgtgcgcgcaccccaagtctacgttcttcctcccccagaagaggagatgaccaaaaagcaggtcaccctgacgtgtatggtgactgattttatgcccgaagatatttacgtggaatggacaaacaacggtaaaacagagctgaactacaagaatactgagcccgtgctggactccgacggctcttactttatgtatagcaagctgagagttgagaagaaaaattgggtagagagaaattcttactcatgttctgttgtacatgaaggactgcacaatcatcacaccacaaagagtttcagtagaactccaggtaaa

SEQ ID NO: 53 - protein, 557 aa, Artificial Sequence
  source: /note=“Description of Artificial Sequence Synthetic polypeptide”
FQPVLCNKEVPVSSREGYCGPCPNNWICHRNNCYQFFNEEKTWNQSQASCLSQNSSLLKIYSKEEQDFLKLVKSYHWMGLVQIPANGSWQWEDGSSLSYNQLTLVEIPKGSCAVYGSSFKAYTEDCANLNTYICMKRAVRSGGGSGGGSFQPVLCNKEVPVSSREGYCGPCPNNWICHRNNCYQFFNEEKTWNQSQASCLSQNSSLLKIYSKEEQDFLKLVKSYHWMGLVQIPANGSWQWEDGSSLSYNQLTLVEIPKGSCAVYGSSFKAYTEDCANLNTYICMKRAVGGGGSGGGGSGGGGSGGGGSGGGGSGGGGSISAMVRSPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 54 - DNA, 1671 bp, Artificial Sequence
  source: /note=“Description of Artificial Sequence Synthetic polynucleotide”
tttcaacctgtcctgtgtaacaaagaggttccagtgagttcaagagagggatactgcgggccatgtccgaataactggatctgccacaggaacaactgctatcagtttttcaacgaagaaaagacatggaaccagagtcaggcttcctgtttgtctcagaactccagtctgctgaagatttactcaaaggaggaacaggacttcctcaaactggtaaagagctatcattggatgggactggttcagatcccagccaatggctcttggcagtgggaggatggaagctctctcagctacaaccagcttaccctggtggaaattcccaaaggaagctgcgcagtgtatggttcaagtttcaaagcctacacagaagattgcgctaacctgaatacatacatttgcatgaaaagggctgtcagaagtggaggcgggtctggaggcgggtctttccaacctgtcctgtgcaacaaagaagtgcccgtcagttccagagagggctactgtgggccatgtcccaataattggatttgccacagaaacaactgctaccagttctttaatgaggagaaaacttggaaccagtcacaagcctcttgtctgtcacaaaacagcagcttgctgaaaatctacagcaaggaggagcaggactttctgaagctggtcaaatcttatcactggatgggtctggtgcagatccccgccaatggatcttggcaatgggaggacggctcttctctctcctataatcagctgacactggtagaaatccccaaagggagctgtgcagtgtacgggagcagttttaaggcctataccgaggattgcgcaaatttgaacacttacatctgcatgaagagggccgtcggtggagggggctccggaggtgggggcagcgggggaggtgggtccggtgggggaggctctggcggaggtgggtctggaggcggtgggagcatatcagctatggtccgctcaccaagaggaccaaccattaagccttgcccgccatgcaagtgccccgctccaaaccttctgggagggccgtctgtgttcatttttcctcccaaaatcaaagatgtgctgatgattagcctgtctccaatcgtgacctgtgttgtcgtggacgtgtcagaggatgaccccgatgtgcagataagttggtttgtaaacaatgtagaggtgcacacggcacagacacagacccatagggaggattacaattctaccctgcgggtcgttagcgccctgccgatccagcaccaggactggatgtcaggtaaagaatttaagtgcaaggtgaataataaggatctgcccgctcctattgagagaacgattagcaagcccaagggctccgtgcgcgcaccccaagtctacgttcttcctcccccagaagaggagatgaccaaaaagcaggtcaccctgacgtgtatggtgactgattttatgcccgaagatatttacgtggaatggacaaacaacggtaaaacagagctgaactacaagaatactgagcccgtgctggactccgacggctcttactttatgtatagcaagctgagagttgagaagaaaaattgggtagagagaaattcttactcatgttctgttgtacatgaaggactgcacaatcatcacaccacaaagagtttcagtagaactccaggtaaa

SEQ ID NO: 55 - protein, 527 aa, Artificial Sequence
  source: /note=“Description of Artificial Sequence Synthetic polypeptide”
ISAMVRSPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGKFQPVLCNKEVPVSSREGYCGPCPNNWICHRNNCYQFFNEEKTWNQSQASCLSQNSSLLKIYSKEEQDFLKLVKSYHWMGLVQIPANGSWQWEDGSSLSYNQLTLVEIPKGSCAVYGSSFKAYTEDCANLNTYICMKRAVRSGGGSGGGSFQPVLCNKEVPVSSREGYCGPCPNNWICHRNNCYQFFNEEKTWNQSQASCLSQNSSLLKIYSKEEQDFLKLVKSYHWMGLVQIPANGSWQWEDGSSLSYNQLTLVEIPKGSCAVYGSSFKAYTEDCANLNTYICMKRAV

SEQ ID NO: 56 - DNA, 1581 bp, Artificial Sequence
  source: /note=“Description of Artificial Sequence Synthetic polynucleotide”
atatcagctatggtccgctcaccaagaggaccaaccattaagccttgcccgccatgcaagtgccccgctccaaaccttctgggagggccgtctgtgttcatttttcctcccaaaatcaaagatgtgctgatgattagcctgtctccaatcgtgacctgtgttgtcgtggacgtgtcagaggatgaccccgatgtgcagataagttggtttgtaaacaatgtagaggtgcacacggcacagacacagacccatagggaggattacaattctaccctgcgggtcgttagcgccctgccgatccagcaccaggactggatgtcaggtaaagaatttaagtgcaaggtgaataataaggatctgcccgctcctattgagagaacgattagcaagcccaagggctccgtgcgcgcaccccaagtctacgttcttcctcccccagaagaggagatgaccaaaaagcaggtcaccctgacgtgtatggtgactgattttatgcccgaagatatttacgtggaatggacaaacaacggtaaaacagagctgaactacaagaatactgagcccgtgctggactccgacggctcttactttatgtatagcaagctgagagttgagaagaaaaattgggtagagagaaattcttactcatgttctgttgtacatgaaggactgcacaatcatcacaccacaaagagtttcagtagaactccaggtaaatttcaacctgtcctgtgtaacaaagaggttccagtgagttcaagagagggatactgcgggccatgtccgaataactggatctgccacaggaacaactgctatcagtttttcaacgaagaaaagacatggaaccagagtcaggcttcctgtttgtctcagaactccagtctgctgaagatttactcaaaggaggaacaggacttcctcaaactggtaaagagctatcattggatgggactggttcagatcccagccaatggctcttggcagtgggaggatggaagctctctcagctacaaccagcttaccctggtggaaattcccaaaggaagctgcgcagtgtatggttcaagtttcaaagcctacacagaagattgcgctaacctgaatacatacatttgcatgaaaagggctgtcagaagtggaggcgggtctggaggcgggtctttccaacctgtcctgtgcaacaaagaagtgcccgtcagttccagagagggctactgtgggccatgtcccaataattggatttgccacagaaacaactgctaccagttctttaatgaggagaaaacttggaaccagtcacaagcctcttgtctgtcacaaaacagcagcttgctgaaaatctacagcaaggaggagcaggactttctgaagctggtcaaatcttatcactggatgggtctggtgcagatccccgccaatggatcttggcaatgggaggacggctcttctctctcctataatcagctgacactggtagaaatccccaaagggagctgtgcagtgtacgggagcagttttaaggcctataccgaggattgcgcaaatttgaacacttacatctgcatgaagagggccgtc

SEQ ID NO: 57 - protein, 537 aa, Artificial Sequence
  source: /note=“Description of Artificial Sequence Synthetic polypeptide”
ISAMVRSPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGKGGGGSGGGGSFQPVLCNKEVPVSSREGYCGPCPNNWICHRNNCYQFFNEEKTWNQSQASCLSQNSSLLKIYSKEEQDFLKLVKSYHWMGLVQIPANGSWQWEDGSSLSYNQLTLVEIPKGSCAVYGSSFKAYTEDCANLNTYICMKRAVRSGGGSGGGSFQPVLCNKEVPVSSREGYCGPCPNNWICHRNNCYQFFNEEKTWNQSQASCLSQNSSLLKIYSKEEQDFLKLVKSYHWMGLVQIPANGSWQWEDGSSLSYNQLTLVEIPKGSCAVYGSSFKAYTEDCANLNTYICMKRAV

SEQ ID NO: 58 - DNA, 1611 bp, Artificial Sequence
  source: /note=“Description of Artificial Sequence Synthetic polynucleotide”
atatcagctatggtccgctcaccaagaggaccaaccattaagccttgcccgccatgcaagtgccccgctccaaaccttctgggagggccgtctgtgttcatttttcctcccaaaatcaaagatgtgctgatgattagcctgtctccaatcgtgacctgtgttgtcgtggacgtgtcagaggatgaccccgatgtgcagataagttggtttgtaaacaatgtagaggtgcacacggcacagacacagacccatagggaggattacaattctaccctgcgggtcgttagcgccctgccgatccagcaccaggactggatgtcaggtaaagaatttaagtgcaaggtgaataataaggatctgcccgctcctattgagagaacgattagcaagcccaagggctccgtgcgcgcaccccaagtctacgttcttcctcccccagaagaggagatgaccaaaaagcaggtcaccctgacgtgtatggtgactgattttatgcccgaagatatttacgtggaatggacaaacaacggtaaaacagagctgaactacaagaatactgagcccgtgctggactccgacggctcttactttatgtatagcaagctgagagttgagaagaaaaattgggtagagagaaattcttactcatgttctgttgtacatgaaggactgcacaatcatcacaccacaaagagtttcagtagaactccaggtaaaggtggagggggctccggaggcggtgggagctttcaacctgtcctgtgtaacaaagaggttccagtgagttcaagagagggatactgcgggccatgtccgaataactggatctgccacaggaacaactgctatcagtttttcaacgaagaaaagacatggaaccagagtcaggcttcctgtttgtctcagaactccagtctgctgaagatttactcaaaggaggaacaggacttcctcaaactggtaaagagctatcattggatgggactggttcagatcccagccaatggctcttggcagtgggaggatggaagctctctcagctacaaccagcttaccctggtggaaattcccaaaggaagctgcgcagtgtatggttcaagtttcaaagcctacacagaagattgcgctaacctgaatacatacatttgcatgaaaagggctgtcagaagtggaggcgggtctggaggcgggtctttccaacctgtcctgtgcaacaaagaagtgcccgtcagttccagagagggctactgtgggccatgtcccaataattggatttgccacagaaacaactgctaccagttctttaatgaggagaaaacttggaaccagtcacaagcctcttgtctgtcacaaaacagcagcttgctgaaaatctacagcaaggaggagcaggactttctgaagctggtcaaatcttatcactggatgggtctggtgcagatccccgccaatggatcttggcaatgggaggacggctcttctctctcctataatcagctgacactggtagaaatccccaaagggagctgtgcagtgtacgggagcagttttaaggcctataccgaggattgcgcaaatttgaacacttacatctgcatgaagagggccgtc

SEQ ID NO: 59 - protein, 547 aa, Artificial Sequence
  source: /note=“Description of Artificial Sequence Synthetic polypeptide”
ISAMVRSPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGKGGGGSGGGGSGGGGSGGGGSFQPVLCNKEVPVSSREGYCGPCPNNWICHRNNCYQFFNEEKTWNQSQASCLSQNSSLLKIYSKEEQDFLKLVKSYHWMGLVQIPANGSWQWEDGSSLSYNQLTLVEIPKGSCAVYGSSFKAYTEDCANLNTYICMKRAVRSGGGSGGGSFQPVLCNKEVPVSSREGYCGPCPNNWICHRNNCYQFFNEEKTWNQSQASCLSQNSSLLKIYSKEEQDFLKLVKSYHWMGLVQIPANGSWQWEDGSSLSYNQLTLVEIPKGSCAVYGSSFKAYTEDCANLNTYICMKRAV

SEQ ID NO: 60 - DNA, 1641 bp, Artificial Sequence
  source: /note=“Description of Artificial Sequence Synthetic polynucleotide”
atatcagctatggtccgctcaccaagaggaccaaccattaagccttgcccgccatgcaagtgccccgctccaaaccttctgggagggccgtctgtgttcatttttcctcccaaaatcaaagatgtgctgatgattagcctgtctccaatcgtgacctgtgttgtcgtggacgtgtcagaggatgaccccgatgtgcagataagttggtttgtaaacaatgtagaggtgcacacggcacagacacagacccatagggaggattacaattctaccctgcgggtcgttagcgccctgccgatccagcaccaggactggatgtcaggtaaagaatttaagtgcaaggtgaataataaggatctgcccgctcctattgagagaacgattagcaagcccaagggctccgtgcgcgcaccccaagtctacgttcttcctcccccagaagaggagatgaccaaaaagcaggtcaccctgacgtgtatggtgactgattttatgcccgaagatatttacgtggaatggacaaacaacggtaaaacagagctgaactacaagaatactgagcccgtgctggactccgacggctcttactttatgtatagcaagctgagagttgagaagaaaaattgggtagagagaaattcttactcatgttctgttgtacatgaaggactgcacaatcatcacaccacaaagagtttcagtagaactccaggtaaaggtggagggggcagcggaggtgggggcagcgggggaggtgggtccggaggcggtgggagctttcaacctgtcctgtgtaacaaagaggttccagtgagttcaagagagggatactgcgggccatgtccgaataactggatctgccacaggaacaactgctatcagtttttcaacgaagaaaagacatggaaccagagtcaggcttcctgtttgtctcagaactccagtctgctgaagatttactcaaaggaggaacaggacttcctcaaactggtaaagagctatcattggatgggactggttcagatcccagccaatggctcttggcagtgggaggatggaagctctctcagctacaaccagcttaccctggtggaaattcccaaaggaagctgcgcagtgtatggttcaagtttcaaagcctacacagaagattgcgctaacctgaatacatacatttgcatgaaaagggctgtcagaagtggaggcgggtctggaggcgggtctttccaacctgtcctgtgcaacaaagaagtgcccgtcagttccagagagggctactgtgggccatgtcccaataattggatttgccacagaaacaactgctaccagttctttaatgaggagaaaacttggaaccagtcacaagcctcttgtctgtcacaaaacagcagcttgctgaaaatctacagcaaggaggagcaggactttctgaagctggtcaaatcttatcactggatgggtctggtgcagatccccgccaatggatcttggcaatgggaggacggctcttctctctcctataatcagctgacactggtagaaatccccaaagggagctgtgcagtgtacgggagcagttttaaggcctataccgaggattgcgcaaatttgaacacttacatctgcatgaagagggccgtc

SEQ ID NO: 61 - protein, 557 aa, Artificial Sequence
  source: /note=“Description of Artificial Sequence Synthetic polypeptide”
ISAMVRSPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGKGGGGSGGGGSGGGGSGGGGSGGGGSGGGGSFQPVLCNKEVPVSSREGYCGPCPNNWICHRNNCYQFFNEEKTWNQSQASCLSQNSSLLKIYSKEEQDFLKLVKSYHWMGLVQIPANGSWQWEDGSSLSYNQLTLVEIPKGSCAVYGSSFKAYTEDCANLNTYICMKRAVRSGGGSGGGSFQPVLCNKEVPVSSREGYCGPCPNNWICHRNNCYQFFNEEKTWNQSQASCLSQNSSLLKIYSKEEQDFLKLVKSYHWMGLVQIPANGSWQWEDGSSLSYNQLTLVEIPKGSCAVYGSSFKAYTEDCANLNTYICMKRAV

SEQ ID NO: 62 - DNA, 1671 bp, Artificial Sequence
  source: /note=“Description of Artificial Sequence Synthetic polynucleotide”
atatcagctatggtccgctcaccaagaggaccaaccattaagccttgcccgccatgcaagtgccccgctccaaaccttctgggagggccgtctgtgttcatttttcctcccaaaatcaaagatgtgctgatgattagcctgtctccaatcgtgacctgtgttgtcgtggacgtgtcagaggatgaccccgatgtgcagataagttggtttgtaaacaatgtagaggtgcacacggcacagacacagacccatagggaggattacaattctaccctgcgggtcgttagcgccctgccgatccagcaccaggactggatgtcaggtaaagaatttaagtgcaaggtgaataataaggatctgcccgctcctattgagagaacgattagcaagcccaagggctccgtgcgcgcaccccaagtctacgttcttcctcccccagaagaggagatgaccaaaaagcaggtcaccctgacgtgtatggtgactgattttatgcccgaagatatttacgtggaatggacaaacaacggtaaaacagagctgaactacaagaatactgagcccgtgctggactccgacggctcttactttatgtatagcaagctgagagttgagaagaaaaattgggtagagagaaattcttactcatgttctgttgtacatgaaggactgcacaatcatcacaccacaaagagtttcagtagaactccaggtaaaggtggagggggctccggaggtgggggcagcgggggaggtgggtccggtgggggaggctctggcggaggtgggtctggaggcggtgggagctttcaacctgtcctgtgtaacaaagaggttccagtgagttcaagagagggatactgcgggccatgtccgaataactggatctgccacaggaacaactgctatcagtttttcaacgaagaaaagacatggaaccagagtcaggcttcctgtttgtctcagaactccagtctgctgaagatttactcaaaggaggaacaggacttcctcaaactggtaaagagctatcattggatgggactggttcagatcccagccaatggctcttggcagtgggaggatggaagctctctcagctacaaccagcttaccctggtggaaattcccaaaggaagctgcgcagtgtatggttcaagtttcaaagcctacacagaagattgcgctaacctgaatacatacatttgcatgaaaagggctgtcagaagtggaggcgggtctggaggcgggtctttccaacctgtcctgtgcaacaaagaagtgcccgtcagttccagagagggctactgtgggccatgtcccaataattggatttgccacagaaacaactgctaccagttctttaatgaggagaaaacttggaaccagtcacaagcctcttgtctgtcacaaaacagcagcttgctgaaaatctacagcaaggaggagcaggactttctgaagctggtcaaatcttatcactggatgggtctggtgcagatccccgccaatggatcttggcaatgggaggacggctcttctctctcctataatcagctgacactggtagaaatccccaaagggagctgtgcagtgtacgggagcagttttaaggcctataccgaggattgcgcaaatttgaacacttacatctgcatgaagagggccgtc

SEQ ID NO: 63 - protein, 239 aa, Artificial Sequence
  source: /note=“Description of Artificial Sequence Synthetic polypeptide”
ISAMVRSPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 64 - protein, 107 aa, Homo sapiens [sequence omitted]

SEQ ID NO: 65 - protein, 330 aa, Artificial Sequence
  source: /note=“Description of Artificial Sequence Synthetic polypeptide”
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 66 - protein, 4 aa, Artificial Sequence
  source: /note=“Description of Artificial Sequence Synthetic peptide”
GGGS

SEQ ID NO: 67 - protein, 284 aa, Artificial Sequence
  source: /note=“Description of Artificial Sequence Synthetic polypeptide”
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQG

SEQ ID NO: 68 - protein, 227 aa, Artificial Sequence
  source: /note=“Description of Artificial Sequence Synthetic polypeptide”
DKTHTCPPCPAPELLGGPSVFLFPPKPKDTLMISRTPEVTCVVVAVSHEDPEVKFNWYVDGVEVHNAKTKPREEQYASTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 69 - protein, 503 aa, Artificial Sequence
  source: /note=“Description of Artificial Sequence Synthetic polypeptide”
NQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVRSGGGSGGGSNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVDKTHTCPPCPAPELLGGPSVFLFPPKPKDTLMISRTPEVTCVVVAVSHEDPEVKFNWYVDGVEVHNAKTKPREEQYASTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 70 - DNA, 1509 bp, Artificial Sequence
  source: /note=“Description of Artificial Sequence Synthetic polynucleotide”
aaccaggaagtgcagatccccctgaccgagagctactgcggcccctgccccaagaactggatctgctacaagaacaactgctaccagttcttcgacgagagcaagaattggtacgagagccaggccagctgcatgagccagaacgccagcctgctgaaggtgtacagcaaagaggaccaggatctgctgaagctcgtgaagtcctaccactggatgggcctggtgcacatccccaccaatggcagctggcagtgggaggacggcagcatcctgagccccaacctgctgaccatcatcgagatgcagaagggcgactgcgccctgtacgccagcagcttcaagggctacatcgagaactgtagcacccccaacacctacatctgtatgcagcggaccgtcagaagtggaggcgggtctggaggcggatccaaccaggaagtgcagatccccctgaccgagagctactgcggcccctgccccaagaactggatctgctacaagaacaactgctaccagttcttcgacgagagcaagaattggtacgagagccaggccagctgcatgagccagaacgccagcctgctgaaggtgtacagcaaagaggaccaggatctgctgaagctcgtgaagtcctaccactggatgggcctggtgcacatccccaccaatggcagctggcagtgggaggacggcagcatcctgagccccaacctgctgaccatcatcgagatgcagaagggcgactgcgccctgtacgccagcagcttcaagggctacatcgagaactgcagcacccccaacacctacatttgcatgcagcgtacggttgacaagacccacacctgccctccctgtccagcccctgaactgctgggaggccctagcgtgttcctgttccccccaaagcccaaggacaccctgatgatcagccggacccccgaagtgacctgtgtggtggtggccgtgtctcacgaggaccctgaagtgaagtttaattggtacgtggacggcgtggaagtgcacaacgccaagaccaagcccagagaggaacagtacgccagcacctaccgggtggtgtccgtgctgacagtgctgcaccaggactggctgaacggcaaagagtacaagtgcaaggtgtccaacaaggccctggccgctcccatcgagaaaaccatcagcaaggccaagggccagccccgcgaaccccaggtgtacacactgccccctagcagggacgagctgaccaagaaccaggtgtccctgacctgcctcgtgaagggcttctacccctccgatatcgccgtggaatgggagagcaacggccagcccgagaacaactacaagaccaccccccctgtgctggactccgacggctcattcttcctgtacagcaagctgaccgtggacaagtcccggtggcagcagggcaacgtgttcagctgctccgtgatgcacgaggccctgcacaaccactacacccagaagtccctgagcctgagccccggcaaa

SEQ ID NO: 71 - protein, 6 aa, Artificial Sequence
  source: /note=“Description of Artificial Sequence Synthetic peptide”
FLNSLF

SEQ ID NO: 72 - protein, 5 aa, Artificial Sequence
  source: /note=“Description of Artificial Sequence Synthetic peptide”
GGGGA

SEQ ID NO: 73 - protein, 5 aa, Artificial Sequence
  source: /note=“Description of Artificial Sequence Synthetic peptide”
GGGGP

SEQ ID NO: 74 - protein, 4 aa, Artificial Sequence
  source: /note=“Description of Artificial Sequence Synthetic peptide”
GGGA

SEQ ID NO: 75 - protein, 10 aa, Artificial Sequence
  source: /note=“Description of Artificial Sequence Synthetic peptide”
RSGGGSGGGS

SEQ ID NO: 76 - protein, 20 aa, Artificial Sequence
  source: /note=“Description of Artificial Sequence Synthetic peptide”
GGGGSGGGGSGGGGSGGGGS